(19) United States
(12) Patent Application Publication
(10) Pub. No.: US 2025/0127529 A1
(43) Pub. Date: Apr. 24, 2025

(54) METHODS AND APPARATUS FOR PLAQUE DISRUPTION

(71) Applicant: Elixir Medical Corporation, Milpitas, CA (US)

(72) Inventors: Motasim SIRHAN, Los Altos, CA (US); Benjamyn SERNA, Gilroy, CA (US); John YAN, Los Gatos, CA (US); Bozena TREADWELL, Aptos, CA (US); Russell PONG, Newark, CA (US)

(73) Assignee: Elixir Medical Corporation, Milpitas, CA (US)

(21) Appl. No.: 18/918,647

(22) Filed: Oct. 17, 2024

Related U.S. Application Data
(63) Continuation-in-part of application No. PCT/US23/74810, filed on Sep. 21, 2023.
(60) Provisional application No. 63/591,433, filed on Oct. 18, 2023.
(60) Provisional application No. 63/409,419, filed on Sep. 23, 2022.
(60) Provisional application No. 63/411,095, filed on Sep. 28, 2022.
(60) Provisional application No. 63/421,940, filed on Nov. 2, 2022.
(60) Provisional application No. 63/386,637, filed on Dec. 8, 2022.
(60) Provisional application No. 63/387,636, filed on Dec. 15, 2022.
(60) Provisional application No. 63/514,079, filed on Jul. 17, 2023.
(60) Provisional application No. 63/579,487, filed on Aug. 29, 2023.

(51) Int. Cl. A61B 17/22; A61B 17/00

(52) U.S. Cl. CPC A61B 17/2202; A61B 17/00234; A61B 2017/00238; A61B 2017/00305; A61B 2017/00831; A61B 2017/22051

(57) ABSTRACT

Balloon catheters, sleeves, cages, cylindrical structures and endoluminal prostheses are provided with stress-applying and spacing features coupled to expandable surfaces thereof. At least one or at least some stress-applying features are immobilized onto the surface or into pre-formed indentations formed in the expandable surface. The stress-applying and spacing features may have blunt and/or rounded contact regions which contact tissue or calcified regions in the vasculature. The contact regions dent or fracture occlusive material on the wall of a vascular lumen and/or patient valve leaflets when expanded. The spacing of features can permit blood, drug, and contrast perfusion past structures expanded in the vasculature, particularly balloon catheters.

### CROSS-REFERENCE TO RELATED APPLICATIONS

This application claims the benefit of U.S. Provisional No. 63/591,433 (Attorney Docket No. 32016-726.112), filed Oct. 18, 2024; this application is also a continuation-in-part of PCT Application No. PCT/US2023/074810 (32016-726.602), filed on Sep. 21, 2023, which claimed the benefit of U.S. Provisional No. 63/409,419 (Attorney Docket No. 32016-726.105), filed Sep. 23, 2022, of U.S. Provisional No. 63/411,095 (Attorney Docket No. 32016-726.106), filed Sep. 28, 2022, of U.S. Provisional No. 63/421,940 (Attorney Docket No. 32016-726.107), filed Nov. 2, 2022, of U.S. Provisional No. 63/386,637 (Attorney Docket No. 32016-726.108), filed Dec. 8, 2022, of U.S. Provisional No. 63/387,636 (Attorney Docket No. 32016-726.109), filed Dec. 15, 2022; of U.S. Provisional No. 63/514,079 (Attorney Docket No. 32016-726.110), filed Jul. 17, 2023, and of U.S. Provisional No. 63/579,487 (Attorney Docket No. 32016-726.111), filed Aug. 29, 2023, the full disclosures of which are incorporated herein by reference.

The disclosure of this provisional application is also related to U.S. patent application Ser. No. 17/863,265, filed on Jul. 22, 2022 (Attorney Docket No. 32106-726.301), which is a continuation of PCT/US2022/022213 (Attorney Docket No. 32106-726.601), filed on Mar. 28, 2022, which claims the benefit of provisional application 63/322,372 (Attorney Docket No. 32106-726.101), filed on Mar. 22, 2022, of 63/287,813 (Attorney Docket No. 32106-726.103), filed on Dec. 9, 2021, of provisional application 63/240,811 (Attorney Docket No. 32106-726.102), filed on Sep. 3, 2021, and of provisional application 63/200,794 (Attorney Docket No. 32106-726.101), filed on Mar. 29, 2021, the full disclosures of which are incorporated herein by reference.

### BACKGROUND OF THE INVENTION

### 1. Field of the Invention

The present invention relates generally to medical devices and methods. More particularly, the present invention relates to devices comprising or consisting of expandable structures, including vascular devices, vascular catheters, expandable sleeves, expandable cages, balloon catheters, scaffolds, stents, vascular grafts, implantable vascular prostheses configured to open, expand, delivery drugs, and/or fracture calcified and/or hardened lesions in a blood vessel and/or valve, and/or body lumen.

Balloons, cages, stents, grafts, and other prostheses and devices are commonly used to provide or maintain patency in blood vessels and heart and venous valve structures which have been narrowed by lesions or other disease conditions. In cases where the lesion is hardened by plaque, calcium, and the like, a “cutting balloon” may be used for the initial treatment step to break the plaque and calcifications to permit the balloon to widen the lesion prior to stent placement. Optionally, a stent may be post-dilated using a non-compliant angioplasty balloon to ensure good apposition to the blood vessel wall. The post-dilation balloon is taken to higher atmospheric pressures using a balloon inflation device. Stenting after angioplasty with a cutting balloon, however, can be problematic in several respects, particularly when followed by post dilation at higher pressures. In some instances, the blood vessel wall can be injured, or such injury propagates. In others, debris generated by the cutting balloon can be released as emboli.

The use of cutting balloons, cutting cages, and cutting stents has been proposed for disrupting vascular calcifications with various issues including being less deliverable, tend to be bulky limiting access, or patient injury. The cutting stent was proposed to be used in a “primary stenting” procedure without use of a pre-dilation angioplasty balloon to widen the area to be stented. Primary stenting reduces or eliminates the need to perform two or three sequential interventions, which in turn reduces the risk of vessel injury and emboli release as discussed above. However, cutting stents have similar limitations such as being bulky, risking injury, and being still and difficult to deliver.

Heart and venous valve function can be adversely affected by the presence of calcifications on the valve leaflets. Valvuloplasty procedures rely on disrupting the lesions by expanding a balloon inside of the opposed leaflet surfaces in order to crack the calcifications. The use of cutting and caged balloons for enhancing such valvuloplasty procedures has been proposed but has not found widespread use as it increases the risk of damaging the valve and/or the valve function.

While a significant potential improvement in many instances, such cutting balloons, cages, and stents typically employ sharpened blades which are oriented radially outwardly and present a risk of injury to the patient.

In addition, cutting blades can make a device bulky and less deliverable, often limiting access to distal vessel regions. Cutting blades also tend to rupture the blood vessel wall beneath calcified/hardened plaque lesions, particularly at higher inflation pressures, which may cause patient injury.

Another challenge in performing angioplasty and valvuloplasty procedures is the temporary blockage of the of the vessel lumen or valve annulus by balloon expansion. While temporary blockage of blood flow is often acceptable, a more serious concern can be the loss of contrast medium flow downstream of the balloon. Even a short loss of contrast medium can make fluoroscopic imaging of the procedure more difficult. While a variety of perfusion catheters have been proposed, they often require an additional flow lumen through the balloon, making the catheter larger and less useful in many circumstances.

A still further challenge in using known cutting balloons is that the elongated blades on the balloon stiffen the balloon so that it can be difficult to deploy the balloons in tortuous anatomy.

For these reasons, it would be desirable to provide improved methods and apparatus for opening, expanding, delivering drugs, improved visualization, and/or treating calcified or hardened lesions with a reduced risk to the patient and equivalent or improved efficacy in treating the calcifications or hardened lesions. The improvements would preferably apply to a wide variety of vascular and cardiac devices, including expandable structures, such as vascular catheters, expandable sleeves, expandable cages, balloon catheters, stents, implantable vascular prostheses, drug delivery devices, and in particular to plaque-disrupting balloon designs. It would be still further desirable if the devices and procedure could improve the perfusion of both blood and/or contrast medium during the procedure. In addition, it would be desirable to provide plaque-disrupting balloon designs with limited or no loss of flexibility due to the present of the plaque-disrupting elements on the balloons. At least some of these advantages will be provided by the present invention.

### 2. Listing of Background Art

U.S. Pat. No. 7,662,163 describes a stiffened balloon apparatus with one or more stiffeners having sharpened and other projections thereon. US2009/0105687; US2006/0129229; US2005/0137621; U.S. Pat. Nos. 9,370,644; 9,119,944; 8,992,553; 8,882,790; 8,523,887; 8,323,325; 7,799,043; 6,197,013; 5,242,397; and EP2919707; patents and printed publications describing expandable scaffolds having plaque disruptive and other features include: U.S. Pat. Nos. 8,876,882; 7,494,497; 9,724,121; 7,731,744; 5,591,197; US2006/122684; US2014/277562; US2001/037146; US2006/271161; US2020/0323545; U.S. Pat. Nos. 10,143,452; 9,717,513; 8,398,662; 8,092,470; 8,080,026; 7,803,168; 6,047,700; 5,443,446; and patents and printed publications describing multiple surface valvuloplasty include: US2021/0378744; U.S. Pat. No. 11,000,299: U.S. Pat. Nos. 10,758,255; 10,478,202; 9,827,096; 8,187,223; US20210393281; US20200197033; U.S. Pat. Nos. 11,000,299; 10,980,553; 10,342,962; 10,245,419; 9,504,807; 9,375,555; 4,986,830; EP13526772; EP1480709; KR20200077682; WO2020014515; WO2012040225; WO2003/084594; and WO2013126779. Parent application U.S. Ser. No. 17/863,265 has been published as US 2022/0338889 and WO2022/212290.

### SUMMARY OF THE INVENTION

Devices according to the present invention comprise stress-applying features or force-applying features which may have any one of a variety of specific designs and geometries selected to fracture, dent, or otherwise disrupt regions of calcified or otherwise hardened plaque, expand, deliver drugs, improve visualization, and/or enlarge lesions in a patient's vasculature with low to minimum risk to underlying patient tissue. The devices of the present invention will often find use in angioplasty, stent placement, drug delivery, enhanced visualization, and other interventions in the arterial or venous vasculature. In addition, the devices of the present invention will find use in treating or modifying cardiac and venous valve structures, for example in performing valvuloplasty procedures in a patient's aortic valve.

The phrase “stress-applying feature” is intended to encompass a variety of specific force-applying structures such as plaque-disrupting features, plaque-engaging features, calcium-engaging features, and these terms may be used interchangeably herein and in the claims. Other suitable descriptors for these features include plaque-denting features, and stress-inducing features, and unless otherwise stated, these terms and phrases will be used interchangeably. The stress-applying features of the present invention may have any one of a variety of designs as described in this invention.

A first type of stress-applying features will typically comprise a blunt contact region configured to engage the plaque or other hardened or calcified material to expand, fracture, dent, otherwise disrupt that material while minimizing risk to underlying tissue or vascular wall, the valve annulus, or other patient tissue which would be at risk if pressure will be applied by a blade or other sharpened structure. A variety of specific designs for the stress-applying features of the present invention are described below, including discs, plates, balls, spheres, hemispheres, partial spheres, ellipsoidal solids, oblong, other, and the like. The stress-applying features maybe solid or comprise a hollow interior. The stress-applying features of the present invention may be (1) pre-formed and attached to the device such as balloon, a sleeve, a stent, or a cage or (2) fabricated as in integral part or component part of a balloon, a sleeve, a stent, or a cage, as described in more detail in the present invention, or (3) a combination of (1) and (2).

The devices of the present invention will usually further comprise an apparatus or structure for radially advancing or deploying the stress-applying features within a patient target site, such as a blood vessel, valve annulus, or other body lumen or cavity. In some instances, pre-formed stress-applying features may be directly attached or coupled to an outer surface of any one of a variety of expandable structures, such as balloons, stents/scaffolds, stents, cages, sleeves, valve prostheses, valvuloplasty balloon catheters, and the like. In other instances, stress-applying features may be fabricated directly as an integrated part of such expandable structures. In still other instances, the stress-applying features may be attached to or fabricated as part of an intermediate structure which, while itself not being configured to expand, may be placed over a separate expandable structure. For example, the stress-applying features of the present invention may be attached to or formed as a part of sleeves, sheaths, covers, meshes, or other supporting structures which may be placed to surround or otherwise be supported by an expandable structure, such as an elastic sleeve being placed over an expandable balloon.

Exemplary stress-applying features of the present invention will have a circumscribed “footprint,” or base typically having a maximum length, width, diameter, or other dimension of 4 mm or less, often being 3 mm or less, more often being no more than 1 mm, and frequently being no more than 0.75 mm, and sometimes being no more than 0.5 mm, or being no more than 0.25 mm. In another example, the base of the stress-applying features may have a length, width, diameter, or configuration ranging from 0.1 mm to 4 mm, preferably ranging from 0.2 mm to 2 mm, more preferably ranging from 0.3 mm to 0.75 mm. Footprint refers to the maximum coverage or “contact” area dimension of a stress-applying feature at the base over an underlying support surface, such as an outer surface of balloon, sleeve, scaffold, cage, or stent. The contact region or coverage area dimension of the stress-applying feature may be equal to that of the base, such as in the case of discs, balls, spheres, or may be greater than that of the base when the feature tapers radially outwardly as in the case of inverted cones or inverted hemispheres, or inverted partial-spheres, or maybe smaller than that of the base when the feature tapers radially outwardly as in the case of cones, hemisphere, or partial-spheres. In one example, the contact region of the stress-applying features may have a length, width, diameter, or configuration ranging from 0.01 mm to 4 mm, preferably ranging from 0.1 mm to 2 mm, more preferably ranging from 0.1 mm to 0.75 mm. In another example, the stress-applying features are spheres, or the like, wherein the contact region and/or base have a length, width, diameter, or configuration ranging from zero or near zero to 0.1 mm, more often ranging from 0.001 to 0.1 mm. The stress-applying features of the present invention will typically not be axially extended elongated members, such as blades, or similar elongate cutters, but rather have a circumferential elongation or configuration and axial elongation or configurations being about the same. In a preferred example, the footprint at the base of the stress-applying features has a maximum axial length to maximum circumferential width ranges from 0.5:1 to 1:0.5, more preferably ranges from 0.75:1 to 1:0.75, and most preferably about 1:1. In another preferred example, the dimensions at the base of the stress-applying features are substantially the same as the dimensions of the contact coverage area dimensions, as in the case of balls, hemisphere, partial sphere, spheres, or square for example. Limiting the length of the stress-applying features in an axial direction (and optionally maximizing the extent of the features in a circumferential direction) is advantageous as it reduces stiffness and maximizes flexibility and bendability of the balloon to facilitate insertion and removal in tortuous anatomy.

A preferred stress-applying feature will comprise, consist essentially of, or consist of a rigid structure formed from a hard material, usually a metal or metal alloy. The rigid structure will have a convex upper rounded surface, e.g., formed as a convex rounded upper apex, convex upper apex, convex rounded upper apex, or the like, and will typically have a radius of curvature ranging from 0.1 mm to 1 mm. The rigid structure may have any of a variety of geometries, such as spherical, hemispherical, partial sphere, ellipsoidal, hemi-ellipsoidal, partial ellipsoidal, or similar geometry. While metals and/or metal alloys, particularly steels, stainless steels, tungsten, tungsten carbide, cobalt, cobalt chrome, platinum, are preferred materials, other metals or metal alloys and other hard materials such as minerals, ceramics, hardened polymers, other, or and the like having a Mohs hardness greater than 4, preferably greater than 5, and more preferably greater than 6 may also be suitable for use. In other specific examples, stress-applying features may comprise, consist, of metal and/or metal alloys including palladium, rhodium, titanium, and nickel.

The rounded convex upper surfaces of the stress-applying features will typically have a radius of curvature ranging from 0.1 mm to 3 mm, preferably from 0.1 mm to 2 mm, more preferably from 0.1 mm to 1 mm, and most preferably from 0.1 to 0.5 mm. The radius will be uniform in the case of spherical, hemispherical and partial spherical convex surfaces. In contrast, the radius of curvature will typically vary for non-spherical, e.g. ellipsoidal, asymmetrical, or irregular convex upper surfaces wherein at least one side of the upper surface would have a radius of curvature ranging from 0.1 mm to 3 mm, preferably from 0.1 mm to 2 mm, more preferably from 0.1 mm to 1 mm, and most preferably from 0.1 to 0.5 mm, having various types of shaped bases such as asymmetrical, irregular, or non-circular bases, or in rare instances having circular or symmetrical bases. Such non-circular or irregular shaped bases may have a maximum: minimum dimensional ratio (e.g. ratio of length to width) of 5:1 or less, usually 3:1 or less, and more usually 1.9:1 or less where preferably the length is oriented in a circumferential direction on the balloon outer surface while the width is oriented in an axial direction in the balloon outer surface. Similarly, non-circular or irregular shaped upper surfaces may have a maximum: minimum dimensional ratio (e.g. ratio of length to width) of 5:1 or less, usually 3:1 or less, and more usually 1.9:1 or less where preferably the length is oriented in a circumferential direction on the balloon outer surface while the width is oriented in an axial direction on the balloon outer surface. Upper surface typically refers to the apex region of the features.

The rounded convex upper surfaces of the stress-applying features will typically be smooth with few or no irregularities or singularities. The surface may be formed or fabricated to be smooth and rounded, for example by casting, molding, machining, or other standard fabrication methods. Alternatively, the smooth rounded surfaces can be formed by coating or sputtering or otherwise depositing a harder material, such as a metal, over a core structure attached to the outer balloon wall surface. In some other instances, the smooth rounded surfaces can be formed by coating or sputtering or otherwise depositing a material, such as a metal/metal alloy, polymeric, ceramic, or other material that would cover a core structure attached to the outer balloon wall surface to smoothen, make rounded convex, or make the upper surface atraumatic.

The stress-applying features will typically be molded, machined or otherwise formed into their preferred spherical, hemispherical (including partial sphere), ellipsoidal, hemi-ellipsoidal (including partial ellipsoidal), or similar shapes and will have a bottom configured for direct attachment to the outer balloon surface as described elsewhere herein, usually being flat or slightly concaved, or contoured to conform to the outer balloon surface. The stress-applying features will usually be formed as a monolithic structure without internal seams or breaks separating the stress-applying feature into two or more components attached to each other to form the feature, and wherein the features are attached to an outer surface of the balloon. However, in other cases the stress-applying features are formed into spherical, hemispherical (including partial sphere), ellipsoidal, hemi-ellipsoidal (including partial ellipsoidal), or similar shapes by joining two or more portions, wherein each portion is formed separately and is then joined together after fabrication or after attachment to the outer balloon surface to form the feature shape so long as the desired spherical, hemispherical, partial sphere having a convex upper apex, ellipsoidal, hemi-ellipsoidal, partial ellipsoid having a convex upper apex, or similar shape is maintained. i.e., the base (which is attached to the outer balloon surface) has a configuration, or shape and size which is equal to or larger than maximum cross-sectional configuration or size and shape of the upper regions of the feature.

The stress-applying and other rigid features of the present invention may be monolithic, i.e. having a continuous, typically homogeneous structure usually formed by molding, machining, casting, or other conventional processes. In other instances, however, the stress-applying and other rigid features of the present invention may be “polylithic,” comprising, consisting, or consisting essentially of two or more different parts, sections, elements, laminates, coatings, attachments, and the like, often comprising a base and an upper region or segment. The base may comprise parts or slices of the features, for example in a horizontal plane or in a vertical plane and may be attached or integrated together to form a structure preferably having a convex upper smooth surface, more preferably having a smoothly rounded convex upper surface with no atraumatic portions.

In some instances, such structure includes a base and an upper segment where the periphery of the base preferably does not protrude beyond the periphery of the upper segment, i.e., no edges are formed.

In some instances, the base transitions or “contours” smoothly with the feature upper segment and/or slices of features that are joined adhesively or by welding together to form smooth rounded features. Such features may be formed from any one or more of the materials described elsewhere herein, including but not limited to polymer, ceramic or mineral materials.

Such features may have one or more convex upper surface(s), rounded upper surface(s), or the like, which may have a radius of curvature in at least one direction in a range from 0.05 mm to 0.5 mm, preferably from 0.05 mm to 0.25 mm. At least one upper surface is preferably convex, rounded, smoothed, or otherwise made atraumatic so as not to cause injury to non-calcified lesions and healthy vessel wall. Exemplary stress-applying and other rigid features include spheres, hemispheres, and partial spheres having rounded upper surfaces.

The features, and in particular the upper rounded or other segments of the features, may be initially formed or fabricated to have smooth surfaces or may be initially formed or fabricated to have rough or otherwise irregular surfaced and smoothed after initial fabrication, for example by coating or sputtering with the same or another material. In preferred instances, the features may be covered with a harder material.

In some instances, features having sharpened, penetrating or other exposed “traumatic” elements may be covered, coated, polished or otherwise modified to provide both traumatic and atraumatic regions on the upper segment or elsewhere on the element for a therapeutic purpose. For example, a cutting, penetrating or other traumatic element may be encased in and/or surrounded by a rounded, convex, and/or smooth upper surface region so that the traumatic element would disrupt calcium, but the surrounding atraumatic surface would inhibit injury adjacent plaque. The surrounding cover would typically be softer than traumatic element of the feature to allow the traumatic element to protrude or exert force through surrounding softer region when expanded against calcified plaque or the vessel wall. Alternatively, such a traumatic element or component of the feature may protrude or exert force when the adjacent softer cover is pushed against a vessel wall, causing the surrounding cover to compress and allow the traumatic element to protrude and engage hardened plaque on the vessel wall. In other instances, the surrounding cover may be formed from a hard material that does not compress when pushed against the vessel wall. In those instances, the traumatic element may be configured to fixedly protrude from the cover so that the elements will engage the vessel wall before the surrounding cover engages the vessel wall.

Exemplary stress-applying features of the present invention will also typically have a height measured from the base attached to the supporting surface or substrate to the contact coverage area (contact region), typically being at least 0.05 mm, 0.1 mm, 0.15 mm, 0.2 mm, 0.25 mm, or more, and often not exceeding 1 mm, 0.5 mm, 0.4 mm, 0.3 mm, 0.25 mm, 0.15 mm, 0.1 mm or less, including all ranges between the minimum and maximum recited heights. In a preferred example, the height of the features ranges from 0.1 mm to 1 mm, preferably ranges from 0.2 mm to 0.75 mm, and more preferably ranges from 0.25 to 0.5 mm. The features may have the same height along the entire structure or different heights along the circumference and/or axial length of the substrate.

In a preferred example, the stress-applying features are discrete features to enhance flexibility and deliverability of the device in a patient vasculature or body lumen. Such discrete stress-applying features will typically be distributed over an expandable surface at a density when inflated or otherwise expanded of from 0.005 to 20 features/mm² of outer balloon surface, preferably from 0.005 to 5 features/mm², more preferably from 0.01 to 5 features/mm², typically 0.01 to 3 features/mm², more typically 0.01 to 1 features/mm², and most typically 0.01 to 0.1 features/mm².

In addition to maintaining feature density, it will also be preferable to maintain a minimum axial spacing between circumferentially adjacent stress-applying features bases or between all stress applying features bases along the circumference of the expandable balloon when expanded, in order to enhance flexibility and bendability of the balloon as it is introduced through the vasculature. In particular, the minimum axial distance between the bases of circumferentially adjacent (or between all stress applying features bases along the circumference of the expandable balloon) stress-applying features should be at least 0.05 mm, preferably being at least 0.1 mm, while maximum axial spacings will be 3 mm, usually 2.5 mm, with the spacings typically being in a range from 0.05 mm to 3 mm, more typically from 0.1 mm to 1 mm.

In a preferred example, at least one segment or at least one region of the expandable structure outer surface (or expandable balloon outer surface) having stress-applying features being spheres, hemispheres, or partial spheres having a rounded convex upper surfaces distributed over the expandable surface of the expandable structure (such as the expandable balloon) at a 31 when the balloon is inflated or otherwise expanded ranging from 0.01 to 0.1 features/mm² of the outer surface, and wherein all circumferentially adjacent stress applying features along the circumference of the expanded structure (or inflated balloon outer surface) having a minimum axial spacing between their bases ranging from 0.05 mm to 2 mm, and wherein the stress applying features upper surfaces having a radius of curvature ranging from 0.1 mm to 1 mm.

In a preferred example, the stress-applying features are discrete, independent, and/or separated from one another features to enhance flexibility and deliverability of the device in a patient vasculature or body lumen. Such stress-applying features will typically be distributed over an expandable surface at a density when inflated or otherwise expanded from 0.1 to 20 features/mm², preferably 0.1 to 5 features/mm², more preferably from 0.2 to 4 features/mm², more preferably from 0.25 to 3 features/mm². In a preferred example, each of the features apply an independent or focal force to the plaque, vessel wall, tissue, hardened plaque, or calcified lesion.

In a preferred example, the stress-applying features are discrete, wherein each of the features contact region, body, and/or base are dull, rounded, smooth, and/or atraumatic, to enhance flexibility and deliverability of the device in a patient vasculature or body lumen. Such stress-applying features will typically be distributed over an expandable surface at a density when inflated or otherwise expanded ranging from 0.1 to 100 features/mm², preferably ranging from 0.1 to 20 features/mm², more preferably ranging from 0.1 to 5 features/mm², or in some instances ranging from 0.2 to 4 features/mm², more preferably ranging from 0.25 to 3 features/mm². In another example, the features shape comprising the contact region, base, and body, are rounded, dull, and atraumatic, to enhance flexibility and deliverability of the device in a patient vasculature or body lumen, while being able to disrupt, dent, or expand a hardened and/or calcified plaque.

In yet another example, the stress-applying features are coated with one or more material to provide roundness, smooth surface, dullness, and/or atraumatic surface, to enhance flexibility and deliverability of the device in a patient vasculature or body lumen, while being able to disrupt, dent, or expand a hardened and/or calcified plaque. In a preferred example, the material comprises one or more of a metallic material, a ceramic material, a polymeric material, an adhesive material, a hydrophilic material, and the like. The material is deposited, soldered, coated, plated, dipped, heat treated, hardened, or otherwise applied to the surface of the stress-applying features. The material may be degradable or non-degradable in a physiologic environment. In yet another instance, the coating material provides further attachment means of the stress-applying features to the expandable structure surface, wherein the coating covers the features and a portion of the expandable structure surface adjacent to the feature.

In preferred examples, at least most of, and preferably all of, the surface of the stress-applying feature which is exposed above the outer surface of the expandable member or other supporting surface will be rounded free from irregularities and imperfections that might inhibit advancement though a target body lumen and will typically be configured to present low friction smoothed surfaces free from edges to the walls of the vasculature or other body lumen as the expandable structure is advanced therethrough. By “at least most of” it is meant that at least 50%, preferably at least 60%, more preferably at least 70%, still more preferably at least 80%, and most preferably at least 90% of the exposed surface of the stress-applying feature will be rounded, smoothed, free from edges, or otherwise configured to present low friction. It has been found by the inventors herein that such smoothed, rounded features will still be able provide the force necessary to dent/disrupt even hardened plaque when deployed as described herein.

Pre-formed stress-applying features according to the present invention may be attached to an expandable or non-expandable support structure, usually being an expandable structure, e.g. a polymeric balloon, or intermediate structure, e.g. a scaffold or sleeve or cage, by one or more of soldering, use of an adhesive (gluing), typically an acrylic or other polymer adhesive, heat bonding, fusing, welding, threaded attachment, riveting, crimping, press fit, other, and the like. Stress-applying features formed integrally as part of the balloon, scaffold, stent, cage, or sleeve may at a late or final process or location, e.g. by molding, a deposition process to build-up a structure on the outer surface of the scaffold, balloon, or sleeve or, in a preferred alternative, may be formed as a tab or other element projecting from a component of the scaffold, such as a crown, strut, or link, and folded over the outer surface of the component after forming scaffold but before implantation. Specific examples of such features are described in more detail as appurtenant features in the present invention. Pre-formed stress-applying features according to the present invention may be attached to an expandable structure, e.g., a balloon, or intermediate structure, e.g. a scaffold or sleeve or cage, by one or more of soldering, use of an adhesive (gluing), heat bonding, fusing, welding, threaded attachment, riveting, crimping, press fit, other, and the like. Stress-applying features formed integrally as part of the balloon, scaffold, stent, cage, or sleeve may at a late or final process or location, e.g. by molding, a deposition process to build-up a structure on the outer surface of the scaffold, balloon, or sleeve or, in a preferred alternative, may be formed as a tab or other element projecting from a component of the scaffold, such as a crown, strut, or link, and folded over the outer surface of the component after forming scaffold but before implantation. Different individual stress-applying features on a single support structure may have the same or different footprint(s), shape(s), height(s), configuration(s), and the like, so that adjacent stress-applying features may be the same or different. Specific examples of such features are described in more detail as appurtenant features in the present invention.

It will often be preferred to choose the polymer adhesives and other polymer layers which are formed over the outer wall surface of the of the inflatable polymeric balloon to have a hardness which is less than that of the balloon polymer or other material. A lower hardness typically corresponds to a greater compliance. In preferred examples, the inflatable balloon has a Shore D hardness in a range from 60D to 80D (typical of nylons) and the polymer adhesive(s) have a Shore D hardness in a range from 50D to 65D when cured (typical of acrylics). For comparison, the rigid stress-applying features will typically have a Mohs hardness greater than 4, usually being greater than 8.

In a preferred example, the expandable structure comprises one or more of an inflatable polymeric balloon, a stent, a sleeve, a cage, a drug delivery balloon, or the like. The stress-applying features may be applied to one or more surfaces or surface regions of the expandable structure, wherein the surfaces may comprise outer surfaces, inner surfaces, and side surfaces of the expandable structure. When the features are applied to an inner surface of an expandable structure, the features protrude radially outwardly to engage the hardened plaque upon expansion or inflation of the expandable structure.

In some instances, the stress-applying features may be applied to one or more outer surface regions of an inflatable polymeric balloon, such as a working length of the balloon (typically a cylindrical or other tapered or non-tapered tubular surface central region of the polymeric inflatable balloon) and/or a conical or other tapered end region of the balloon. Additionally or alternatively, the stress-applying features may be applied to opposed side walls or surfaces, such as those formed by gaps or annular indentations created in a wall of an inflatable polymeric balloon configured to capture a valve leaflet for performing valvuloplasty, as described in detail herein below.

The phrase “outer surface” of an inflatable polymeric balloon, as used herein and in the claims, includes both the outer surface of the inflatable polymeric balloon itself as well as any outer surface of a base or other layer formed and fixedly attached or adhered over the outer surface of the inflatable polymeric balloon. For example, the phrase “outer surface” of the inflatable polymeric balloon will specifically include the outer surface of any base layer formed over an underlying balloon wall which base layer may comprise, consist essentially of, or consist of one, two, three, or more sheets, coatings, or films applied simultaneously or successively.

In particular instances, the stress-applying features may be affixed to an expandable structure at least partially to an outer surface of one or more elements of, for example, a scaffold ring such as on a crown and/or strut. Such features can also be affixed at least o to a link where the link connects two adjacent rings of the scaffold. The stress-applying feature can be formed from the same material or from a different material or materials from the scaffold. The stress-applying features can be 3D printed (deposited) or be laser cut from a tube, either separately from the scaffold fabrication or as part of the scaffold fabrication. In either case, the stress-applying feature protrudes radially outwardly from a scaffold element, such as crown, strut, or link. The stress-applying features may be formed as part of the scaffold and positioned in place after forming and before implantation, e.g. fixed by solder, welding, adhesive (such as an epoxy), press fit, mechanical fit, tying or other technique as described previously, where the stress-applying feature may be fixed or attached to the outer surface of the scaffold of optionally be positioned in a recess, hole or other receptacle formed in or through the outer surface the scaffold.

Affixing the stress-applying feature comprises one or more of coating, adhesive, fusing, solder, welding, vapor or chemical deposition, laser deposition, constriction with a sleeve, press fit or mechanical lock onto the scaffold outer surface (onto an indented scaffold surface, a recess, or a hole through the scaffold where portion of the stress-applying feature is forced into or through), epoxy, or combination thereof, or other.

The scaffold can be formed from a degradable material or a non-degradable material. In a preferred example, the scaffold formed from a metal or metal alloy or other non-degradable material having a Mohs hardness of 2.5 or greater, preferably 3.5 or greater, and more preferably 4.5 or greater. In a preferred example, the stress-applying feature will have a Mohs hardness equal to or greater than the Mohs hardness of the scaffold or other expandable structure. In other preferred example, the stress-applying feature will have a Mohs hardness greater than the Mohs hardness of the scaffold or other expandable structure. For example, the stress-applying features may be formed from material to have a Mohs hardness in a range from 2 to 10, usually from 2.5 to 10, more usually from 3 to 10, 3.5 to 10, 4 to 10, 4.5 to 10, 5 to 10, 5.5 to 10, 6 to 10, or 6.5 to 10.

In a preferred example, the expandable structure comprises stress-inducing features having a Mohs hardness of 2.5 or greater, preferably 3.5 or greater, and more preferably 4.5 or greater, often 5.5 or greater, and most preferably 6.5 or greater. In yet another example, the stress inducing features have a Mohs hardness ranging from 2 to 9, preferably ranging from 3 to 9, more preferably ranging from 4 to 9. In yet another example, the Mohs hardness of the stress-applying features will be greater than the Mohs hardness of the expandable structure supporting said features.

In a preferred example, the stress-applying features, spacer features, and all other protruding features and elements as described herein comprise at least one material from the following list: metallic, polymeric, ceramic, glass, metal alloy, or the like. The at least one material maybe coated with another material. For example, a stainless-steel hemisphere may be coated with an adhesive and/or a polymeric material, wherein the adhesive and/or polymeric material has a Mohs hardness that is less than the Mohs hardness of the stainless-steel hemisphere. In a preferred example, the stress-applying features comprise a metal or other hard core as disclosed above covered partially or fully by a coating where the coating preferably has a Mohs hardness less than the Mohs hardness of the core. The softer coating can provide any one of roundness, dullness, lubricity, smoothness, and the like without sacrificing the stress-applying feature's ability to dent or fracture hardened plaque. In yet another example, the Mohs hardness of the stress-applying features will be at least two times greater than the Mohs hardness of the expandable structure supporting said features. In a preferred example, the expandable structure comprises or consists of polymeric material, while the stress-inducing features comprise or consist of metal or metal alloy material.

In further preferred examples, the stress-applying features, spacer features, and all other protruding features and elements as described will possess some degree of radiopacity to assist in viewing under fluoroscopy, either inherently as is the case with many metals, as a result of as combining, alloying, plating, or coating with a radiopaque material or filler, or both. Suitable radiopaque fillers suitable for combination with polymers include salts or particles made from tungsten, gold, platinum, iridium, bismuth, barium, and/or iodine salts. Metals having a high radiopacity suitable for coating or plating metal and non-metal features include bismuth, gold, platinum, tungsten, and iridium.

In other examples, the core may be polymeric and be coated with a metallic coating having a Mohs hardness larger than a Mohs hardness of the polymeric feature. Specific combinations of a core Mohs hardness and a coating Mohs hardness may be selected to provide sufficient force to dent, disrupt, or otherwise expand a hardened or calcified tissue or lesion, while being sufficiently flexible and smooth to be safely advanced through the vasculature or other anatomy.

When layering or mixing of multiple adhesives, one adhesive material may be softer than an adjacent material. Alternatively or additionally, a first adhesive material may be more compatible with the material of the expandable member and be layered directly over the member surface, while one or more additional materials may be layer over the first layer to provide bonding to the stress-applying feature or other propertied to enhance the overall bonding of the stress-applying feature to the expandable member.

The material(s) of the stress-applying features are typically selected and configured to compress by 0.2 mm or less, by 0.1 mm or less, by 0.05 mm or less, and/or by 0.01 mm or less, when deployed against calcified or other plaque by a balloon, sleeve, or other expandable member. In other words, the material compressibility against a hard surface when pressurized through an expandable structure range from 0.001 mm to 0.2 mm, preferably from 0.001 mm to 0.1 mm, and more preferably from 0.001 mm to 0.01 mm. In other examples, the stress-applying feature has a material compressibility which is less than the compliance of the material of the expandable member when pressurized to a nominal inflation pressure.

The stress-applying features in another example have a fracture toughness of at least 10 MPa·m^{1/2}, preferably at least 20 MPa·m^{1/2}, more preferably at least 50 MPa·m^{1/2}, and most preferably at least 100 MPa·m^{1/2}. In a preferred example, the stress applying features have a Mohs hardness being greater than 4 and a fracture toughness being greater than 50 MPa·m^{1/2}. In a preferred example, the stress applying features consist of a metal or metal alloy. In a preferred example, the stress applying features consist of or comprises a radiopaque material that provides radiopacity under x-ray/fluoroscopy. In yet another example, the stress applying features consist of or comprises a radiopaque material configured to provide radiopacity sufficient to visualize the features under fluoroscopy, preferably sufficient to visualize the features under fluoroscopy without the aid of radiopaque contrast material. In yet another preferred example, the stress applying features are attached to an expandable balloon catheter outer surface wherein the features consist of or comprises a radiopaque material configured to provide radiopacity sufficient to visualize the features under fluoroscopy, wherein the visualization is equal to or greater than the visualization of the contrast filled balloon under fluoroscopy. These advantages provide accurate sizing of the vessel, enhanced boundaries definition of the expandable structure such as an expanded/inflated balloon, and/or provides enhanced detection of hardened plaque or plaque morphology.

The stress-applying features of the present invention present a generally rounded, dull, and atraumatic structure above the surface of the expandable structure to facilitate advancement through the vasculature while still providing the force necessary to dent/disrupt the hardened plaque when expanded by the expandable member. This is advantageous as many of the cutting features of the prior art are sharp and would catch on the vasculature as the catheters are being advanced. Thus, in preferred examples of the present invention, at least the entire surface above the substrate and sometimes including the feature surface attached to the substrate) is rounded, smooth, and presents minimal friction as it is advanced.

The stress-applying features of the present invention in another example present a generally rounded, dull, free from peripheral edges, and atraumatic structure above the surface of the expandable structure to facilitate advancement through the vasculature while still providing the force necessary to dent/disrupt the hardened plaque when expanded by the expandable member. The stress-applying features of the present invention in another example present a generally rounded, dull, free from peripheral edges, free from rounded (beveled) peripheral edges, and atraumatic structure above the surface of the expandable structure to facilitate advancement through the vasculature while still providing the force necessary to dent/disrupt the hardened plaque when expanded by the expandable member. The plaque disrupting feature in yet another example may have a peripheral edge or a rounded (beveled) peripheral edge. However, in a preferred example, the stress-applying features are free from a peripheral edge. In yet another preferred example, the stress-applying feature may be free from a peripheral edge or peripheral beveled edge.

In some instances, the stress-applying features of the present invention may comprise a sharp or potentially traumatic core which is covered by or coated with another material that provides a rounded, dull, or otherwise atraumatic engagement surface to avoid injury to the vasculature or other body lumen at the expandable member is advanced therethrough. In such instances, at least the potentially injurious contact regions of the features are covered or coated.

The expandable structure such as a balloon or scaffold can have any one of various shapes or configurations comprising one or more of cylindrical, substantially cylindrical, hourglass, dog bone, tapered, elliptical, oblong, or other shape, exterior shape, profile, or configuration. In many instances, the balloon, sleeve, cage, or scaffold will be expandable from a crimped or small configuration to a deployed or larger configuration by applying an interior expansion force, usually by fluid or balloon expansion as commonly employed for vascular devices and stents. Alternatively, the scaffold can be constrained and deployed from the constrained configuration by removing or withdrawing the constraint. Constraint may be provided by a catheter, a sleeve, a sheath, or other conventional or novel constraining structures. Such designs are commonly referred to as “self-expanding.”

The stress-applying features can be formed from one or more of a metal, a metal alloy, a ceramic, a mineral, a polymer, a polymer blend, a mixture of polymers, an epoxy, a diamond, or combination thereof, including both degradable and non-degradable materials. Suitable metals and metal alloys include stainless steel, cobalt chrome, platinum chromium, platinum-iridium, silver, nickel-titanium alloy (NiTi), tungsten, tungsten carbide, palladium, cobalt, gold, platinum, iridium, titanium carbide, zirconium, chromium, magnesium or magnesium alloys such as magnesium-zinc and magnesium yttrium, zinc or zinc alloys such as zinc-calcium and zinc-lithium, and the like. In a specific example, which is particularly useful when place over balloons and sleeves, a chrome-plated steel ball may be used which is corrosion resistant and harder than an underlying metal, typically having a hardness comparable to that of a solid chromium ball.

In some examples, the plaque disrupting features comprise one or more material coating, covering, plated, or otherwise attached to one or more of features contact region, feature base, and/or features outer surface. In a preferred example, the material provides one or more of round, dull, smoothness, slipperiness, less friction, convexity, atraumatic surface, and/or lubricity to the surface of the feature, base, or contact region. In some examples, the material is polymeric comprising one or more of Poly-Para-Xylylene such as Parylene, Parylene N, and Parylene C, silicone such as polydimethylsiloxane, poly(diphenyl) siloxane, poly(methyl-co-phenyl) siloxane, poly(methyltrifluoropropyl) siloxane, poly(methyl-co-methyltrifluoropropyl) siloxane, or the like, polyurethanes and their copolymers such as tecoflex, pellathane, chronoflex, chronoprene, chronothane, chronosil, polyblend, or the like, polyethylene-vinyl acetate, polyvinylidene fluoride, polyvinylidene fluoride-co-hexafluoropropylene, polybutylmethacrylate, poly(styrene-butadiene-styrene), poly-lactide, hydrophilic material, or the like, or combinations thereof. Metal or metal alloy examples to coat providing hard or harder surface yet smooth, rounded, convex, and/or dull, applied to for example a polymeric features by evaporative, sputtering, vapor deposition, or plasma coating to the surface of the feature using a metal or alloy such as titanium, Ti-6Al-4V alloy, titanium-magnesium, stainless steel such as 316, 304, 420, or the like, magnesium alloys such as yttrium-zirconium-magnesium, chromium, cobalt, cobalt-chrome, CoCrMo, nitinol, tungsten, gold, platinum, silver, zinc, palladium, iridium, ruthenium, rhodium, indium, tin, molybdenum, iron, vanadium, nickel, niobium, zirconium, or the like, or combination thereof. Examples of polymeric features hardened by fillings of fine glass, quartz or silica fibers or particles such as 40% glass-filled Nylon, carbon fibers, carbon nanospheres, carbon nanotubes, carbon nanofibers, carbon nanotubes, talc, aramid or the like, are another example.

In a preferred example, adhesives are used to attach, cover, secure, couple, and/or bond the feature or feature base the expandable structure surface. Examples include but not limited to one or more of light-cured materials such as Henkel Loctite 3943, 3973, 3972, 3321, 3311, 3526, Permabond UV610, UV670, UB7141, or the like, moisture resistant light-cure material such as Loctite EA 3335, 4310, 3525, 3494, or the like, epoxy such as 5 min epoxy, Epo-tek MED-353ND, Epo-tek MED-HYB-353ND, Masterbond EP41SMed, Henkel Loctite 3981, polyurethanes such as Permabond PT321, PT326, and PT328, epoxy-polyurethane blends, and cyanoacrylates such as HB Fuller M2240-05, Permabond ET5393, Permabond 2011, Infinity CA-110-M, Loctite 4014 with or without primer such as HB Fuller 6070 or Loctite 713, structural acrylic adhesives such as Permabond TA430, TA435, TA437, TA49, TA459. TA4246 with or without initiator 41 or 46, or the like, or combination thereof. In a preferred example, the adhesive material covers a footprint at least the size of the footprint of the feature (or feature base) contacting the expandable structure surface, larger footprint than and including the footprint of the feature or feature base contacting the expandable structure surface, at least some of the feature surface above the expandable structure surface, or at least the entire outer surface of the feature. In yet another example, the adhesive covers at least an inner surface of a hollow feature.

In a preferred example, the stress-applying features have a blunt contact region of which acts to concentrate the force applied to the occlusive material, resistant material, plaque, or calcified plaque on or within the wall of the blood as the expandable structure or scaffold expands in the blood vessel or other body lumen.

In a preferred example, the plaque disrupting features contact region is symmetrical. Symmetrical configurations provide for the smooth navigation through the vasculature without hanging up or causing a vessel injury. In other examples, the plaque contact region is asymmetrical.

Individual stress-applying features will typically possess a single blunt contact region, but in some instances may possess two, three, or more separately formed blunt contact regions each defined by a continuous peripheral boundary. Individual blunt contact regions will typically have an area in a range from 0.0001 mm² to 5 mm², preferably from 0.001 mm² to 2 mm², more preferably from 0.001 mm² to 0.2 mm², and most preferably from 0.01 mm² to 0.2 mm². In one example, the total outer surface area of the sinusoidal scaffold ring or balloon will typically have an area in a range from 0.05 mm² to 10 mm², preferably from 0.5 mm² to 2.5 mm², more preferably from 0.5 mm² to 1.5 mm². Depending on both the total number of blunt contact regions and contact areas of the individual blunt contact regions, the force per unit area (pressure) applied by the stress-applying features will be increased by a factor in a range from 1 to 1000, often from 1 to 100, preferably from 1 to 50.

The radial distance of the blunt contact region of the stress-applying feature above the outer surface of the expandable structure such as scaffold or other structures may be in a range from 0.05 mm to 1 mm, preferably 0.15 mm to 0.5 mm. The blunt contact regions may have a width or diameter (in the case of circular blunt contact regions) in a range from 10 μm to 2.5 mm, preferably ranging from 30 μm to 250 μm. In some instances, the blunt contact regions may have a width to length ratio in a range from 1:3 to 3:1, often 1:2 to 2:1, and about 1:1, often being circular.

The stress-applying feature will typically be positioned directly over the outer surface of the scaffold, i.e., the feature will be integrally formed, subsequently deposited, or other located so that it extends or protrudes radially away from the outer surface of a strut, crown, link, or other primary component of the scaffold. In other instances, however, the stress-inducing feature can be formed as an integral but appurtenant component of the scaffold, e.g., has an arm or other integral connector (similar to a link between adjacent rings) extending from and connecting the feature to a primary scaffold element. Examples include stress-applying features attached to a scaffold crown, strut, or link by an arm. In some examples, a single appurtenant stress-applying feature may be attached to two or more primary scaffold components (e.g., crowns, struts, or links) by two or more individual arms.

Such appurtenant stress-applying features may be formed to have a radially outwardly protruding blunt contact region and be useful with further reconfiguring or deformation. In this way, the blunt contact regions can be positioned laterally away from the outer surfaces primary scaffold components. In other instances, the appurtenant features can be folded, typically over by bending the attachment arms, so that they overlie an outer surface of an adjacent primary scaffold component. Optionally, such folded appurtenant features can be further attached to the primary component by any of soldering, welding, gluing, press fit, mechanical fit, tying or other technique, as described previously.

In some examples, the stress-applying features are placed on opposite surface regions on an expandable structure such as a balloon surface or a scaffold circumferential ring, such as on substantially opposite crowns and/or struts, i.e., being separated by 180°. In other examples, individual stress-applying features may be distributed over the circumference of a balloon, sleeve, cage, or an individual ring by other equal angular separations, e.g., by 30°, 45°, 60°, 90°, or 120°, typically being located on the surface of a crown, strut, axial link or bridging two or more of such scaffold structures. In these and other instances, the stress-applying features on an expandable structure or on an individual ring may be circumferentially offset from those on axial length or one or more axially separated rings, e.g, by about 10°, 200, 300, 400, 60°, or 90°. In still further examples, the stress-applying features may be disposed in a helical pattern along a partial or entire length of the expandable structure or scaffold. In other example, the stress-applying features on an expandable structure, a stent, or an individual ring may be circumferentially and axially offset from each other within said structure, stent, or ring and/or may be circumferentially and axially offset from those on regions of the expandable structure, or one or more other rings. In yet another example, at least one end of the expandable structure or scaffold may have fewer (compared to a middle structure or scaffold region) or no stress-applying features, over at least one, two, three, four, five, six, or more terminal length as measured in mm or cm or circumferential rings on either or both ends of the scaffold, often being free of stress-applying features over these terminal ends or end rings.

In preferred examples, the stress-applying features may be placed on crown regions only; the stress-applying features may be oriented circumferentially around the surfaces of some or all individual rings; the number of stress-applying features per ring may range ranges from 1 to 5, preferably ranges from 2 to 5, and more preferably ranges from 2 to 3 stress-applying features; the stress-applying features may be located only on crown regions, only on link regions, or only on crown and link regions; the stress-applying features may be located only on diametrically opposed crown regions, only on diametrically opposed link regions, or only on diametrically opposed crown and link regions; or the stress-applying features may be located only on crown regions, link regions, or crown and link regions circumferentially separated by 30°, 45°, 60°, 90°, 120°, or 180° on the same ring or on axially adjacent or separated rings. In still other examples, the stress-applying features may be present at a ratio of “stress-applying features” to “crowns” within an individual circumferential ring in a range from 1:1 to 1:4, often from 1:2 to 1:3.

The stress-applying features of the present invention may have any one of a variety of specific shapes and configurations characterized by a blunt contact region at a location spaced-radially outwardly from the outer surface of the expandable structure or scaffold. For example, they may have a disk-like shape, truncated conical shape, a sphere, a ball, an ellipse, a truncated prism-like shape, a truncated tear-drop shape, or the like. The base of the stress-applying feature may contact the outer surface of the expandable structure or scaffold with a footprint having an elliptical, triangular, circular, polygonal, or irregular periphery with the blunt end positioned radially outwardly from the outer surface of the expandable structure or scaffold. In some instances, the stress-applying features can be attached to a base layer affixed to the outer surface of the expandable structure or scaffold, and one, two, three or more additional layers may be formed or attached over the base layer. The layers can be formed from the same or different materials and can be applied or deposited in situ or be pre-formed and attached by any of the methods described previously and can have the same or different shapes, lengths, widths, or height. The stress-applying features will typically be symmetrical but in some instances may be constructed asymmetrically with respect a circumferential and/or axial line or plane.

The blunt contact regions will typically be circular, but in some instances may be square, rectangular, polygonal, and/or elongated in an axial or lateral direction. For example, the blunt contact region may have a length and a width with an aspect ratio in a range from 5:1 to 1:10, preferably ranging from 3:1 to 1:10, more preferably 2:1 to 1:10. In a preferred example, the length to width ratio of the blunt contact region ranges from 2:1 to 1:2, about 1:1, or about 1:2. In yet another preferred example, the width of the stress-applying feature or stress-applying feature base is longer than the width of the stress-applying feature. In yet another example, the height of the blunt contact region (radially spaced distance from the outer surface of the expandable structure or scaffold at the point of attachment of the stress-applying figure) is greater than the length or width of the blunt contact region, greater than both said length and width, greater than said length but less that said width, or less than said length but greater than said width. In yet another example, the circumferential width is equal or longer than the axial length of at least some of the stress inducing features.

In most instances where the expandable structure is a stent (scaffold), the base of the stress-applying feature does not extend beyond the edges of the outer surface of a single crown region, a single strut region, or a single link region, but in other instances the base may extend beyond the edges of the scaffold surface and/or may span two or more adjacent crowns, struts, and/or links.

The stress-applying feature will be configured to shear, fracture, break, disrupt, dent, or fragment occlusive material on or in an inner wall of a blood vessel, valve, or body lumen, including both arteries and veins in the cardiac and peripheral vasculature. The occlusive material may comprise calcified lesions often in the form of calcified plaque partially or fully occluding or partially or fully encircling the blood vessel, valve leaflet, or lumen. In a preferred example, the features are composed of or comprised of blunt contact regions which are configured to shear, fracture, dent, break, disrupt, or fragment occlusive material on or in an inner wall of the vessel or body lumen when the expandable structure, such as a balloon, stent, sleeve, or the like, is inflated or expanded to its radially expanded, deployed configuration. For example, the blunt contact region may also have a peripheral edge which shears, fractures, breaks, disrupts, or otherwise fragments the occlusive material while the surface of the blunt contact region inhibits the peripheral edge from unintended cutting or otherwise causing substantial injury, such as dissection, to the blood vessel wall, valve leaflet, or body lumen. In yet another example, the stress-applying feature contact region and peripheral edges are blunt, which expands, dents, shears, fractures, breaks, disrupts, or otherwise fragments the plaque or occlusive material when the blunt contact surface and periphery are forced or pressed against the vessel wall, hardened plaque, valve annulus, valve leaflet or body lumen. In this example, the peripheral edge is dulled by beveling the edges or polishing said edges or coating said edges or feature surface. In yet another example, the stress-applying feature surfaces comprising the contact region, the peripheral regions, the base, and the feature body are blunt which dents, shears, fractures, breaks, disrupts, or otherwise fragments the plaque or occlusive material when the blunt surfaces are forced or pressed against the vessel wall, hardened plaque, valve annulus, valve leaflet or body lumen. In yet another example, the stress-applying feature surfaces comprising the contact region, the base, and the feature body being blunt and free from peripheral edges which dents, shears, fractures, breaks, disrupts, or otherwise fragments the plaque or occlusive material when the blunt surfaces are forced or pressed against the vessel wall, hardened plaque, valve annulus, valve leaflet or body lumen. In yet another example, the stress-applying features on one or more surfaces are blunt, dull, or otherwise causes minimal to no injury to the blood vessel, body lumen, valve annulus, valve leaflet, when the expandable structure containing said features is radially or axially expanded or deployed against a vessel wall, body lumen, or valve annuals/leaflet. In yet another example, the stress-applying features surfaces have the same degree of bluntness, or different degrees of bluntness.

In a preferred example, the stress-applying features comprise one or more of applying force to the tissue, resistant tissue, plaque, calcified plaque, and/or fibrotic plaque to disrupt vessel occlusion, dent vessel tissue and/or occlusion, and/or enlarge a vessel lumen, or body lumen.

In a preferred example, stress-applying features on a contact region may comprise one or more of the following configurations: blunt, atraumatic, dull, wherein the feature contact region of the stress-applying feature shears, dents and/or disrupts and/or fractures hardened vessel tissue and/or calcified plaque to enlarge a vessel lumen.

In another example, the stress-applying feature has one body. In another examples the stress-applying feature comprises a base wherein the base is separate from the feature body and wherein the feature and the base are attached together.

In another example, the stress-applying features are formed on a balloon, on an expandable member, on a sleeve, on a cage, or other devices. In other examples, the stress-applying feature is formed on an outer surface of a balloon, an expandable member, or a sleeve, or cage, or is formed on an inner surface of a balloon, expandable member, or a sleeve, or cage. In one example, the stress-applying features are formed on an inner surface of a balloon, sleeve, cage, or expandable member, said features are configured to protrude radially outwardly above the surface of said balloon, sleeve, cage, or expandable member, upon expansion of the balloon, sleeve, cage, or expandable member, from a crimped or small configuration to an expanded configuration. In another example, the stress-applying features are formed on an inner surface of a balloon, sleeve, cage, or expandable member, and then the balloon, sleeve, cage, or expandable member is inverted inside out providing the features onto the outer surface of the balloon, sleeve, cage, or expandable member prior to expansion of the balloon, sleeve, cage, or expandable member, from a crimped or small configuration to an expanded configuration.

The stress inducing features will be adhered to the outer balloon surface by one or more layers or “spots” of an adhesive polymer having a compliance equal to or less than that of the balloon polymer, for example as described with reference to FIGS. 16E-A to 16E-G herein, to maintain flexibility of the expandable balloon during navigation of the balloon catheter in the crimped configuration. Layers of the adhesive polymer may be applied by spraying, dipping, painting or other conventional techniques, typically to a thickness in a range from 1 μm to 50 μm, usually from 1 μm to 20 μm. A spot of adhesive will typically be applied or dispensed as a small droplet, typically having a volume in the range from 0.1 μl to 1 μl, usually from 0.1 μl to 0.5 μl, at the location where the stress-applying feature to be adhered (attached) to the outer surface of the balloon.

The balloon wall will typically consist of or consist essentially of a single layer of the polymer, copolymer, or polymer blend having a thickness which depends on the materials of fabrication and intended inflation pressure, For example, nylons will usually require a thicker wall than balloons formed from harder materials, such as PET. Nylon and nylon blend balloons will typically have a double wall thickness in a range from 0.02 to 0.1 mm, usually from 0.02 mm to 0.07 mm, while PET balloons will typically have a double wall thickness in a range from 0.01 mm to 0.025 mm, usually from 0.01 mm to 0.015 mm. The balloon wall thickness will typically be, but not necessarily always be, uniform, or substantially uniform, over most or over all of the balloon surface or balloon working length surface, typically but not necessarily cylindrical. For example, the balloon having a uniform, or substantially uniform, wall thickness over at least a cylindrical middle portion of the balloon surface when inflated or when the balloon is deflated, usually having a uniform mean wall thickness wherein the wall thickness varies from said mean by no more than +20%, more usually by no more than +10% over the length of the cylindrical surface when the balloon is inflated or when the balloon is deflated.

Adhesive and other polymer layers formed over the balloon surface will typically have a thickness which is 75% or less (for single layers) than the thickness of the balloon wall, usually 60% or less, and preferably 50% or less. The cumulative thickness of multiple layers, however, may be greater, for example being 150% or less for two layers, usually 120% or less, and preferably 100% or less.

The balloon wall will typically consist of or consist essentially of a single layer of the polymer, copolymer, or polymer blend, free from any additional material, additive, or features that would alter, or that would substantially alter, the balloon properties including balloon compliance, expansion force, or flexibility.

The balloon wall will typically consist of or consist essentially of a single layer of the polymer, copolymer, or polymer blends configured to maximize flexibility to navigate vascular anatomy. In this example, the balloon surface consists of a single layer free from features or material that would stiffen the balloon or balloon surface.

The stress inducing features may be arranged in any one or more of a variety of regular and irregular (random) patterns on the outer surface of the balloon, and the pattern(s) may be the same or may vary over different regions of the outer surface. Usually, the stress inducing features will be arranged in straight rows which are aligned with a central balloon axis (referred to herein as “axial rows”) which are circumferentially spaced-apart over at least a cylindrical center region of the outer balloon surface and often over the entire outer balloon surface. In such instances, it will often be advantageous to have at least of some of the stress-applying features in one axial row “axially offset” relative to those in other axial rows to reduce circumferential overlap of the stress-applying features after the balloon is folded to provide a lower entry profile, as described in more detail below. As a result, no two stress-applying features will lie on a common circumference of the balloon when inflated, i.e., each pair of axially adjacent stress-applying features will be centered on a circumferential line (a circular ring) which is axially spaced apart from the circumferential line upon which the closest adjacent stress-applying feature. Preferably, depending on the diameters or widths of the stress-applying features, the centers of the adjacent stress-applying features will be axially spaced-apart sufficiently so that the features will have spaces or “gap” therebetween, i.e., the features centers and/or base will not axially overlap, thus reducing potential circumferential overlap of the features when the balloon is deflated. Most preferably, no two features on the outer surface of the balloon will axially or circumferentially overlap when the balloon is deflated and folded. Broad, exemplary, and preferred dimensional ranges are set forth in Table 1 below.

TABLE 1

| | | Exemplary | |
|---|---|---|---|
| | Broad Range | Range | Preferred Range |
| Feature Width | 0.15 to 1 | 0.2 to 1 | 0.3 to 0.6 |
| (mm) | | | |
| Center Spacing | 0.3 to 2 | 0.4 to 1.5 | 0.5 to 1 |
| (mm) | | | |
| Gap | 0 to 3 | 0 to 2 | 0.05 to 0.4 |
| (mm) | | | |

As the preferred balloons of the present invention will have limited compliance (stretchability at high inflation pressures), their nominal size when inflated will be at most slightly larger than when uninflated, thus typically necessitating that the balloons be folded or pleated for delivery on the deployment catheter. The number of folds or pleats will typically depend on the number of axial rows of stress-applying features on the outer balloon surface. For example, a balloon having three axial rows of stress-applying features will typically be folded to have three folds or pleats, where each row of stress-applying features is disposed between each circumferentially adjacent pair of axial rows of pleats. In another example, a balloon having four axial rows of stress-applying features may have four folds or pleats, where each row of stress-applying features disposed between each circumferentially adjacent pair pleats. In other alternative examples, some or all of the axial rows of stress-applying features may be disposed over a fold or pleat of the balloon when folded prior to inflation.

The balloons of the present invention will typically be free from stiffening members or other components that would affect the compliance or other property of the balloon to differ in one segment of the balloon when compared to other segments of the balloon. Thus, the balloons will usually have uniform compliance and other physical properties, including but not limited to compliance (stretchability) and flexibility (ability to bend, navigate the anatomy, and/or fold without breaking), over at least their circumferential centers and usually over their entire structures.

The balloons of the present invention will typically be inflated to relatively high pressures to enable the stress-applying features to fracture calcified plaque, usually being inflated to a pressure of at least 2 atm, usually at least 5 atm, more usually to at least 8 atm, still more usually to at least 12 atm, and often to 15 atm or higher.

While in many embodiments and examples, the stress-applying features of the present invention are intended to be placed and attached directly to the outer surfaces of expandable structures such as balloons, stent and graft structures, in other instances they may be placed on an outer and/or inner surface of an expandable sleeve or similar support structure that that may be placed over a conventional balloon or stent or vascular graft. In still other instances, the stress-applying features of the present invention may be placed directly on an angioplasty balloon as a supplement or alternative to the blades/elements of a conventional cutting or scoring balloon. In some instances, a balloon having stress-applying features as described herein may be used to expand a stent or vascular graft and the balloon and stress-applying features removed from the stent or vascular graft after expansion.

A wide variety of stress- and force-applying features are described and claimed herein, such as blunt, domed, those having sharp elements, and the like, and a wide variety of expandable structures, or components, or substrates are described and claimed herein, such stents, grafts, balloons, sleeves, cages, and the like. The present invention will include each and every individual type of stress- or force-applying element in combination with each and every expandable structure/component, individually and in combination

Additionally, in some instances, the preferred stress-applying features of the present invention may be incorporated on scaffold components that open out radially outwardly from an outer surface of a stent when the stent is radially expanded. In another instance, the stent or scaffold with stress-applying features may be radially contracted after being radially outwardly expanded.

In still other instances, the surfaces of stress-applying features, including the blunt contact regions, can be roughened or otherwise modified to enhance adherence to the surface of the vessel, or calcified lesion, for example having one or more tissue interfacing features such as a texture, polish, friction, barbs, spikes, wedges, microstructure patterns, or the like on, on, over, or adjacent to the surface, features to stably engage the vessel wall tissue before, during, or once occlusive material has been fractures to minimize mispositioning or sliding of the expandable structure such as balloon, sleeve, cage, or of the scaffold at or adjacent to the stress-applying features. In yet another example, the base of the feature maybe roughened, etched, grooved, patterned, sandblasted, or micropatterned to enhance bonding of the base to an expandable structure surface.

In still other instances, the stress-applying features may comprise a sharp element projecting outwardly from the blunt contact region. The sharp element, e.g., a shaft or other body having a sharp tip or sharp edge, is typically configured to concentrate stress when engaged against the occlusive material on the wall of a vascular lumen when the blunt contact region is pressed against a surface of the occlusive material. The height of the sharp element will be selected to be sufficient to enhance or “nucleate” fracturing of a hardened lesion while reducing or eliminating the risk of injury to the arterial wall, both underlying the lesion and away from the lesion. For example, the blunt surface may extend above a surface of the balloon or scaffold by a first distance and the sharp element projects from the surface of the blunt contact region by a second distance equal to 0.05 to 0.1 mm of the first distance. Typically, the sharp element will have a height or length of at least 0.01 mm, typically being in a range from 0.01 mm to 0.2 mm, usually being in a range from 0.01 mm to 0.1 mm.

In a first aspect, the present invention provides an endoluminal prosthesis comprising a scaffold and a plurality of stress-applying features coupled to an outer surface of the scaffold. The scaffold is composed at least partly of a non-degradable material and configured to expand from a crimped configuration to an expanded, deployed configuration. At least some of said stress-applying features comprise a blunt contact region spaced outwardly from said outer surface where the blunt contact region is configured to fracture occlusive material in the wall of a vascular lumen when the scaffold is expanded from the crimped configuration to the expanded configuration in the vascular lumen. The scaffold in other examples may be composed of degradable materials, such as degradable polymeric materials or degradable metallic materials, or may be composed of non-degradable material such as non-degradable metallic or metallic alloy materials, wherein the materials are configured to expand from a crimped configuration to an expanded, deployed configuration.

In preferred example, the plaque disrupting features are applied to a non-degradable scaffold structure. In other examples, the plaque disrupting features are applied to a degradable scaffold structure in a physiologic environment, wherein the degradable material comprises a degradable polymeric material or a degradable metallic or metallic alloy material.

In specific examples, the scaffold or expandable structure may have a tubular geometry, for example having a cylindrical shape, an ellipsoidal shape, a tapered profile, an hourglass shape, a dog-bone shape, other shapes, or the like.

In specific examples, at least some of the blunt contact regions comprise a peripheral edge configured to concentrate stress when engaged against the occlusive material on the wall of a vascular lumen when the expandable structure such as a scaffold is expanded from the crimped configuration to the expanded configuration in the vascular lumen. Such concentrated stresses will shear, fracture, break, disrupt, or otherwise fragment the occlusive material which is contacted by the blunt contact regions. The peripheral edge may be formed by an intersection between the blunt contact region and a peripheral wall at least partially surrounding the blunt contact region. The blunt contact region may have a variety of shapes or surfaces such as flat, rounded, convex, or concave.

In specific examples, the blunt contact region may be flat, and the blunt contact region may be parallel to the outer surface of the scaffold. Alternatively, the blunt contact region may be inclined relative to the outer surface of the expandable structure or scaffold. The peripheral wall may be oriented at an angle in a range from 75° to 105° relative to the blunt contact region. A peripheral edge may extend fully or partially about the blunt contact region and may have a width in a range from 10 μm to 200 μm. In some instances, the peripheral edge may be circular, and the width comprises a diameter.

In specific examples, at least some of the plurality of stress-applying features comprise one or more plates having a total thickness in a range from 0.1 mm to 1 mm, 0.15 mm to 1 mm, or 0.25 mm to 1 mm and a width when attached to the surface of the expandable structure or tubular scaffold in a range from 0.05 mm to 2 mm or 0.1 mm to 2 mm. At least some of the plates may be configured as disks, stacked disks, truncated cones, disks stacked with truncated cones, ellipsoidal disks, asymmetric cones, and the like. In other example the stress-applying feature may comprise one or more spheres, balls, hemispheres, partial spheres, or the like forming various shapes a “snowman” shape, or other configurations, or combination thereof.

In specific examples, the scaffolds may be formed as conventional intravascular stents, typically comprising a plurality of struts joined by crowns. The struts and crowns may be formed into circumferential rings, and in some instances the plurality of struts joined by crowns may be joined into a plurality of successive, adjacent circumferential rings joined by axial links and in other cases the rings may be joined in a helical or other pattern.

In preferred instances, at least some of the stress-applying features may be located at or adjacent to crowns and optionally at least some of the crowns carrying stress-applying features may be not joined to adjacent rings. Often, each of the stress-applying features will be located at or adjacent to a crown.

In alternative instances, at least some of the stress-applying features may be located on the struts between the crowns or may be located on one or more links joining adjacent rings.

In some instances, at least some of the stress-applying features may be arranged in diametrically opposed pairs and optionally successive diametrically opposed pairs of crowns may be circumferentially offset. Such successive diametrically opposed pairs of crowns may be circumferentially offset by an angle from 45° to 90°.

In alternative instances, at least some of the stress-applying features may be arranged in groups of three, four, or five, which may be circumferentially separated about a circumference or circle on the surface of the expanded structure or tubular scaffold by about 120°, 90°, or 72°, respectively. In another instances, at least some of the stress-applying features may be arranged in groups of three, four, or five, which may be circumferentially separated about a circumference or circle on the surface of the expanded structure or tubular scaffold by about 120°, 90°, or 72°, respectively along the length of the expanded structure or stent.

In still other instances, the stress-applying features may be arranged in other regular and/or random patterns. For example, in some patterns, successive axially spaced-apart stress-applying features will be circumferentially offset by an angle in a range from 5° to 15°. Alternatively, or additionally, at least some successive circumferentially spaced apart stress-applying features may be axially offset by the same or a different angle in the range from 5° to 15°.

In a specific example, the scaffolds of the present invention may be formed by patterning a tubular substrate, laser cutting a tubular substrate, rolling a cut substrate, bending a wire, by three-dimensional printing, or by other known stent fabrication techniques. The stress-applying features may be pre-formed and attached by gluing, soldering, welding, threaded attachment, riveting, crimping, or the like. For example, the stress-applying features may comprise preformed plates glued to the scaffold with an adhesive. Alternatively, the stress-applying features may be formed in situ by three-dimensional printing, chemical vapor deposition, electrostatic deposition, molding, or folding of a component of the scaffold. For example, the stress-applying features may comprise tabs attached to the scaffold and folded over onto the outer surface of the scaffold.

In specific examples, the scaffold may comprise a vascular stent or stent-graft. In other examples, the scaffold may comprise a prosthetic valve, a valvuloplasty device, a sleeve, or the like. In each of such instances, the scaffold may be balloon expandable or self-expanding.

In a second aspect, the present invention provides a method for fracturing calcified plaque in a patient's vasculature. A scaffold as in any one of the embodiments described previously is expanded from a crimped configuration to an expanded configuration in a calcified body vascular lumen. A plurality of stress-applying features fixed to an outer surface of the scaffold are caused to dent, expand, open, or fracture occlusive material on or in the wall of a vascular lumen when the tubular scaffold is expanded from the crimped configuration to the expanded configuration. The occlusive material typically comprises hardened plaque or calcification.

In specific instances, expanding the scaffold comprises expanding a balloon to expand the scaffold or alternatively allowing the scaffold to self-expand. For example, expanding the scaffold may comprise expanding a prosthetic heart valve in a heart valve annulus, wherein the scaffold comprises a structural support of the heart valve annulus, e.g., expanding the prosthetic heart valve may comprise expanding a balloon to expand the prosthetic heart valve in the heart valve annulus or may comprise removing a crimped elastic scaffold from radial constraint after positioning in the heart valve annulus.

In other examples, expanding the scaffold comprises expanding a valvuloplasty device in a heart valve annulus. The scaffold of the valvuloplasty device may comprise an expandable cage and expanding the valvuloplasty device comprises expanding the cage in the heart valve annulus.

Cages according to the present invention may be formed from elastic and/or malleable metals, metal alloys, and polymers, including but not limited to any of the materials described herein for the fabrication of scaffolds and stents. Such cages may be self-expanding, balloon expandable, or the like, and self-expanding cages may be configured to self-expand when released from radial constraint and/or to radially expand in response to mechanical actuation, e.g., by axial foreshortening. Each of these radial expansion mechanisms is well known and need not be described further.

In a third aspect, the present invention provides a method for fabrication of a vascular scaffold. A tubular scaffold comprises a plurality of struts joined by crowns within a tubular envelope. A plurality of tabs extending outwardly from the struts, crowns, and/or links within the tubular envelope are folded over an outer surface of the tubular envelope to form a plurality of stress-applying features on an outer surface of the tubular scaffold.

In specific instances, pairs of adjacent tabs folded one over the other to form stacked stress-applying features. The adjacent tabs in the pairs may be arranged side-by-side on the scaffold prior to folding. Alternatively, the adjacent tabs in the pairs may be arranged in tandem on the scaffold prior to folding. As a further alternative, the adjacent tabs in the pairs may be arranged on opposite sides of a strut prior to folding.

The stress-applying features can also be placed on extensions to a crown, strut, link or other structural element of the scaffold. The extension can be supported or connected to the same or a different structural element on the same ring or a diametrically opposite ring, and preferably the extension has a free terminal end wherein the stress-inducing feature is placed on or about such terminal end. Such an extension can have a width which is the same as or different from that of the element from which the extension extends and can have any one of various shapes and configurations.

Stress-inducing features may be placed on struts, strut extensions, crowns, crown extensions, links and/or link extensions. Preferably, the stress-inducing features are placed on a structural element that is deflectable in a radially outward direction when the scaffold is expanded from a crimped configuration to a deployed configuration, for example being located at a terminal or other free end or side structure of a crown, strut, link, or extension that is free to deflect as a “cantilevered” element. In a further preferred example, the stress inducing feature is placed on a terminal end of an extension or on hinge element wherein the hinge element comprising a crown or part of a link such as a Z, M, W, U, V, S-shaped link.

In some instances, a stress-inducing feature can be located on an inner surface of the scaffold, for example on a cantilevered end of a crown region or extension, a strut region or extension, and/or link region or extension. In such instances, expansion of a balloon or other expandable element within the scaffold will deflect the cantilevered end radially outwardly relative to the reminder of the outer surface of the scaffold (the feature on the inner surface will act as a spacer to preferentially expand the outer surface), thus acting as if the feature had been placed on the outer surface of the scaffold. In such instances, the feature on the inner surface need not be configured to fracture occlusive material (preferably being configured not to damage the expanding balloon) but the outer surface of the crown, strut, link or extension, which is deflected will have a peripheral edge configured to fragment or crack plaque, calcium, or other occlusive material.

In some instances, the stress-inducing features may be held on the outer or inner surface of the scaffold by an arm, clamp, or other connecting element which is fabricated together with the scaffold. Such stress-inducing features may be coupled and positioned in place (for example by bending an attaching arm) but not affixed. In such cases, the stress-inducing feature may contact the surface of the scaffold or there may be a gap left between the feature and the surface of the scaffold. As with other cases, these stress-inducing features will contact the occlusive material upon expansion of the scaffold.

In some examples, a plurality of stress-inducing features may be circumferentially and axially offset within a single row or more of features on a circumferential ring or a circumference of an expanded structure. For example, three stress-inducing features placed on three crowns within a ring will be circumferentially offset but may or may not be axially offset. They will be axially offset if the crowns are axially offset and/or certain of the stress-inducing features are located on struts.

While the stress-applying features of the present invention will usually have blunt contact regions, as described previously, in alternative instance they may comprise cones or pyramids which are not truncated and may or may not comprise a blunt contact region.

In a further aspect of the present invention, apparatus for treating hardened plaque, or calcification on or in a wall in a patient's body vessel or lumen or valve annulus or leaflet, comprises a catheter including a catheter body having a proximal end and a distal segment, an expandable structure disposed at or near the distal segment of the catheter, said expandable structure having an outer surface configured to be displaced radially outwardly toward an inner surface of the vessel wall, body lumen wall, or valve annulus, and a plurality of stress-applying features distributed over the outer or inner surface of the expandable structure, wherein at least some of the stress-applying features are attached to the outer or inner surface of the expandable structure and have, in one example, a convex rounded upper or base surfaces configured to dent, open, expand, or fracture, the calcification or vessel or body lumen while minimizing damage to the vessel or body lumen when the expandable structure is expanded within the vessel or body lumen. The expandable structure maybe advanced into the target vessel or body lumen, prior to advancing a balloon catheter into the expandable structure to expand said expandable structure to disrupt a hardened plaque.

Alternatively, the expandable structure is advanced over a balloon catheter which is already in place at the target site, the expandable structure is then bridged over (placed over) the balloon segment, prior to expansion of the balloon to expand the expandable structure to disrupt a hardened plaque. In yet a third example, the expandable structure is placed over (bridged over) the balloon segment of the balloon catheter prior to insertion in a patient body and the system is introduced together into the patient body. In yet a fourth alternative, the expandable structure is advanced distally to the target site, the balloon catheter is advanced to the target site, the expandable structure is retracted to bridge over (placed over) the expandable balloon segment, in order to expand the expandable structure. In one preferred example, the expandable structure comprises an elastic tubular body being expandable from a crimped, folded, or other reduced-width configuration to an expanded configuration. In another example, the expandable structure comprises a cage comprising two or more elongated members that are axially and circumferentially separate connecting the distal end of the catheter to a proximal end of the catheter distal segment, said strip are not connected to adjacent strips except at said proximal and distal ends, wherein the elongated members are expandable from a small configuration to an expanded larger configuration.

In another example, apparatus as in at least some of the examples may be configured for treating a blood vessel, a valve annulus, a venous valve, or AV shunt, a body lumen, where hardened plaque or calcification is typically located on or within an inner wall, an intimal layer, a medial layer, an adventitial layer, a valve leaflet, valve annulus, venous filter, or an implant.

In some examples, the expandable structure may be less rigid when not expanded and more rigid when fully expanded.

In some examples, the outer surface of the expandable structure may be generally cylindrical when fully or partially deployed or deployed or expanded. In other examples, the expandable structure may be oblong, convex, hour-glass shape, tapered, cone shaped, ellipsoid shape, or other shapes or configurations, when expanded or deployed to the expanded configuration.

In some examples, the stress-applying features comprise convex rounded upper surfaces of plurality of stress-applying features may extend radially outwardly beyond the outer surface of the expandable structure when fully expanded. For example, the convex rounded upper surfaces of plurality of stress-applying features may extend radially outwardly beyond the outer surface of the expandable structure by a distance in a range from 0.25 mm to 3 mm, preferably ranging from 0.5 mm to 3 mm when partially or fully expanded.

In some examples, the convex rounded upper surfaces of the stress-applying features may be free from edges and irregularities which could damage the wall when the expandable structure is expanded within the body lumen.

In some examples, at least some of the stress-applying features may have a single convex rounded upper surface and a lower base independently attached to the outer surface of the expandable structure.

In some examples, at least some of the stress-applying features may have a convex rounded tissue-contacting surface (usually an upper surface) and a base (usually a lower base) attached to the surface of the expandable structure. The base usually has a lower surface which is flat or otherwise configured to be directly or indirectly attached to the outer surface of the balloon by a polymeric adhesive.

In some examples, at least some of the stress-applying features may have a concave tissue contacting surface and a base attached to the surface of the expandable structure. In other examples, at least some of the stress-applying features may have a convex tissue contacting surface and a base attached to the surface of the expandable structure.

In some examples, at least some of the stress-applying features may have a convex rounded tissue contacting surface and a base attached to the surface of the expandable structure, wherein the base has the maximum configuration or maximum dimension of the feature maximum configuration or maximum dimension.

In some examples, at least some of the stress-applying features are formed from a metal or metal alloy. In other examples, the features are formed from polymeric material. In yet a third example, the features are formed from a polymeric material and coated with a harder material such as metal or metal alloy material to provide the necessary hardness to disrupt hardened plaque. In yet another example, the material forming the features maybe ceramic. In yet another example, the material forming the feature maybe coated with a harder material to provide sufficient strength to disrupt hardened plaque. In yet another example, the material forming the feature maybe coated with a softer (less hardness) material to provide roundness, convexity, lubricity, less friction, or dullness, such as in the example of a metallic material forming the features and such material coated with an adhesive material, polymeric material, and/or hydrophilic coating. In yet another example, the material forming the features maybe coated with a similar hardness material to provide one or more of roundedness, convexity, dullness, and/or atraumatic surface.

In another example, the plaque disrupting features along the circumference and/or axial length of the expandable structure may have same or different height, width, length, diameter, shape, and/or configuration.

In some examples, at least some of the stress-applying features is composed of or comprise a quadratic surface, configuration, or shape. In some examples, the base surface of the quadratic shaped feature attached to the expandable structure surface is flat, oval, rounded, square, rectangle, or contoured to the surface of the expandable structure. In some examples, the maximum height from the contact region to the base of the quadratic shaped feature is about equal to half the maximum width, length, or diameter of the base of said feature. In other examples, the maximum height from the contact region to the base of the quadratic shaped feature is smaller than half of the maximum width, length, or diameter, of the base of said feature. In yet other examples, the maximum height from the contact region to the base of the quadratic shaped feature is larger than half of the maximum width, length, or diameter, of the base of said feature.

In some examples, at least some of the stress-applying features comprise a quadratic shape or configuration comprising at least one a spherical, an ellipsoidal, an oblated, or a prolated structure.

In some examples, at least some of the stress-applying features are composed of or comprise partial sphere, partial ellipsoid, partial oblate, or partial prolate structure.

In a preferred example, at least some of the metal, hard polymer, ceramic, or other plaque disrupting features have a contact region surface formed having rounded, contoured, dull, smooth, convex, symmetric, asymmetric, concave, regular, irregular, faceted, polyhedral and/or other surface geometries. Polyhedral faceted surfaces may comprise from 4 to 100 or more facets, where individual facets may be triangular, square, rectangular, pentagonal, hexagonal, septaganol, octagonal, or the like. The exposed surfaces of the plaque disrupting features may be irregular, e.g., having grooves, crevices, scoring, or the like, over all or a portion thereof. The exposed surfaces of the plaque disrupting features may be textured, e.g., sandblasted, to impart the desired surface irregularities.

Optionally, the irregular surfaces of the metal, hard polymer, ceramic, or other plaque disrupting features, as just described, may be partially or fully coated to partially smooth and/or lubricate the surface sufficiently to facilitate advancement of balloons or other expandable elements carrying these feature through tortuous anatomy of the vasculature or elsewhere.

In other examples, the plaque disrupting features contact region surface are coated, covered, or plated with a material to provide rounded, contoured, dull, smooth, convex, and/or atraumatic surface. In one example, the material coated, covered, or plated is metallic, polymeric, ceramic, or other type material configured to provide rounded, contoured, dull, smooth, convex, and/or atraumatic surface.

In a preferred example, at least some of the plaque disrupting features have their entire surface(s) above the base surface attached to the expandable structure surface have rounded, contoured, dull, smooth, convex, and/or atraumatic surface as formed or as plated, as coated, or as covered.

In a preferred example, at least some of the plaque disrupting features have their entire surface(s) including the base surface attached to the expandable structure surface being rounded, dull, smooth, convex, rounded, and/or atraumatic surface as formed, as plated, as coated, and/or as covered.

In some examples, at least some of the stress-applying features may comprise spheres or ellipsoids.

In other examples, at least some of the stress-applying features are formed from spheres, partial spheres, or hemispheres. In one example, the partial spheres maximum height ranges from 0.1 mm to 3 mm, preferably ranges from 0.25 mm to 2 mm. In one example, the partial spheres maximum diameter ranges from 0.1 mm to 2 mm, preferably ranges from 0.25 mm to 1 mm. In yet another preferred example, the partial spheres have a configuration ranging from 10% to 90% of a configuration of a sphere, preferably ranging from 25% to 75% of a sphere configuration. In a preferred example, the partial sphere is a hemisphere (half a sphere). In a preferred example, the plaque disrupting features comprise a plurality of spheres wherein the spheres diameter ranges from 0.20 mm to 2 mm, preferably ranges from 0.25 mm to 1 mm. In another example, the plaque disrupting features comprise a plurality of partial spheres or hemispheres. In one example, the partial spheres or hemispheres have a maximum diameter ranging from 0.2 mm to 2 mm, preferably ranging from 0.25 mm to 2 mm. In another example, the plurality of spheres, partial spheres, or hemispheres have the same size, height, and/or diameter along the expandable structure circumference and/or axial length. In other example, the plaque disrupting spheres, partial spheres, or hemispheres have different size, height, and/or diameter along the expandable structure circumference and/or axial length. The partial sphere in one example maybe directly attached to the expandable structure surface. In another example, the partial sphere may have a separate base attached to the expandable structure surface and attached to the partial sphere base. In yet another example, the partial sphere may have an integral base to the partial sphere that is attached to expandable structure surface.

In some examples, at least some of the stress-applying features may comprise hemispheres having lower surfaces attached to the outer surface of the expandable structure. For example, the lower surfaces may be flat or may be contoured to the expandable structure surface.

In some examples, at least some of the stress-applying features may comprise posts having hemispherical upper surfaces and lower surfaces attached to the outer surface of the expandable structure.

In some examples, the expandable structure may comprise an inflatable balloon. For example, the inflatable balloon may have a distensibility below or not more than 10% when inflated to a pressure of at least 8 atm, at least 10 atm, at least 12 atm, at least 16 atm, at least 18 atm, or at least 20 atm.

In some examples, the stress-applying features are attached to the outer surface of the expandable structure by at least one of an adhesive bonding, ultrasonic welding, fusion, heat welding, a fastener, solvent bonding, bonding with a polymeric material, or combinations thereof. In a preferred example, the plaque disrupting features are bonded using 2 or more adhesives one adhesive adheres better or is more compatible with the expandable structure material while the other adhesive adheres better or is more compatible to the feature material.

In some examples, the inflatable balloon has a central surface region, a distal tapered surface region, and a proximal tapered surface region where the stress-applying features or stress-applying features are present on one or more of these surface regions. In some examples, the inflatable balloon has a central surface region, distal and/or proximal flat regions, one or more radially protruding surface regions, one or more hour-glass region, and/or one or more oblong-shaped region, where the stress-applying features or stress-applying features are present on one or more of these surface regions. Usually, the stress-applying features or stress-applying features are present on at least the central region. More usually, the stress-applying features are present on at least one of the distal and/or proximal flat or tapered regions, or radially as formed protruding surface regions, and sometimes, the stress-applying features are present on both of the distal and proximal tapered regions. In some instances, the stress-applying features are present on at least the central region, wherein the central region adjacent to the proximal and/or distal taper of the balloon are void of stress-applying features. In some instances, the stress-applying features are present on at least the central region, wherein the central region near to the proximal and/or distal taper of the balloon are void of stress-applying features, said region ranging in length from 0.1 mm to 3 mm are void of stress-applying features.

In some examples, the apparatus may further comprise an outer sleeve having plaque disrupting features on the inner surface or on the outer surface of the sleeve, wherein the sleeve is advanced or retracted over an expandable structure such as a balloon typically in-vivo, where the outer sleeve features protrude radially outwardly before, upon advancing or retracting the sleeve over the balloon, or upon inflation of the balloon to the expanded configuration. The sleeve conforms to the stress-applying features when the expandable structure is expanded, wherein the elastomeric tubular member is configured to expand and contract with the expandable structure expansion and contraction.

In another example, an elastomeric tubular member may be placed over and expandable structure such as a balloon containing plaque disrupting features, where the sleeve shields the stress-applying features as the apparatus is advanced or retracted in a body vessel or lumen. The sleeve may be laminated or attached to at least a portion of the outer surface of the expandable structure. For example, the elastomeric tubular member may be attached to the expandable balloon segment, to a segment distal to the expandable balloon segment, or to a segment proximal to the balloon segment. Alternatively, the outer sleeve may comprise an elastic, a non-distensible or semi-compliant sheath covering or folded over the balloon before the balloon is inflated. In most instances, the outer sleeve fully covers the clot disruption features on the outer surface of the expandable structure, typically comprising a polymer. In some examples, the outer sleeve comprises perforations sufficient to allow at least some of the stress-applying features protrude outwardly radially through said perforations when the expandable structure is expanded. In a preferred example, said perforations permit the contact surface region of the stress-applying features to protrude through said perforations.

The sleeve may be formed from a non-compliant polymer, a semi-compliant polymer, and/or from a shape memory polymer, where the polymer preferably has a glass transition temperature below body temperature. Such polymer sleeves can be reinforced with an expandable/retractable metal or metallic alloy frame such as shape memory nitinol or superelastic nitinol, supported by an internal metal or metallic alloy frame such as shape memory nitinol or superelastic nitinol, or combination thereof. If the metal or metallic alloy are not self-expandable, these polymer sleeves with reinforcement can first be opened by expanding the inner balloon to an appropriate diameter. The balloon is then deflated and advanced into the aortic valve. The sleeve can be folded or crimped such that it self-expands in the body above its glass transition temperature of at least 30° C. Polymer sleeves with nitinol reinforcement can have any glass transition temperature since the metal or metallic alloy reinforcement affords the expansion of the sleeve. Polymer materials include but not limited to high durometer silicone such as polydimethylsiloxane, poly(diphenyl) siloxane, poly(methyl-co-phenyl) siloxane, poly(methyltrifluoropropyl) siloxane, poly(methyl-co-methyltrifluoropropyl) siloxane, or the like, polyethylene, polypropylene, polyamide, Pebax, polyurethanes and their copolymers such as bionate, desmocoll, Texalan, Neusoft, tecoflex, pellathane, chronoflex, chronoprene, chronothane, chronosil, polyblend, or the like, polyethylene-vinyl acetate, polyvinylidene fluoride, polyvinylidene fluoride-co-hexafluoropropylene, polybutylmethacrylate, poly(styrene-butadiene-styrene), or the like.

For example, at least some of the stress-applying or stress-applying features may be attached to an inner surface of the elastomeric tubular member or expandable balloon. In particular instances, at least some of the stress-applying features may be formed as a protrusion from the inner surface of the elastomeric tubular member and are pushed to protrude radially outwardly over the elastomeric tubular member or balloon when the expandable structure is advanced or retracted inside said tubular member and/or when the expandable structure is expanded inside the elastic tubular member, or when the balloon is inflated.

In some examples, the apparatus may further comprise an outer sleeve or a balloon member having radially outwardly facing protrusion formed the same material of the sleeve or balloon, where the features would be hollow at their base or comprise a hole at their base, and where the features cover, encapsule, or sit onto the outer surface of the sleeve or balloon protrusions. The protrusions provide a larger surface area to attach to the features. The protrusions typically have a configuration that would fit within or inside the feature, where the fit ranges from lose fit to tight fit. The height of the protrusions ranges from 10% of the height of the feature to 100% of the height of the feature.

In some examples, the apparatus may further comprise an outer sleeve or a balloon member having radially inwardly facing protrusion, crater, or indentation formed the same material of the sleeve or balloon, where the features would sit in the outer surface of the sleeve or balloon indentation, and where the indentation covers partially the feature surface ranging from covering 10% to 60% or more of the feature surface. The indentation provides a larger surface area to attach to the features to secure it when the sleeve or balloon are advanced and/or retracted in a patient body, or when the sleeve and/or balloon are expanded to disrupt a hardened plaque. The indentations typically have a configuration that would partially contain the feature. In some examples, the feature fits snugly within the indentation. In other examples, the feature fits tightly within the indentation. In some other examples, the feature or feature base stretches the indentation. In yet another example, the indentation has a shape or configuration that contours to the shape of the feature or feature base. In indentation typically is coupled to or attached to the feature by press fit and/or one or more adhesive material covering the surface of the indentation and/or feature surface and/or feature base surface.

In a further aspect, apparatus for treating hardened lesions and/or calcifications on a wall in a patient's body lumen in accordance with the present invention comprise a catheter including a catheter body having a proximal end and a distal segment. An expandable structure, typically an inflatable balloon, more typically a non-distensible balloon of the type used in angioplasty and valvuloplasty procedures, is disposed on or near the distal segment of the catheter body and has an outer surface configured to be displaced radially outwardly toward an inner surface of the body lumen wall and/or configured to be displaced radially and axially, and/or configured to be displaced axially. One or more, typically a multiplicity of indentations (e.g., cavities, recesses, wells, voids, pockets, sockets, or other concavities) are distributed over at least a portion of the outer surface of the expandable structure, and a plurality of stress-applying features are received, typically being cradled and immobilized, in at least one, more typically in at least some of the individual indentations.

In some instances, the stress-applying features have a distribution density over at least a portion of the outer surface of the expandable structure in a range from 0.001 to 5 features/mm², often from 0.1 to 5 features/mm², preferably from 0.2 to 4 features/mm², more preferably from 0.25 to 3 features/mm² when the expandable structure is expanded.

In some instances, at least some of the stress-applying features have a convex rounded apex protruding above the outer surface, said convex rounded apex being configured to fracture the calcification while minimizing damage to the body lumen when the expandable structure is expanded within the body lumen.

In some instances, convex rounded apex of the stress-applying feature has a radial height above the outer surface of the expandable structure in a range from a minimum of 0.05 mm, 0.1 mm, 0.15 mm, 0.2 mm, 0.25 mm, 0.5 mm, and 0.75 mm to a maximum of 1 mm, 0.5 mm, 0.4 mm, 0.3 mm, or 0.25 mm.

In some instances, at least some of the stress-applying features have an upper surface flush with the outer surface, optionally being secured by an adhesive, press fitting, encapsulation, ultrasonic welding, and/or combinations thereof.

In some instances, at least some of the stress-applying features have an upper surface recessed below the outer surface of the expandable structure.

In some instances, at least a portion of the outer surface of the expandable structure and the stress-applying features is free from any covering structure.

In some instances, an encapsulation layer covers at least a portion of the outer surface of the expandable structure and the stress-applying features to immobilize the stress-applying features in a desired pattern on the outer surface of the expandable structure.

In some instances, the indentations have an average width and/or depth in a range from 0.05 mm to 1.5 mm, preferably from 0.1 mm to 0.5 mm, more preferably ranging from 0.1 mm to 0.25 mm.

In some instances, at least some of the indentations in balloon wall are configured to inhibit changes in dimensions as the balloon is inflated. For example, at least some of the indentations in balloon wall may be configured to inhibit changes in dimensions as the balloon is inflated to nominal diameter. Alternatively, at least some of the indentations in balloon wall may configured to inhibit changes in dimensions as the balloon is inflated to maximum rated diameter.

In some instances, at least some of the indentations in balloon wall are reinforced.

In some instances, at least some of the indentations in balloon wall are configured to constrict a neck of the indentation as the balloon is inflated to nominal diameter. For example, at least some of the indentations in balloon wall are configured to constrict a neck of the indentation as the balloon is inflated. Alternatively, at least some of the indentations in balloon wall may be configured to constrict a neck of the indentation as the balloon is inflated to maximum rated diameter.

In some instances, the indentations are staggered or patterned along a length and/or circumference of the balloon.

In some instances, the balloon has one or more of cylindrical surfaces, conical surfaces, and opposed surfaces, and the stress-applying features are disposed over one, some or all of these surfaces.

In some instances, the stress-applying features are harder than the outer surface of the expandable structure.

In some instances, the stress-applying features comprise at least one of a metallic, polymeric, or ceramic material.

In some instances, the stress-applying features may be atraumatic, atraumatic with a dull, blunt, smoothed, atraumatic or other coating, encapsulation al or other encasement.

In some instances, the stress-applying features may be roughened by sand blasting or other means to enhance fracture or enhance adhesive or enhance encapsulation of the material.

In some instances, the stress-applying features comprises a magnet or magnetizable material.

In some instances, the stress-applying features comprise one or more of spheres, hemispheres, sections of spheres, disks, cylinders, and cones.

In some instances, the stress-applying features have a base and crown, wherein the bases of at least some of the stress-applying features are disposed in at least some of the plurality of pre-formed indentations.

In some instances, at least some of the stress-applying features comprise a core material encapsulated in a hardened material.

In some instances, at least a portion of the base of at least some of the stress-applying features is encapsulated in the hardened material.

In some instances, at least a portion of the crown of at least some of the stress-applying features is encapsulated in the hardened material. For example, at least portions of both the base and the crown of at least some of the stress-applying features are encapsulated in the hardened material. In other examples, an entire outer surface of at least some of the stress-applying features is encapsulated in the hardened material. Typically, the core material comprises at least one of a polymeric, a metallic, and a ceramic material and the hardened material comprises at least one of a polymeric, a metallic, and a ceramic material having hardness greater than the that of core material.

In some instances, the stress-applying features are distributed partially or fully over a surface of at least one section of the inflatable balloon selected from a group of sections selected from a central cylindrical section, a central depressed, a central waist section, a flat end section, a tapered end section, and a conical end section.

In some instances, at least one surface of at least one section of the inflatable balloon selected from the group of sections selected from a central cylindrical section, a central depressed, a central waist section, a flat end section, a tapered end section, and a conical end section is free from stress-applying features distributed thereover.

In some instances, the inflatable balloon comprises a segmented balloon structure disposed at the distal end of the catheter body, said segmented balloon structure having opposed internal walls configured to be expanded on opposite surfaces of valve leaflets of a calcified valve to disrupt calcification on the calcified valve.

Individual stress-applying features may be cradled and immobilized or otherwise received in at least some of the individual indentations after fully expanding the expandable structure to the maximum expanded configuration.

Individual stress-applying features may be cradled and immobilized or otherwise received in individual indentations after at least partially expanding the expandable structure to an expanded configuration.

Individual stress-applying features may be cradled and immobilized or otherwise received in individual indentations or at sites where such indentations had previously been present after expanding the expandable structure to an expanded configuration and may be secured or attached to the expandable structure outer surface by adhesive bonding, fusing, or other means to of attachment.

While either or both of the opposed internal wall surfaces of the valvuloplasty balloon or other inflatable or expandable segmented structures of the present invention will usually comprise stress-applying features, as described previously, in some instances, either or both the opposed internal wall surfaces may be free from stress-applying features. In such instances, capture and optional pressing of the calcified leaflets between the opposed will be sufficient to fracture calcifications on the valve leaflets even without the presence of the stress-applying features.

In some instances, the valvuloplasty catheter may comprise non-expandable segmented structures disposed at the distal end of the catheter body. Such non-expandable segmented structures will typically have opposed internal walls configured to be drawn together on opposite surfaces of valve leaflets of a calcified valve to disrupt calcification on the calcified valve. The segmented structures may comprise solid bodies, rigid hollow shells, or the like. While either of both the opposed internal wall surfaces may comprise stress-applying features as described above for the expandable structures, in some instances, either or both of the opposed surfaces will be free from stress-applying features. These segmented, non-expandable structures may be formed from polymeric materials, metallic materials, ceramic materials, or other material, or combinations thereof.

In a still further aspect, apparatus for treating calcifications on a wall in a patient's body lumen in accordance with the present invention comprise a catheter including a catheter body having a proximal end and a distal segment. An expandable structure is disposed at the distal segment of the catheter body, said expandable structure has an outer surface configured to be displaced radially outwardly toward an inner surface of the body lumen wall. A plurality of stress-applying features are distributed over the outer surface of the expandable structure, and an energy source is configured to deliver energy to or through the plaque disrupting feature to enhance plaque disruption.

The energy source may deliver any type of energy that can be delivered to the stress-applying features so that the features can focus, concentrate, transduce or otherwise convert the energy into mechanical, electrical, and/or heat energy that can enhance fragmentation of plaque in contact with the stress-applying features. For example, the energy sources may comprise any one or more of ultrasound or other mechanical vibration sources, laser or other light or optical energy sources, high voltage electrical and other electrical energy sources (e.g. capable of generating an arc with or across a stress-applying features). The energy may be delivered through the balloon inflation media, for example from internal or external ultrasound sources. In other cases, the energy may be delivered through conductors, conduits, or the like, chosen for the type of energy to be delivered to the stress applying features (e.g., electrical conductors for electrical energy, optical fibers or wave guides for laser and other optical energy.

The energy source may be disposed within the balloon or other expandable structure and may be configured to deliver mechanical or electrical energy through the balloon medium to the stress-applying features to enhance plaque fragmentation. For example, an ultrasound transducer may be positioned to deliver ultrasound energy, typically from kHz 0.5 kHz to 50 kHz, more typically from 2 kHz to 25 kHz, into the balloon inflation medium, such as saline, so that the energy propagates through the inflation medium to cause the stress-applying features to vibrate. Plaque fragmentation may be enhanced directly by the vibration of the plaque (mechanically), by heat resulting from the vibration (thermally), or by a combination of both mechanical and thermal effects.

The ultrasound transducers will typically be positioned inside the balloon to deliver vibrational energy directly into the inflation medium. In other instances, an ultrasound transducer can be attached to an exterior surface of the balloon or other expandable structure and be configured to deliver the vibration energy through the balloon wall and into the inflation medium.

In still other instances, it would be possible to deliver a focused ultrasound from a source external to the patient.

In still other instances, the stress-applying features themselves may be configured to vibrate or resonate with the direct or indirect delivery of electrical or other energy. For example, the stress-applying features may be formed as piezoelectric structure that vibrate with the delivery of electrical current.

The stress-applying features of the present invention may be combined with or incorporated into the balloon structures of known cavitation-inducing “lithotripsy” catheters, such as those described in PCT Publication Nos. WO2013/070750; WO2015/017499; WO2018/194752; WO2021/061451; WO2020/256949; WO2021/18367; and WO2022/216488, the full disclosures of which are incorporated herein by reference. In particular, outer balloon or other expandable member surfaces of such lithotripsy catheters can incorporate any of the stress-applying features described herein, where the features will enhance the calcification disruption performance of the catheters.

In yet another aspect, apparatus for treating hardened plaque or calcifications on a wall in a patient's body lumen in accordance with the present invention comprise a catheter including a catheter body having a proximal end and a distal segment. An expandable structure is disposed at the distal segment of the catheter and has an outer surface configured to be displaced radially outwardly toward an inner surface of the body lumen wall, and/or configured to be displaced axially. One or more (a plurality) of stress-applying features are distributed over the outer surface of the expandable structure, wherein at least some of the stress-applying features are harder than the outer surface of the expandable structure.

In some instances, the stress-applying features comprise at least one of a metallic, polymeric, or ceramic material.

In some instances, the stress-applying features is atraumatic, being coated, dull, or blunt.

In some instances, all or a portion of the stress-applying feature may be roughened by sand blasting or other means to enhance fracture or enhance adhesive or enhance encapsulation of the material.

In some instances, the stress-applying features may comprise a magnet or

magnetizable material.

In some instances, the stress-applying features may comprise spheres, hemispheres, sections of spheres, disks, cylinders, and cones.

In some instances, the stress-applying features may comprise a core covered by a hardened shell.

Individual or groups of stress-applying feature(s) may be mounted and typically immobilized on protrusions molded or otherwise formed into the outer surface of the balloon after fully expanding the expandable structure to the maximum expanded configuration.

Individual or groups of stress-applying feature(s) may be mounted and typically immobilized on protrusions molded or otherwise formed into the outer surface of the balloon after at least partially expanding the expandable structure to an expanded configuration.

Individual or groups of stress-applying feature(s) may be mounted on (e.g., immobilized on protrusions), molded on, or otherwise secured or attached to an outer surface of an expandable structure by adhesive bonding, fusing, or other means to of attachment, or otherwise formed into the outer surface of the balloon after expanding the expandable structure to the expanded configuration.

In an additional aspect, apparatus for treating calcifications on a wall in a patient's body lumen in accordance with the present invention comprise a catheter including a catheter body having a proximal end and a distal segment. A balloon has plurality of pre-formed indentations formed over at least a portion of an outer surface thereon. The balloon is attached on or to the distal segment of the catheter body, and bases of a multiplicity of stress-applying features are disposed in at least some of the plurality of pre-formed indentations, and crowns of at least some of the stress-applying features are exposed above, on, or beneath the outer surface of the balloon when the balloon is at least partially or when the balloon is fully expanded.

In some instances, at least some of the stress-applying features comprise a core material encapsulated in a hardened material.

In some instances, at least a portion of the base of at least some of the stress-applying features is encapsulated in the hardened material.

In some instances, at least a portion of the crown of at least some of the stress-applying features is encapsulated in the hardened material.

In some instances, at least portions of both the base and the crown of at least some of the stress-applying features are encapsulated in the hardened material.

In some instances, an entire outer surface of at least some of the stress-applying features is encapsulated in the hardened material.

In some instances, the core material comprises at least one of a polymeric material and a ceramic material and the hardened material comprises at least one of a metallic material, a polymeric material, and a ceramic material having a hardness greater than that of the core material.

In some examples, at least some of the stress-applying features may have a base attached to the outer surface of the expandable structure, said base having a width in an axial direction (Wa) and a width in a circumferential direction (Wc) with a width ratio Wa:Wc in a range from 1:0.5 to 1:5; usually from 1:1 to 1 to 1:5; more usually from 1:1 to 3:1. For example, at least some of the bases may have a circular periphery or may have an oval periphery. In other examples, the Wa:Wc is in a range from 3:1 to 1:3, usually ranging from 2:1 to 1:2, and more usually ranging from 1.5:1 to 1:1.5, and most usually about 1:1. In some examples, the base is an integral part attached to the stress-applying features, and is attached to the expandable structure surface. In other examples, the base is a separate part from the plaque disrupting feature and is attached to the plaque disrupting feature and attached to the expandable structure surface.

In some examples, at least some of the stress-applying features may be arranged in diametrically opposed pairs. For example, successive diametrically opposed pairs of stress-applying features may be circumferentially offset. For example, the successive diametrically opposed pairs of stress-applying features may be circumferentially offset by an angle from 45° to 90°.

In some examples, at least some of the stress-applying features may have a base attached to the outer surface of the expandable structure, wherein the base of each feature in the group of features about the expandable structure circumference in the expanded configuration do not overlap one another. In other examples, the base of each of said features do not overlap with other base of features located along a certain axial length of the expandable structure, or along the length of the expandable structure axial length. In yet another example, the base of each feature does not overlap another feature base along the circumference of an expandable structure when expanded and does not overlap an axially adjacent feature along the axial length of the expandable structure when the structure is expanded.

In some examples, at least some of the stress-applying features may be arranged in groups of three which are circumferentially separated about a circle on the surface of the expandable structure by about 120°.

In some examples, at least some of the stress-applying features may be arranged in groups of 2 which are circumferentially separated about a circle on the surface of the expandable structure by about 1800.

In some examples, at least some of the stress-applying features may be arranged in groups of 4 which are circumferentially separated about a circle on the surface of the expandable structure by about 90°.

In some examples, at least some of the stress-applying features may be arranged in groups of 2-4 each which are circumferentially separated about a circle on the surface of the expandable structure by about 90° to 180°, wherein each group forms a helical pattern along the length of the expandable structure when the structure is in the expanded configuration.

In some examples, at least some of the stress-applying features maybe arranged in groups of 3 to 10 features per group which are circumferentially separated about a circle on the surface of the expanded structure by about 36° to 120° wherein each feature footprint or base about said circle overlaps no more than one other feature footprint or base along said circle.

In some examples, at least some of the stress-applying features maybe arranged in groups of 3 to 10 features per group which are circumferentially separated about a circle on the surface of the expanded structure by about 36° to 120° wherein each feature footprint or base about said circle overlaps no more than two other feature footprint or base along said circle, and wherein each feature about said circle overlaps no more than one to five other features along an axial length path of the expandable structure when the structure is expanded, preferably along the entire length of the expanded structure.

In some examples, at least some of the stress-applying features maybe arranged in groups of 3 to 10 features per group which are circumferentially separated about a circle on the surface of the expanded structure by about 36° to 120° wherein each feature footprint or base from each group about said circle does not overlap other features footprint or base from the same group along said circle, and wherein each feature about said circle also does not overlap other features from other groups along an axial length path of the expandable structure when the structure is expanded, preferably along the entire length of the expanded structure.

In some examples, at least some of the stress-applying features maybe arranged in groups of 3 to 10 features per group which are circumferentially separated about a circle on the surface of the expanded structure by about 36° to 120° wherein each feature footprint or base from each group about said circle has a gap when the underlying structure is fully expanded between said feature and other features footprint or base of the same group said gap ranging from 0.05 mm to 2.5 mm, preferably ranging from 0.1 mm to 1.5 mm. In a preferred example, the gap between any two features of the same group is measured along an axial length between said two features footprint or base circles.

In some examples, at least some of the stress-applying features maybe arranged in groups of 3 to 10 features per group which are circumferentially randomly separated about a circle on the surface of the expanded structure by about 10° to 180°.

In some examples, the stress-applying features maybe arranged in a helical pattern along a length of the expandable structure, preferably along the entire length of the expandable structure when the structure is in the expanded configuration. In some instances, the helical pattern of at least some stress-applying features completes one to five 360° turns along said length, or along the entire length of the expanded structure. In some examples, the plaque disrupting features or features bases do not overlap about the circumference of the expandable structure and/or axial length.

In some examples, the stress-applying features maybe arranged in a straight pattern along a length of the expandable structure, preferably along the entire length of the expandable structure when the structure is in the expanded configuration.

In some examples, the stress-applying features may be arranged in one region of the expandable structure. For example, the plaque disrupting features may be arranged in a mid-region of the expandable structure, such as a cylindrical or other working length of the structure, such as an inflatable balloon.

In other examples, the inflatable balloon coupled to plaque disrupting features maybe arranged to prevent the outer surface of the expandable structure surface such as balloon outer surface from contacting hardened plaque or tissue, or are arranged to have the outer surface of said balloon not contact hardened plaque before a minimum threshold pressure, wherein said minimum threshold pressure maybe any one of the following nominal or rated balloon inflated pressures: 3 atm, 5 atm, 7 atm, 10 atm, 12 atm, 15 atm, or other.

In other examples of the present invention, the expandable structure such as inflatable balloons may be further configured to deliver drugs and other medicaments. For example, the inflatable balloon may be configured to release an inflation medium comprising a medicament in response to an inflation pressure above a minimum threshold value, e.g., where the minimum threshold value is above 1 atm, 3 atm, 5 atm, or 7 atm. In specific instances, the inflatable balloon may comprise a plurality of ports or perforations within the balloon material surface adjacent to at least some features which open in response to the inflation pressure above the minimum threshold pressures. In some other examples, the inflatable balloon may have a separate a distal conduit formed from an elongated tubular body extending from proximally to said distal conduit to outside the patient to infuse medicaments while the balloon is in the expanded configuration. In yet another example, the medicaments are coated onto one or more of the stress-applying features surfaces, more particularly the engaging or contact surface region of the stress-applying features, to provide medicaments to the tissue when the features contact the vessel or annulus or leaflet tissue. In one example, the medicaments are coated or sprayed directly onto at least the contact region surface of at least some of the features. In other examples, the medicaments are mixed with a polymeric material and the mixture is then coated or sprayed onto at least the contact region of the stress-applying features. In yet another example, the medicaments are sprayed or coated onto the outer surface of the expandable structure outer surface such as balloon and/or stress inducing features. In yet another example an expandable structure comprising an expandable balloon segment wherein the balloon segment is covered by an outer sleeve comprising an elastomeric member, and wherein the balloon and/or elastomeric member comprises at least some stress-applying features attached to the outer surface of the balloon, the outer surface of the elastomeric member, and/or attached to the inner surface of the elastomeric member, wherein the outer surface of the elastomeric member is coated with a medicament comprising one or more drugs. The drugs covering the at least some features embed into the vessel wall, body lumen, or valve annulus, when the balloon is inflated to embed at least part of its medicament content into the adjacent tissue. In a preferred example, the medicaments comprise one or more drugs of an anti-proliferative drug, an m-tor inhibitor drug, a taxol or analogues drug, a direct thrombin inhibitor drug, and a factor Xa inhibitor drug. In yet another example, the medicament is located in spaces between the balloon segment outer surface and the elastomeric segment inner surface and is allowed to penetrate the elastomeric member through apertures or perforation in the elastomeric member when the balloon is expanded to release said medicaments into the vessel wall, body lumen, or valve annulus.

In a particular example, an expandable structure comprises a balloon having an outer surface. The outer surface typically comprises a plurality of stress-applying features as described elsewhere herein. The outer surface of the balloon and/or the stress-applying features are coated with a formulation of one or more drugs comprising an anti-proliferative, an anticoagulant, and an anti-platelet agent. Examples of anticoagulants comprise a direct factor Xa inhibitors, a direct factor IIa inhibitors, and the like. Examples of antiproliferative comprise rapamycin, analogues, and derivatives of rapamycin, Taxol, analogues, and derivatives of Taxol. The formulation of one or more drugs may further comprise or contain one or more excipient(s); one or more plasticizer(s); and and one or more of contrast agent(s) (such as Iopromide); one or more polymeric material(s) such as PLLA or PLGA, or PCL; Acetyle tributyl citrate; one or more cationic surfactant(s), such as urea, polyethyleneimine; Butryl-tri-hexyl citrate; a UV-cured material, such as PVP hydrogel; a non-ionic surfactant such as polysorbate/sorbitol; an amphilic polymer, such as PEG: an encapsulated drug, such as an encapsulated nano-particle drug; a micelle-encapsulated drug, and phospholipid-encapsulated drug; PLGA microspheres; and combination thereof.

In still other aspects, the present invention provides a method for treating calcification on a wall in a patient's body lumen, where the method comprises positioning an expandable structure at a treatment site proximate the calcification to be treated and radially outwardly expanding the expandable structure to radially outwardly press a plurality of stress-applying features against the calcification. The stress-applying features are distributed over the outer surface of the expandable structure and at least some of the stress-applying features are independently attached to the outer surface of the expandable structure and have convex rounded upper surfaces and wherein radially outwardly pressing the plurality of stress-applying fractures into the calcification fractures the calcification while reducing damage to the wall.

In still other aspects, the present invention provides a method for treating calcification on a patient's valve having calcified leaflets, where the method comprises positioning an expandable structure at a treatment site proximate the calcification to be treated (such as within or though calcified leaflets) and radially outwardly expanding the expandable structure radially outwardly such that the stress-applying feature press the calcified leaflets against the wall of the annulus fracturing the calcified leaflets. The stress-applying features are distributed over the outer surface of the expandable structure and at least some of the stress-applying features are independently attached to the outer surface of the expandable structure and have convex rounded upper surface.

In yet another aspect of the present invention, an apparatus for treating a patient valve having calcified leaflets comprises a catheter body and a segmented balloon structure. The catheter body has a proximal end and a distal end. The segmented balloon is disposed at the distal end of the catheter body and has opposed internal walls (side walls) configured to be expanded on opposite surfaces of the calcified leaflets in a manner which disrupts calcifications on the calcified valve leaflets. In one example, the internal side walls (or axially facing walls) of the segmented balloon are flat or protruding axially outwardly when the balloon is expanded pressing against the calcified leaflets surfaces from opposite sides disrupting calcification on the calcified valve. In yet another example, at least one of the segmented balloon internal side walls comprise one or more stress-applying features coupled to said side walls and configured to engage the calcified valve leaflet when the balloon is expanded to disrupt said calcification, wherein the features press said calcification against the opposite internal side wall of the segmented balloon. In yet another example, the segmented balloon internal side walls comprise one or more stress-applying features coupled to the walls and configured to engage the calcified valve leaflet from opposite sides when the balloon is expanded to disrupt said calcification. In yet another example, the number of stress-applying features range from 0.1 to 100 features/mm², preferably range from 0.1 to 30 features/mm² of the internal side walls surface, more preferably range from 1 to 20 features/mm² of the internal side walls surface. In yet another example, the stress-applying features may be arranged over the side surface of the expandable structure in a variety of configurations such as circular pattern, circle within circle pattern, spiral pattern, valve leaflet conforming or contouring pattern, or other patterns. In another example, at least some of the stress-applying features on one side of the segmented balloon internal wall are configured (or arranged) to oppose other stress-applying features on the opposite internal wall. In another example, at least some of the stress-applying features have blunt contact regions on one side of the segmented balloon internal wall are configured (or arranged) to oppose other stress-applying features blunt contact region on the opposite internal wall when the balloon is expanded, disrupting calcification between said opposing blunt contact regions. In another example, at least some of the stress-applying features contact region configured to have a convex shape on one side of the segmented balloon internal wall are configured (or arranged) to oppose other stress-applying features contact region on the opposite internal wall configured to have a concave shape to fit into the convex opposing shaped features when the balloon is expanded, disrupting calcification between said opposing blunt contact regions.

In yet another example, at least some features on one side of the internal wall are configured to invaginate, or at least partially invaginate the space between two, three, four, or more features on the opposite internal side wall of the segmented balloon when the balloon is inflated, disrupting calcification in a valve leaflet. In another example, the side and/or internal walls maybe covered by an elastomeric member covering at least some of the features and optionally said elastomeric member is attached to a balloon segment or other segment of the balloon catheter and is configured to expand when the balloon is expanded.

In another example, the internal side walls and/or the elastomeric member are configured to expand radially and/or axially when the balloon is inflated (or expanded fully) to press against calcified valve leaflet and disrupt calcification of the leaflet. In alterative examples, segments of the balloon structure slidably attached to or mounted on a shaft of the catheter and are configured to draw the internal side walls together after inflation, optionally being configured to nest when the balloon structure is expanded, typically having nesting conical surfaces.

In still other examples, the opposed internal walls may comprise flat surfaces that are configured to close against each other when the balloon structure is inflated and/or expanded. For example, the segmented balloon structure may comprise a pair of opposed conical balloons having flat bases which comprise the flat surfaces.

In some examples, the calcification-disrupting features on the opposed internal wall surfaces are axially aligned as the balloon segments are drawn together, this applying force to opposed surfaces of the valve leaflet. In other instances, the calcification disruption features on the opposed surfaces are laterally offset so that they do not align axially as the balloon segments are drawn together. In still other instances, the calcification-disrupting features may be on only one of the two the opposed internal wall surfaces.

In yet another example, at least one region of the internal segmented balloon side walls and/or at least one region of the elastomeric member cover and/or at least some of the features are coated with one or more coating comprising a polymeric material, adhesive material, an anti-proliferative agent, a factor Xa inhibitor agent, and a factor IIa inhibitor agent, wherein the coating material is configured to deliver a drug, affix debris of the disrupted calcium to the leaflet or to the internal wall of the segmented balloon, or repair a perforated leaflet. In another example, the coated material is configured to attach to the leaflet, transfer material or drug to the leaflet surface, adhere to the leaflet surface, repair a perforation in the leaflet surface, or hold the leaflet together.

In another example, at least some of the features on opposite sides of the internal side walls of the segmented balloon fit into one another such as a concave and convex contact surface regions, a ball and socket contact surface regions, or other. In some examples, the calcium disrupting features on the expandable structure such as an expandable balloon maybe arranged on one or more of the expandable structure surfaces comprising side surface, internal surface, or axially facing surface. In some examples, the expandable structure such as an expandable balloon comprise one or more shapes comprising tubular shape, donut shape, hourglass shape, tapered shape, oblong shape, rectangular shape, square shape, or other. In yet another example, the axially facing region of the expandable structure may have various shapes comprising one or more of flat, convex, concave, donut, or other.

In another example of the present invention, an apparatus for treating a patient valve having calcified leaflets comprises a catheter body having at least two expandable structures, where the at least two expandable structures in one example are configured to expanded together such as a dual balloon configuration where the dual balloon share the same inflation lumen and the same guide wire lumen. In another example, the at least two expandable structures are configured to expanded independently such as at least two balloon structures having separate inflation lumen and same or separate guide wire lumen to the at least two balloon structures. In a preferred example, a dual expandable structure is axially movable over a common axial tubular structure.

In specific instances, the valve calcification treatment apparatus will further comprise a plurality of calcification disruption features distributed over at least one of the opposed internal walls of the segmented balloon structure, preferably over both of the opposed internal walls (and/or axial facing wall). The calcification disruption features may comprise any of the stress-applying and calcification disrupting features described in the present application, typically comprising rounded features, including those hemispherical, balls, and spherical features as described herein.

Preferred disruption features will usually have convex rounded leaflet engaging surfaces configured to fracture the calcification while minimizing damage to the leaflet when the balloon structure is expanded in the patient valve. Usually, the convex rounded upper surfaces of the stress-applying features will be configured to extend from the opposed internal walls so as to engage the leaflets when they are captured between the walls. The rounded surfaces of the features that engage the leaflet will typically have a height or width in a range from 0.01 mm to 3 mm, from 0.1 mm to 3 mm, and usually from 0.5 mm to 2 mm, when the balloon is fully inflated. Most often, the convex rounded upper surfaces of the calcification disruption features will be free from edges and irregularities which could damage the leaflets when the balloon structure is inflated within the patient valve.

In some instances, however, the calcification disruption features may be provided with a sharp element projecting outwardly from the convex rounded upper surface where the sharp element is configured to concentrate stress when engaged against the calcification on the valve leaflet as the balloon surface presses against the surface of the valve leaflet. The sharp features will have a very minimal height or depth so that they will engage and disrupt calcifications while substantially avoiding any injury to the valve leaflet. The individual balloon segments may be fixed to the catheter body so that they engage and capture the leaflets when the balloon structure is inflated without any further manipulation. In other instances, however, the balloon segments may be configured to axially translate relative to each other on the catheter body to provide a variable spacing between the internal walls. In such instances, a first of the segments may be inflated and engaged against the valve leaflets and the second segment inflated and then drawn against the opposed surface of the valve leaflet in order to effect disruption.

In yet another aspect, the present invention provides a method for disrupting a calcification or plaque at a lesion comprising advancing a sleeve over a wire through the lesion, advancing an expandable member over the wire and into an interior of the sleeve, and expanding the expandable member within the sleeve to outwardly radially displace features on an inside and/or outside of the sleeve against the lesion to disrupt the calcification or plaque.

In some examples, the method further comprises removing the expandable member from the sleeve and removing the expandable member and the sleeve over the wire, where the expandable structure may comprise either one of a balloon or other expandable member or a stent and wherein the sleeve is left in place between the stent and the lesion after the stent is expanded. The stress-applying features may protrude radially outwardly from the sleeve into the vessel wall when the expandable structure displaces them radially outwardly upon expansion.

In a still further aspect, the present invention provides a method for disrupting a calcification or plaque at a lesion comprising advancing a cage or basket over a wire across the lesion and expanding the cage of basket to radially displace stress-applying features on the cage or basket against the lesion to disrupt the calcification or plaque.

In some examples, expanding the cage or basket comprises mechanically reorienting structural components of the cage or basket. In other examples, expanding the cage or basket comprises inflating a balloon within the cage or basket, where the balloon may be advanced to the lesion together with the cage or basket or after the cage or basket.

In yet another aspect of the present invention, apparatus for treating calcification on a wall in a patient's body lumen comprises a catheter including a catheter body having a proximal end and a distal segment. An expandable structure is disposed at the distal segment of the catheter body and has an outer surface configured to be displaced radially outwardly toward an inner surface of the body lumen wall. A plurality of stress-applying features are distributed over at least a portion of the outer surface of the expandable structure, where at least some of the stress-applying features are disposed on the outer surface of the expandable structure and have a convex rounded apex configured to fracture the calcification while minimizing damage to the body lumen when the expandable structure is expanded within the body lumen.

In specific instances, the convex rounded apex at least some of the stress-applying features have a radial height above the outer surface of the expandable structure in a range from a minimum of 0.05 mm, 0.1 mm, 0.15 mm, 0.2 mm, or 0.25 mm to a maximum of 1 mm, 0.5 mm, 0.4 mm, 0.3 mm, or 0.25 mm, and a distribution density in a range from 0.1 to 5 features/mm², preferably from 0.2 to 4 features/mm², more preferably from 0.25 to 3 features/mm² when the expandable structure is expanded.

In other specific instances, the stress-applying features may have a footprint with a maximum width, diameter, or other lateral dimension of 4 mm or less, often being 3 mm or less, more often being no more than 1 mm, and frequently being no more than 0.75 mm, and sometimes being no more than 0.5 mm.

The stress-applying features may have any one of or combination of characteristics, for example comprising any one or more of balls, spheres, hemispheres, partial spheres, domes, and ellipsoidal geometries; being solid; being hollow, being coated, being uncoated, having textured surfaces, having smooth surfaces; may be discrete bodies; may comprise a metal, polymer, or combinations thereof.

In preferred aspects, an encapsulation layer may cover a portion or all of the outer surface of the expandable structure and/or the stress-applying features to immobilize the stress-applying features in a desired pattern on the outer surface of the expandable structure.

In specific examples, the stress-applying features may be immobilized solely by encapsulation layer or may be immobilized by an adhesive between the feature and the outer surface in addition to the encapsulation layer. The encapsulation layer may encapsulate the entire stress-applying feature including the convex rounded apex or the encapsulation layer may encapsulate only a lower portion of the stress-applying feature excluding the convex rounded apex.

In preferred examples, the stress-applying feature may be cradled in an indentation, cavity, recess, receptacle, or other depression in the outer surface of the expandable structure. Such cradling helps secure and stabilize the stress-applying features during introduction and use.

The encapsulation layer may comprise one or more polymers selected from the group consisting of thermoplastic fluoropolymers (PVDF), butyl methacrylate (PBMA), and thermoplastic polyesters (PLLA), and the like.

The encapsulation layer is applied over the outer surface and the stress-applying features by any one of coating, direct fluid application, laminating, and fusing.

The encapsulation layer may have a thickness in a range from 0.01 mm to 0.1 mm (0.5 mil to 5 mil), often being 0.01 mm to 0.05 mm (0.4 mil to 2 mil), and more often being 0.01 mm to 0.02 mm (0.4 mil to 0.8 mil).

The stress-applying features may be constrained over the outer surface of the expandable structure by an elastic sleeve.

The stress-applying features are typically attached to the outer surface of the expandable structure but in some instances may be attached to an inner surface of the elastic sleeve. In some instances, the stress-applying features are hollow and mounted on a post projecting radially outwardly from the outer surface of the expandable structure.

In another aspect, an apparatus for treating calcification on a wall in a patient's body lumen in accordance with the present invention comprises a catheter including a catheter body having a proximal end and a distal segment. An expandable structure is disposed at the distal segment of the catheter body and has an outer surface configured to be displaced radially outwardly toward an inner surface of the body lumen wall. A plurality of stress-applying features distributed over the outer surface of the expandable structure, wherein at least some of the stress-applying features are present on the outer surface of the expandable structure and have an upper surface configured to fracture the calcification while minimizing damage to the body lumen when the expandable structure is expanded within the body lumen. An encapsulation layer covering at least a portion of the outer surface of the expandable structure and the stress-applying features to immobilize the stress-applying features in a desired pattern on the outer surface of the expandable structure.

In some instances, at least some of the upper surfaces of the stress-applying features comprise a convex rounded apex and the encapsulation layer may cover the entire outer surface of at least some the stress-applying features including the upper surface. Alternatively, the encapsulation layer may cover only a lower portion of the outer surface of at least some of the stress-applying features. Often, the stress-applying feature will be disposed in an indentation, recess, receptacle or other indentation on the outer surface of the expandable structure. Alternatively, the stress-applying feature may comprise a hemisphere with a flat bottom adhered to the outer surface of the expandable structure.

In yet another aspect, the present invention provides a method for treating a lesion on a wall in a patient's body lumen comprising providing a catheter having an expandable structure disposed at a distal end thereof, where expandable structure has an outer surface configured to be displaced radially outwardly toward an inner surface of the body lumen wall and the outer wall has a plurality of space-separating “spacer” features distributed over the outer surface of the expandable structure. The expandable structure is expanded in the patient's body lumen such that a radially outward force is applied by the outer surface and the features against the wall while the features maintain a gap between the outer surface of the expandable structure and the inner wall.

The spacer features are typically configured to create and/or maintain the one or more gaps by separating the lesion from the outer surface of the expandable structure adjacent to the features when the structure is in the expanded configuration. The spacer feature typically comprise a plurality of features positioned in a configuration around the circumferential length and/or axial length of the expandable structure to provide, create, or maintain said gaps.

In one instance, the spacer features have axially aligned through holes permitting the passage of fluids, such as contrast media, blood, and/or drug (medicament) solutions, therethrough. Typically, the gap allows fluid perfusion therethrough and past the expandable structure while the expandable structure is expanded. For example, a drug may be perfused into the gap while the expandable structure is expanded, e.g., the expandable structure comprises a balloon and the drug is perfused through a wall of the balloon. In alternative instances, at least some of the space-separating features comprise a drug which is released into the gap.

In particular instances, expanding the expandable structure creates one or more gaps between the outer surface of the expandable structure and the inner wall under physiological pressure to allow fluid perfusion through the one or more gaps.

In preferred instances, the spacer features may comprise a convex rounded apex as described previously with respect to stress-applying and plaque disrupting features of the present invention.

The spacer features may have a radial height above the outer surface of the expandable structure in a range from a minimum of 0.05 mm, 0.1 mm, 0.15 mm, 0.2 mm, or 0.25 mm to a maximum of 1 mm, 0.5 mm, 0.4 mm, 0.3 mm, or 0.25 mm, and a distribution density in a range from 0.1 to 5 features/mm², preferably from 0.2 to 4 features/mm², more preferably from 0.25 to 3 features/mm² when the expandable structure is expanded.

In such perfusion methods, the expandable structure is typically expanded with a force sufficient to create and/or maintain one or more gaps against a physiologic pressure from 0.5 psi to 5 psi, preferably from 1 psi to 3 psi.

In yet another aspect, the present invention provides method securing stress-applying features on an outer surface of a balloon, such as an angioplasty balloon, a valvuloplasty balloon, or the like. The method comprises providing a balloon having an outer surface and optionally forming a plurality of indentations over at least a portion of the outer surface of the balloon. Optionally, at least a cylindrical or other working length of the outer surface of the balloon and/or a side surface of the balloon is coated with a base layer of an elastic polymer. An elastic adhesive is dispensed onto the balloon outer surface or into individual indentations, over the base layer, and stress-applying features are placed onto the elastic adhesive dispensed onto the outer surface of the balloon or into the individual indentations, causing a portion of the elastic adhesive to be displaced onto the outer surface of the balloon surrounding each plaque disrupting feature. Each stress-applying feature is optionally covered with a spot layer of an elastic adhesive, where the spot layer forms a seal with the elastic adhesive displaced onto the outer surface of the balloon surrounding each plaque disrupting feature. Optionally, a cover layer of an elastic polymer is then formed over at least the working length of the outer surface of the balloon and/or the side surface of the balloon. Usually, one or more of the elastic polymer, the elastic adhesive, the plaque disrupting features, the spot adhesive, and the cover layer are coated, dispensed, or placed after expanding the balloon to the expanded configuration.

In some preferred embodiments, at least one of the base layer and the cover layer will be formed as part of the fabrication method. In other preferred embodiments, both of the base layer and the cover layer will be formed as part of the fabrication method.

In specific aspects, coating the outer surface of the balloon with a base layer of an elastic polymer comprises coating the outer surface with a curable elastic adhesive and curing said elastic adhesive.

In specific aspects, the elastic adhesive dispensed onto the outer surface of the balloon and/or into the indentation comprises a light curable acrylic adhesive.

In specific aspects, the spot layer comprises a light curable acrylic adhesive. Usually, the elastic adhesive dispensed onto the outer surface of the balloon or into the indentation and the spot layer comprise a chemically similar light curable acrylic adhesive, where the two adhesives fuse together when cured.

In a still further aspect, the present invention provides apparatus for treating a patient valve having calcified leaflets, where the apparatus comprises a catheter body having a proximal end and a distal end and a segmented balloon structure. The segmented balloon structure is disposed at the distal end of the catheter body and has opposed internal walls configured to be deployed on opposite surfaces of the leaflets to disrupt calcification on the calcified valve. Features on either or both the opposed internal walls are configured to disrupt calcified plaque on the valve leaflets as the opposed internal walls are deployed.

In some instances, the opposed internal walls are configured to close together when the balloon structure is expanded. In other instances, the segments are configured to be drawn together after the balloon structure is expanded to capture the calcified valve leaflets therebetween.

In some instances, the features comprise plates. In other instances, the features comprise protrusions on one of said opposed internal walls and cavities on the other of said opposed internal walls, wherein the protrusion are configured to nest in the cavities when the opposed surfaces are deployed.

In yet another aspect, the present invention provides methods for securing stress-applying features on an outer surface of a balloon, such as an angioplasty balloon, a valvuloplasty balloon, or the like. The methods comprise providing a balloon having an outer surface and optionally forming a plurality of indentations over at least a portion of the outer surface of the balloon. An elastic adhesive is dispensed onto the balloon outer surface and/or into individual indentations, and stress-applying features are placed onto the elastic adhesive dispensed onto the outer surface of the balloon and/or into the individual indentations, causing a portion of the elastic adhesive to be displaced onto the outer surface of the balloon surrounding each plaque disrupting feature. Each stress-applying feature is optionally covered with a spot layer of an elastic adhesive, where the spot layer forms a seal with the elastic adhesive displaced onto the outer surface of the balloon surrounding each plaque disrupting feature. A cover layer of an elastic polymer is then formed over at least the working length of the outer surface of the balloon and/or the side surface of the balloon. Usually, one or more of the cover layer, the elastic adhesive, the plaque disrupting features, and the spot adhesive is/are coated, dispensed, or placed after expanding the balloon to the expanded configuration.

In specific aspects, coating the outer surface of the balloon with a cover layer of an elastic polymer comprises coating the outer surface with a curable elastic adhesive and curing said elastic adhesive.

In specific aspects, the elastic adhesive dispensed onto the outer surface or into the indentation comprises a light curable acrylic adhesive.

In specific aspects, the spot layer comprises a light curable acrylic adhesive. Usually, the elastic adhesive dispensed onto the outer surface or into the indentation and the spot layer comprise a chemically similar light curable acrylic adhesive, where the two adhesives fuse together when cured.

In yet another aspect, the present invention provides method for resisting balloon rupture in a calcified vessel comprising an outer surface of a balloon, such as an angioplasty balloon, a valvuloplasty balloon, or the like. The method comprises providing a balloon having an outer surface. At least a working length of the outer surface of the balloon and/or a side surface of the balloon is coated with one or more base layers of an elastic polymer, said elastic layer resist rupture of the balloon when the balloon is expanded in a calcified and/or hardened vessel or body lumen. In specific aspects, coating the outer surface of the balloon with a base layer of an elastic polymer comprises coating the outer surface with a curable elastic adhesive and curing said elastic adhesive.

In a still further aspect, the present invention provides apparatus for treating a patient valve having calcified leaflets, where the apparatus comprises a catheter body having a proximal end and a distal end and a segmented expandable or non-expandable structure. The structure is disposed at the distal end of the catheter body and has opposed internal walls configured to be deployed or positioned on opposite surfaces of the leaflets to disrupt calcification on the calcified valve when expanded and/or axially drawn together. Features on either or both the opposed internal walls are configured to disrupt calcified plaque on the valve leaflets as the opposed internal walls are deployed and/or axially drawn together.

In some instances, the opposed internal walls are configured to close together when the structure is expanded. In other instances, the segments are configured to be drawn together to capture the calcified valve leaflets therebetween.

In yet another further aspect, the present invention provides apparatus for treating a patient valve having calcified leaflets. The apparatus comprises a catheter body having a proximal end and a distal end, and a segmented balloon structure disposed at the distal end of the catheter body. The segmented balloon structure has opposed internal walls configured to be deployed on opposite surfaces of the leaflets to disrupt calcification on the calcified valve. The two balloon segments are initially spaced-apart on the catheter body and are configured unfold so that the opposed internal walls converge to capture a calcified leaflet therebetween as the balloon segments are inflated.

In a still further aspect of the present invention, the stress-applying features may be attached to an inflatable balloon or other expandable structures such as a cylindrical expandable structure or a conically shaped expandable structure, or the stress-applying features may be attached to non-expandable structures using a “carrier” template. The carrier template may be fabricated, for example, by rolling or otherwise forming a thin, cylindrical a thin base layer sized to be placed over the balloon or other cylindrical expandable or non-expandable structure in its deployed configuration, i.e., inflated or otherwise expanded for expandable structures. The base layer may be is typically formed from polymeric material, usually an elastic, acrylic adhesive material as described elsewhere herein. Alternatively, for non-expandable stress-applying features, the carrier template could be formed of non-elastic structures, including metals, ceramics, non-distensible polymers, and the like. The stress-applying features are attached to an outer surface of the carrier template, for example by applying or dispensing an adhesive at locations where the stress-applying features are to be positioned and pressing the features into the adhesives at these locations. The carrier template is then placed over on the outer surface of the structure, and the template secured to the outer surface, typically using an adhesive, but alternatively by heat welding, ultrasonic fusion, or other conventional techniques.

In a further aspect, the present invention provides apparatus for treating an aortic valve having calcified leaflets extending between a sinotubular junction and a valve annulus. The apparatus comprises a sleeve deployment catheter having a proximal end, a distal end, and a lumen extending through at least a distal region thereof. A leaflet-capture sleeve is coupled to the distal end of the deployment catheter, and the leaflet-capture sleeve has a distal edge configured to engage an aortic side of the calcified valve leaflets, i.e., the side located at the sinotubular junction. The lumen of the sleeve deployment catheter is configured to receive a valvuloplasty catheter, and a balloon or other expandable valvuloplasty element on the sleeve deployment catheter is configured be expanded against a ventricular side of the calcified valve leaflets while the distal edge of the leaflet-capture sleeve remains engaged against the aortic side of calcified valve leaflets.

In specific instances, the distal edge of the leaflet-capture sleeve may be circular.

In specific instances, the distal edge of the leaflet-capture sleeve may have undulations.

In specific instances, the distal edge of the leaflet-capture sleeve may have protrusions configured to be advanced past the sinotubular junction toward the valve annulus between commissures on the aortic side of the calcified valve leaflets.

In specific instances, the leaflet-capture sleeve or a region of the sleeve deployment catheter proximal of the leaflet-capture sleeve may comprise an embolic filter configured to allow blood from the aortic valve to flow out of the lumen while containing potentially harmful embolic material released as a result of expansion of the valvuloplasty element.

In specific instances, the filter element may comprise a mesh structure formed as at least a part of the leaflet-capture sleeve.

In specific instances, the filter element may comprise fenestration formed in a wall of the catheter.

In specific instances, the filter element may comprise a mesh structure formed in a wall of the catheter.

In specific instances, the sleeve deployment catheter may comprise stress-applying features formed on at least a portion of an inner surface of the leaflet-capture sleeve.

In another aspect, the apparatus of the present invention may comprise system including any one of the sleeve deployment catheters describe and claimed herein in combination with a valvuloplasty catheter having a valvuloplasty element.

In specific instances, the valvuloplasty element comprises a balloon structure disposed at a distal end of the catheter.

In other instances, the valvuloplasty element comprises a mechanically expandable cage structure disposed at a distal end of the catheter.

In specific instances, the valvuloplasty catheter further comprises stress-applying features on an outer surface of the valvuloplasty element.

In another aspect, the present invention provides a method for treating an aortic valve having calcified leaflets extending between a sinotubular junction and a valve annulus. A distal edge of a leaflet-capture sleeve is advanced from an aortic arch toward the sinotubular junction to engage said distal edge against an aortic side of at least some of the calcified valve leaflets. A valvuloplasty element is advanced through a lumen of the leaflet-capture sleeve, and the valvuloplasty element on the valvuloplasty catheter is expanded against a ventricular side of at least some of the calcified valve leaflets while the distal edge of the leaflet-capture sleeve remains engaged against the aortic side of at least some of the calcified valve leaflets.

In specific aspects, advancing the distal edge of the leaflet-capture sleeve comprises advancing a deployment catheter having a proximal end, a distal end, and a lumen extending through at least a distal region thereof, wherein the leaflet-capture sleeve is coupled to the distal end of the deployment catheter.

In specific instances, the distal edge may be circular.

In specific instances, the distal edge may have undulations.

In specific instances, the distal edge may have protrusions which extend past the sinotubular junction toward the valve annulus between commissures on the aortic side of the calcified valve leaflets when the distal edge of the leaflet-capture sleeve is advanced from the aortic arch toward the sinotubular junction.

In specific instances, the methods of the present invention may further comprise providing an embolic filter on an aortic side of the leaflet-capture sleeve to allow blood from the aortic valve to flow out of the lumen while containing potentially harmful embolic material released as a result of expansion of the valvuloplasty element.

In specific instances, the filter element may comprise a mesh structure formed as at least a part of the leaflet-capture sleeve.

In specific instances, such filters may comprise fenestrations formed in a wall of the catheter.

In specific instances, such filters may comprise a mesh structure formed in a wall of the catheter.

In specific instances, the methods of the present invention may further comprise providing stress-applying features on an inner surface of the leaflet-capture sleeve.

In specific instances, expanding the valvuloplasty element may comprise inflating a balloon structure.

In other instances, expanding the valvuloplasty element may comprise mechanically expanding a cage structure.

In specific instances, the methods of the present invention may further comprise providing stress-applying features on an outer surface of the valvuloplasty element.

In a further aspect, the present invention provides apparatus for treating calcification on a wall in a patient's body lumen comprising a catheter, an expandable structure, and a plurality of needle-like stress-applying features distributed over at least a portion of the outer surface of the expandable structure where at least a distal portion of at least some of the needle-like stress-applying features have an atraumatic cover over a distal tip thereof. The catheter typically includes a catheter body having a proximal end and a distal segment. The expandable structure is disposed at the distal segment of the catheter body and has an outer surface configured to be displaced radially outwardly toward an inner surface of the body lumen wall. In specific instances, the expandable structure comprises an inflatable balloon.

In specific instances, at least some of the needle-like stress-applying features have sharpened distal tips.

In specific instances, the atraumatic cover is compressible to expose the sharpened distal tip when the cover is pressed against calcified plaque.

In a further aspect, the present invention provides apparatus for treating calcifications on a wall in a patient's body lumen. The apparatus comprises a catheter, an expandable structure, and a plurality of elongate, blade-like stress-applying features. The catheter includes a catheter body having a proximal end and a distal segment, and the expandable structure is disposed at the distal segment of the catheter body. The expandable structure has an outer surface configured to be displaced radially outwardly toward an inner surface of the body lumen wall, and the plurality of elongate, blade-like stress-applying features is distributed over at least a portion of the outer surface of the expandable structure. At least at least some of the blade-like stress-applying features have a compressible, atraumatic cover thereover.

In specific instances, the expandable structure comprises an inflatable balloon.

In specific instances, the atraumatic cover covers the entire blade-like stress-applying features including the sharpened edge prior to the cover being compressed.

In other instances, the sharpened edge is exposed over a surface of the atraumatic cover prior to the cover being compressed.

The illustrative aspects, examples, or embodiments describes are not meant to be limiting. For example, the examples provided for an implantable scaffold comprising a scaffold structure having a surface configured to be expanded in the patient's body can also apply to other devices described in this application such as sleeves, balloon, cages, or the like.

In still further aspects of the present invention, apparatus for treating calcifications on a wall in a patient's body lumen comprises a catheter, an inflatable polymeric balloon, and a plurality of discrete stress-applying features distributed over at least a portion of an outer surface of the balloon. The catheter body has a proximal end and a distal end, and the inflatable polymeric balloon is attached to the distal end of the catheter body. The inflatable polymeric balloon has a hardness and an outer surface, and the plurality of discrete stress-applying features each have a hardness, a bottom, and a rounded convex upper surface, where the discrete stress-applying features are distributed over at least a portion of the outer surface. At least a first polymeric adhesive layer is disposed between the bottoms of said discrete stress-applying features and the outer surface of the inflatable polymeric balloon.

In specific instances, the first polymeric adhesive layer comprises a base layer configured to cover a continuous surface area of the outer balloon surface, where the continuous surface area is sufficiently large to lie beneath at least a multiplicity of the plurality of discrete stress-applying features which are preferably distributed over the continuous surface area in both axial and circumferential directions.

Usually, the continuous surface area is sufficiently large to lie beneath at least a majority of the plurality of discrete stress-applying features, and preferably the continuous surface area is sufficiently large to lie beneath all of the plurality of discrete stress-applying features.

In some instances, the continuous surface area includes at least a cylindrical region of the outer surface of the balloon (sometimes referred to as a working length or working surface). For example, the cylindrical region of then continuous surface area may be located between tapered or conical end regions and/or curved transition regions of the outer surface of the balloon.

In some instances, the continuous surface area may include at least one helical strip disposed over the cylindrical and/or tapered or conical regions of the balloon.

In some instances, the continuous surface area may include at least one axial strip disposed over the cylindrical and/or tapered or conical regions of the balloon.

In some instances, the continuous surface area includes at least one circumferential band disposed over one or more of the cylindrical and/or tapered or conical regions of the balloon.

In some instances, the continuous surface area comprises a random two-dimensional pattern.

In some instances, the apparatus further comprises a continuous surface of the balloon including a cylindrical surface and the plurality of discrete stress-applying features are arranged in a multiplicity of circumferentially adjacent bands axially spaced-apart along the cylindrical surface.

In some instances, the apparatus further comprises at least a second polymeric adhesive layer disposed between the bottoms of said discrete stress-applying features and the outer surface of the inflatable polymeric balloon. For example, the first and the second polymeric adhesive layers may both cover the same continuous surface area of the outer balloon surface. Alternatively or additionally, the first and second polymeric adhesive layers may cover a different continuous surface area of the outer balloon surface.

In some instances, the first and second adhesive layers each have a thickness no greater than 50% of a wall thickness of the inflatable polymeric balloon.

In some instances, the second adhesive layer may comprises a plurality of adhesive spots. For example, each adhesive spot may lie beneath an individual, discrete stress-applying feature and over an outer surface of the balloon.

In other instances, the adhesive spots may lie over the first polymeric adhesive layer and beneath the discrete stress-applying features.

In still other instances, the adhesive spots may lie beneath both the first polymeric adhesive layer and the discrete stress-applying features and over the balloon outer surface.

In some instances, the first and second polymer adhesive layers comprise the same adhesive polymeric material.

In some instances, the first and second polymer adhesive layers comprise different adhesive polymeric materials.

In some instances, the adhesive layer which is directly attached to the balloon outer surface is softer than the adhesive layer which is directly attached to the bottoms of the stress-applying features.

In some instances, the first polymer adhesive layer and/or the second polymer adhesive layer comprise one or more adhesive materials.

In some instances, apparatus of the present invention may further comprise a first polymeric cover layer. For example, the first polymeric cover layer may cover the outer surface of the balloon. For example, the first polymeric covers layer may cover at least some of the plurality of discrete stress-applying features. For example, the first polymeric cover layer may cover at least some of the first polymeric adhesive layer. For example, the first polymeric cover layer covers at least some of the first polymeric adhesive layer.

In some instances, the first polymeric cover layer comprises a polymeric adhesive.

In some instances, the first polymeric adhesive layer when cured may have a hardness less than a harness of the wall of the inflatable polymeric balloon and a hardness less than that of the discrete stress-applying features, where the first polymeric adhesive is configured to accommodate differential expansion between the bottoms of the discrete stress-applying features and the outer surface of the inflatable polymeric balloon as the balloon is inflated. For example, the discrete stress-applying features have a hardness of at least 4 Mohs, the polymeric balloon wall has a Shore hardness in a range from 60D to 90D, and the first polymeric adhesive has a Shore hardness in a range from 50D to 70D.

In some instances, the discrete stress-applying features may comprise at least one of a metal, a metal alloy, a mineral, a ceramic, and a hardened polymer. For example, the discrete stress-applying features may comprise a metal or metal alloy including at least one of iron, platinum, cobalt, chromium, rhodium, titanium, tungsten, and nickel.

In some instances, the polymeric balloon may comprise at least one of a nylon, a polyamide block copolymer, and a polyethylene terephthalate (PET).

In some instances, any one or more of the first polymeric adhesive layer, the second polymeric adhesive layer, and the first polymeric cover layer may comprise at least one of a polymethacrylate, a polyurethane-methacrylate, a polyisobornyl acrylate, an acrylic urethane methacrylate, a methacrylate ester acrylic, a modified methacrylate ester, a polyester, an epoxy adhesive, a phenolic adhesive, a polyvinyl acetate, a polyethylene-vinyl acetate, a polyethylene-methyl acrylate, a polyethylene, an acrylic, a cyanoacrylate, a hybrid cyanoacrylate/epoxy adhesive, a urea-formaldehyde, a polyimide, a natural or synthetic rubber modified with a tackifying resin, a styrene-butadiene rubber latex, a silicone rubber, an anaerobic glue, a mussel adhesive protein, a polydopamine-clay-polyacrylamide, a caulobacter crescentus, Delo Monopox, or combinations thereof.

In some instances, the apparatus of the present invention may further comprising at least a second polymeric adhesive layer disposed between the bottoms of said discrete stress-applying features and the outer surface of the inflatable polymeric balloon, wherein the at least a second polymeric adhesive layer when cured has a hardness greater than or equal to the hardness of the first polymeric adhesive layer, typically having a Shore hardness in a range from 50D to 70D.

In some instances, the first and second polymer adhesive layers may have the same hardness.

In other instances, the first and second polymer adhesive layers may have different hardnesses.

In some instances, the second polymer adhesive may comprise spot adhesives. For example, the spot adhesives may be formed over the first polymeric adhesive layer. Alternatively or additionally, the spot adhesives may be formed under the first polymeric adhesive layer.

In some instances, the first polymeric cover layer may have a Shore hardness in a range from 50D to 70D.

In some instances, the apparatus of the present invention may further comprise a second polymeric cover layer formed over the outer surface of the balloon and covering the plurality of discrete stress-applying features. For example, the second polymeric cover layer comprises a polymeric adhesive.

In such instances, the first and second polymeric cover layers typically each have a thickness no greater than 50% of the wall thickness of the inflatable polymeric balloon.

In such instances, the second polymeric cover layer typically has a Shore hardness in a range from 50D to 70D.

In preferred instances, the balloon wall consists of a single layer of a polymeric material. For example, the balloon wall consist of a single polymeric material having a hardness ranging from 55D to 90D.

In some instances, the polymeric material of the balloon is homogeneous composition material, i.e., having a uniform composition throughout most or all of the balloon structure.

In some instances, at least some of the plurality of discrete stress-applying features are formed as monolithic structures.

In other instances, at least some of the plurality of discrete stress-applying features are formed as polylithic structures.

In some instances, at least one of the first polymeric adhesive layer, the second polymeric adhesive layer, first polymeric cover layer, and the second polymeric adhesive cover layer comprises a homogeneous polymeric material, i.e., having a uniform composition throughout most of the balloon structure.

In some instances, at least one of the first polymeric adhesive layer, the second polymeric adhesive layer, first polymeric cover layer, and the second polymeric adhesive cover layer comprises reinforcement, a filler, a cross-linker, or an additive.

In some instances, the first polymeric adhesive layer and/or the second polymeric adhesive layer attach the bottoms of the discrete stress-applying features to the outer surface of the inflatable polymeric balloon.

In some instances, the first polymer adhesive layer, the second polymer adhesive layer, the first polymer adhesive cover, and/or the second polymer adhesive cover each comprise at least one polymer selected from a group consisting of a polymethacrylate, a polyurethane-methacrylate, a polyisobornyl acrylate, an acrylic urethane methacrylate, a methacrylate ester acrylic, a modified methacrylate ester, a polyester, an epoxy adhesive, a phenolic adhesive, a polyvinyl acetate, a polyethylene-vinyl acetate, a polyethylene-methyl acrylate, a polyethylene, an acrylic, a cyanoacrylate, a hybrid cyanoacrylate/epoxy adhesive, a urea-formaldehyde, a polyimide, a natural or synthetic rubber modified with a tackifying resin, a styrene-butadiene rubber latex, a silicone rubber, an anaerobic glue, a mussel adhesive protein, a polydopamine-clay-polyacrylamide, a caulobacter crescentus, Delo Monopox, and combination thereof.

In a still further aspect, the present invention provides apparatus for treating calcifications on a wall in a patient's body lumen comprising a catheter, an inflatable polymeric balloon, a plurality of discrete stress-applying features distributed over at least a portion of the outer surface, and a polymeric layer disposed over the outer surface of the inflatable polymeric balloon. The catheter body has a proximal end and a distal end, and the inflatable polymeric balloon is attached to the distal end of the catheter body. The inflatable polymeric balloon has a hardness and an outer surface configured to be displaced radially outwardly toward an inner surface of the body lumen wall, and the plurality of discrete stress-applying features each having a hardness, a bottom, and a rounded convex upper surface. The polymeric layer is disposed between the bottoms of said discrete stress-applying features and the outer surface of the inflatable polymeric balloon, and the polymeric layer covers a continuous surface area of the outer balloon surface an is sufficiently large to lie beneath at least a multiplicity of the plurality of discrete stress-applying features which are distributed over the continuous surface area in both axial and circumferential directions. A plurality of adhesive spots are deposited over said polymeric layer to enhance attachment of the bottoms of each of the plurality of discrete stress-applying features to the polymeric layer.

In some instances, the polymeric layer is configured to both (a) adhere to the outer balloon surface and (b) accommodate differential expansion between the bottoms of the discrete stress-applying features and the outer surface of the inflatable polymeric balloon as the balloon is inflated.

In some instances, adhesive spots are configured to adhere to both the outer surface of the balloon and to the bottoms stress-applying features.

In some instances, the adhesive spots are configured to both (a) adhere to the polymeric layer and attach the stress-applying features to the outer balloon surface and (b) further accommodate differential expansion between the bottoms of the discrete stress-applying features and the outer surface of the inflatable polymeric balloon as the balloon is inflated.

In some instances, the adhesive spots are configured to adhere to the outer surface of the balloon and to cover the stress-applying features.

In some instances, the polymeric layer is disposed between the bottoms of said discrete stress-applying features and the outer surface of the inflatable polymeric balloon the plurality of stress-applying features are distributed over the continuous surface area in both axial and circumferential directions.

For example, the plurality of stress-applying features may be arranged over the continuous surface area in one or more axial strips, one or more circumferential bands, two or more helical lines, in two or more axial strips, in two or more circumferential bands, two or more helical lines, and/or in a random two-dimensional grid.

In particular examples, the stress-applying features may be arranged in a grid pattern with both circumferential bands and axial strips with spacing or clearance between the features in both circumferential and axial directions. Such spacing may be uniform or different, regular of random.

In some instances, the continuous surface area is sufficiently large to lie beneath at least a majority of the plurality of discrete stress-applying features.

In some instances, the continuous surface area is sufficiently large to lie beneath all of the plurality of discrete stress-applying features.

In some instances, the polymeric layer is inseparable from the balloon outer surface.

In some instances, the polymeric layer is adhered to the balloon outer surface by one or more of heat, fusion, welding, deposition, gluing, and use of an adhesive.

In some instances, the polymeric layer may comprise, consist essentially of, or consist of a polymeric adhesive material.

In some instances, the polymeric layer may comprise a combination of an adhesive polymeric material and a non-adhesive polymeric material.

In some instances, the adhesive spots may comprise, consist of, or consist essentially of an adhesive polymeric material.

In some instances, the adhesive spots comprise a combination of a polymeric adhesive material and a non-adhesive polymeric material.

In yet another aspect, the present invention provides apparatus for treating calcifications on a wall in a patient's body lumen comprising a catheter, an inflatable polymeric balloon, a plurality of discrete stress-applying features distributed over at least a portion of an outer surface of the inflatable polymeric balloon, a first polymeric layer, and a second polymeric material. The catheter includes a catheter body having a proximal end and a distal end, and the inflatable polymeric balloon is attached to the distal end of the catheter body. The inflatable polymeric balloon has a hardness, and the outer surface is configured to be displaced radially outwardly toward an inner surface of the body lumen wall when the balloon is inflated. Each of the plurality of discrete stress-applying features has a hardness, a bottom, and a rounded convex upper surface, wherein said discrete stress-applying features are distributed over at least a portion of the outer surface area in both axial and circumferential directions. The first polymeric layer is disposed between the bottoms of said discrete stress-applying features and the outer surface of the inflatable polymeric balloon, and the first polymeric layer covers a continuous surface area of the outer balloon surface and is sufficiently large to lie beneath at least a multiplicity of the plurality of discrete stress-applying features. The second polymeric material encapsulates at least the bottoms of each of the plurality of stress-applying features and attaches to the first polymeric layer.

In some instances, wherein the second polymeric material is attached to the first polymeric material by one or more of heat, adhesive, fusing, soldering, or combinations thereof.

In some instances, the first polymeric layer and the second polymeric material comprise the same material.

In some instances, the first polymeric layer and the second polymeric material comprise different materials.

In still another aspect, the present invention provides apparatus for treating calcifications on a wall in a patient's body lumen comprising a catheter, an inflatable polymeric balloon, a plurality of discrete stress-applying features distributed over at least a portion of an outer surface of the inflatable polymeric balloon, at least one layer comprising plurality of discrete polymeric adhesive spots, a first polymeric layer, and a second polymeric material. The catheter includes a catheter body having a proximal end and a distal end, and the inflatable polymeric balloon is attached to the distal end of the catheter body. The inflatable polymeric balloon has a hardness, and the outer surface is configured to be displaced radially outwardly toward an inner surface of the body lumen wall when the balloon is inflated. Each of the plurality of discrete stress-applying features has a hardness, a bottom, and a rounded convex upper surface, and the discrete stress-applying features are distributed over at least a portion of the outer surface. The discrete polymeric adhesive spots are disposed between the bottoms of said discrete stress-applying features and the outer surface of the inflatable polymeric balloon, and at least the first polymeric adhesive layer disposed over the outer surface of the inflatable polymeric balloon which overlaps at least a portion of the at least one layer of plurality of discrete polymeric adhesive. The discrete polymeric adhesive spots and first polymeric adhesive are configured to accommodate differential expansion between the bottoms of the discrete stress-applying features and the outer surface of the inflatable polymeric balloon as the balloon is inflated.

In some instances, the plurality of discrete adhesive spots extends beyond the periphery of the plurality of stress applying features bottoms.

In some instances, the first polymeric adhesive layer covers the at least one layer of plurality of discrete polymeric adhesive spots.

In some instances, the first polymeric adhesive layer covers at least a portion of the plurality of discrete stress-applying features surface.

In some instances, the plurality of discrete adhesive spots and the first polymeric adhesive layer comprise the same polymeric adhesive.

In some instances, the plurality of discrete adhesive spots and the first polymeric adhesive layer comprise different polymeric adhesives.

In some instances, at least a first polymeric adhesive cover layer covers at least part of the plurality of discrete stress-applying features surface.

In some instances, the first polymeric adhesive cover layer covers at least a portion of the first polymeric adhesive layer.

In some instances, the first polymeric adhesive cover layer covers at least part of the plurality of discrete stress-applying features surface and at least portion of the first polymeric adhesive layer.

In some instances, at least a first adhesive polymeric layer covers some or all of the outer surface of a single layer, homogeneous composition polymeric balloon with a multiplicity of stress-applying features attached to the outer balloon surface by either the adhesive polymeric layer and/or an adhesive spot layer. The at least first adhesive polymeric layer and the adhesive spot layer(s) are typically both softer than the balloon, and there are preferably no stiffening members or other reinforcements attached to or embedded in the balloon. Nor will the balloon itself have portions or regions which are stiffer or otherwise different than the remainder of the balloon. The balloon is optionally modified by adding adhesive polymer layers and/or adhesive spots and stress-applying feature as described herein.

In some instances, the balloon consists of a single layer composition or material structure, which may be composed of a single polymer, a copolymer, or a homogeneous mixture of polymers with metal or other hard, rigid stress-applying features attached to the balloon through an adhesive polymer, such as the adhesive polymer layers and/or adhesive polymeric spots described herein. There are no stiffening members attached to or embedded in the balloon and there are no portions of the balloon itself which are different or stiffer than the remainder of the balloon.

Typically, the stress-applying features will be discrete metal structures having rounded upper surfaces and bottoms, typically but not always flat, which are adhesively attached directly to the outer surface of the single layer balloon that has not been modified by stiffening members or extra layers of balloon material. The polymeric adhesive used to attach the stress-applying features is usually softer than the polymer of the polymeric balloon.

In some instances, the discrete stress-applying features may be directly adhesively attached to the outer surface of the single layer polymeric balloon.

In some instances, at least one polymeric layer may be formed on the surface of the polymeric balloon under the spot adhesive which attaches the discrete stress-applying features to the balloon. Each of these polymeric layers is softer than the balloon and singly or together, they seal the interface between the stress-applying features and the balloon, forming a buffer between the hard discrete stress-applying features and the balloon. The one or more polymeric layers applied to the balloon form an accommodating layer or barrier, allowing flexibility of the balloon as it traverses the body lumen and reducing the possibility of moisture getting under the stress-applying feature. These single or multiple intervening layers maintain flexibility of the balloon allowing it to conform to the passages of the body lumen and stabilize the structure to maintain the discrete features in their positions on the balloon during use in a physiologic environment.

While the polymeric balloon will usually consist of a single, unmodified homogeneous polymeric layer free from stiffeners, reinforcement, and other non-homogeneities, regions of the balloon wall which do not underlie a stress-applying feature may in some instances have multiple layers, reinforcements, or other non-homogeneities. Those balloon regions underlying the stress-applying features, however, will preferably be free from all such non-homogeneities in preferred embodiments.

In still further aspects, the present invention provides apparatus for treating calcifications on a wall in a patient's body lumen comprising a catheter, an inflatable polymeric balloon, and a plurality of discrete, rigid stress-applying features. The catheter includes a catheter body having a proximal end and a distal end, and the inflatable polymeric balloon is attached to the distal end of the catheter body and has an outer balloon surface. Each of the plurality of discrete, rigid stress-applying features is attached to the outer balloon surface and are arranged in a first multiplicity of circumferential bands spaced-apart in an axial direction along a length of the outer surface of the polymeric balloon. Each stress-applying feature within a band is circumferentially spaced apart, and at least some of the stress-applying features are axially offset from at least one (and usually two) adjacent stress-applying feature within the same band when the polymeric balloon is inflated.

In some instances, the plurality of discrete, rigid stress-applying features are further arranged in a second multiplicity of axially oriented strips disposed along a length of the outer surface of the balloon, where at least some of the stress-applying features in some or all of the axially oriented strip may be axially offset from stress-applying features in a circumferentially adjacent axially oriented strip when the balloon is inflated.

In some instances, stress-applying features in some or all of the axially oriented strips may be axially offset from stress-applying features in a circumferentially adjacent axially oriented strip when the balloon is inflated.

In some instances, stress-applying features on at least some axially oriented strips have the same axial spacing.

In some instances, stress-applying features within all axially oriented strips have the same axial spacing.

In some instances, at least some of the stress-applying features within at least some circumferential bands may be axially offset from others of the stress-applying features within that circumferential band to enhance disruption force for each stress-applying feature and/or to avoid stacking of the stress applying features when the balloon is deflated. Often, all of the stress-applying features within at least some circumferential band are axially offset from others of the stress-applying features within that circumferential band. For example, each of the stress-applying features within each circumferential band may be axially offset from others of the stress-applying features within that circumferential band.

In some instances, each of the stress-applying features within the same circumferential band have the same circumferential spacing between them.

In some instances, the stress-applying features within at least some circumferential bands have the same circumferential spacing therebetween.

In some instances, at least some stress-applying features in at least one axial strip are axially offset from at least one stress-applying feature in a circumferentially adjacent axial strip.

In some instances, all stress-applying features are axially offset from at least some stress-applying features in at least one circumferentially adjacent axial strip. Often, all stress-applying features are axially offset from the circumferentially adjacent stress-applying features in that circumferential band.

In some instances, all stress-applying features in each axial strip are axially offset from the stress-applying features in circumferentially adjacent axial strips.

In some instances, the stress-applying features have spacing from other stress-applying features on the balloon in both an axial and circumferential direction. This spacing may be the same or different in either or both directions.

In some instances, the multiplicity of stress-applying features attached to the balloon in circumferential bands and axial strips may form a pattern that is regular, irregular, random, helical or a combination thereof. The spacing between stress-applying features may be uniform across the length of the balloon with the offset distance being uniform or the spacing may differ along the length of the balloon with different offsets.

The discrete stress-applying features may be attached to the balloon in a grid having a variety of patterns. Axial strips of stress-applying features may be straight and/or parallel to each other along the length of the balloon or they may have some features not in a straight line along the longitudinal length of the balloon. Having axial strips or rows of discrete stress-applying features slightly axially offset from the stress-applying features in circumferentially adjacent axial strips improves the effectiveness of the features to break or crush the occlusions in the body lumen because the offsets of the discrete stress-applying features better distribute the force of the features than if they were positioned in circumferentially aligned bands. The offset of the features provides more force, better distribution of the force, and a lower profile of the features to reduce the overlap if the features when the balloon is deflated. However, if the offset is too great, the ability to crack the occlusions is diminished or eliminated. Ideally, the spacing of the features is 0.05 mm to 0.5 mm to optimize the performance of the balloon in breaking calcifications in a body lumen.

In some instances, at least some of the stress-applying features have a convex rounded upper surface and a round base surrounding the center.

In some instances, the stress-applying features may have a width or diameter in a range from 0.15 mm to 1 mm, preferably from 0.2 mm to 1 mm, usually from 0.3 mm to 0.6 mm.

In some instances, the stress-applying features may be axially offset by a distance in a range from 0.3 mm to 2 mm, preferably from 0.4 mm to 1.5 mm, usually from 0.5 mm to 1 mm.

In some instances, circumferentially adjacent stress-applying features are sufficiently axially space-apart so that peripheral edges of said circumferentially adjacent stress-applying features do not axially overlap, wherein peripheral edges of the circumferentially adjacent stress applying features will have a gap therebetween in a range from 0 to 3 mm, usually from 0 to 2 mm, and preferably from 0.05 mm to 0.4 mm.

In some instances, all stress-applying features are sufficiently space-apart so that peripheral edges of said stress-applying features do not axially overlap, having a gap therebetween in a range from 0 to 3 mm, usually from 0 to 2 mm, and preferably from 0.05 mm to 0.4 mm, when the inflatable polymeric balloon is deflated. For example, the width or diameter of each stress-applying feature, axial offset between circumferentially adjacent stress-applying feature, and circumferential offset between axially adjacent stress-applying are constant for all stress-applying features when the inflatable polymeric balloon is inflated.

In some instances, a center of the stress-applying feature comprises a center of a bottom surface of the stress-applying feature.

In some instances, a center of the stress-applying feature comprises a center of an upper surface of the stress-applying feature.

In some instances, axial and circumferential offsets are measured with respect to the centers of adjacent stress-applying features.

In some instances, the density of circumferential band along an axial length of the ranges from 0.2 strips/mm of axial balloon length to 2 strips/mm of axial balloon length, preferably ranging from 0.3 strips/mm of axial balloon length to 1 strip/mm of axial balloon length, and most preferably ranging from 0.4 strips/mm of axial balloon length to 1 strip/mm of axial balloon length.

In some instances, bottoms of axially adjacent stress-applying features within at least some axially oriented strips are axially spaced apart by distance ranging from 0.5 mm to 3 mm, usually from 1 mm to 2.5 mm, and preferably ranges from 1.5 to 2.5 mm.

In some instances, at least some of the discrete stress-applying features are formed as a sphere, hemisphere, partial sphere, ellipsoid, or other shape having convex rounded upper surface.

In still another aspect, the present invention provides apparatus for treating calcifications on a wall in a patient's body lumen comprising a catheter, an inflatable polymeric balloon, and a plurality of discrete, rigid stress-applying features. The catheter includes a catheter body having a proximal end and a distal end, and the inflatable polymeric balloon is attached to the distal end of the catheter body and has an outer balloon surface. The rigid stress-applying features are each attached to the outer balloon surface, where at least some of the stress-applying features have a convex rounded upper surface and a base attached directly or indirectly to the balloon surface. Most and preferably all peripheral edges of the stress-applying features do not overlap when the balloon is deflated.

In some instances, the peripheral edges of adjacent stress-applying features when the polymeric balloon is deflated will have a gap therebetween in a range from 0 to 3 mm, usually from 0 to 2 mm, and preferably from 0.05 mm to 0.4 mm.

In yet a further aspect, the present invention provides apparatus for treating calcifications on a wall in a patient's body lumen comprising a catheter, an inflatable polymeric balloon, and a plurality of discrete, rigid stress-applying features. The catheter includes a catheter body having a proximal end and a distal end, and the inflatable polymeric balloon is attached to the distal end of the catheter body and has an outer surface. The plurality of stress-applying features attached to the outer surface of the polymeric base layer, and the stress-applying features have distribution density in a range from 0.1 to 5 features/mm² of outer surface area or a portion thereof, such as a cylindrical central section of the balloon, preferably from 0.2 to 4 features/mm², more preferably from 0.25 to 3 features/mm² over at least an expanded region of the inflatable polymeric balloon when the inflatable polymeric balloon is fully inflated.

In still another aspect, the present invention provides apparatus for treating calcifications on a wall in a patient's body lumen comprising a catheter, an inflatable polymeric balloon, and a plurality of discrete, rigid stress-applying features. The catheter including a catheter body having a proximal end and a distal end, and the inflatable polymeric balloon is attached to the distal end of the catheter body. The inflatable polymeric balloon has an outer surface, and the plurality of stress-applying features is attached to the outer surface of the inflatable polymeric balloon, where the stress-applying features have a distribution density in a range from 0.1 to 5 features/mm², preferably from 0.2 to 4 features/mm², more preferably from 0.25 to 3 features/mm² over at least an expanded region of the inflatable polymeric balloon when the inflatable polymeric balloon is fully inflated.

In some instances, the expanded region of the inflatable polymeric balloon comprises the entire expandable surface area of the balloon.

In some instances, the expanded region of the inflatable polymeric balloon comprises a central area of the balloon excluding tapered end regions of the balloon.

In yet a further aspect, the present invention provides apparatus for treating calcifications on a wall in a patient's body lumen comprising a catheter, an inflatable polymeric balloon, and a plurality of discrete, rigid stress-applying features. The catheter includes a catheter body having a proximal end and a distal end. The inflatable polymeric balloon is attached to the distal end of the catheter body, and the inflatable polymeric balloon has an outer surface. The plurality of stress-applying features is attached to the outer surface of the polymeric balloon, and the stress-applying features each have a base region in contact with the outer surface of the inflatable polymeric balloon. A ratio of (1) a cumulative area of all the base regions in contact with the outer surface of the inflatable polymeric balloon to (2) a total area of the outer surface of the inflatable polymeric balloon is in a range from 1:100 to 5:100; usually from 2:100 to 5:100, and preferably from 3:100 to 4:100.

In some instances, the outer surface of the inflatable polymeric balloon may comprise the entire expandable surface area of the balloon.

In other instances, the outer surface of the inflatable polymeric balloon comprises a central area of the balloon excluding tapered end regions of the balloon.

In some instances, the stress-applying features have a convex rounded upper surface and a round base region in contact with the outer balloon surface.

In some instances, the stress-applying features all have the same dimensions.

In some instances, the stress-applying features are distributed evenly over outer surface of the inflatable polymeric balloon.

In yet one additional aspect, the present invention provides apparatus for treating calcifications on a wall in a patient's body lumen comprising a catheter, an inflatable polymeric balloon, and a plurality of discrete, rigid stress-applying features. The catheter includes a catheter body having a proximal end and a distal end. The inflatable polymeric balloon is attached to the distal end of the catheter body, and the inflatable polymeric balloon has an outer surface. The plurality of stress-applying features is attached to the outer surface of the polymeric balloon, a catheter including a catheter body having a proximal end and a distal end, and the inflatable polymeric balloon having an outer surface with a central region, a tapered distal region, a tapered proximal region, a distal transition region between the distal tapered region and the central region, and a proximal transition region between the proximal tapered region and the central region. Individual rigid features are attached to the outer surface of the inflatable polymeric balloon, where at least some of the rigid features are distributed over at least a portion of one of (a) the distal transition region, (b) the proximal transition region, (c) the distal tapered region, (d) the proximal tapered region, (e) a distal terminal 2 mm length of the central region, and (f) a proximal terminal 2 mm length of the central region of the outer surface of the inflatable polymeric balloon.

In some instances, at least some of the rigid features have a convex rounded upper surface.

In some instances, the rigid features have a width or diameter in a range from 0.15 mm to 1 mm, preferably from 0.2 mm to 1 mm, usually from 0.3 mm to 0.6 mm.

In some instances, the discrete rigid features comprise a metal or metal alloy comprising at least one of iron, platinum, cobalt, chromium, rhodium, titanium, tungsten, and nickel.

In some instances, the inflatable polymeric balloon comprises a semi-compliant balloon having a nominal inflation pressure and a rated burst pressure, wherein a diameter of the central region of the balloon increases by a percentage in a range from 1% to 20%, usually from 5% to 20%, and preferably from 5% to 15%, as the balloon is inflated from its nominal inflation pressure to its rated burst pressure.

In some instances, the polymeric balloon comprises at least one of a nylon, a polyamide block copolymer, and a polyethylene terephthalate (PET).

In some instances, the inflatable polymeric balloon comprises a non-compliant balloon having a nominal inflation pressure and a rated burst pressure, wherein a diameter of the central region of the balloon increases by a percentage less than or equal to 5% as the balloon is inflated from its nominal inflation pressure to its rated burst pressure.

In some instances, at least some of the rigid features are distributed over at least a portion of both the distal transition region and the proximal transition region of the outer surface of the inflatable polymeric balloon.

In some instances, at least some of the rigid features are also distributed over at least portions of both the tapered distal region and the tapered proximal region of the outer surface of the inflatable polymeric balloon.

In some instances, at least some of the rigid features are distributed over at least portions of both the proximal and distal terminal 1 mm lengths of the central region of the outer surface of the inflatable polymeric balloon.

In some instances, the rigid features are arranged in circumferential bands over the outer surface of the inflatable polymeric balloon. For example, the circumferential bands may each include from 2 to 8 rigid features, usually from 2 to 6 rigid features, and preferably from 3 to 5 rigid features.

In certain instances, some of the rigid features are distributed over the central region of the outer surface of the inflatable polymeric balloon and additional features are distributed over one or more of (a) the distal transition region, (b) the proximal transition region, (c) the distal tapered region, (d) the proximal tapered region. Distributing the stress-applying features on or over a transition region and/or a tapered region of the balloon allows the stress-applying features to fracture or crack the calcifications while inhibiting (eliminating or minimizing) damage to the adjacent vessel due to diminished, reduced, or eliminated “dog boning” of the balloon adjacent to the calcifications. Having the stress-applying features present in areas of the balloon which have a smaller circumference than the rest of the balloon allows the balloon to bend around the calcification without excessive bulging or “ballooning” of portions of the balloon to a larger size and potentially angle the stress-applying features against the vessel wall. Stress-applying features on the transition and tapered regions still provide sufficient force to break the calcifications but with reduced damage to the vessel wall.

In some instances, all the rigid features have the same shape and dimensions.

In some instances, the rigid features on the central region have different shapes and/or dimensions than the shapes and/or dimensions of the rigid features on the one or more of (a) the distal transition region, (b) the proximal transition region, (c) the distal tapered region, (d) the proximal tapered region.

In some instances, the rigid features are arranged in axial strips and circumferential bands in any or all of the regions.

In some instances, each axial strip consists of from 2 to 8 rigid features.

In some instances, each circumferential band consists of from 2 to 8 rigid features.

These and other embodiments are described in further detail in the following clauses and description related to the appended drawing figures.

Clause 1. An endoluminal prosthesis comprising:
a scaffold composed at least partly of a non-degradable material and configured to expand from a crimped configuration to an expanded configuration; and
a plurality of stress-applying features coupled to an outer surface of the scaffold;
wherein at least some of said stress-applying features comprise a blunt contact region having a contact surface spaced outwardly from said outer surface of the scaffold and configured to fracture occlusive material in the wall of a vascular lumen when the scaffold is expanded from the crimped configuration to the expanded configuration in the vascular lumen.

Clause 2. The endoluminal prosthesis of clause 1, wherein the scaffold has a tubular geometry.

Clause 3. The endoluminal prosthesis of clause 2, wherein the tubular scaffold has a cylindrical shape, an ellipsoidal shape, a tapered profile, an hourglass shape, or a dog-bone shape.

Clause 4. The endoluminal prosthesis of clause 1 to 3, wherein at least some of the blunt contact regions comprise a peripheral edge circumscribing the contact surface and configured to concentrate stress when engaged against the occlusive material on the wall of a vascular lumen when the scaffold is expanded from the crimped configuration to the expanded configuration in the vascular lumen.

Clause 5. The endoluminal prosthesis of clause 4, wherein the peripheral edge is formed by an intersection between the blunt contact region and a peripheral wall at least partially surrounding the blunt contact region.

Clause 6. The endoluminal prosthesis of clause 1 to 5, wherein the contact surface of the blunt contact region is flat, convex, rounded, or concave.

Clause 7. The endoluminal prosthesis of clause 6, wherein the contact surface of the blunt contact region is parallel to the outer surface of the scaffold.

Clause 8. The endoluminal prosthesis of clause 6, wherein the contact surface of the blunt contact region is inclined relative to the outer surface of the scaffold.

Clause 9. The endoluminal prosthesis of clause 8, wherein the contact surface of the blunt contact region is inclined an angle in the range from 5° to 45°, preferably from 10° to 30°.

Clause 10. The endoluminal prosthesis of clause 1 to 10, wherein the peripheral wall is oriented at an angle in a range from 75° to 105° relative to the contact surface of the blunt contact region.

Clause 11. The endoluminal prosthesis of clause 4 to 10, wherein the peripheral edge extends fully about the contact surface of the blunt contact region and has a width in a range from 10 μm to 200 μm.

Clause 12. The endoluminal prosthesis of clause 11, wherein the peripheral edge is circular, and the width comprises a diameter.

Clause 13. The endoluminal prosthesis of clause 1 to 12, wherein at least some of the plurality of stress-applying features comprise one or more plates having a total thickness in a range from 0.25 mm to 1 mm and a width where attached to the surface of the tubular scaffold in a range from 0.1 mm to 2 mm.

Clause 14. The endoluminal prosthesis of clause 13, wherein at least some of the plates are configured as disks, stacked disks, truncated cones, stacked disks and truncated cones, ellipsoidal disks, and asymmetric cones.

Clause 15. The endoluminal prosthesis of clause 1 to 12, wherein the scaffold comprises a plurality of struts joined by crowns.

Clause 16. The endoluminal prosthesis of clause 15, wherein the plurality of struts joined by crowns are joined into a plurality of circumferential rings.

Clause 17. The endoluminal prosthesis of clause 15, wherein the plurality of struts joined by crowns are joined in a helical pattern.

Clause 18. The endoluminal prosthesis of clause 13 to 17, wherein at least some of the stress-applying features are located at or adjacent to crowns.

Clause 19. The endoluminal prosthesis of clause 18, wherein at least some of the crowns carrying stress-applying features are not joined to adjacent rings.

Clause 20. The endoluminal prosthesis of clause 18 or 19, wherein each of the stress-applying features is located at or adjacent to a crown.

Clause 21. The endoluminal prosthesis of clause 13 to 17, wherein at least some of the stress-applying features are located on the struts between the crowns or are located on one or more links joining adjacent rings.

Clause 22. The endoluminal prosthesis of clause 1 to 21, wherein at least some of the stress-applying features are arranged in diametrically opposed pairs.

Clause 23. The endoluminal prosthesis of clause 22, wherein successive diametrically opposed pairs of crowns are circumferentially offset.

Clause 24. The endoluminal prosthesis of clause 23, wherein the successive diametrically opposed pairs of crowns are circumferentially offset by an angle from 45° to 90°.

Clause 25. The endoluminal prosthesis of clause 1 to 21, wherein at least some of the stress-applying features are arranged in groups of three which are circumferentially separated about a circle on the surface of the tubular scaffold by about 1200.

Clause 26. The endoluminal prosthesis of clause 1 to 20, wherein at least some successive axially spaced-apart stress-applying features are circumferentially offset by an angle in the range from 5° to 15°.

Clause 27. The endoluminal prosthesis of clause 26, wherein at least some successive circumferentially spaced-apart stress-applying features are axially offset by an angle in the range from 5° to 15°.

Clause 28. The endoluminal prosthesis of clause 1 to 27, wherein the scaffold formed by patterning a tubular substrate, laser cutting a tubular substrate, rolling a cut substrate, bending a wire, or three-dimensional printing.

Clause 29. The endoluminal prosthesis of clause 1 to 28, wherein stress-applying features pre-formed and attached by gluing, soldering, welding, fusion, threaded attachment, riveting, or crimping.

Clause 30. The endoluminal prosthesis of clause 29, wherein the stress-applying features comprise preformed plates glued to the scaffold with an adhesive.

Clause 31. The endoluminal prosthesis of clause 1 to 28, wherein the stress-applying features are formed in situ by three-dimensional printing, chemical vapor deposition, electrostatic deposition, molding, or folding of a component of the scaffold.

Clause 32. The endoluminal prosthesis of clause 1 to 28, wherein the stress-applying features comprise tabs attached to the scaffold and folded over onto the outer surface of the scaffold.

Clause 33. The endoluminal prosthesis of clause 1 to 32, wherein the scaffold comprises a vascular stent or stent-graft.

Clause 34. The endoluminal prosthesis of clause 1 to 32, wherein the scaffold comprises a prosthetic valve.

Clause 35. The endoluminal prosthesis of clause 1 to 32, wherein the scaffold comprises a valvuloplasty device.

Clause 36. The endoluminal prosthesis of clause 1 to 35, wherein the scaffold is balloon expandable.

Clause 37. The endoluminal prosthesis of clause 1 to 35, wherein the scaffold is self-expanding.

Clause 38. The endoluminal prosthesis of clause 1 to 37, wherein the stress-applying features are configured to preferentially contact the occlusive material in the wall.

Clause 39. The endoluminal prosthesis of clause 1 to 38, wherein the scaffold comprises a sleeve configured to be placed over a stent or a balloon or to be self-expanding.

Clause 40. The endoluminal prosthesis of clause 1 to 39, wherein the stress-applying features comprise a sharp element projecting outwardly from the blunt contact region and wherein the sharp element is configured to concentrate stress when engaged against the occlusive material on the wall of a vascular lumen when the blunt contact region is pressed against a surface of the occlusive material.

Clause 41. The endoluminal prosthesis of clause 40, wherein the blunt surface extends above a surface of the scaffold by a first distance and the sharp element projects from the surface of the blunt contact region by a second distance equal to 0.05 mm to 0.1 mm of the first distance.

Clause 42. The endoluminal prosthesis of clause 41, wherein the second distance is in a range from 0.01 mm to 0.2 mm, or 0.01 mm to 0.1 mm.

Clause 43. The endoluminal prosthesis of clause 40 to 42, wherein the sharp element comprises a point.

Clause 44. The endoluminal prosthesis of clause 40 to 42, wherein the sharp element comprises an edge.

Clause 45. A method for fracturing calcified plaque in a patient's vasculature, said method comprising:
expanding a scaffold composed at least in part of a non-degradable material from a crimped configuration to an expanded configuration in a calcified body vascular lumen;
wherein said scaffold comprises a plurality of stress-applying features fixed to an outer surface thereof;
wherein at least some of said stress-applying features comprise a blunt contact region spaced outwardly from said outer surface and having a peripheral edge configured to fracture occlusive material on the wall of a vascular lumen when the tubular scaffold is expanded from the crimped configuration to the expanded configuration in the vascular lumen; and
wherein the stress-applying features fracture the occlusive material as the scaffold is expanded.

Clause 46. The method of clause 45, wherein the occlusive material comprises hardened plaque or calcification.

Clause 47. The method of clause 45 or 46, wherein expanding the scaffold comprises expanding a balloon within the scaffold or allowing the scaffold to self-expand.

Clause 48. The method of clause 45, wherein expanding the scaffold comprises expanding a prosthetic heart valve in a heart valve annulus, wherein the scaffold comprises a structural support of the heart valve annulus.

Clause 49. The method of clause 48, wherein expanding the prosthetic heart valve comprises expanding a balloon to expand the prosthetic heart valve in the heart valve annulus.

Clause 50. The method of clause 45, wherein expanding the scaffold comprises expanding a valvuloplasty device in a heart valve annulus.

Clause 51. The method of clause 50, wherein the scaffold of the valvuloplasty device comprises an expandable cage and expanding the valvuloplasty device comprises expanding the cage in the heart valve annulus.

Clause 52. The method of clause 45 to 51, wherein the tubular scaffold has a cylindrical shape, an ellipsoidal shape, a tapered profile, an hourglass shape, or a dog-bone shape.

Clause 53. The method of clause 45 to 52, wherein the peripheral edge is formed by an intersection between the blunt contact region and a peripheral wall at least partially surrounding the blunt contact region.

Clause 54. The method of clause 45 to 53, wherein the blunt contact region is flat.

Clause 55. The method of clause 54, wherein the blunt contact region is parallel to the outer surface of the tubular scaffold.

Clause 56. The method of clause 55, wherein the blunt contact region is inclined relative to the outer surface of the tubular scaffold.

Clause 57. The method of clause 45 to 56, wherein the peripheral edge is formed by an intersection between the blunt contact region and a peripheral wall at least partially surrounding the blunt contact region.

Clause 58. The method of clause 57, wherein the blunt contact region is planar.

Clause 59. The method of clause 57, wherein the peripheral wall is oriented at an angle in a range from 75° to 105° relative to the blunt contact region.

Clause 60. The method of clause 57 to 59, wherein the peripheral edge has a width in a range from 10 μm to 200 μm

Clause 61. The method of clause 57 to 60, wherein at least some of the plurality of stress-applying features comprise one or more plates having a total thickness in a range from 0.5 mm to 1 mm and a width of a surface attached to the surface of the tubular scaffold in a range from 0.01 mm to 2 mm.

Clause 62. The method of clause 61, wherein at least some of the plates are configured as disks, stacked disks, truncated cones, stacked disks and truncated cones, ellipsoidal disks, and asymmetric cones.

Clause 63. The method of clause 45 to 57, wherein the scaffold comprises a plurality of struts joined by crowns.

Clause 64. The method of clause 63, wherein the plurality of struts joined by crowns are joined into a plurality of circumferential rings.

Clause 65. The method of clause 63, wherein the plurality of struts joined by crowns are joined in a helical pattern.

Clause 66. The method of clause 63 to 65, wherein at least some of the stress-applying features are located at or adjacent to crowns.

Clause 67. The method of clause 66, wherein each of the stress-applying features is located at or adjacent to a crown.

Clause 68. The method of clause 63 to 65, wherein at least some of the stress-applying features are located on the struts between the crowns.

Clause 69. The method of clause 63 to 68, wherein at least some of the stress-applying features are arranged in diametrically opposed pairs.

Clause 70. The method of clause 63 to 69, wherein successive diametrically opposed pairs of crowns are rotationally offset relative to a longitudinal axis of the scaffold.

Clause 71. The method of clause 70, wherein the successive diametrically opposed pairs of crowns are rotationally offset by an angle from 75° to 105° relative to a longitudinal axis of the scaffold.

Clause 72. A method for fabrication a vascular scaffold, said method comprising:
patterning a tubular scaffold comprising a plurality of struts joined by crowns within a tubular envelope, said tubular scaffold having a plurality of tabs extending outwardly from the struts and/or crowns within the tubular envelope; and
folding the plurality of tabs over an outer surface of the tubular envelope to form a plurality of stress-applying features on an outer surface of the tubular scaffold.

Clause 73. The method of clause 72, wherein pairs of adjacent tabs folded one over the other to form stacked stress-applying features.

Clause 74. The method of clause 73, wherein the adjacent tabs in the pairs of are arranged side-by-side on the scaffold prior to folding.

Clause 75. The method of clause 73, wherein the adjacent tabs in the pairs of are arranged in tandem on the scaffold prior to folding.

Clause 76. The method of clause 73, wherein the adjacent tabs in the pairs of are arranged on opposite sides of a strut prior to folding.

Clause 77. Apparatus for treating calcification on a wall in a patient's body lumen, said system comprising:
a catheter including a catheter body having a proximal end and a distal segment;
an expandable structure disposed at the distal segment of the catheter, said expandable structure having an outer surface configured to be displaced radially outwardly toward an inner surface of the body lumen wall; and
a plurality of stress-applying features distributed over the outer surface of the expandable structure, wherein at least some of the stress-applying features are present on the outer surface of the expandable structure and have convex rounded upper surfaces configured to fracture the calcification while minimizing damage to the body lumen when the expandable structure is expanded within the body lumen.

Clause 78. Apparatus as in clause 77, wherein the body lumen comprises a blood vessel, a valve annulus, a venous valve, or AV shunt.

Clause 79. Apparatus as in clause 77 or 78, wherein the calcification is located within an inner wall, an intimal layer, a medial layer, an adventitial layer, a valve leaflet, valve annulus, venous filter, or an implant.

Clause 80. Apparatus as in any one of clauses 77 to 79, wherein the expandable structure is less rigid when not expanded and more rigid when fully expanded.

Clause 81. Apparatus as in any one of clauses 77 to 80, wherein the outer surface of the expandable structure is generally cylindrical when fully expanded.

Clause 82. Apparatus as in any one of clauses 77 to 81, wherein the convex rounded upper surfaces of plurality of stress-applying features extend radially outwardly beyond the outer surface of the expandable structure when fully expanded.

Clause 83. Apparatus as in clause 82, wherein the convex rounded upper surfaces of plurality of stress-applying features extend radially outwardly beyond the outer surface of the expandable structure by a distance in a range from 0.15 mm to 3 mm, preferably range from 0.25 mm to 3 mm, more preferably ranging from 0.5 mm to 3 mm when fully expanded.

Clause 84. Apparatus as in any one of clauses 77 to 83, wherein the convex rounded upper surfaces of the stress-applying features are free from edges and irregularities which could damage the wall when the expandable structure is expanded within the body lumen.

Clause 85. Apparatus as in any one of clauses 77 to 84, wherein at least some of the stress-applying features have a single convex rounded upper surface and a lower base independently attached to the outer surface of the expandable structure.

Clause 86. Apparatus as in any one of any one of clauses 77 to 85, wherein at least some of the stress-applying features comprise spheres, hemispheres, truncated spheres, or ellipsoids.

Clause 87. Apparatus as in any one of clauses 77 to 86, wherein at least some of the stress-applying features are independently attached to the outer surface of the expandable structure.

Clause 88. Apparatus as in any one of clauses 77 to 87, wherein at least some of the stress-applying features comprise hemispheres having lower surfaces attached to the outer surface of the expandable structure.

Clause 89. Apparatus as in clause 88, wherein the lower surfaces are flat.

Clause 90. Apparatus as in clause 88, wherein the lower surfaces are contoured.

Clause 91. Apparatus as in any one of clauses 77 to 88, wherein at least some of the stress-applying features comprise posts having hemispherical upper surfaces and lower surfaces attached to the outer surface of the expandable structure.

Clause 92. Apparatus as in clause 77 to 91, wherein the stress-applying features comprise a sharp element projecting outwardly from the convex rounded upper surface and wherein the sharp element is configured to concentrate stress when engaged against the calcification on the wall of a vascular lumen when the convex rounded upper surface is pressed against a surface of the calcification.

Clause 93. Apparatus as in clause 92, wherein the convex rounded upper surface extends above a surface of the scaffold by a first distance and the sharp element projects from the surface of the convex rounded upper surface by a second distance equal to 0.05 to 0.1 of the first distance.

Clause 94. Apparatus as in clause 92 or 93, wherein the second distance is in a range from 0.01 mm to 0.2 mm, or 0.01 mm to 0.1 mm.

Clause 95. Apparatus as in clause 92 to 94, wherein the sharp element comprises a point.

Clause 96. Apparatus as in clause 92 to 94, wherein the sharp element comprises an edge.

Clause 97. Apparatus as in any one of clauses 77 to 96, wherein the expandable structure comprises an inflatable balloon.

Clause 98. Apparatus as in clause 97, wherein the inflatable balloon has a central region, a distal tapered region, and a proximal tapered region and wherein the stress-applying features are present on one or more of these regions.

Clause 99. Apparatus as in clause 97 or 98, wherein the stress-applying features are present on at least the central region.

Clause 100. Apparatus as in clause 97 to 99, wherein the stress-applying features are present on at least one of the distal and proximal tapered regions.

Clause 101. Apparatus as in clause 100, wherein the stress-applying features are present on both of the distal and proximal tapered regions.

Clause 102. Apparatus as in clause 97 to 101, wherein the inflatable balloon has a distensibility below 10% when inflated to a pressure of at least 8 atm, at least 10 atm, at least 12 atm, at least 16 atm, at least 18 atm or at least 20 atm.

Clause 103. Apparatus as in any one of clauses 77 to 102, wherein the stress-applying features are attached to the outer surface of the expandable structure by at least one of adhesive bonding, ultrasonic welding, fusion, heat welding, press fitting, solvent bonding, bonding with a polymeric material, use of a fastener, and combinations thereof.

Clause 104. Apparatus as in any one of clauses 77 to 103, further comprising an outer sleeve positioned over the stress-applying features on the outer surface of the expandable structure.

Clause 105. Apparatus as in clause 104, wherein the outer sleeve comprises a retractable sheath configured to shield the stress-applying features as the apparatus is advanced and/or retracted through the body lumen.

Clause 106. Apparatus as in clause 105, wherein the outer sleeve comprises an elastomeric tubular member positioned over the outer surface of the expandable structure and conforming to the stress-applying features when the expandable structure is expanded, wherein the elastomeric tubular member is configured to expand and contract with the expandable structure.

Clause 107. Apparatus as in clause 104, wherein the elastomeric tubular member is laminated or attached to at least a portion of the outer surface of the expandable structure.

Clause 108. Apparatus as in clause 104, wherein the outer sleeve comprises a non-distensible or semi-compliant sheath folded over the balloon before the balloon is inflated.

Clause 109. Apparatus as in clause 104 to 108, wherein the outer sleeve fully covers the clot disruption features on the outer surface of the expandable structure.

Clause 110. Apparatus as in any one of clauses 104 to 109, wherein the outer sleeve comprises a polymer.

Clause 111. Apparatus as in clause 106 to 110, wherein at least some of the stress-applying features are attached to an inner surface of the elastomeric tubular member.

Clause 112. Apparatus as in clause 111, wherein at least some of the stress-applying features are formed as a protrusion from the inner surface of the elastomeric tubular member.

Clause 113. Apparatus as in any one of clauses 77 to 112, wherein at least some of the stress-applying features have a base attached to the outer surface of the expandable structure, said base having a width in an axial direction (Wa) and a width in a circumferential direction (Wc) with a width ratio Wa:Wc in a range from 1:0.5 to 1:5; usually from 1:1 to 1 to 1:5; more usually from 1:1 to 3:1.

Clause 114. Apparatus as in clause 113, wherein at least some of the bases have a circular periphery.

Clause 115. Apparatus as in clause 113, wherein at least some of the bases have an oval periphery.

Clause 116. Apparatus of any one of clauses 77 to 115, wherein at least some of the stress-applying features are arranged in diametrically opposed pairs.

Clause 117. Apparatus of clause 116, wherein successive diametrically opposed pairs of stress-applying features are circumferentially offset.

Clause 118. Apparatus of clause 117, wherein the successive diametrically opposed pairs of stress-applying features are circumferentially offset by an angle from 45° to 90°.

Clause 119. Apparatus of any one of clauses 77 to 115, wherein at least some of the stress-applying features are arranged in groups of three which are circumferentially separated about a circle on the surface of the expandable structure by about 1200.

Clause 120. Apparatus of any one of clauses 77 to 119, wherein the inflatable balloon is configured to release an inflation medium comprising a medicament in response to an inflation pressure above a minimum threshold value.

Clause 121. Apparatus of clause 120, wherein the minimum threshold value is above 3 atm, 5 atm, or 7 atm.

Clause 122. Apparatus of any one of clauses 120 to 121, wherein the inflatable balloon comprises a plurality of ports which open in response to the inflation pressure above the minimum threshold value.

Clause 123. A method for treating calcification on a wall in a patient's body lumen, said method comprising:
positioning an expandable structure at a treatment site proximate the calcification to be treated;
radially outwardly expanding the expandable structure to radially outwardly press a plurality of stress-applying features against the calcification, wherein the stress-applying features are distributed over the outer surface of the expandable structure and at least some of the stress-applying features and have convex rounded upper surfaces and wherein radially outwardly pressing the plurality of stress-applying fractures into the calcification fractures the calcification while reducing damage to the wall.

Clause 124. A method as in clause 123, wherein the body lumen comprises a blood vessel, a valve annulus, a venous valve, or AV shunt.

Clause 125. A method as in clause 123 or 124, wherein the calcification is located within an inner wall, an intimal layer, a medial layer, an adventitial layer, a valve leaflet, a valve annulus, a venous filter, or an implant.

Clause 126. A method as in any one of clauses 123 to 125, wherein the convex rounded upper surfaces of the stress-applying features are free from edges and irregularities which could damage the vascular wall when the expandable structure is expanded within the body lumen.

Clause 127. A method as in clause 123 to 126, wherein the stress-applying features comprise a sharp element projecting outwardly from the convex rounded upper surface and wherein the sharp element is configured to concentrate stress when engaged against the calcification on the wall of a vascular lumen when the convex rounded upper surface is pressed against a surface of the calcification.

Clause 128. A method as in clause 127, wherein the convex rounded upper surface extends above a surface of the scaffold by a first distance and the sharp element projects from the surface of the convex rounded upper surface by a second distance equal to 0.05 to 0.1 of the first distance.

Clause 129. A method as in clause 127 or 128, wherein the second distance is in a range from 0.01 mm to 0.2 mm, or 0.01 mm to 0.1 mm.

Clause 130. A method as in clause 127 to 129, wherein the sharp element comprises a point.

Clause 131. A method as in clause 127 to 129, wherein the sharp element comprises an edge.

Clause 132. A method as in any one of clauses 123 to 131, wherein expanding comprises inflating a balloon having the plurality of stress-applying features independently attached to an outer surface of the balloon.

Clause 133. A method as in any one of clauses 123 to 132, wherein at least some of the stress-applying features comprise spheres or ellipsoids having lower surfaces attached to the outer surface of the expandable structure.

Clause 134. A method as in any one of clauses 123 to 132, wherein at least some of the stress-applying features comprise hemispheres having lower surfaces attached to the outer surface of the expandable structure.

Clause 135. A method as in any one of clauses 123 to 132, wherein at least some of the stress-applying features comprise posts having hemispherical upper surfaces and lower surfaces attached to the outer surface of the expandable structure.

Clause 136. A method as in any one of clauses 123 to 135, wherein the lower surfaces are attached directly to the outer surface of the expandable structure.

Clause 137. A method as in any one of clauses 123 to 135, wherein the lower surfaces comprise bases which are attached directly to the outer surface of the expandable structure.

Clause 138. A method as in any one of clauses 123 to 137, wherein at least some of the stress-applying features have a base attached to the outer surface of the expandable structure, said base having a width in an axial direction (Wa) and a width in a circumferential direction (Wc) with a width ratio Wa:Wc in a range from 1:0.5 to 1:5; usually from 1:1 to 1 to 1:5; more usually from 1:1 to 3:1.

Clause 139. A method as in clause 138, wherein at least some of the bases have a circular periphery.

Clause 140. A method as in clause 138, wherein at least some of the bases have an oval periphery.

Clause 141. A method of any one of clauses 123 to 140, wherein at least some of the stress-applying features are arranged in diametrically opposed pairs.

Clause 142. A method of clause 141, wherein successive diametrically opposed pairs of stress-applying features are circumferentially offset.

Clause 143. A method of clause 142, wherein the successive diametrically opposed pairs of stress-applying features are circumferentially offset by an angle from 45° to 90°.

Clause 144. A method of any one of clauses 123 to 140, wherein at least some of the stress-applying features are arranged in groups of three which are circumferentially separated about a circle on the surface of the expandable structure by about 120°.

Clause 145. A method as in any one of clauses 123 to 144, wherein radially outwardly expanding the expandable scoring structure comprises inflating an inflatable balloon.

Clause 146. A method as in clause 145, wherein the inflatable balloon has a distensibility below 10% when inflated to 8 atm, at least 10 at, at least 12 atm, at least 16 atm, at least 18 atm or at least 20 atm.

Clause 147. A method of any one of clauses 145 to 146, wherein the inflatable balloon is inflated to a pressure that presses the convex rounded upper surfaces of the stress-applying features against the inner wall of the body lumen while not engaging the outer surface the inflatable balloon against the inner wall of the body lumen.

Clause 148. A method of any one of clauses 145 to 147, wherein prior to inflation, the inflatable balloon remains sufficiently flexible to be advanced through the body lumen.

Clause 149. A method as in any one of clauses 123 to 148, wherein the stress-applying features are attached to the outer surface of the expandable structure by at least one of an adhesive, ultrasonic welding, heat welding, a fastener, solvent bonding, bonding with a polymeric material, or combinations thereof.

Clause 150. A method as in any one of clauses 123 to 149, further comprising an outer sleeve positioned over the stress-applying features on the outer surface of the expandable structure.

Clause 151. A method as in clause 150, wherein the outer sleeve comprises a retractable sheath configured to shield the stress-applying features as the expandable member is advanced and/or retracted through the body lumen.

Clause 152. A method as in clause 150, wherein the outer sleeve comprises an elastomeric tubular member positioned over the outer surface of the expandable structure and conforming to the stress-applying features, wherein the elastomeric tubular member is configured to expand and contract with the expandable structure and the stress-applying features remain configured to disrupt plaque when expanded against plaque.

Clause 153. A method as in clause 152, wherein the elastomeric tubular member is laminated to at least a portion of the outer surface of the expandable structure.

Clause 154. A method as in clause 150, wherein the outer sleeve comprises a non-distensible or semi-compliant sheath folded over the balloon before the balloon is inflated.

Clause 155. A method as in clause 123 to 154, wherein the outer sleeve fully covers the clot disruption features on the outer surface of the expandable structure.

Clause 156. A method as in clause 153 to 155, wherein at least some of the stress-applying features plaque are attached to an inner surface of the elastomeric tubular member.

Clause 157. A method as in clause 156, wherein at least some of the stress-applying features are formed as a protrusion from the inner surface of the elastomeric tubular member.

Clause 158. A method as in any one of clauses 123 to 155, wherein at least some of the stress-applying features have a base attached to the outer surface of the expandable structure, said base having a width in an axial direction (Wa) and a width in a circumferential direction (Wc) with a width ratio Wa:Wc in a range from 1:0.5 to 1:5; usually from 1:1 to 1 to 1:5; more usually from 1:1 to 3:1.

Clause 159. A method as in clause 158, wherein at least some of the bases have a circular periphery.

Clause 160. A method as in clause 158, wherein at least some of the bases have an oval periphery.

Clause 161. A method of any one of clauses 123 to 160, wherein at least some of the stress-applying features are arranged in diametrically opposed pairs.

Clause 162. A method of clause 161, wherein successive diametrically opposed pairs of stress-applying features are circumferentially offset.

Clause 163. A method of clause 162, wherein the successive diametrically opposed pairs of stress-applying features are circumferentially offset by an angle from 45° to 90°.

Clause 164. A method of any one of clauses 123 to 160, wherein at least some of the stress-applying features are arranged in groups of three which are circumferentially separated about a circle on the surface of the expandable structure by about 120°.

Clause 165. A method of any one of clauses 123 to 164, wherein stress-applying features are arranged in a multiplicity of circumferential ring patterns, wherein each ring pattern includes from 1 to 10 features, preferably ranges from 2 to 5 features, and more preferably ranges from 3 to 4 features.

Clause 166. A method of clause 165, wherein the circumferential ring patterns are axially spaced-apart over a length of the expandable structure and separated by gaps in a range from 0.1 mm to 3 mm.

Clause 167. A method of clauses 165 or 166, wherein the number of features ranges from 2 to 200 per mm of axial length.

Clause 168. A method of any one of clauses 123 to 167, further comprising releasing an inflation medium comprising a medicament through the inflatable balloon in response to an inflation pressure above a minimum threshold value.

Clause 169. A method of clause 168, wherein the minimum threshold value is above 3 atm, 5 atm, or 7 atm.

Clause 170. A method of clauses 168 or 169, wherein the inflatable balloon comprises a plurality of ports which open in response to the inflation pressure above the minimum threshold value.

Clause 171. Apparatus for treating a patient valve having calcified leaflets, said apparatus comprising:
a catheter body having a proximal end and a distal end; and
a segmented balloon structure disposed at the distal end of the catheter body, said segmented balloon structure having opposed internal walls configured to be expanded on opposite surfaces of the leaflets to disrupt calcification on the calcified valve.

Clause 172. Apparatus of clause 171, wherein the opposed internal walls are configured to close together when the balloon structure is expanded.

Clause 173. Apparatus of clause 172, wherein the opposed internal walls are configured to nest when the balloon structure is expanded.

Clause 174. Apparatus of clause 173, wherein the nested opposed internal walls comprise nesting conical surfaces.

Clause 175. Apparatus of clause 172, wherein the opposed internal walls comprise flat surfaces that are configured to close against each other when the balloon structure is expanded.

Clause 176. Apparatus of clause 175, wherein the segmented balloon structure comprises a pair of opposed conical balloon having flat bases which comprise the flat surfaces.

Clause 177. Apparatus of clause 171 to 176, further comprising a plurality of calcification disruption features distributed over at least one of opposed internal walls of the segmented balloon structure.

Clause 178. Apparatus of clause 177, wherein the calcification disruption features are distributed over both of the opposed internal walls of the segmented balloon structure.

Clause 179. Apparatus of clause 177 or 178, wherein at least some of the calcification disruption features have convex rounded leaflet engaging surfaces configured to fracture the calcification while minimizing damage to the leaflet when the balloon structure is expanded in the patient valve.

Clause 180. Apparatus as in clause any one of clauses 177 to 179, wherein the convex rounded upper surfaces of plurality of stress-applying features are configured to extend from the opposed internal walls of the expandable balloon when said balloon is inflated.

Clause 181. Apparatus as in clause 180, wherein the convex rounded upper surfaces extend from the opposed internal walls by a distance in a range from 0.1 mm to 3 mm, preferably ranging from 0.25 mm to 3 mm, more preferably ranging from 0.5 mm to 3 mm, when said balloon is fully inflated.

Clause 182. Apparatus as in any one of clauses 204 to 206, wherein the convex rounded upper surfaces of the calcification disruption features are free from edges and irregularities which could damage the leaflets when the balloon structure is inflated within the patient valve.

Clause 183. Apparatus as in clause 179 to 182, wherein the calcification disruption features comprise a sharp element projecting outwardly from the convex rounded upper surface and wherein the sharp element is configured to concentrate stress when engaged against the calcification on the valve leaflet when pressed against a surface of the valve leaflet.

Clause 184. Apparatus as in any one of clauses 171 to 183, wherein the balloon segments are fixed on the catheter body with a fixed spacing between the opposed internal walls.

Clause 185. Apparatus as in any one of clauses 171 to 183, wherein the balloon segments are configured to axially translate relative to each other on the catheter body with a variable spacing between the opposed internal walls.

Clause 186. Apparatus as in any one of clauses 171 to 185, wherein the calcification disruption features on the opposing surfaces are axially aligned as the balloon segments are drawn together.

Clause 187. Apparatus as in any one of clauses 171 to 186, wherein the calcification disruption features on the opposing surfaces are laterally offset so that they do not align axially as the balloon segments are drawn together.

Clause 188. A method for treating a patient valve having calcified leaflets, said method comprising:
providing a catheter body having a segmented balloon structure disposed at a distal end thereof;
intravascularly advancing the segmented balloon structure to the patient valve; and
expanding opposed internal walls of the segmented balloon on opposite surfaces of the leaflets to disrupt calcification on the calcified valve.

Clause 189. The method of clause 188, wherein the opposed internal walls are configured to close together when the balloon structure is expanded.

Clause 190. The method of clause 189, wherein the opposed internal walls are configured to nest when the balloon structure is expanded.

Clause 191. The method of clause 189, wherein the nested opposed internal walls comprise nesting conical surfaces.

Clause 192. The method of clause 189, wherein the opposed internal walls comprise flat surfaces that are configured to close against each other when the balloon structure is expanded.

Clause 193. The method of clause 192, wherein the segmented balloon structure comprises a pair of opposed conical balloon having flat bases which comprise the flat surfaces.

Clause 194. The method of clause 188 to 193, wherein a plurality of calcification disruption features are distributed over at least one of opposed internal walls of the segmented balloon structure.

Clause 195. The method of clause 194, wherein the calcification disruption features are distributed over both of the opposed internal walls of the segmented balloon structure.

Clause 196. The method of clause 188 to 195, wherein at least some of the calcification disruption features have convex rounded leaflet engaging surfaces configured to fracture the calcification while minimizing damage to the leaflet when the balloon structure is expanded in the patient valve.

Clause 197. The method as in clause any one of clauses 194 to 196, wherein the convex rounded upper surfaces of plurality of stress-applying features are configured to extend from the opposed internal walls of the expandable balloon when said balloon is inflated.

Clause 198. The method as in clause 197, wherein the convex rounded upper surfaces of extend from the opposed internal walls by a distance in a range from 0.25 mm to 3 mm, preferably ranging from 0.5 mm to 3 mm, when said balloon is fully inflated.

Clause 199. The method as in any one of clauses 196 to 198, wherein the convex rounded upper surfaces of the calcification disruption features are free from edges and irregularities which could damage the leaflets when the balloon structure is inflated within the patient valve.

Clause 200. The method as in clause 196 to 198, wherein the calcification disruption features comprise a sharp element projecting outwardly from the convex rounded upper surface and wherein the sharp element is configured to concentrate stress when engaged against the calcification on the valve leaflet when pressed against a surface of the valve leaflet.

Clause 201. The method as in any one of clauses 188 to 200, wherein the balloon segments are fixed on the catheter body with a fixed spacing between the opposed internal walls.

Clause 202. The method as in any one of clauses 188 to 200, wherein the balloon segments are configured to axially translate relative to each other on the catheter body, further comprising moving the balloon segments together to compress the walls surfaces against the valve leaflets after the balloon structure is inflated.

Clause 203. A method for disrupting a calcification or plaque at a lesion, said method comprising:
advancing a sleeve over a wire through the lesion;
advancing an expandable member over the wire and into an interior of the sleeve; and
expanding the expandable member within the sleeve to outwardly radially displace features on an inside and/or outside of the sleeve against the lesion to disrupt the calcification or plaque.

Clause 204. A method as in clause 203, further comprising removing the expandable member from the sleeve and removing the expandable member and the sleeve over the wire.

Clause 205. A method as in clause 204, wherein the expandable structure is a balloon or other expandable member.

Clause 206. A method as in clause 203, wherein the expandable structure comprises a stent and wherein the sleeve is left in place between the stent and the lesion after the stent is expanded.

Clause 207. A method as in any one of clauses 203 to 206, wherein the stress-applying
features protrude radially outwardly from the sleeve into the vessel wall when the expandable structure displaces them radially outwardly upon expansion.

Clause 208. A method for disrupting a calcification or plaque at a lesion, said method comprising:
advancing a cage or basket over a wire across the lesion; and
expanding the cage of basket to radially displace stress-applying features on the cage or basket against the lesion to disrupt the calcification or plaque.

Clause 209. The method of clause 208, wherein expanding the cage or basket comprises mechanically reorienting structural components of the cage or basket.

Clause 210. The method of clause 208, wherein expanding the cage or basket comprises inflating a balloon within the cage or basket.

Clause 211. The method of clause 210, wherein the balloon is advanced to the lesion together with the cage or basket.

Clause 212. The method of clause 210, wherein the balloon is advanced to the lesion together after the cage or basket.

Clause 213. Apparatus for treating calcification on a wall in a patient's body lumen, said apparatus comprising:
a catheter including a catheter body having a proximal end and a distal segment;
an expandable structure disposed at the distal segment of the catheter body, said expandable structure having an outer surface configured to be displaced radially outwardly toward an inner surface of the body lumen wall; and
a plurality of stress-applying features distributed over the outer surface of the expandable structure, wherein at least some of the stress-applying features are disposed on the outer surface of the expandable structure and have a convex rounded apex configured to fracture the calcification while minimizing damage to the body lumen when the expandable structure is expanded within the body lumen;
wherein the convex rounded apex of the stress-applying features has a radial height above the outer surface of the expandable structure in a range from a minimum of 0.05 mm, 0.1 mm, 0.15 mm, 0.2 mm, or 0.25 mm to a maximum of 1 mm, 0.5 mm, 0.4 mm, 0.3 mm, or 0.25 mm, and a distribution density in a range from 0.1 to 5 features/mm², preferably from 0.2 to 4 features/mm², more preferably from 0.25 to 3 features/mm² when the expandable structure is expanded.

Clause 214. Apparatus as in clause 213, wherein the stress-applying features have a footprint having a maximum width, diameter, or other lateral dimension of 4 mm or less, often being 3 mm or less, more often being no more than 1 mm, and frequently being no more than 0.75 mm, and sometimes being no more than 0.5 mm.

Clause 215. Apparatus as in clause 213 or 214, wherein the stress-applying features comprise any one or more of balls, spheres, hemispheres, partial spheres, domes, and ellipsoidal solids.

Clause 216. Apparatus as in clause 213 to 215, wherein the stress-applying features are solid.

Clause 217. Apparatus as in clause 213 to 215, wherein the stress-applying features are hollow.

Clause 218. Apparatus as in clause 213 to 217, further comprising an encapsulation layer covering the outer surface of the expandable structure and the stress-applying features to immobilize the stress-applying features in a desired pattern on the outer surface of the expandable structure.

Clause 219. Apparatus as in clause 218, wherein the stress-applying features comprise discrete bodies.

Clause 220. Apparatus as in clause 219, wherein the discrete bodies comprise a metal.

Clause 221. Apparatus as in clause 218 to 220, wherein the stress-applying features are immobilized solely by encapsulation layer.

Clause 222. Apparatus as in clause 218 to 220, wherein the stress-applying features are immobilized by an adhesive between the feature and the outer surface in addition to the encapsulation layer.

Clause 223. Apparatus as in clause 218 to 222, wherein the encapsulation layer encapsulates the entire stress-applying feature including the convex rounded apex.

Clause 224. Apparatus as in clause 218 to 222, wherein the encapsulation layer encapsulates only a lower portion of the stress-applying feature excluding the convex rounded apex.

Clause 225. Apparatus as in clause 213 to 224, wherein the stress-applying feature is cradled in an indentation in the outer surface of the expandable structure.

Clause 226. Apparatus as in clause 213 to 225, wherein encapsulation layer comprises a polymer selected from the group consisting of thermoplastic fluoropolymers (PVDF), butyl methacrylates (PBMA), and thermoplastic polyesters (PLLA).

Clause 227. Apparatus as in clause 218 to 226, wherein the encapsulation layer is applied over the outer surface and the stress-applying features by any one of coating, direct fluid application, laminating, and fusing.

Clause 228. Apparatus as in clause 218 to 227, wherein the encapsulation layer has a thickness in a range from 0.01 mm to 0.1 mm (0.4 mil to 4 mil), often being 0.01 mm to 0.05 mm (0.4 mil to 2 mil), and more often being 0.01 mm to 0.02 mm (0.4 mil to 0.8 mil).

Clause 229. Apparatus as in clause 213 to 228, wherein the stress-applying features are disposed in a recess formed in the outer surface of the expandable structure.

Clause 230. Apparatus as in clause 213 to 229, wherein the stress-applying features are constrained over the outer surface of the expandable structure by an elastic sleeve.

Clause 231. Apparatus as in clause 230, wherein the stress-applying features are further attached to the outer surface of the expandable structure.

Clause 232. Apparatus as in clause 230, wherein the stress-applying features are attached to an inner surface of the elastic sleeve.

Clause 233. Apparatus as in clause 213 to 228, wherein the stress-applying features are hollow and mounted on a post projecting radially outwardly from the outer surface of the expandable structure.

Clause 234. Apparatus for treating calcification on a wall in a patient's body lumen, said apparatus comprising:
a catheter including a catheter body having a proximal end and a distal segment;
   an expandable structure disposed at the distal segment of the catheter body, said expandable structure having an outer surface configured to be displaced radially outwardly toward an inner surface of the body lumen wall;
a plurality of stress-applying features distributed over the outer surface of the expandable structure, wherein at least some of the stress-applying features are present on the outer surface of the expandable structure and have an upper surface configured to fracture the calcification while minimizing damage to the body lumen when the expandable structure is expanded within the body lumen; and
an encapsulation layer covering at least a portion of the outer surface of the expandable structure and the stress-applying features to immobilize the stress-applying features in a desired pattern on the outer surface of the expandable structure.

Clause 235. Apparatus as in clause 234, wherein at least some of the upper surfaces of the stress-applying features comprise a convex rounded apex.

Clause 236. Apparatus as in clause 234 or 235, wherein the encapsulation layer covers the entire outer surface of at least some the stress-applying features including the upper surface.

Clause 237. Apparatus as in clause 234 or 235, wherein the encapsulation layer cover only a lower portion of the outer surface of at least some of the stress-applying features.

Clause 238. Apparatus as in clause 234 to 237, wherein the stress-applying feature is disposed in an indentation on the outer surface of the expandable structure.

Clause 239. Apparatus as in clause 234, wherein the stress-applying feature comprises a hemisphere with a flat bottom adhered to the outer surface of the expandable structure.

Clause 240. Apparatus as in clause 234 to 239, wherein the stress-applying features comprise discrete bodies.

Clause 241. Apparatus as in clause 240, wherein the discrete bodies comprise a metal.

Clause 242. Apparatus as in clause 234 to 240, wherein the stress-applying features comprise any one or more of a ball, a sphere, a hemisphere, a partial sphere, an ellipsoidal solid, and a dome.

Clause 243. Apparatus as in clause 234 to 242, wherein the stress-applying features are immobilized solely by encapsulation layer.

Clause 244. Apparatus as in clause 234 to 243, wherein the stress-applying features are immobilized by an adhesive between the feature and the outer surface in addition to the encapsulation layer.

Clause 245. Apparatus as in clause 234 to 244, wherein encapsulation layer comprises a polymer selected from the group consisting of thermoplastic fluoropolymers (PVDF), butyl methacrylates (PBMA), and thermoplastic polyesters (PLLA).

Clause 246. Apparatus as in clause 234 to 245, wherein the encapsulation layer is applied over the outer surface and the stress-applying features by any one of coating, direct fluid application, laminating, and fusing.

Clause 247. Apparatus as in clause 234 to 246, wherein the encapsulation layer has a thickness in a range from 0.01 mm to 0.1 mm (0.4 mil to 4 mil), often being 0.01 mm to 0.05 mm (0.4 mil to 2 mil), and more often being 0.01 mm to 0.02 mm (0.4 mil to 0.8 mil).

Clause 248. Apparatus as in clause 234 to 247, wherein the convex rounded apex of the stress-applying features has a radial height above the outer surface of the expandable structure in a range from a minimum of 0.05 mm, 0.1 mm, 0.15 mm, 0.2 mm, or 0.25 mm to a maximum of 1 mm, 0.5 mm, 0.4 mm, 0.3 mm, or 0.25 mm, and a distribution density in a range from 0.1 to 5 features/mm², preferably from 0.2 to 4 features/mm², more preferably from 0.25 to 3 features/mm² when the expandable structure is expanded.

Clause 249. Apparatus as in clause 248, wherein the plaque distribution feature has footprint having a maximum width, diameter, or other lateral dimension of 4 mm or less, often being 3 mm or less, more often being no more than 1 mm, and frequently being no more than 0.75 mm, and sometimes being no more than 0.5 mm.

Clause 250. A method for treating a lesion on a wall in a patient's body lumen, said apparatus comprising:
providing a catheter having an expandable structure disposed at a distal end thereof, said expandable structure having an outer surface configured to be displaced radially outwardly toward an inner surface of the body lumen wall, wherein the outer wall has a plurality of space-separating features distributed over the outer surface of the expandable structure; and
expanding the expandable structure in the patient's body lumen such that a radially outward force is applied by the outer surface and the features against the wall while the features maintain a gap between the outer surface of the expandable structure and the inner wall.

Clause 251. A method as in clause 250, wherein the spacer features have axially aligned through holes permitting the passage of contrast media therethrough.

Clause 252. A method as in clause 277 or 278, wherein the gap allows fluid perfusion therethrough and past the expandable structure while the expandable structure is expanded.

Clause 253. A method as in clause 277 or 278, further comprising perfusing a drug into the gap while the expandable structure is expanded.

Clause 254. A method as in clause 253, wherein the expandable structure comprises a balloon and the drug is perfused through a wall of the balloon.

Clause 255. A method as in clause 254, wherein at least some of the space-separating features comprise a drug which is released into the gap.

Clause 256. A method as in clause 250 to 255, wherein expanding the expandable structure creates one or more gaps between the outer surface of the expandable structure and the inner wall under physiological pressure to allow fluid perfusion through the one or more gaps.

Clause 257. A method as in clause 250 to 256, wherein the body lumen comprises a blood vessel and the body fluid perfusion comprises blood.

Clause 258. A method as in clause 257, wherein the body fluid perfusion further comprises at least one of a contrast media and a drug.

Clause 259. A method as in clause 250 to 258, wherein at least some of the features have a convex rounded apex.

Clause 260. A method as in clause 250 to 259, wherein the features have a radial height above the outer surface of the expandable structure in a range from a minimum of 0.05 mm, 0.1 mm, 0.15 mm, 0.2 mm, or 0.25 mm to a maximum of 1 mm, 0.5 mm, 0.4 mm, 0.3 mm, or 0.25 mm, and a distribution density in a range from 0.1 to 5 features/mm², preferably from 0.2 to 4 features/mm², more preferably from 0.25 to 3 features/mm² when the expandable structure is expanded.

Clause 261. A method as in clauses 250 to 260, wherein the expandable structure is expanded with a force sufficient to create and/or maintain one or more gaps against a physiologic pressure from 0.5 psi to 5 psi, preferably from 1 psi to 3 psi.

Clause 262. A method as in clauses 250 to 261, wherein the features are configured to create and/or maintain the one or more gaps by separating the lesion from the outer surface of the expandable structure adjacent to the features when the structure is in the expanded configuration.

Clause 263. A method as in clauses 250 to 262, wherein the feature comprises a plurality of features positioned in a configuration around the circumferential length and/or axial length of the expandable structure to provide, create, or maintain said gaps.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIGS. 1A and 1B illustrate a scaffold having a plurality of stress-applying features distributed over an outer surface thereof in accordance with the principles of the present invention.

FIGS. 2A to 2E illustrate different designs examples for and locations examples of the stress-applying features of the present invention.

FIGS. 2F-1A to 2F-3B illustrate additional configurations examples of the stress-applying features of the present invention.

FIGS. 2G-1A to 2G-4B illustrate examples of the placement of points, edges, and other sharp features on the stress-applying features of the present invention.

FIGS. 2H-1 to 2H-22 illustrate a variety of design examples for the stress-applying features of the present invention.

FIG. 3 illustrates an expandable sleeve example having a helical pattern of stress-applying features on an outer surface thereof.

FIGS. 4A and 4B illustrate a tubular scaffold or sleeve having a first arrangement of stress-applying features on an outer surface thereof.

FIGS. 5A and 5B illustrate a tubular scaffold or sleeve having a second arrangement of stress-applying features on an outer surface thereof.

FIGS. 6A to 6C illustrate a first method for folding integral scaffold elements into stress-applying features on an outer surface of a scaffold.

FIGS. 7A to 7C illustrate a second method for folding integral scaffold elements into stress-applying features on an outer surface of a scaffold.

FIGS. 8A to 8C illustrate a third method for folding integral scaffold elements into stress-applying features on an outer surface of a scaffold.

FIGS. 9A and 9B illustrate an example of how the stress-applying features of the present invention fracture calcified plaque when radially engaged against the plaque.

FIGS. 10A to 10C illustrate an alternative example of the present invention where a plurality stress-applying features are present on an outer surface of an expandable structure, such as an inflatable angioplasty balloon or other medical balloon.

FIGS. 11A to 11C illustrate an example of the present invention having a plurality of stress-applying features similar to those illustrated in FIGS. 10A to 10C but arranged in yet another pattern.

FIGS. 12A to 12C illustrate an example of the present invention having a plurality stress-applying features similar to those illustrated in FIGS. 10A to 10C but arranged in different patterns in accordance with the principles of the present invention.

FIGS. 12D to 12H illustrate yet further variations of the stress-applying feature patterns of the present invention.

FIG. 12H-1A illustrates a first axial spacing pattern between circumferentially adjacent stress-applying features on an outer surface of an inflatable balloon.

FIG. 12H-1B is a detailed view taken along line 1B-1B of FIG. 12H-1A.

FIG. 12H-2A illustrates a second axial spacing pattern between circumferentially adjacent stress-applying features on an outer surface of an inflatable balloon.

FIG. 12H-2B is a detailed view taken along line 2B-2B of FIG. 12H-2A.

FIG. 12H-3A illustrates a third axial spacing pattern between circumferentially adjacent stress-applying features on an outer surface of an inflatable balloon.

FIG. 12H-3B is a detailed view taken along line 3B-3B of FIG. 12H-3A.

FIGS. 12I to 12L illustrate different balloon shapes and stress-inducing feature distribution patterns in accordance with the principles of the present invention

FIGS. 13A and 13B illustrate tubular templates that may be used in positioning a plurality stress-applying features on an outer surface of a balloon or other expandable structure.

FIGS. 14A and 14B illustrate a spherical stress-applying feature in accordance with the principles of the present invention mounted in a circular base (FIG. 14A) and a cylindrical base (FIG. 14B) present on an outer surface of an angioplasty balloon.

FIG. 15 illustrates exemplary peripheral dimensions for the stress-applying feature and/or supporting base of the present invention.

FIGS. 16A to 16H illustrate exemplary attachment methods for spherical, hemispherical and other stress-applying features in accordance with the principles of the present invention.

FIGS. 16D-1 to 16D-6 illustrate exemplary attachment methods for spherical, conical other stress-applying features in accordance with the principles of the present invention, wherein the features are attached in preformed indentations in an outer surface of a balloon or other expandable member which provides a receptacle or “cradle” for immobilizing the feature using one or more adhesive or polymeric materials. FIG. 16D-1 is a perspective view. FIG. 16D-2 is a cross-sectional view of the balloon, and FIGS. 16D-3 to 6 are detailed views of the features in the indentations, taken along lines 1603-1603, 1604-1604, 1605-1605, and 1606-1606 in FIG. 16D-2, respectively. These examples are similar to that illustrated in FIG. 16D.

FIGS. 16E-A to 16E-G Illustrate specific steps for encapsuling a hemispherical feature into an indentation on the outer surface of a stress-applying angioplasty balloon.

FIGS. 16E-1 to 16E-7 illustrate exemplary balloon indentation geometries in accordance with the principles of the present invention with conforming stress-applying shown in broken line.

FIGS. 16F-1 to 16F-4 illustrate exemplary stress-applying features having bases and crowns, where the bases are secured in an indentation in a balloon surface and where the crowns may protrude above the balloon surface, may be flush with the balloon surface, or may be recessed beneath the balloon surface.

FIGS. 16G-1 to 16G-12 illustrate exemplary techniques for attaching the bases of the stress-applying features of the present invention in representative balloon indentation geometries.

FIGS. 16I-1 to 16I-3 illustrate an alternative exemplary technique for attaching the base of a stress-applying features by heat welding to a balloon surface.

FIGS. 17A to 17D illustrate spherical stress-applying features constrained or otherwise held on an outer surface of a balloon using an elastic sleeve.

FIGS. 18A to 18C illustrate attachment of spherical stress-applying features on an inner surface of an elastic sleeve and methods for expansion using an inflatable balloon.

FIG. 19 illustrates stress-applying features present on an outer surface of a drug delivery balloon catheter.

FIGS. 20A and 20B are cross-sectional views of the drug delivery balloon catheter of FIG. 19 shown in pre-inflated and post-inflated configurations, respectively, as used in drug delivery.

FIGS. 21A to 21D illustrate alternative structures and methods for the intravascular delivery of drugs using plaque disrupting features of the present invention. The structures of FIGS. 21C and 21D incorporate elastic sleeves.

FIG. 22 illustrates the stress-applying features of the present invention placed on an exterior of a balloon-expandable scaffold or “cage” of a type intended for temporary placement in a target vascular location for stress-applying and subsequent removal.

FIG. 23A shows the placement of the expandable scaffold of FIG. 22 on a balloon catheter prior to expansion of the balloon catheter.

FIG. 23B shows the expandable scaffold of FIG. 23A on the balloon catheter following expansion of the balloon catheter.

FIG. 24 is an end view an expandable structure, such as a balloon catheter, having features, such as metal spheres, contacting and/or expanding plaque tissue, showing spaces created for fluid and/or contrast material to pass through between an outer surface of the expanded structure and an inner surface of the vessel (or plaque) by the features such as metal spheres.

FIGS. 24A to 24D are cross-sectional views of an expandable structures, such as a balloon catheter, having features, such as metal spheres, contacting and/or expanding plaque tissue, showing different distribution patterns for the features, for example having spaces created for fluid and/or contrast material to pass through between an outer surface of the expanded structure and an inner surface of the vessel (or plaque) by the features, such as metal spheres.

FIGS. 24D-1 to 24D-3 illustrate features of the type illustrated in FIG. 24D having holes though the features which further promote perfusion of contrast media past balloons when inflated in the vasculature.

FIG. 25 shows a conventional balloon angioplasty catheter in a mock blood vessel (clear plastic tube) being perfused with a colored or contrast fluid where the flow is blocked by the expanded balloon.

FIG. 26 a mock blood being perfused with a colored or contrast fluid where the flow bypasses the expanded balloon through spaces created between an outer surface of the expanded balloon and an inner surface of the mock blood vessel by the metal spheres or other features.

FIGS. 27, 28, 29, 30A, 30B, 31A to 31F, 32, 33A to 33D, 34, and 35A-35C illustrate different embodiments of a catheter having a segmented balloon design or multi-balloon design with opposed surfaces with stress-applying feature configured to capture cardiac or other valve leaflets to disrupt calcification of the patient valve.

FIGS. 36-38 illustrate different magnetic catheter embodiments for retrieving with stress-applying features that are lost from the catheters of the present invention.

FIGS. 39-42 are images of the steps in fabricating a balloon catheter having stress-applying features encased in multiple elastic polymer layers on a balloon surface.

FIG. 43 illustrates a balloon of a balloon catheter having stress-applying features covered by or coated over with a polymeric or other material.

FIGS. 44A-44D illustrate different embodiments of the covered stress-applying features of FIG. 43.

FIG. 45 illustrates a balloon of a balloon catheter having elongated cutting stress-applying features covered by or coated over with a polymeric or other material.

FIG. 46 illustrate a balloon of a balloon catheter having elongated cutting stress-applying features partially covered by or coated over with a polymeric or other material.

FIG. 46A is a detailed, cross-sectional view taken along line 46A-46A of FIG. 46.

FIGS. 47A and 47B illustrate a single valvuloplasty balloon comprising distal and proximal segments with a transversely oriented waist (annular channel) between the segments configured to capture valve leaflets therebetween.

FIGS. 48A and 48B illustrate a double valvuloplasty balloon structure including separately inflatable distal and proximal balloons with a transversely oriented gap therebetween and configured to capture valve leaflets between opposed, transversely oriented opposed surfaces thereof.

FIGS. 49A, 49B, 50, and 51 illustrate opposed balloon surface configurations suitable for the balloon structures of FIGS. 47A, 47B, 48A, and 48B.

FIG. 52 illustrates a valvuloplasty system comprising an inner valvuloplasty catheter and an outer sleeve catheter to capture and disrupt calcifications on cardiac and other valves.

FIGS. 53A-53F illustrate alternative embodiments of an expandable capture sleeve of the outer sleeve catheter of FIG. 52.

FIGS. 54A and 54B illustrate the leaflets of an aortic valve in diastole with the leaflets closed (FIG. 54A) to allow filling of the left ventricle and systole with the leaflets open to allow the left ventricle to pump blood into the ascending aorta (FIG. 54B).

FIGS. 55A-55C illustrate use of the system of FIG. 52 with the expandable capture sleeve embodiment of FIG. 53D in a procedure for to capture and disrupt calcifications on leaflets of an aortic valve.

FIG. 56A illustrates a balloon catheter carrying both a plurality elongate scoring elements and a multiplicity of discrete or point-like stress-applying features disposed between the elongate scoring elements.

FIG. 56B is a cross-sectional view taken along line 56B-56B in FIG. 56A.

FIG. 57A illustrates a balloon catheter carrying a plurality elongate scoring elements having a multiplicity of discrete or point-like stress-applying features disposed along radially outward surfaces of the elongate scoring elements.

FIG. 57B is a cross-sectional view taken along line 57B-57B in FIG. 57A.

FIGS. 58A and 58B illustrate a balloon catheter carrying a plurality spherical stress-applying features disposed in two circumferential rings over a distal region of an outer surface of a valvuloplasty balloon.

FIGS. 59A and 59B illustrate a balloon catheter carrying a plurality rectangular stress-applying features disposed in two circumferential rings over a distal region of an outer surface of a valvuloplasty balloon.

FIGS. 60A and 60B illustrate a balloon catheter carrying a pair of continuous, zig-zag stress-applying features disposed in two circumferential rings over a distal region of an outer surface of a valvuloplasty balloon.

FIGS. 61A to 61F illustrate a balloon catheter having rigid features distributed over portions of one of one or more of (a) a distal transition region, (b) a proximal transition region, (c) a distal tapered region, (d) a proximal tapered region, (e) a distal terminal 2 mm length of the central region, and (f) a proximal terminal 2 mm length of a central region of the outer surface of an inflatable polymeric balloon.

### DETAILED DESCRIPTION OF THE INVENTION

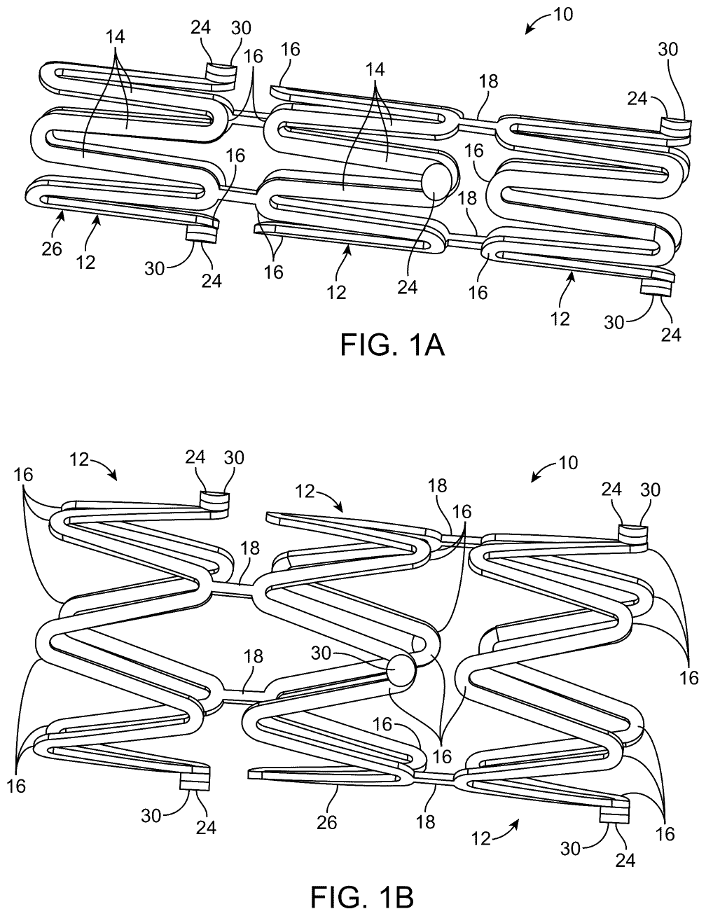

Referring to FIGS. 1A and 1B, an expandable structure comprising an endoluminal prosthesis comprises a radially expanding scaffold 10, illustrated in the form of a stent including a plurality of ring structures 12. The stent scaffold 10, as illustrated in FIGS. 1A and 1B, includes three discrete circumferential rings 12 joined by axial links 18 which together form a cylindrical or other envelope having free ends. While three rings are illustrated, it will be appreciated that stent scaffolds according to the present invention may comprise anywhere from a single circumferential ring structure to as many as 5, 10, or even more ring structures. The ring structures are usually joined into cylinders having generally circular cross-sectional shapes, but may other tubular geometries such as ellipsoidal, tapered, hourglass shaped, dog-bone shaped, oblong, other, and the like, as are well known in the art of vascular and other intraluminal scaffolds.

Each of the ring structures 12 comprises a plurality of struts, 14, usually straight struts, joined by U-shaped or other crowns 16. While such “serpentine” ring structures are most common in the fabrication of vascular stents, other geometries including zig-zag rings, spiral wire, helical wire, wire bent, diamond-shaped cells, other, and the like, are also well known in the vascular stent art and could be used in the present invention.

As shown in FIG. 1A, scaffold 10 will initially be in a “crimped” or small configuration, i.e., having a narrow profile suitable for introduction to a patient's blood vessel or other body lumen. Once in place, scaffold 10 will be caused to assume a radially expanded profile, as shown in FIG. 1B. Such radial expansion can be affected in any conventional manner, typically by balloon expansion or by “self-expansion” where an elastic stent is initially radially compressed and subsequently released from compression to allow expansion at a target location in the vasculature or other body lumen.

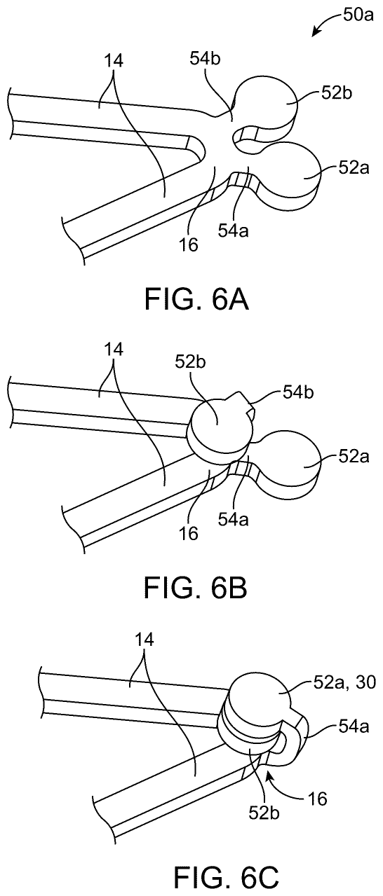

Of particular interest to the present invention, the scaffolds 10 of the present invention will have a plurality of stress-applying features 24 over their outer surfaces 26. The stress-applying features 24 may be pre-formed and attached to the outer surface of the scaffold after the scaffold has been separately fabricated, e.g., by gluing with an adhesive, soldering, welding, mechanical crimping, press fit, threaded attachment, or the like. Alternatively, the stress-applying features may be formed as part of the scaffold during fabrication of the scaffold, e.g., by cutting, machining, deposition, deformation, or combinations thereof. Particular methods for forming the stress-applying features are described with reference to FIGS. 6A to 6C, 7A to 7C, and 8A to 8C.

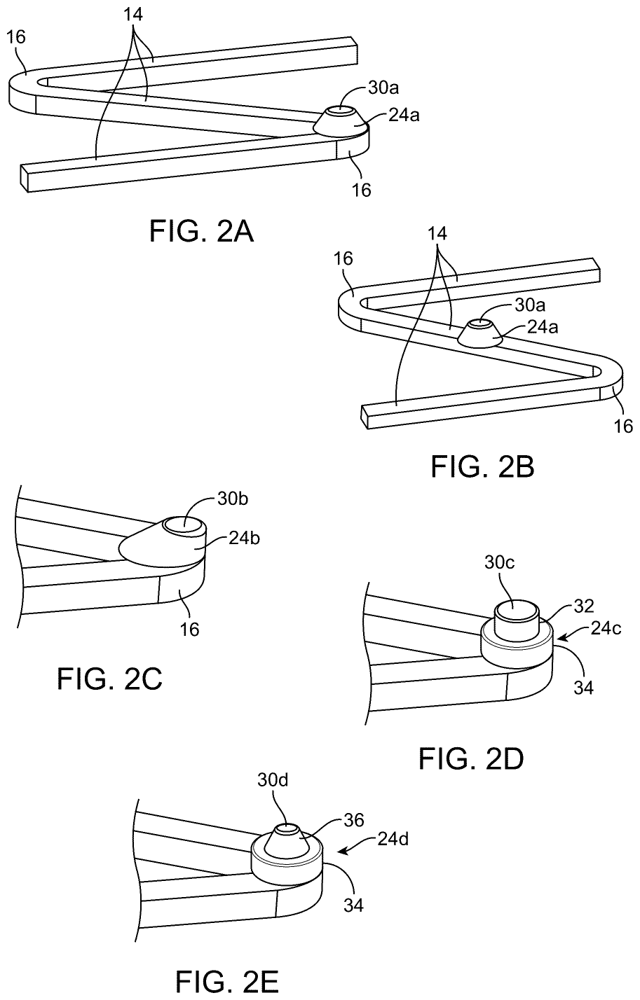

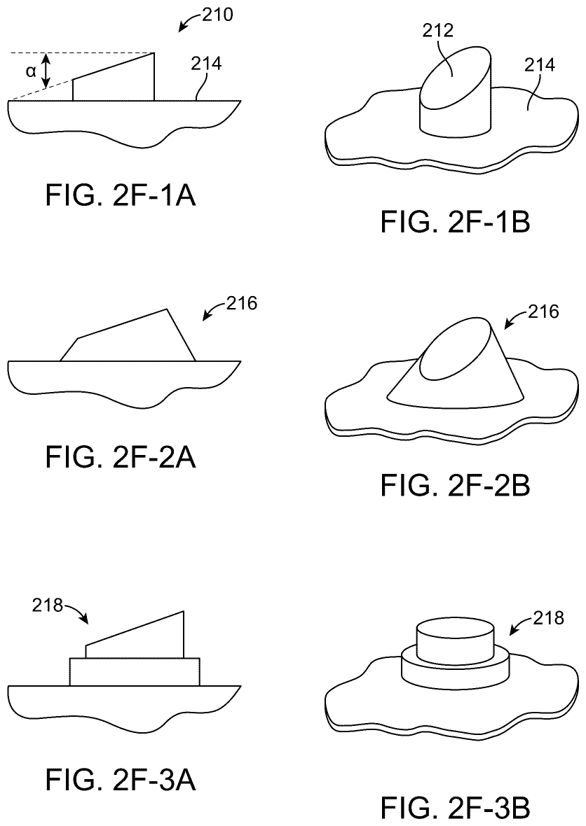

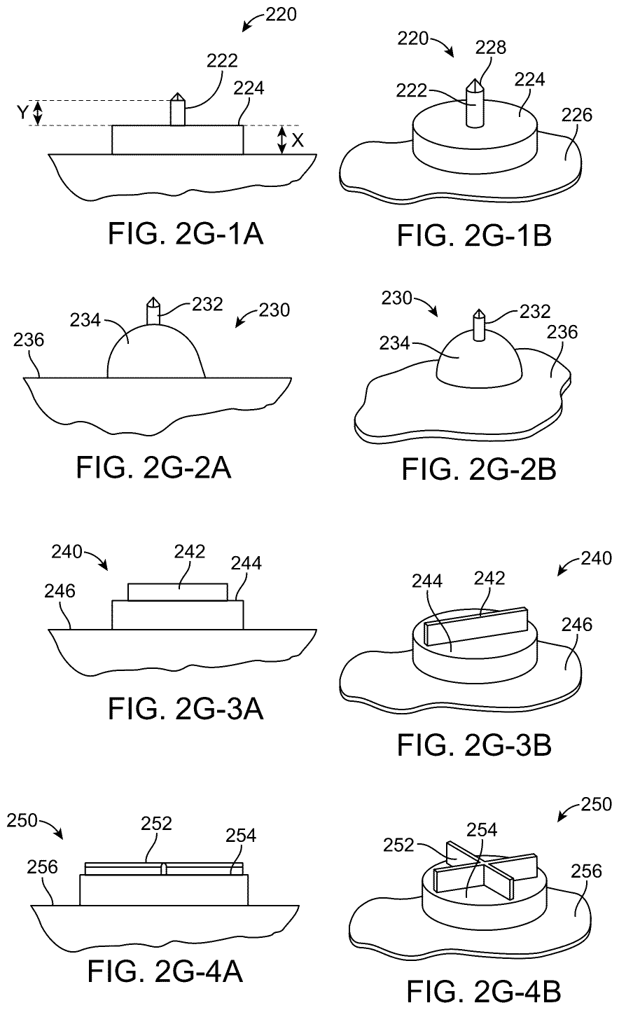

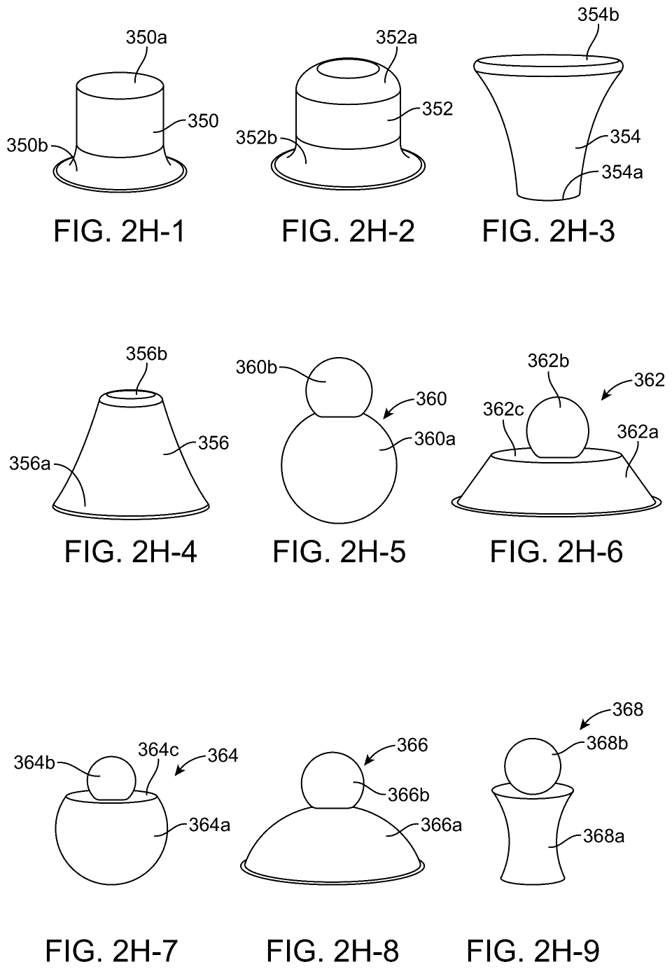

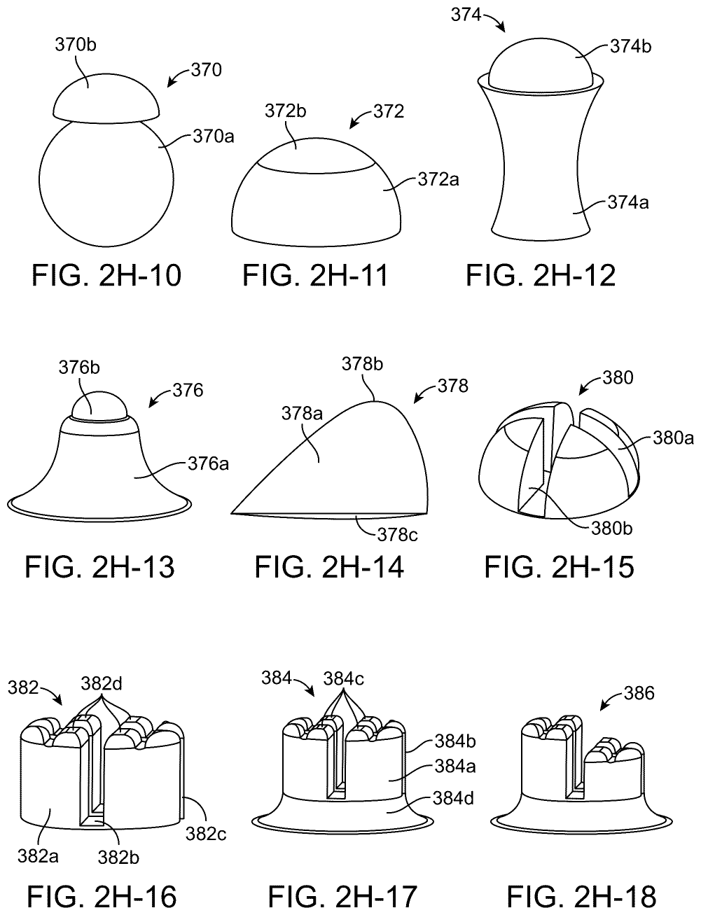

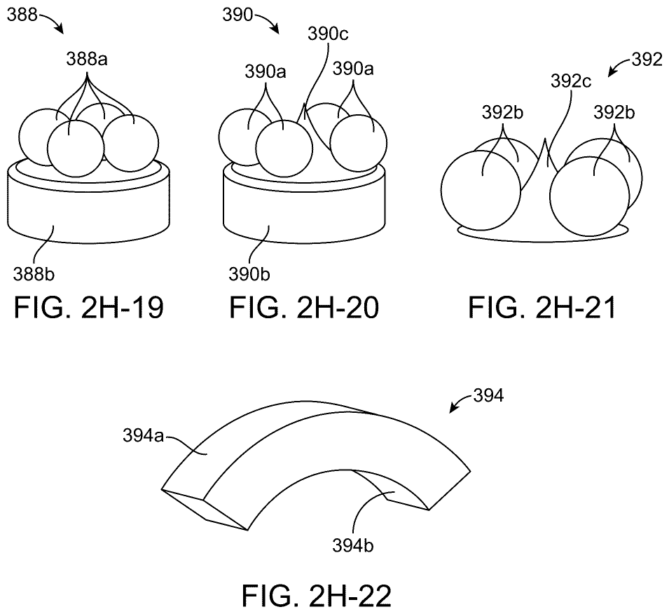

As shown FIGS. 1A and 1B, the stress-applying features 24 are disk-shaped and have generally circular blunt contact regions 30 on their radially outwardly exposed surfaces. Although generally preferred, such stress-applying figures may have a variety of other geometries some of which are described throughout the present invention with reference to FIGS. 2A to 2E, FIGS. 2F, FIG. 2G, and FIG. 2F.

While some of the stress-applying features and/or stress-applying features described herein are illustrated together with specific radially expanding scaffolds, such as stents, sleeves, balloons, valvuloplasty balloons, and the like, it should be appreciated that the stress-applying features and stress-applying features described herein will be useful with and/or apply to at least some or any radially expanding structure, including at least balloons, stents, sleeves, valvuloplasty balloons and the like.

As further shown in FIGS. 1A and 1B, the stress-applying features 24 are attached over “free” ends of crowns 16 located on opposite sides of a ring structure 12. By “free” ends, it is meant that the crowns are free from attachment to adjacent crowns or other structure of the scaffold 10.

Successive pairs of stress-applying features 24 are located 180 apart on each ring structures 12, but the alignment is typically staggered by 90° on each successive ring 12. While a preferred arrangement, it will be appreciated that the number of and arrangements of individual stress-applying features 24 may vary widely as described previously in the present application.

As shown in the examples of FIGS. 2A through 2E, the geometry and position of the stress-applying feature may vary significantly. As shown in FIG. 2A, a stress-applying feature 24a may be shaped as a truncated cone with the truncation forming a blunt contact region 30a.

As shown in the example of FIG. 2B, the truncated cone 24a may be positioned on a strut 14, e.g., at a center of the strut. Additionally or alternatively, stress-applying features can be positioned anywhere along a strut length as well as at different locations on an individual scaffold 10, including crowns, struts, links, and the distribution of individual stress-applying features may vary over different axial and circumferential regions of the outer scaffold surface.

Referring now to FIGS. 2C through 2E, the geometry of individual stress-applying features 24b to 24d may vary. For example, as shown in FIG. 2C, a stress-applying feature 24b may be formed asymmetrically as a truncated cone having a blunt contact region 30b displaced into alignment with an outer distal tip of the crown 16. As shown in FIG. 2D, a stress-applying feature 24c may be formed as two or more stacked disks or other stacked features having other shapes, where each of the stack features may have the same or a different configuration, for example including a larger diameter base disk 34 and a smaller diameter upper disk 32 with a blunt contact region 30c on the exposed surface of a smaller diameter upper disk 32. As shown in FIG. 2E, the larger diameter base disk 34 can be combined with a smaller upper truncated cone 36 with blunt contact region 30d formed by the truncation. The one or more features may also be convex, rounded, blunt, and/or atraumatic type shaped as described in this invention.

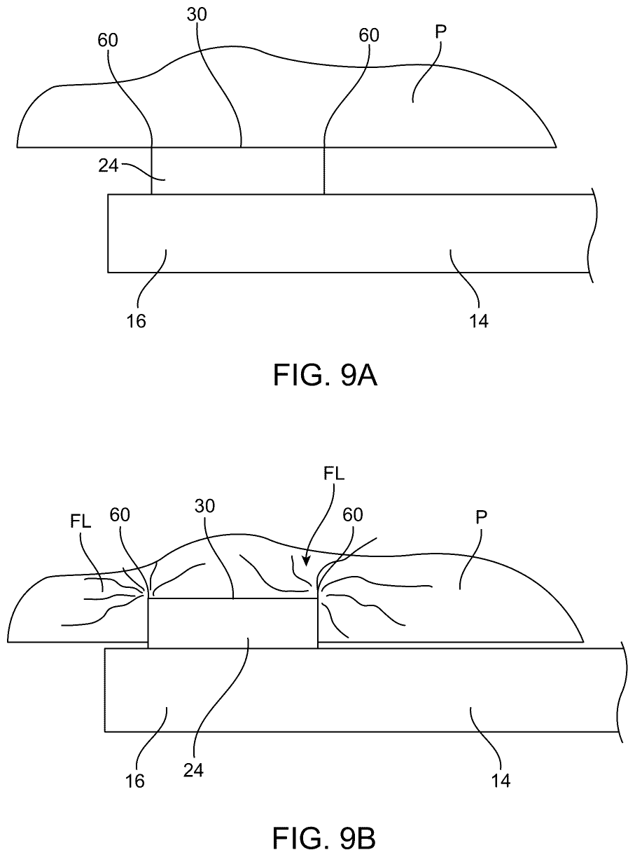

In most cases, the blunt contact region 30 is located at or near a top of the stress-applying feature 24, i.e., the most radially outward location on the outer surface of the scaffold (or other expandable balloon, sleeve, or the like, as discussed below). As such, the blunt contact regions 30 will be the first regions on a scaffold 10 to engage any occlusive material present on the inner wall of the blood vessel or other body lumen when the scaffold is radially expanded therein, as explained further with reference to FIGS. 9A and 9B, below.

As described at this point, the stress-applying features of the present invention have been shown to have blunt contact regions which are generally parallel the surfaces upon which they are mounted. In other instances, the blunt contact regions may be inclined relative the surfaces upon which they are mounted. As shown in FIGS. 2F-1A/1B, an exemplary disk-like stress-applying feature 210 has a blunt contact region 212 inclined at an angle α relative to the surface 214 upon which is mounted. Angle α is typically in a range from 5° to 45°, usually being from 10° to 35°. A conical stress-applying feature 216 as well as a stacked stress-applying feature 218 may also have inclined contact regions, as shown in FIGS. 2F-2A/2B and 3A/3B, respectively.

As shown in FIGS. 2G-1A and 2G-1B, an exemplary stress-applying feature 220 includes a sharp element 222 mounted on and projecting upwardly from a blunt contact region 224. The stress-applying feature 220 may be mounted on a surface 226 of any one of a scaffold, a sleeve, or an angioplasty or other balloon, as described in detail elsewhere herein. The sharp element 222 typically comprises a short shaft having a pointed tip 228 configured to initiate disruption of plaque or calcification as the stress-applying feature is pressed against the plaque or calcification. Preferred dimensions for the sharp element are provided elsewhere herein

As shown in FIGS. 2G-2A and 2G-2B, a stress-applying feature 230 comprises a dome-like or hemispherical body with a rounded upper surface 234 for engaging plaque or calcification, as described in detail elsewhere herein. A sharp element 232 projects upwardly from the rounded upper surface 234, typically from the apex of the rounded upper surface. The stress-applying feature 230 is mounted on a surface to 236 of a scaffold, sleeve, balloon, or other structure as described in more detail elsewhere herein. The sharp element 232 is a short shaft having a pointed distal tip, similar to the sharp element 222.

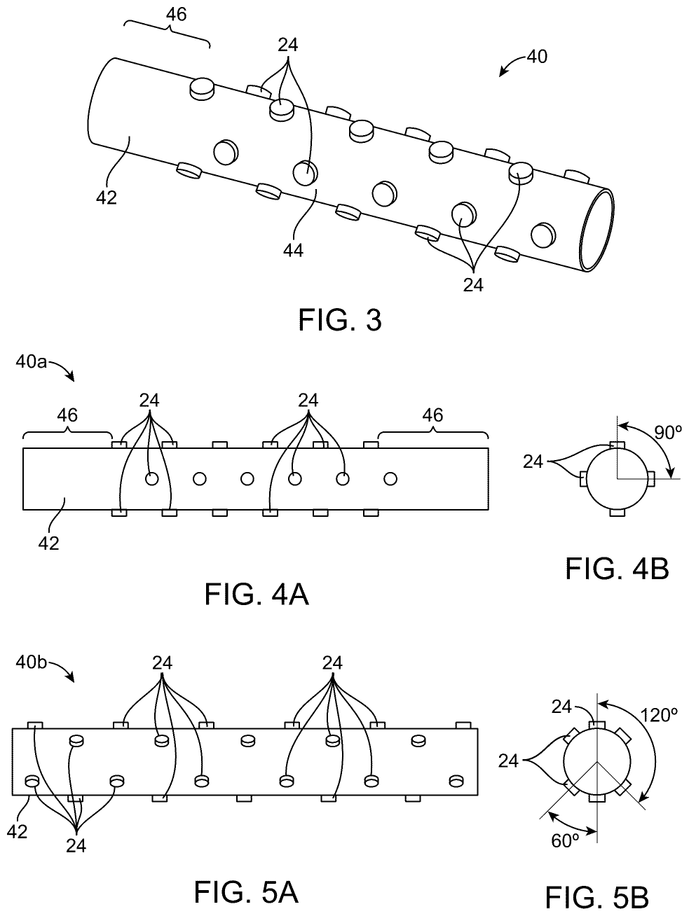

The sharp elements may also comprise blades having one or more elongate sharp edges for initiating the calcification disruption. As shown in FIGS. 2G-3A and 3B, a stress-applying feature 240 comprises a disc-like blunt contact region 244 having a sharp element 242 projecting upwardly therefrom. The sharp element 242 consists of a single blade having a sharp edge, and the stress-applying feature is mounted on a surface 246 of a scaffold, balloon, cage, or sleeve as with other embodiments herein. As shown in FIGS. 2G-4A and 4B, a stress-applying feature 250 may comprise a disk-like blunt contact region 254 with a sharp element 252 including a pair of sharpened blades. As with previous embodiments, the stress-applying feature 250 is configured to be mounted on a surface 256 of a scaffold, balloon, cage, or sleeve.

The stress-applying features of the present invention may have any one of a variety of forms and designs, including blunt tissue-engagement surfaces, curved tissue-engagement surfaces, stacked-tissue engagement structures, segmented tissue-engagement structures, and the like, and a number of specific designs have already been shown and described. A number of additional, exemplary designs are shown in FIGS. 2H-1 to 2H-22, as will now be described. All specific stress-applying feature designs illustrated herein, including but not limited to those shown in FIGS. 2F-1A through 2H-22. May be used with any of the expandable structures described herein, including but not limited to scaffolds, balloons, sleeves, cages, prosthetic valves, cages, and the like.

Stress-applying features comprising cylindrical posts are shown in FIGS. 2H-1 and 2H-2. FIG. 2H-1 illustrates a cylindrical post 350 having a flat cylindrical top 350a and a flared base 350b configured for attachment to an outer surface of a radially expanding structure. The flared base increases the surface area available for attachment to the surface of the expandable structure. The flat cylindrical top may have a peripheral edge (as shown) but in other instances may have rounded or beveled peripheral edge (not shown). The contact region of the cylindrical top maybe coated with a material to provide one or more of an adherent (increased friction) surface, a smooth surface, a low-friction surface, or the like. Material can be bonded, fused, coted, laminated, or otherwise added to the surface to modify the surface geometry, e.g., make the surface flatter, more rounded, add texture, or the like. FIG. 2H-2 illustrates a cylindrical post 352 having a domed top 352a and a flared base 352b configured for attachment to an outer surface of a radially expanding structure.

Exemplary stress-applying features comprising tapered cylindrical posts are shown in FIGS. 2H-3 and 2H-4. FIG. 2H-3 illustrates a post 354 having a flat, circular bottom 354a which tapers radially outwardly in an upward direction to a flat circular top 354b that is larger than the bottom. FIG. 2H-4 illustrates a post 356 having a flat, round bottom 356a which tapers radially inwardly in an upward direction to a flat round top 356b that is smaller than the bottom.

Exemplary stress-applying features comprising stacked structures having an upper spherical or near-spherical plaque-engaging element and a variety of lower supporting elements are illustrated in FIGS. 2H-5 to 2H-9. FIG. 2H-5 illustrates a stress-applying feature 360 having a lower spherical support element 360a and an upper spherical plaque-engaging element 360b. FIG. 2H-6 illustrates a stress-applying feature 362 having a lower truncated conical element 362a and an upper spherical plaque-engaging element 362b. The truncated conical element 326a may provide an additional stress-applying feature where a truncated top 362c can have a flat surface or other type of surface to contact a hardened plaque tissue. FIG. 2H-7 illustrates a stress-applying feature 364 having a lower truncated spherical support element 364a and an upper spherical plaque-engaging element 364b. A truncated top 364c of the truncated spherical element 364a may provide an additional stress-applying feature to contact a hardened plaque tissue. FIG. 2H-8 illustrates a stress-applying feature 366 having a lower domed support element 366a and an upper spherical plaque-engaging element 366b. FIG. 2H-9 illustrates a stress-applying feature 368 having a lower support post 368a which has a reduced waist diameter (a “concaved” midsection) and an upper spherical plaque-engaging element 368b.

Stress-applying features comprising stacked structures having an upper dome-like plaque-engaging element and a variety of lower supporting elements are illustrated in FIGS. 2H-10 to 2H-13. FIG. 2H-10 illustrates a stress-applying feature 370 having a lower spherical support element 370a and an upper dome-like plaque-engaging element 370b. FIG. 2H-11 illustrates a stress-applying feature 372 having a lower truncated hemispherical element 372a and an upper partial spherical plaque-engaging element 372b which together form an integrated dome structure. Typically, the upper and lower elements will be formed from the different materials and/or will have different hardnesses or other material properties, but in some instances may be formed from the same materials having similar or identical material properties. FIG. 2H-12 illustrates a stress-applying feature 374 having a lower support post 374a which is tapered about its midsection and an upper dome-like plaque-engaging element 374b. FIG. 2H-13 illustrates a stress-applying feature 376 having a lower truncated, tapered conical supporting element 376a and an upper dome-like plaque-engaging element 376b.

FIG. 2H-14 illustrates a stress-applying feature 378 which comprises a monolithic dome 378a having rounded top 378b asymmetrically positioned relative to a circular or ovoid-perimetered base 378c.

Stress-applying features having channeled plaque-engaging element and a variety of lower supporting elements are illustrated in FIGS. 2H-15 to 2H-18. FIG. 2H-15 illustrates a stress-applying feature 380 having an integrated structure, similar to that shown in FIG. 2H-11, with orthogonally oriented channels 380a and 380b dividing the structure into four symmetric quadrants. FIG. 2H-16 illustrates a stress-applying feature 382 comprising a cylindrical post 382a with orthogonally oriented channels 382b and 382c dividing the structure into four symmetric quadrants. The top of each quadrant includes smaller features 382d which assist in gripping and fracturing clot engaged by the post. FIG. 2H-17 illustrates a stress-applying feature 384 comprising a cylindrical post 384a similar to that illustrated in FIG. 2H-16 with orthogonally oriented channels 384b and 384c dividing the structure into four symmetric quadrants. Such channels, gaps, or spacers may be oriented axially along the axial length of the expandable structure and allow for medicaments or contrast agents to flow past the expandable structure in a direction from a proximal region to a distal region thereof under, at, or above physiologic pressures and conditions when such medicaments or contrast media is injected proximally to the expandable structure when the structure is expanded or inflated to the expanded inflated configuration.

The top of each quadrant includes smaller features 384c which assist in gripping and fracturing clot engaged by the post 384b which is mounted on a flared base 384d. The channels 384b and 384c provide a path for fluid to pass through when the expandable structure is in the expanded configuration. FIG. 2H-18 illustrates a stress-applying feature 386 is identical in all expect to the stress-applying feature 384 of FIG. 2H-17 except that two of the post quadrants have a shorter height than two of the other quadrants. In other embodiments, any two, three, or four quadrants may have the same or a different height, width, diameter, symmetry, asymmetry, or combinations thereof.

Stress-applying features having multiple plaque engaging elements on their upper surfaces are illustrated in FIGS. 2H-19 to 2H-21. FIG. 2H-19 illustrates a stress-applying feature 388 having four spherical plaque-engaging elements 388a on an upper surface of a cylindrical base 388b. FIG. 2H-20 illustrates a stress-applying feature 390 having four spherical plaque-engaging elements 390a supported by a cylindrical base 390b. The four spherical plaque-engaging elements 390a are mounted on a tapered conical support having a plaque-piercing tip 390c that its center. FIG. 2H-21 illustrates a stress-applying feature 392 which is identical to that of FIG. 2H-20, including four spherical plaque-engaging elements 392b surrounding a plaque-piercing tip 392c, but not including a cylindrical or other base. It will be appreciated that the different elements of the stress-applying features of FIGS. 2H-1 to 2H-21 can be combined and substituted in various ways to provide additional designs within the scope of the present invention.

FIG. 2H-22 illustrates a stress-applying feature 394 which comprises a simple arcuate bar 394a having a lower surface 394b which is attached directly or indirectly to an outer surface of a radially expanding structure.

As shown in FIG. 3 and subsequent drawings herein, the stress-applying features of the present invention may be attached to a variety of expandable structures including balloons, cages, sleeves, prosthetic valve bodies, valvuloplasty members, drug delivery members, and the like, in addition to the stents and scaffolds which have been described above. For example, an expandable structure of the present invention may comprise a radially expandable sleeve 40, as shown in FIG. 3. The sleeve may be a tubular elastic membrane 42 configured to be placed over a balloon, a stent, a graft, or any other primary structure configured to be expanded within the lumen of a blood vessel or other body passage or lumen. The stress-applying features 24 may be attached to an outer surface 44 of the sleeve 40 in any one of a variety of configurations. As illustrated in FIG. 3, the stress-applying features 24 are arranged in a helical pattern with one end region 46 of the sleeve being free of stress-applying features.

Referring now to FIG. 4A, a sleeve scaffold 40a comprises a similar tubular elastic membrane 42 having individual stress-applying features 24 arranged in a diametrically opposed pattern on the outer surface of the tubular elastic membrane 42. Each successive pair of diametrically opposed stress-applying features 24 is staggered by 90°, as shown in FIG. 4B.

In other instances, as shown in FIGS. 5A and 5B, an elastic sleeve 40b comprises a tubular elastic membrane 42 having individual stress-applying features 24 arranged in groups of three (“triplets”), with each stress-applying feature in the triplet being spaced-apart from circumferentially adjacent features by 120°. Typically, successive “triplets” are staggered by 60°, as best seen in FIG. 5B.

While the patterns of stress-applying features shown in FIGS. 3, 4A, 4B, 5A, and 5B, are shown on elastic sleeves 40, it would be apparent to one of skill that these feature patterns can be employed on any expandable structure, including stents, grafts, scaffolds, expandable valvuloplasty cages, cages for performing, expandable angioplasty balloons and the like, in addition to the sleeves illustrated.

In some instances, the stress-applying features 24 may be formed as part of a scaffold fabrication process, as shown in FIGS. 6A through 8C. Referring to now FIGS. 6A through 6C, a scaffold may be patterned in any conventional serpentine or other pattern comprising struts 14 joined by U-shaped crowns 16 by laser cutting, photo-chemical etching, or the like. In accordance with the present invention, stress-applying features 50a may be provided by forming projecting elements 52a and 52b on the outer curve of the crown 16 during the initial fabrication of the tubular scaffold body, as shown in FIG. 6A. The projecting elements 52a and 52b are formed as disks joined to the crown by arms 54a and 54b. Disk 54b is first folded over an outer surface of the crown 16 by bending the arm 54b, as shown in FIG. 6B. Thereafter, disk 54a is folded over the exposed surface of disk 52b, as shown in FIG. 6C. In this way, the exposed surface of disk 52a provides the blunt contact region 30. While circular disks 52a and 52b are illustrated, a variety of other plate geometries could be utilized, such as square, rectangular, polygonal, oval, teardrop shaped, irregular, and the like.

As can be seen in FIGS. 6A to 6C, the projecting elements 50a will be formed within a cylindrical envelope of the scaffold as it is fabricated, requiring little departure from convention scaffold fabrication methods. In particular, a tubular blank can be used as the starting structure. The individual disks 52a and 52b then be sequentially folded over onto the outer surface of the crown 16 by bending arm 54 to provide the blunt contact region 30″. In this way the two disks stack upon each other to create a stress-applying feature having a height twice the thickness of the struts 14 and crowns 16. What is initially the lower surface of disk 52a then becomes the blunt contact region 30 of the resulting stress-applying feature, as shown in FIG. 6C.

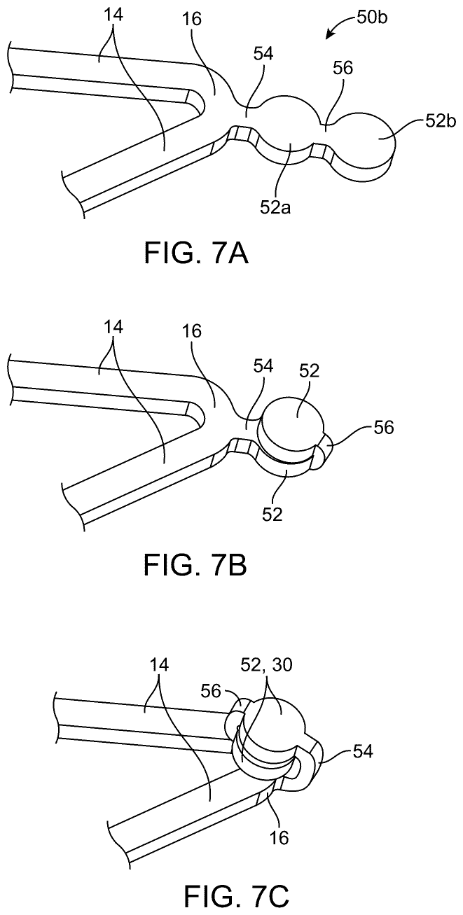

As shown in FIGS. 7A through C, a projecting element 50b includes two disks 52a and 52b join to a crown 16 in tandem by arms 54 and 56. The stress-applying feature is then formed over an outer surface of the crown 16 by first folding the outer disk 52b over the inner disk 52a by bending arm 56 as shown in FIG. 7B. The pair of stacked disks 52a and 52b are then folded over the outer surface of the crown 16 to provide the blunt contact region 30, as shown in FIG. 7C.

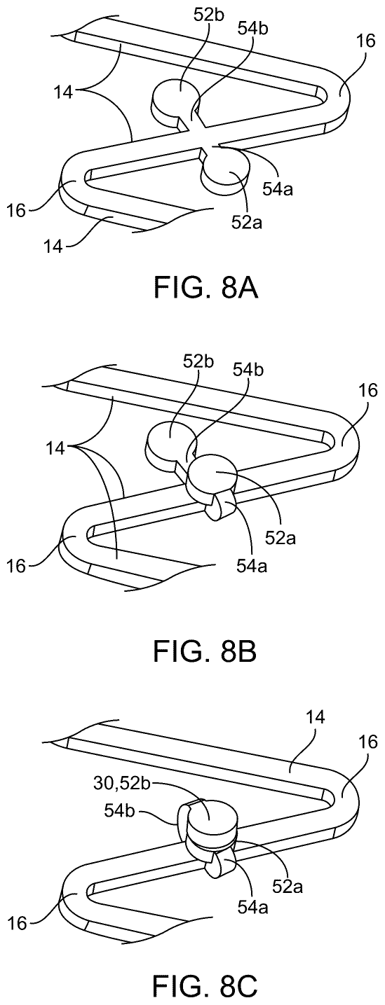

In a third example, as seen in FIGS. 8A to 8C, a pair of disks 52a and 52b are located on opposite sides of a strut 14 and attached by arms 54a and 54b. A first of the disks 52a is then folded over an outer surface of the strut 14, as shown in FIG. 8B, and the second disk 52b is then folded over the first disk 52a, as shown in FIG. 8C. The exposed surface of the second disk 52b thus provides the blunt contact region 30 of the present invention.

As shown in FIGS. 9A and 9B, a blunt contact region 30 of a stress-applying feature 24 is pressed radially outwardly against a region of plaque P or other occlusive material within a blood vessel lumen. As the expandable structure, such as a scaffold strut 14 shown in this figure, is radially expanded, a peripheral edge 60 of the blunt contact region 30 contacts an exposed surface of the plaque region P, as shown in FIG. 9A. As a crown 16 is deflected further by a balloon or other expansive force, the peripheral edge 60 is forced through the surface of the plaque region PR, causing the plaque region to fracture along fracture lines FL the while the blunt contact region 30 prevents injury to vascular wall. In other examples (not shown), the peripheral edge of the plaque disrupting feature maybe beveled or rounded or may be coated with one or more material(s) to provide a beveled, a rounded, or smoother surface. In yet another example (not shown), the stress-applying feature has rounded, convex, dull, or otherwise atraumatic contact region, body, or base, void of any edges that can hinder the advance of system into the body vasculature.

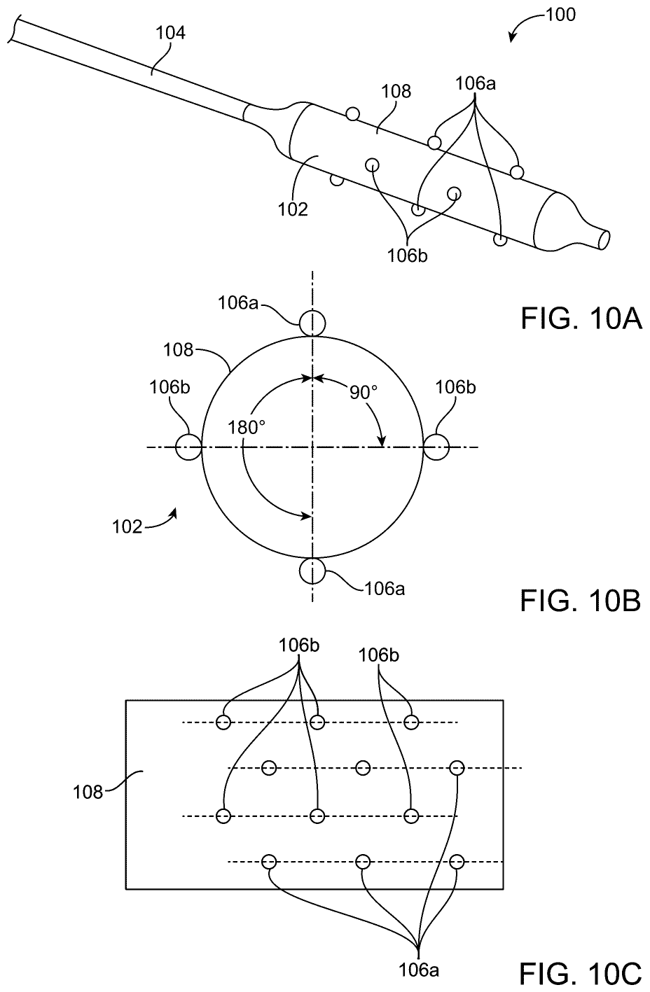

As shown in FIGS. 10A to 10C, a balloon catheter 100 has an inflatable balloon 102 at a distal end of shaft 104. A plurality of spherical stress-applying features 106 are distributed over an outer surface 108 of the inflatable balloon 102. The spherical stress-applying features 106 are arranged with pairs of disruption features 106a and 106b located on opposite sides of the balloon surface 108. Additionally, each pair of features 106a and 106b is rotationally or circumferentially displaced from the adjacent pair by 90°, as best seen in FIGS. 10B and 10C.

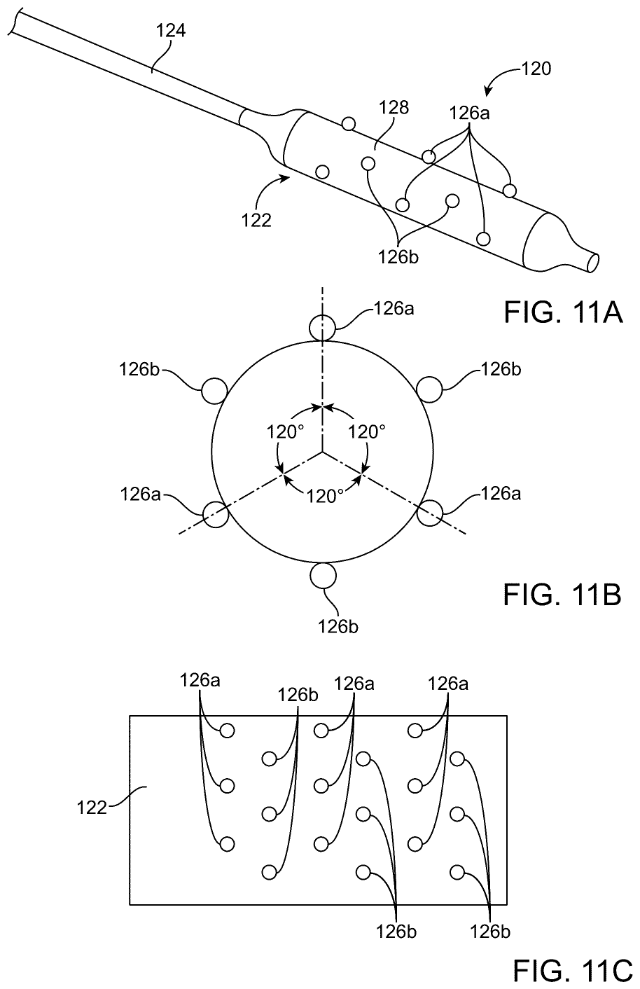

As shown in FIGS. 11A to 11C, a balloon catheter 120 has an inflatable balloon 122 at a distal end of shaft 124. A plurality of spherical stress-applying features 126a and 126b are distributed over an outer surface 128 of the inflatable balloon 122. The spherical stress-applying features 126a-b are arranged with triplets of disruption features 126a and 126b located with a spacing of 120° the balloon surface 128. Additionally, each triplet of features 126a and 126b is rotationally or circumferentially displaced from the adjacent pair by 60°, as best seen in FIGS. 11B and 11C.

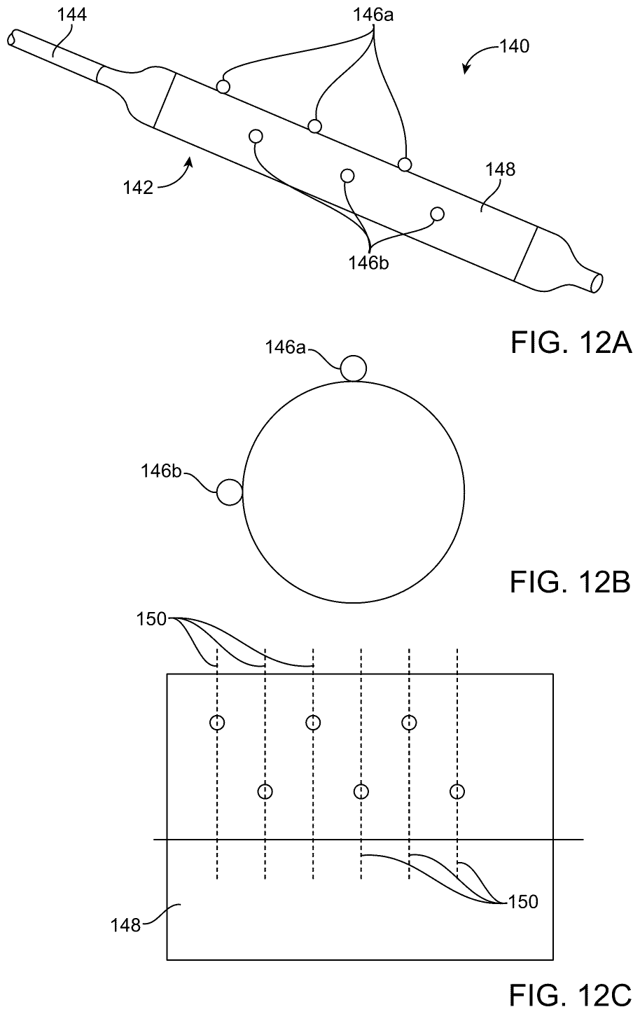

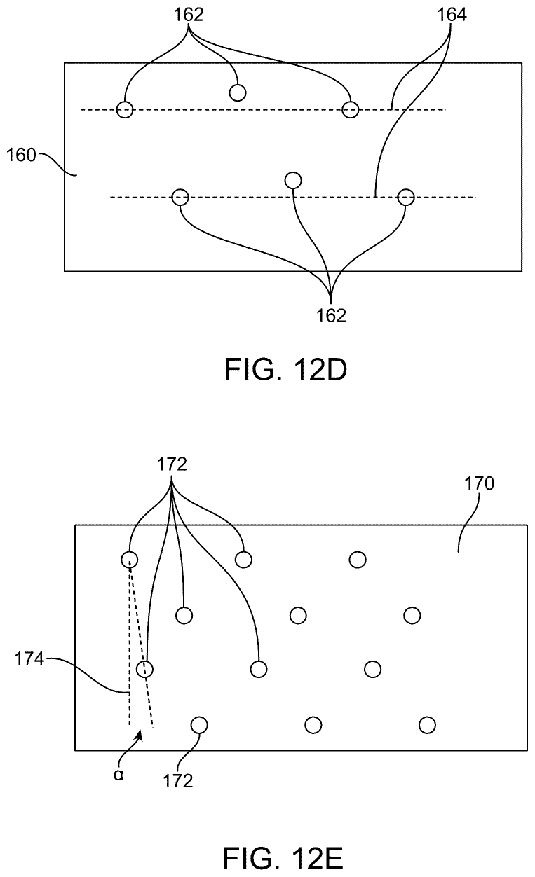

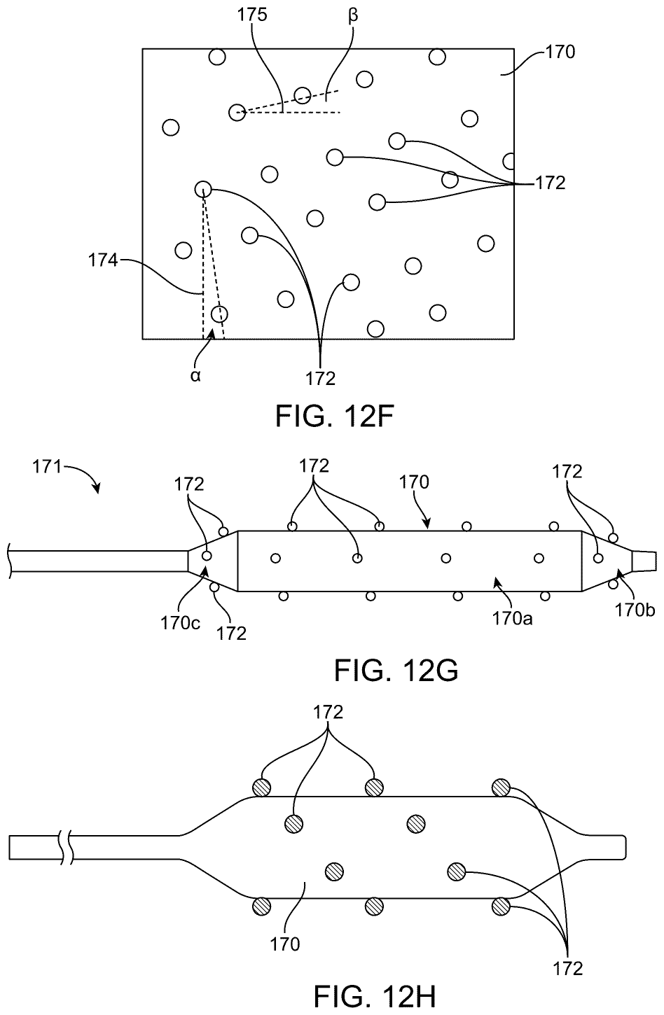

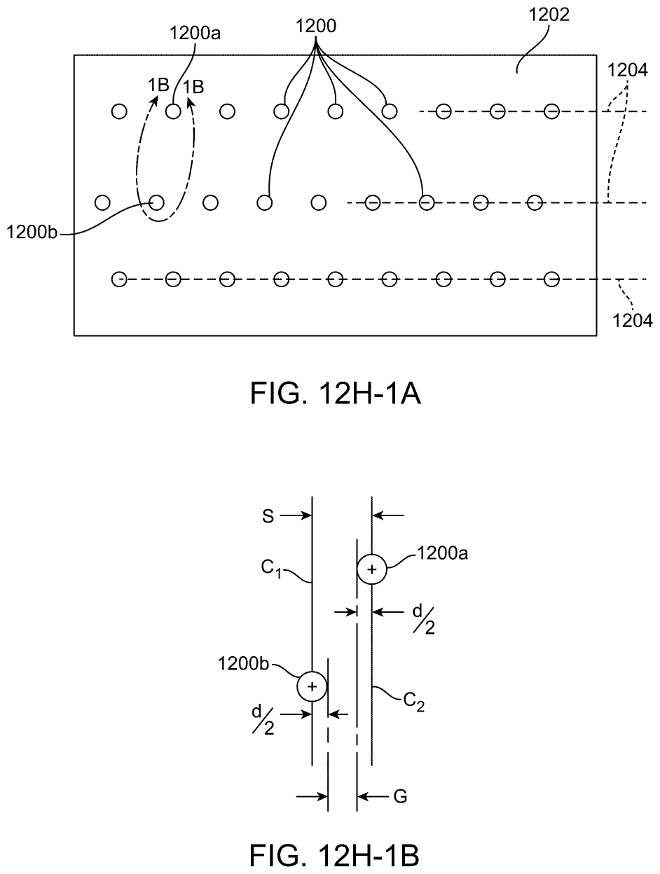

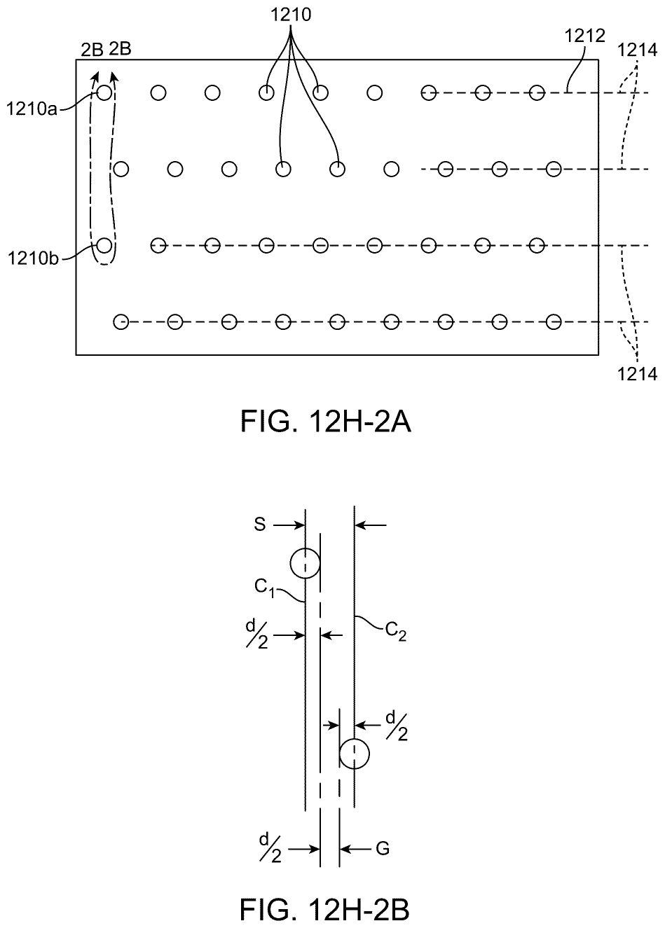

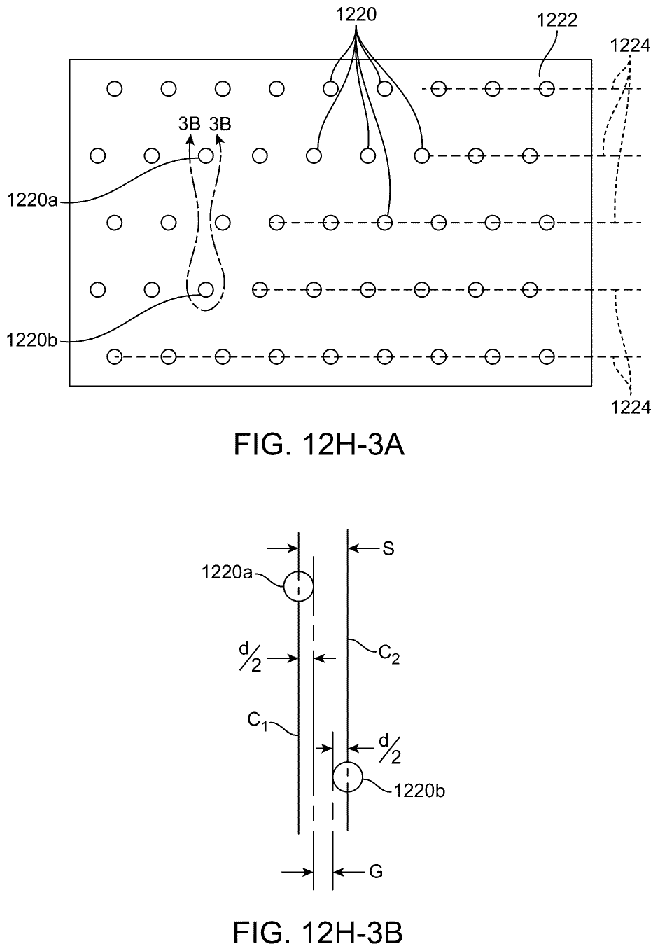

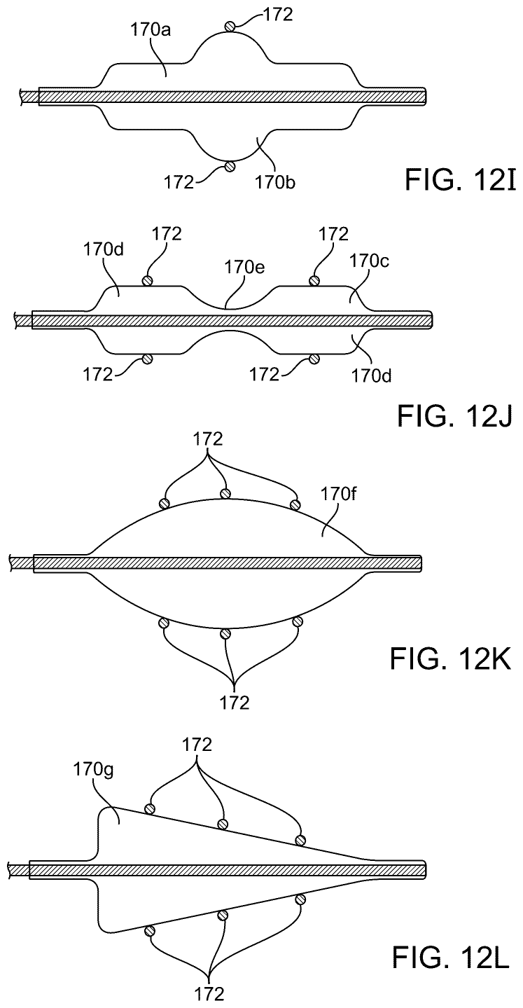

As shown in FIGS. 12A to 12C, a balloon catheter 140 has an inflatable balloon 142 at a distal end of shaft 144. A plurality of spherical stress-applying features 146a and 146b are distributed over an outer surface 148 of the inflatable balloon 142. In contrast to prior environments, however, the stress-applying features 146a/b are not arranged symmetrically. Instead, they are arranged in two axial lines comprising features 146a and 146b, respectively, spaced apart by 90° along the edges of one quadrant of the balloon. The spherical stress-applying features 146 are also axially staggered so that they lie on different, axially spaced apart circumferential lines 150 around the balloon surface 148.

Although specific symmetric and asymmetric disruption feature patterns have been illustrated, it will be appreciated that a wide variety of patterns are possible and that some patterns may be configured fall out of alignment. For example, in FIG. 12D, the disruption features 162 generally positioned along axial lines 164 on a balloon surface 160 (shown rolled out) but may be configured to fall circumferentially away of those lines. As shown in FIG. 12E, disruption features 172 which generally lie on circumferential lines 174 on a balloon surface 170 may circumferentially deviate from those lines by a small angle α. Similarly, as shown in FIG. 12F, those disruption features 172 which are generally positioned along axial lines 175 may axially deviate from those lines by a small angle β. Such small misalignments help space the features apart and minimize or avoid interference between features when the expandable structure is radially crimped or collapsed prior to expansion. For example, if a plurality of features were all attached along a single circumferential line, the features could circumferentially collapse against each other when the expandable member is radially crimped or collapsed prior to expansion. Such interference can be reduced or eliminated by moving some or all of the features off of the circumferential line, even by just a small distance such as a width of the feature.

Referring now to FIGS. 12G and 12H, disruption features 172 may be distributed over a balloon surface 170 of a fully formed balloon catheter 171 in a variety of patterns. The stress-applying features 172 will usually be distributed over all or a portion of at least a central region 170a of the balloon surface. Optionally, the stress-applying features 172 may also be distributed over a tapered distal region 170 b (FIG. 12G) of the balloon surface 170 as well as over a tapered proximal region 170c thereof. Specific distribution patterns may be any of those described elsewhere in this application including at least those shown FIGS. 12G and 12H.

FIGS. 12H-1A and 12H-1B illustrate stress-applying features 1200 disposed in a first distribution pattern of on an outer surface of an inflatable balloon 1202. The first distribution pattern provides optimum axial spacing or “offset” between circumferentially adjacent stress-applying features on smaller balloons having a diameter in a range from 1 mm to 3 mm, usually from 1.5 mm to 2.75 mm, when fully inflated. The stress-applying features 1200 are preferably monolithic hemispherical metal solid structures but can have other structures as described herein and are arranged in three axial rows 1204 where each row has nine features and is circumferentially separated from the other two rows by 1200. The hemispherical stress-applying structures 1200 will usually have a base diameter in a range from 0.2 to 0.6 mm, for example being 0.4 mm, and axially adjacent stress-applying features 1200a and 1200b are located on axially spaced-apart circumferential lines C₁ and C₂, as seen in FIG. 12H-1B. The circumferential lines C₁ and C₂ will have an axial spacing S (offset) which is usually in a range from 0.2 mm to 1 mm, for example being 0.66 mm for stress-applying features 1200 having a diameter of 0.4 mm. In this way, a gap G in a range from 0 mm to 0.5 mm, preferably from 0.1 to 0.5 mm can be maintained to limit interference between the axially adjacent stress-applying features 1200 as the balloon is folded and/or to maximize calcium disruption force. The gap G will be 0.26 mm for stress-applying features 1200 having a diameter on 0.4 mm and a centerline spacing S of 0.66 mm.

FIGS. 12H-2A and 12H-2B illustrate stress-applying features 1210 disposed in a second distribution pattern of on an outer surface of an inflatable balloon 1212. The second distribution pattern provides optimum axial spacing or offsets between circumferentially adjacent stress-applying features on medium-sized balloons having a diameter in a range from 2.5 mm to 3.5 mm, usually from 2.75 mm to 3 mm, when fully inflated. The stress-applying features 1210 are preferably monolithic hemispherical metal solid structures but can have of the other structures described herein and are arranged in four axial rows 1214 where each row has eight features and is circumferentially separated from the adjacent two rows by 90°. The hemispherical stress-applying structures 1210 will usually have a diameter in a range from 0.2 mm to 0.6 mm, for example being 0.4 mm, and axially adjacent stress-applying features 1210a and 1210b are located on axially spaced-apart circumferential lines C₁ and C₂, as seen in FIG. 12H-2B. The circumferential lines C₁ and C₂ will have an axial spacing S which is usually in a range from 0.15 mm to 1 mm, for example being 0.5 mm for stress-applying features 1210 having a diameter of 0.4 mm. In this way, a gap G in a range from 0 mm to 0.4 mm, preferably from 0.1 mm to 0.3 mm can be maintained to limit interference between the axially adjacent stress-applying features 1210 as the balloon is folded. The gap G will be 0.1 mm for stress-applying features 1210 having a diameter on 0.4 mm and a centerline spacing S of 0.5 mm.

FIGS. 12H-3A and 12H-3B illustrate stress-applying features 1220 disposed in a third distribution pattern of on an outer surface of an inflatable balloon 1222. The third distribution pattern provides optimum axial spacing or offsets between circumferentially adjacent stress-applying features on large-sized balloons having a diameter in a range from 3 mm to 4 mm, usually from 3.25 mm to 4 mm, when fully inflated. The stress-applying features 1220 are preferably monolithic hemispherical metal solid structures but can have of the other structures described herein and are arranged in five axial rows 1224 where each row has nine features and is circumferentially separated from the other two rows by 72°. The hemispherical stress-applying structures 1220 will usually have a diameter in a range from 0.2 mm to 0.6 mm, for example being 0.4 mm, and axially adjacent stress-applying features 1210a and 1210b are located on axially spaced-apart circumferential lines C₁ and C₂, as seen in FIG. 12H-3B. The circumferential lines C₁ and C₂ will have an axial spacing S which is usually in a range from 0.15 mm to 1 mm, for example being 0.4 mm for stress-applying features 1220 having a diameter of 0.4 mm. In this way, a gap G in a range from 0 mm to 0.4 mm, preferably from 0 to 0.2 mm can be maintained to limit interference between the axially adjacent stress-applying features 1220 as the balloon is folded. The gap G will be 0 mm (no gap and no overlap) for stress-applying features 1220 having a diameter on 0.4 mm and a centerline spacing S of 0.4 mm.

As shown in FIGS. 12I to 12L, the stress-applying features 172 of the present invention may be placed on balloons and other expandable structures having a variety of different geometries and configurations. For example, as shown in FIG. 12I, the plaque disrupting features 172 may be placed on an oversized middle section 170b of an inflatable support balloon 170a, or on adjacent cylindrical or tapered end sections (not shown). As shown in FIG. 12J, the plaque disrupting features 172 may be placed on adjacent segments 170d of a balloon 170c having a depression, waist 170e at its middle. In some cases, depression 170e could have a plurality of stress-applying features while adjacent segments 170d could be free of stress-applying features. As shown in FIG. 12K, the stress-applying features 172 may be placed on balloons or other expandable supports 170f having ovoid or spheroidal shapes. As shown in FIG. 12L, the stress-applying features 172 may be placed on balloon supports 170g having a tapered conical shape on one side and a flat surface at a proximal end thereof. In some instances, the tapered conical surface of the balloon or other support can have stress-applying features on the flat end (not shown), while the conical tapered segment is void of stress-applying features (not shown).

In such exemplary arrangements, the preferred hemispherical or spherical plaque disrupting features are “discrete,” i.e., separated from each other in the circumferential and axial directions and protruding radially outwardly from the outer surface of the balloon or other expandable structure before and after expansion of the expandable structure. The hemispherical or spherical plaque disrupting features may be placed on the working length of the balloon only (as shown in FIGS. 10A, 11A, 12A, and 12H), on both the working length and the proximal and/or distal regions (FIG. 12G), or in some cases only the proximal and/or distal regions (not shown).

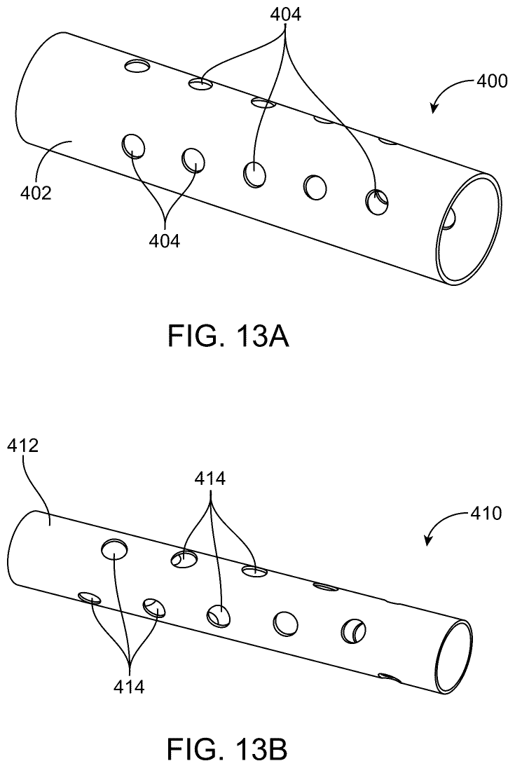

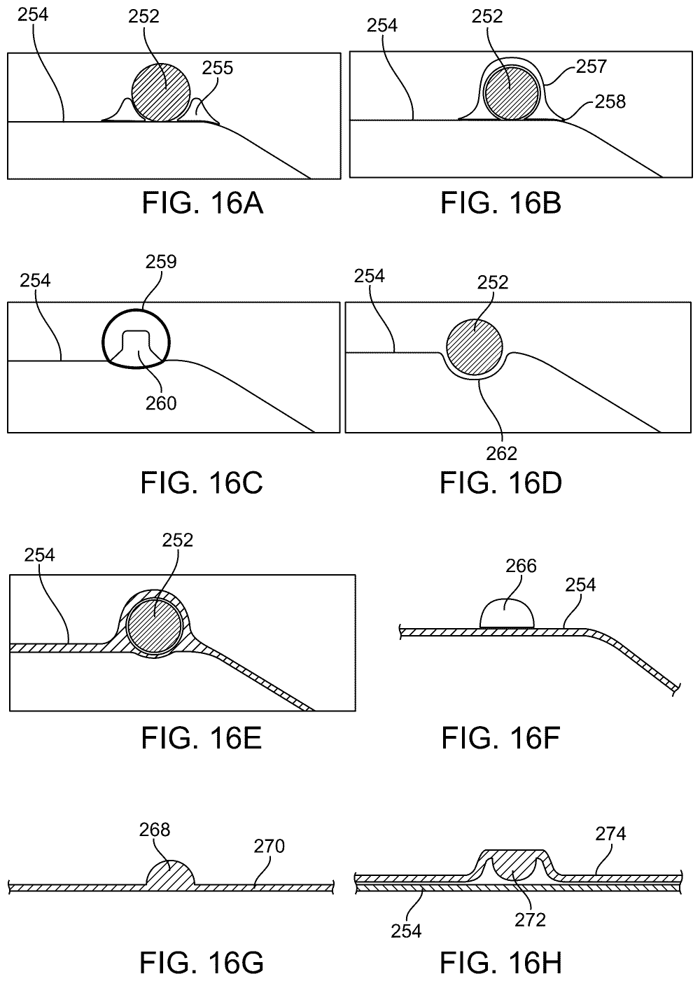

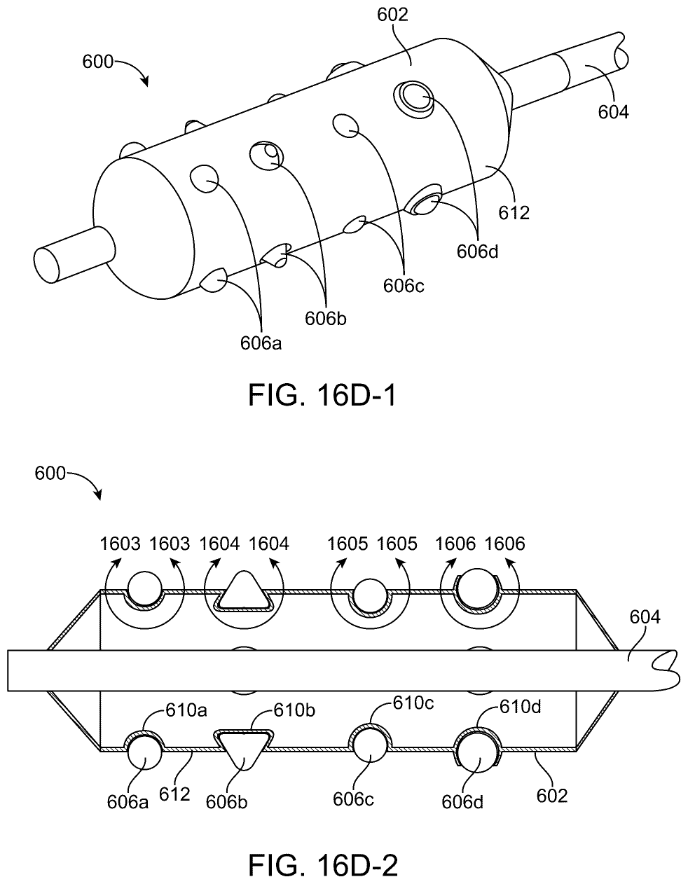

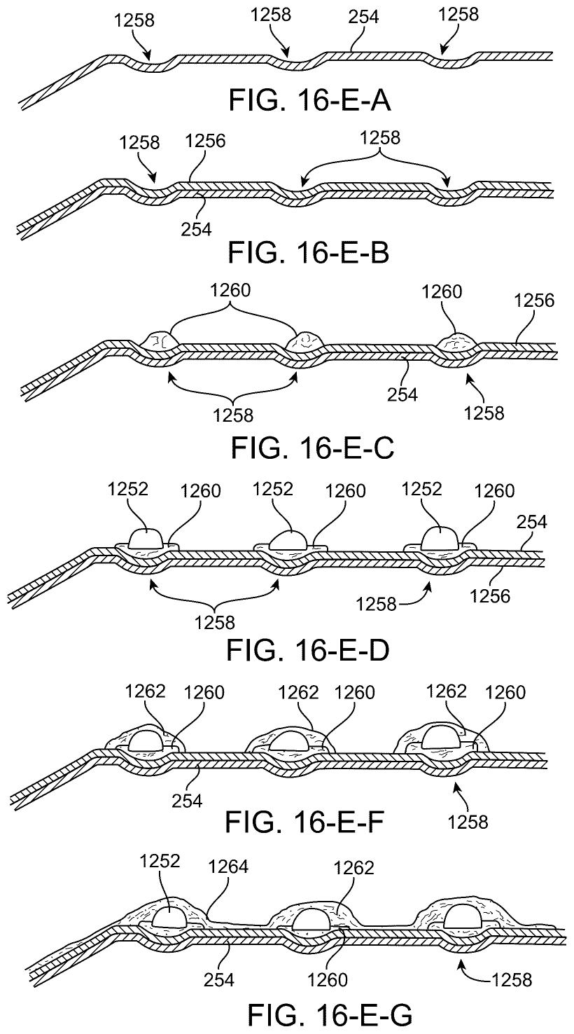

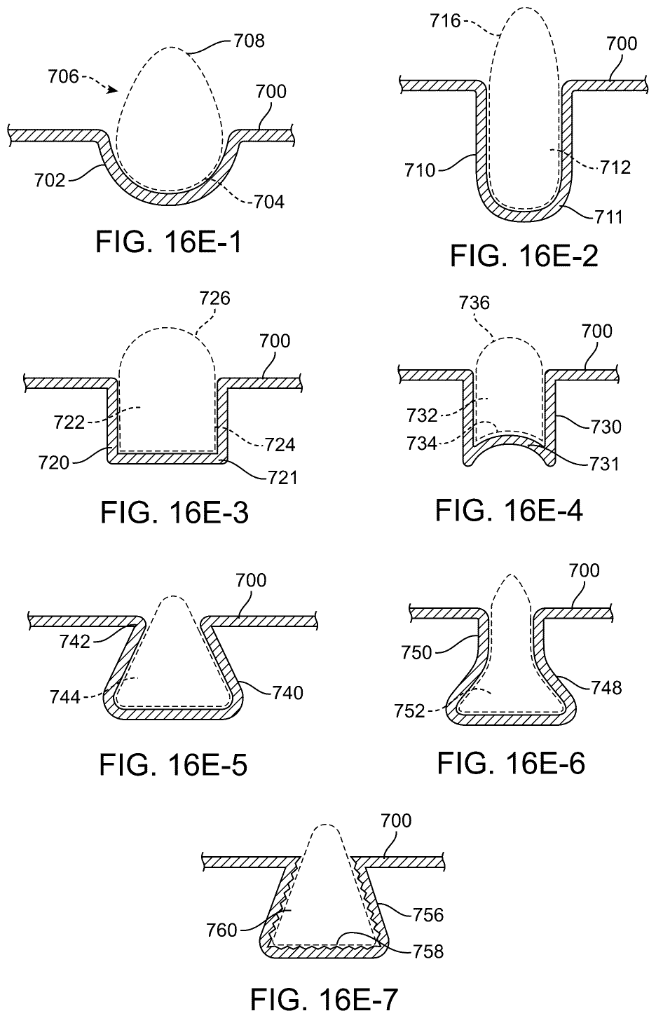

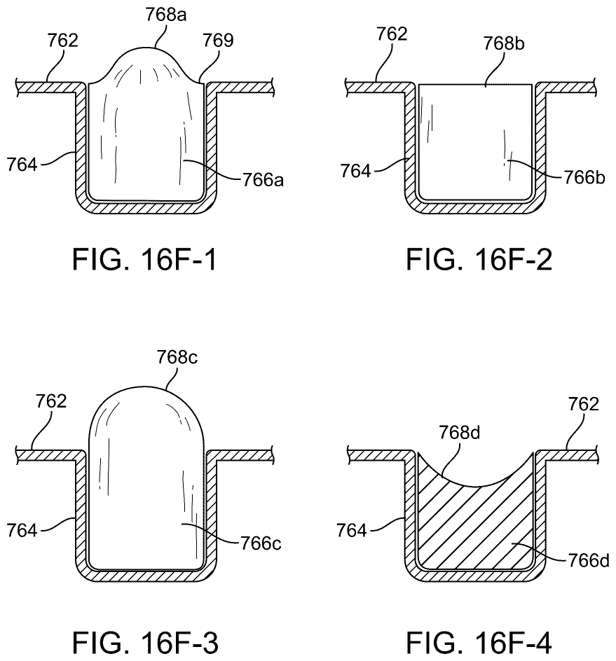

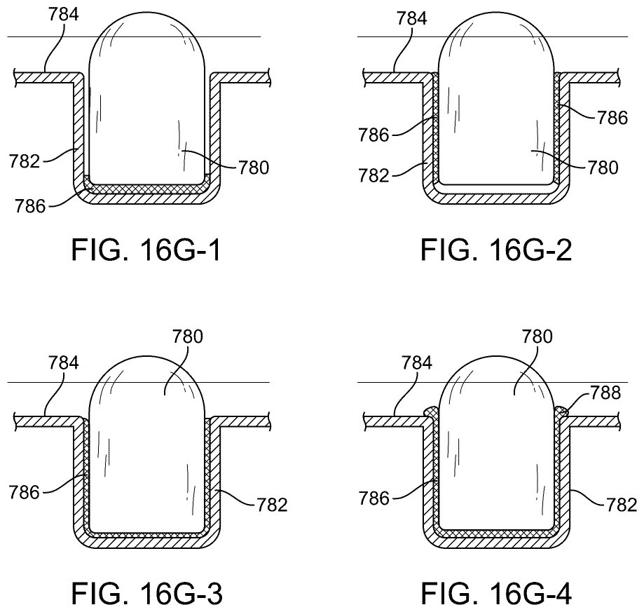

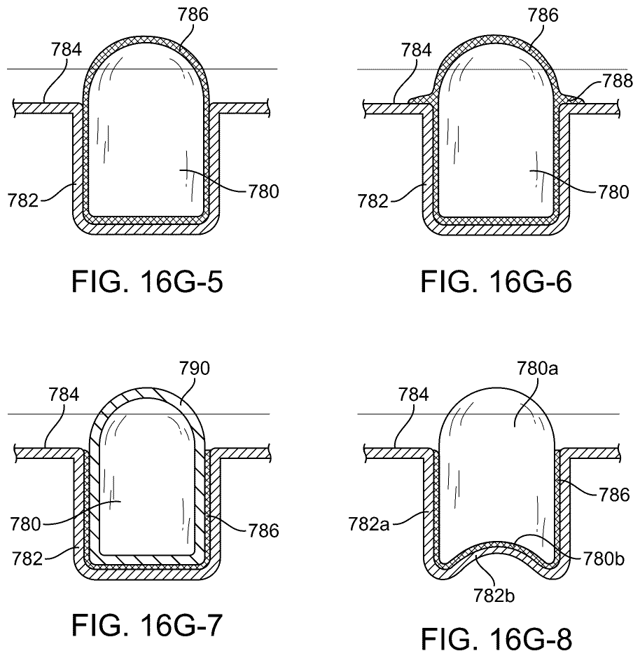

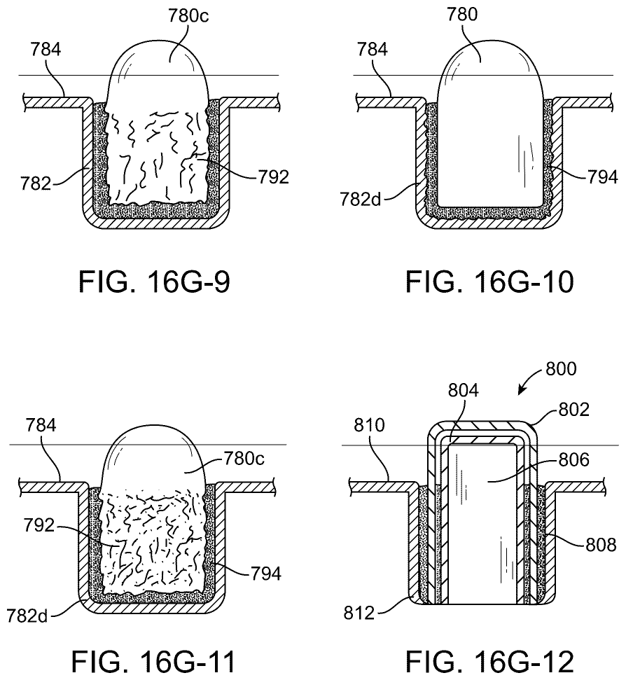

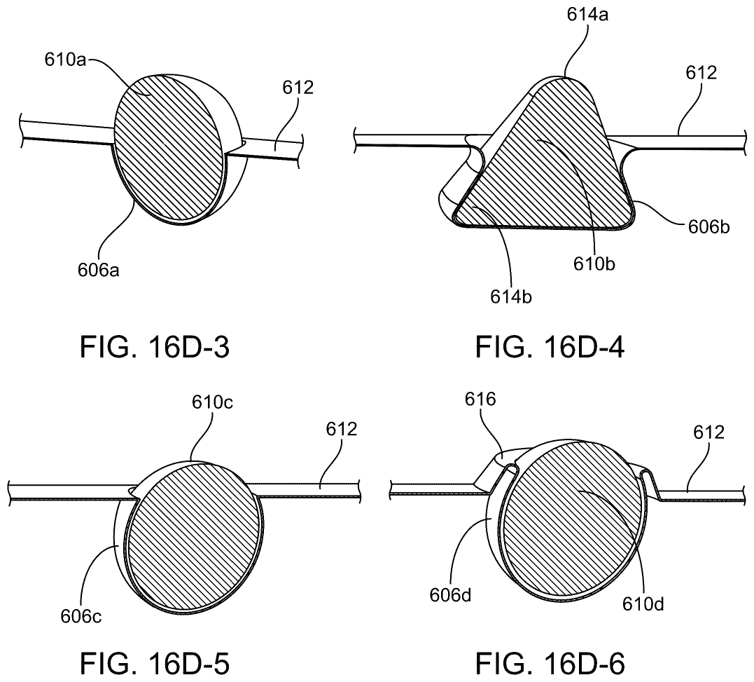

As shown in FIGS. 13A and 13B, plaque disrupting features may be positioned on an outer surface of the balloon, sleeve or other expandable member using a tubular template. Tubular tube 400 shown in FIG. 13A comprises a PTFE sheath 402 with a pre-selected pattern of circular or other cutouts 404 configured to be placed over an outer surface of an expanded balloon or sleeve where the cutouts allow the placement of adhesive material such as adhesive droplets onto the surface of the expandable structure such as an outer surface of a balloon, sleeve or other expandable structure for attachment of the plaque disrupting features, e.g., as shown in FIG. 16A described below. Smaller tubular templates 410 comprising a PTFE or other sheath 412 with circular or other cutouts 414 may be provided for placement over uninflated or unexpanded balloons and sleeves or smaller expanded balloons and sleeves. After placement of the adhesive droplets, the tubular template is removed and the features are placed and press fit on the droplets along the length and circumference of the outer surface expandable member, typically while partially or fully inflated.

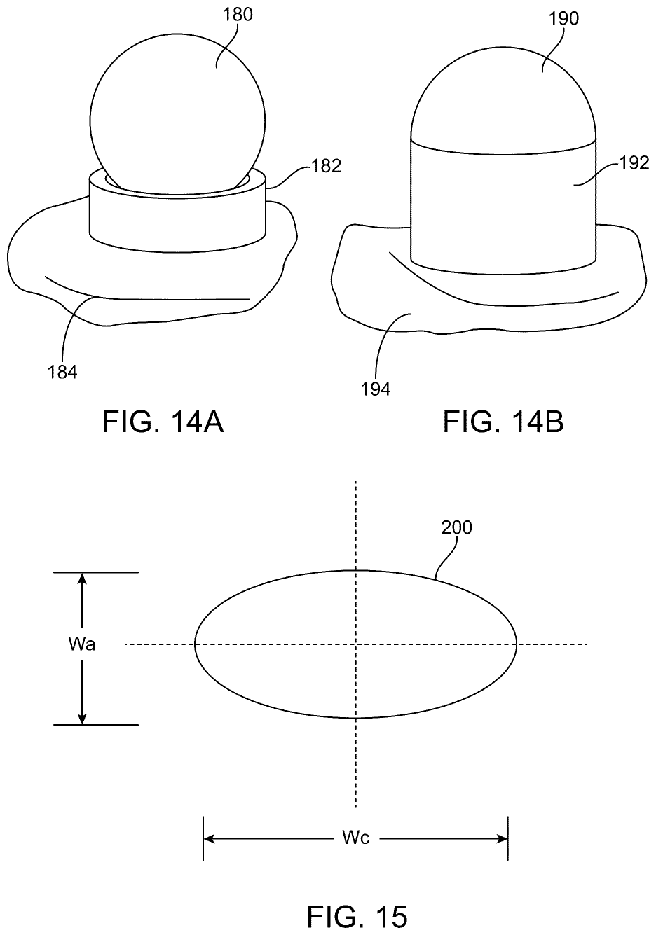

Referring now to FIG. 14A, a spherical stress-applying feature 180 may be secured to a balloon surface 184 using a circular base 182. The circular base 182 helps stabilize the stress-applying feature 180 on the balloon surface and may be secured by adhesive attachment, laser welding, heat welding, or other means known in the art. In addition, the entire structure of the stress-applying feature 180 and the circular base 182 maybe further held in place by placing of a cover (not shown) over the structure as described previously herein.

Referring now to FIG. 14B, a hemispherical stress-applying feature 190 may be secured to an outer surface of an expandable structure, such as a balloon, cage, or sleeve using a cylindrical base or pillar 192. The base 192 stabilizes the stress-applying feature 190 on the balloon surface and also elevates the surface of the stress-applying feature 190 by pre-determined height or elevation above the balloon surface 194. In this way, the radially outward extent of the stress-applying feature can be elevated at any desired distance above the balloon surface, typically in the range is set forth above. It's described previously, the base or pillar 192 and the hemispherical feature 190 may be secured to the balloon service 194 using adhesive attachment, laser welding, heat welding, or other means known in the art. In addition, the entire structure of the stress-applying feature 190 and the base or pillar 192 maybe further held in place by placing of a cover (not shown) over the structure as described previously herein.

Referring now to FIG. 15, the stress-applying features of the present invention will preferably be discrete, as described above, and secured directly to the surface of the expandable structure, preferably to the outer surface of the expandable structure such as balloon, sleeve, cage, or other expendable member. In particular, the stress-applying features and optional bases will not extend any significant axial distance along the surface of the balloon, sleeve, cage, or other expandable member. In contrast, the cutting elements on typical cutting balloons are elongate blades which extend axially over a major portion of the balloon length in order improve cutting performance. The stress-applying features of the present invention preferably do not extend significantly in either the axial or circumferential directions on the surface of the expandable member and are physically independent of each other (i.e., not coupled or linked to each to each other except by the expandable member itself) with relatively small footprints on the balloon surface. In particular, as shown in FIG. 15, the ratio of axial width Wₐ to circumferentially width W_{c} of the peripheral footprint 200 will be in a range from 0.5 to 5, usually from 1 to 5, more usually from 2 to 5, and typically being 1 to 1 which is characteristic of a circular base or footprint. In rare instances, the Wₐ:W_{c} ratio is 3:1 to 1.5:1.

As shown in FIGS. 16A to 16H, stress-applying features may be attached to an outer or inner surface of an expandable structure or other underlying substrate in a variety of ways. For example, a solid or hollow metal or other spherical stress-applying features 252 may be glued, welded, or otherwise adhered to a surface of a substrate 254, where the substrate can be any of a structure the scaffold, an elastic, semi-elastic, or non-distensible membrane of a balloon catheter, an elastic sleeve, cage, valvuloplasty balloon, or the like.

As shown in FIG. 16A, the spherical stress-applying or stress-applying features 252 may be fixed in place by use of an adhesive and/or polymer coating and/or by partially or fully encapsulating the feature. The adhesive/coating material may comprise a single material or a combination of two or more materials. For example, an adhesive, polymer or cement may be formed providing a base or a “cradle” 255 surrounding and adhering to a lower portion of the spherical stress-applying feature 252. The adhesive contours to and partially or fully surrounds the lower portion of the stress-applying feature 252 (as shown) and can extend beyond the footprint of the feature (as shown). The adhesive typically surrounds the feature boundary. In some instances (not shown), the adhesive material can contour to, surrounding, and/or partially cover or encapsulate the outer surface of an upper portion of the feature.

As shown in FIG. 16B, the stress-applying feature 252 may be fixed in place by an adhesive or a polymer coating 257 which covers and encapsulates the entire surface of stress-applying feature 252 exposed over the surface. The adhesive or coating 257 adheres to the substrate 254 about a bottom periphery 258 of the feature.

As shown in FIG. 16C a hollow feature, such as a hollow sphere 259 having a truncated or open bottom, may be mounted on a post 260 which protrudes outwardly from a surface of a substrate, typically the outer surface of the substrate 254. The post may be pre-formed, e.g., molded as part of the balloon or other substrate 254 or may be separately formed and attached to the balloon using an adhesive, cement, ultrasonic bonding, or other conventional procedure. The post will typically be formed to conform to a cavity in the hollow sphere 259 and will also be attached using an adhesive, cement, ultrasonic bonding, or other conventional procedure (as shown). Optionally, the hollow truncated sphere maybe attached to the balloon surface where the adhesive and/or polymeric material covers the inside of the sphere partially or fully (not shown).

In yet another alternative example (not shown), the features being hollow or solid such as a sphere is inserted inside the post and is secured by glue or other adhesive material to the inner surface of the post. This provides additional securing of the feature to the expandable structure surface, while providing hardened features such as formed from a metal or metal alloy to disrupt plaque when the expandable structure is expanded pushing the features against plaque or hardened tissue while the feature being encapsulated by the expandable structure outer surface. In yet another alternative or example (not shown), the feature comprises a hole through the feature extending axially and/or circumferentially to the expandable structure axial or circumferential direction, wherein the features hole forms a cavity in the base of the feature and wherein post 260 of the outer surface of the expandable structure protrudes into said cavity. The post in such example would optionally be glued the cavity surface of the feature to provide further securement of the feature to the expandable structure post. The post may be pre-formed, e.g., molded as part of the balloon or other substrate 254 or may be separately formed and attached to the balloon, or maybe formed or molded in the balloon outer surface when the balloon is in the expanded configuration after forming the balloon.

As shown in FIG. 16D, the spherical stress-applying feature 252 may be attached to the outer surface 254 of the substrate in a preformed indentation 262 which provides a receptacle or “cradle” for immobilizing the feature using one or more adhesive or polymeric materials. The cradle may be formed to conform closely to the feature to improve attachment. The feature may have a slightly wider base than the cradle to provide further support or securement.

FIGS. 16D-1 to 16D-6 illustrate further examples of features attached in preformed indentations in an outer surface of a balloon which provides a receptacle or “cradle” for immobilizing a plurality stress-inducing feature using one or more adhesive or polymeric materials. Although a balloon is illustrated, it will be appreciated that the use of indentations for immobilizing stress-inducing features will be applicable to other expandable members which comprise a polymeric membrane or sheet forming an expandable surface, specifically including but not limited to elastomeric and non-elastomeric sleeves.

A catheter 600 having an angioplasty or other inflatable, interventional balloon 602 at a distal end of a shaft 604 is illustrated in FIGS. 16D-1 and 16D-2. A plurality of stress-inducing features 606a to 606d are held in indentations 610a to 610d formed in an outer surface 612 of the balloon. The indentations in the surface 612 may be pre-formed or molded over a bottom surface of the stress-inducing features, and in both cases will preferably conform closely to the geometry of the lower portion of the feature 606a-606d. The stress-applying features 606a, 606c and 606d are shown as spheres. In contrast, the stress-applying feature 606b has a conical shape with a rounded apex 614a (FIG. 16D-4) protruding from the indentation 606b and over the surface 612. The conical stress-applying feature 610b has an enlarged base 614b which retained in the conforming indentation 606b. All stress-applying features 606a to 606d are shown as solid structures, typically metal or other hard materials as described previously, but could also be formed as hollow structures, as for example shown in FIG. 16C.

In exemplary cases, an adhesive would cover at least a portion of a surface or “interface” of the indentation 610a to 610d between the feature 606a to 606d and balloon 602 or other expandable structure. In other examples, an adhesive could protrude above the expandable structure surface further cradling or encapsulating of at least part of the outer surface of the feature, sometimes encapsulating the entire outer surface of the feature. In some instances, such as shown in FIGS. 16D-4 to 16D-6, a lower portion of the indentation 610b to 610d is larger than a neck of the indentation to provide more protection in holding the feature in place. As shown in FIG. 16D-6, the expandable surface has a lip 616 that protrudes above the surface 612 of the balloon 602 surrounding the neck to enhance retention when the balloon is inflated by pressing the lip against the surface of the feature adjacent to the neck.

As shown in FIG. 16E, the feature 252 may be encapsulated or integrated in the outer surface of the balloon or other substrate 254 by fusing or laminating the feature in one, two or more layers of one or more balloon materials, optionally being fused to form one inseparable layer. For example, as described in detail below in the Example entitled 665—CURABLE ACRYLIC ADHESIVES FOR SECURING HEMISPHERICAL FEATURES TO INDENTATION IN AN OUTER BALLOON SURFACE and referring to FIGS. 16E-A and 16E-B, a bottom or base layer base layer that covers of an elastic polymer, usually an elastic adhesive, such as a light-curable acrylic adhesive, is first formed to cover and encircle most or all of the working length of the balloon 254, including the indented and the non-indented regions of the outer surface of the balloon. Such a base layer strengthens the balloon and decreases the risk of puncture from calcifications, hardened plaque, and the like. The base layer also serves to anchor the plaque disrupting features onto the balloon in combination with additional polymer or adhesive layers as described below.

After curing the base layer 1256, as shown in FIG. 16E-C, indentations 1258 are partially or fully filled with a second layer 1260 of a similar elastic polymer or elastic adhesive, and the spherical or hemispherical features 1252 are placed in the indentations, and the second polymer or adhesive layer cured, as shown in FIGS. 16E-D. Preferably an edge of the second layer will extend beyond the boundary of the indentation providing an annular region which encircles the indentation.

A third “spot” layer 1262 of a similar elastic polymer or elastic adhesive is then applied over the spherical or hemispherical features placed in the indentations, and the third spot layer cured to fully encapsulate the feature, as shown in FIG. 16E-F. Preferably, the third, spot layer fuses directly to the exposed annular edge of the second layer.

Typically, as shown in FIG. 16E-G, to further secure the features to the balloon and provide a fused, integrated structure, a fourth, “top coat” or cover layer 1264 of a similar elastic polymer or elastic adhesive is applied over the entire working length of the balloon and cured to further encapsulate all features and provide an outer surface free from edges and other discontinuities that would increase the risk of individual features being dislodged from the balloon as a result of inflation and/or use.

As shown in FIG. 16F, A solid metal or other hemispherical stress-applying features 266 may be attached to the surface of the membrane 254 in any of the ways just described, typically being fixed in place by use of an adhesive, a polymer coating, and/or by partially encapsulating the feature.

FIGS. 16E-1 to 16E-7 illustrate exemplary balloon indentation geometries in accordance with the principles of the present invention with conforming stress-applying shown in broken line. The indentations are typically formed by blow molding the balloon in a mold cavity having male protrusions corresponding to the shape and dimensions of the desired a surface indentation. For example, as shown in FIG. 16E-1, a hemispherical indentation 702 may be formed in a balloon surface 700 by placing a hemispherical protrusion at a desired location on an inner wall of the mold cavity. The resulting concave cavity 702 is thus configured to receive a mating convex hemispherical base 704 on a stress-applying feature 706. The upper region or “crown” 708 of the stress-applying feature 706 is shown to be conical but could have any geometry illustrated herein or otherwise configured to disrupt plaque.

As shown in FIG. 16E-2, a cylindrical indentation 710 having a hemispherical bottom 711 may be formed in a balloon surface 700 by placing a similarly shaped protrusion at a desired location on an inner wall of the mold cavity. The resulting concave cylindrical cavity 710 is thus configured to receive a mating convex cylindrical base 714 on a stress-applying feature 712. The upper region or “crown” 716 of the stress-applying feature 712 is shown to be conical but could have any geometry illustrated herein or otherwise configured to disrupt plaque.

As shown in FIG. 16E-3, a cylindrical indentation 720 having a flat bottom 721 may be formed in a balloon surface 700 by placing a similarly shaped protrusion at a desired location on an inner wall of the mold cavity. The resulting cylindrical cavity 720 is thus configured to receive a mating convex cylindrical base 724 on a stress-applying feature 722. The upper region or “crown” 726 of the stress-applying feature 722 is shown to be hemispherical but could have any geometry illustrated herein or otherwise configured to disrupt plaque.

As shown in FIG. 16E-4, a cylindrical indentation 730 having an upwardly convex bottom 731 formed in a balloon surface 700 as previously described. The resulting cylindrical cavity 730 is thus configured to receive a mating cylindrical base on a stress-applying feature 732 having a lower concave surface 734 that mates with the convex bottom 731 of the feature. The upper region or “crown” 736 of the stress-applying feature 722 is shown to be hemispherical but could have any geometry illustrated herein or otherwise configured to disrupt plaque

FIGS. 16E-5 and 16E-6 show balloon indentations having a narrow neck that flare into conically expanding cavities. As shown in FIG. 16E-5, a conical indentation 740 has a narrow neck 742 and receives a similarly shaped stress-applying feature 744. As shown in FIG. 16E-6, a conical indentation 748 has an elongated narrow neck 750 and receives a similarly shaped stress-applying feature 752. Typically, the neck portions will have a width which is from 50% to 90% of a maximum width of the lower portion of the cavity.

The conical indentation 756 shown in FIG. 16E-7 is similar to that shown in FIG. 16E-5 except that an inner surface 758 of the indentation has been roughened or otherwise formed to have irregularities to frictionally stabilize the stress-applying feature 760. The stress-applying feature 760 is shown to have smooth surfaces, but in other embodiments could also be roughened to further enhance frictional engagement of the indentation.

FIGS. 16F-1 to 16F-4 illustrate exemplary stress-applying features having bases and crowns, where the bases are secured in an indentation 764 in a balloon surface 762 and where the crowns may protrude above the balloon surface, may be flush with the balloon surface, or may be recessed beneath the balloon surface. As shown in FIG. 16F-1, a stress-applying feature 766a has domed crown 768a which protrudes above the balloon surface 762. The domed crown 768a has a rounded, beveled edge 769 which transitions to the adjacent balloon surface 762. As shown in FIG. 16F-2, a stress-applying feature 766b has a flat surface 768b which is flush with the balloon surface 762. As shown in FIG. 16F-3, a stress-applying feature 766c has a cylindrical crown 768c having a hemispherical upper surface which is elevated above the balloon surface 762. As shown in FIG. 16F-4, a stress-applying feature 766d has a recessed surface 768d which forms a spherical concavity with respect to the balloon surface 762. Exemplary indentation depths are in a range from 0.05 mm to 0.5 mm, usually from 0.1 mm to 0.3 mm. The indentations may optionally be reinforced to otherwise to remain dimensionally stable when inflated to nominal and/or rated balloon inflation pressures, typically from 5 atm to 20 atm.

FIGS. 16G-1 to 16G-12 illustrate exemplary techniques for attaching the bases of the stress-applying features of the present invention in a representative (cylindrical) balloon indentation geometry. Referring specifically to FIGS. 16G-1 to 16G-6, a cylindrical stress-applying feature 780 is received in a cylindrical indentation 782 of a balloon surface 784. At least a portion of the outer surface of the base of the cylindrical stress-applying features 780 will preferably be coupled to the inner wall of the cylindrical indentation 782, typically using an adhesive but optionally by laser welding when the feature is encased in or cladded with a polymeric layer. As shown in FIG. 16G-1, the stress-applying features 780 may be bonded with an adhesive 786 only at its lower end to the bottom of the cylindrical indentation 782. Alternatively, as shown in FIG. 16G-2, the stress-applying features 780 may be bonded only around its circumference to the cylindrical indentation 782. As shown in FIG. 16G-3, the stress-applying features 780 may be bonded over both its lower end and circumference to the entire inner surface of the cylindrical indentation 782. As shown in FIG. 16G-4, the stress-applying features 780 may be bonded over both its lower end and circumference to the entire inner surface of the cylindrical indentation 782 with an additional adhesive bead 788 covering any gap between the balloon surface 780 and circumferential wall of the stress-applying features 780. As shown in FIG. 16G-5, the stress-applying features 780 may be bonded over both its lower end and circumference to the entire inner surface of the cylindrical indentation 782 with an additional adhesive covering the crown of the stress-applying features 780. As shown in FIG. 16G-6, the stress-applying features 780 may be bonded over both its lower end and circumference to the entire inner surface of the cylindrical indentation 782 and over its crown together with an additional adhesive bead 788 covering any gap between the balloon surface 780 and circumferential wall of the stress-applying features 780.

Referring now to FIG. 16G-7, the stress-applying features 780 may be coated or clad in a polymer layer 790 and bonded over both its lower end and circumference to the entire inner surface of the cylindrical indentation 782 with an adhesive bead 786. The polymer cladding can provide desired surface properties to the crown of the stress-applying features 780 and/or allow for polymer welding to the adhesive 786 and/or inner wall of the cylindrical indentation 782.

Referring now to FIG. 16G-8, the stress-applying feature 780a and cylindrical indentation 782a are similar to those described in FIG. 16G-3, above, but have concave and convex lower surfaces 780b and 782b, respectively. The adhesive layer 786 covers both the lower end and the circumference of the stress-applying feature 780a as well the entire inner surface of the cylindrical indentation 782a.

Referring now to FIGS. 16G-9 to 16G-11, the stress-applying features and/or cylindrical indentations may be roughened to enhance bonding, As shown in FIG. 16G-9, an outer surface 792 of the base portion of stress-applying feature 780c may be roughened while the inner wall of the cylindrical indentation 782 remains unroughened. As shown in FIG. 16G-10, an inner surface 794 of cylindrical indentation 782d may be roughened while the outer surface of the base portion of stress-applying feature 780 remains unroughened. As shown in FIG. 16G-11, both the outer surface 792 of the base portion of stress-applying feature 780c and the inner surface 794 of the cylindrical indentation 782d may be roughened, to maximally enhance bonding.

Referring now to FIG. 16G-12, a stress-applying feature 800 may be formed by placing a hollow cap 802 over a protrusion 804 molded into the balloon surface 810. The protrusion 804 is surrounded by an annular well 808 also molded into the balloon surface which receives a lower cylindrical portion (base) of the hollow cap 802, and an adhesive is placed in the well to secure the cap in place. The hollow cap 802 is typically formed from a metal or other hard material, and may be roughened, coated, or otherwise treated to impart different surface characteristics as described previously for solid stress-applying feature embodiments.

Alternatively, hemispherical and other plaque disruption features 268 may be molded over otherwise integrally formed with a polymeric membrane to 270, as shown in FIG. 16G.

As a further alternative, stress-applying features 272 may be molded or otherwise integrally formed as a part of an elastic, constraining sleeve or sheath 274, typically on an inner surface of the sleeve, as shown in FIG. 16H. The sleeve or sheath 274 may be placed over a surface 254 of an expandable member of any of the types described elsewhere herein, typically a balloon, scaffold, or tubular elastic sleeve, so that the feature causes the outer surface of the sleeve to protrude radially outwardly as the expandable member is expanded. The sleeve provides an atraumatic surface or cover to the stress-applying features 272.

Referring to FIGS. 16I-1 to 16I-3, a base of a rigid stress-applying feature 820 may be attached to a polymeric balloon surface 822 by heat welding. A thin film acrylic patch 824 (typically but not necessarily circular) is positioned over the stress-applying feature 820 as shown in FIG. 16I-1. A heating element 826 with a concave heating cavity 828 is pressed against the thin film acrylic patch 824 to conform or “drape” the patch over the stress-applying feature 820 with a annular edge 830 extending outwardly from a periphery of stress-applying element over the balloon surface 822. Heat is then applied to melt the acrylic patch 824 over the stress-applying feature 820 and seal to the balloon surface 822, as shown in FIG. 16I-3. The heat will be applied at a temperature sufficient to melt the acrylic patch, e.g., over 160° C., but below the melting point of the balloon, e.g., 270° C. for nylon balloons. Optionally, a shallow indentation 832 may be formed in the balloon surface 822, as described elsewhere herein, and the acrylic may partially or fully fill the indentation as well as sealing to the surface surrounding the indentation. Such heat bonding may be used with or without other adhesive layers as described elsewhere herein and is advantageous as it could eliminate the need to UV cure at least some adhesive layers (thus simplifying fabrication).

Alternatively, the thin film may first be heat wrapped (shrunk) onto the entire outer surface of a stress applying feature (not shown) including the base region (the entire outer surface of the feature is fully encapsulated with the film), and the wrapped feature is then attached to the balloon outer surface by heat, heat induction, or other means to fuse, melt, or otherwise attach the thin material layer with the outer surface of the balloon. The thin film maybe heat shrunk onto the entire outer surface of the stress applying feature or onto part of the outer surface of the stress applying feature such as the base region. The thin film may be formed from a variety of materials that would attach to the outer surface of the balloon by heating, adhesive or both.

In other instances, an underlayer or pad (not shown) may be positioned beneath some or all of the stress-applying features to provide additional support. Fr example, the pad comprise a layer or film of the balloon material applied after forming of the balloon, or the pad could comprise another polymeric or other material that fuses or melts with the balloon material to provide support (e.g. local strengthening of the balloon wall) for the feature to attach to the balloon where the thickness of said underlayer or pad is typically thinner than the thickness of the balloon layer underneath to maintain flexibility of the outer surface of the balloon and avoid stiffening of the balloon.

The stress-applying features may be attached to the inflatable polymeric balloon in any one or more of several locations, including on an outer surface of the inflatable polymeric balloon, on an inner surface of the inflatable polymeric balloon, on a post secured to the inner or outer surface of the balloon, or on one or more adhesive or other polymeric layers applied to the outside surface of the polymeric balloon, where said layer(s) of adhesive polymer may cover parts or all the surface of the polymeric balloon. In some instances, the adhesive polymer layer(s) may be large enough to encompass area under stress-applying features that are circumferentially and axially adjacent to each other. In a variation, not shown, as noted the stress-applying features may be placed on the inner surface of the balloon or expandable structure so that upon expansion of the balloon, the stress-applying features push against the polymeric balloon and push radially against the inner wall on the body lumen. The balloon (which will often consist of a single layer as described elsewhere herein) may have multiple layers of adhesive or other polymers formed over a portion or all the outer surface of the balloon to strengthen the balloon and prevent piercing of the balloon while it is crushing the calcifications in the body lumen. Alternatively, an elastic sleeve having perforations may be placed over the balloon so, upon expansion of the balloon, the stress-applying features on the inner surface of the balloon protrude against the surface of the balloon and project through the perforations in the elastic sleeve and against the inner wall of the vessel or body lumen.

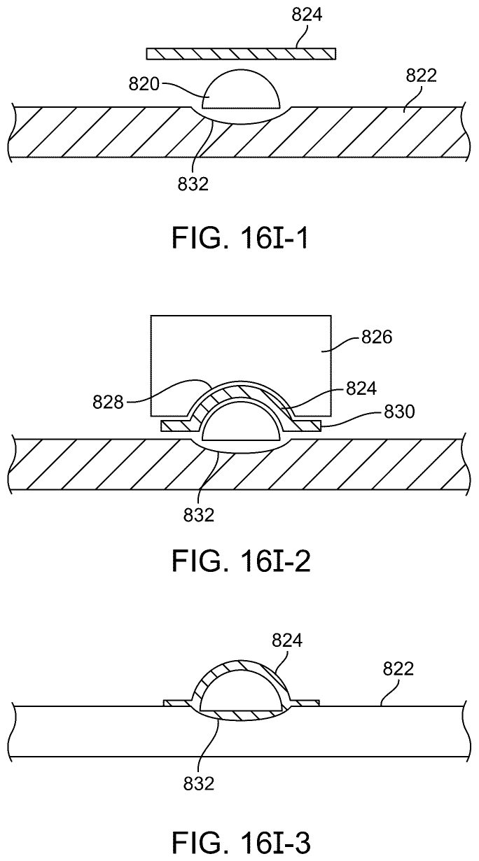

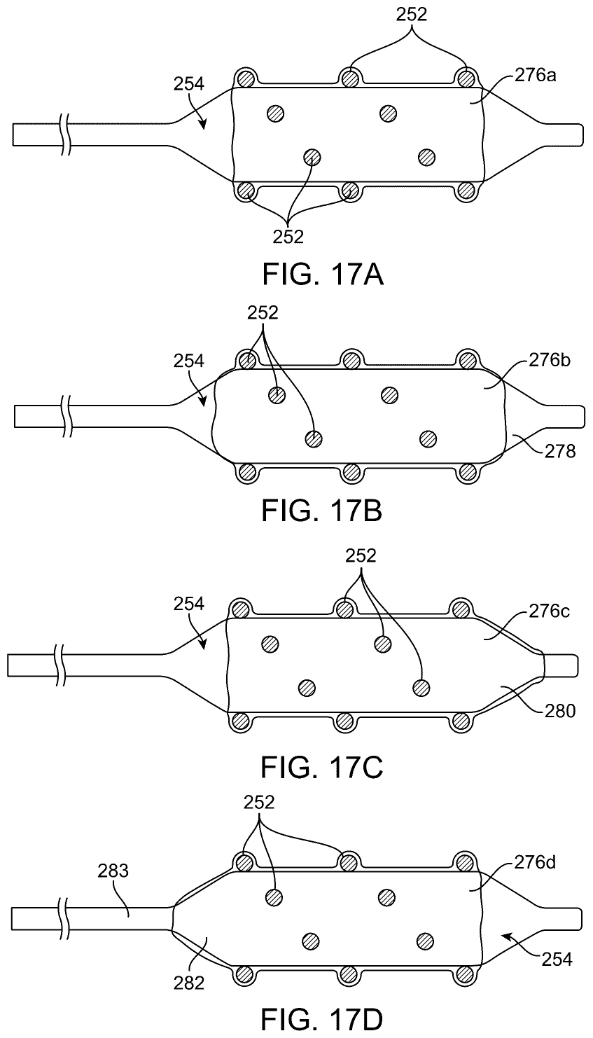

Alternatively or in addition to use of an adhesive, welding, or the like, the stress-applying or stress-applying features of the present invention, such as spherical features 252, may be immobilized on a surface of a substrate 254 using an elastic, constraining sleeve or sheath 276a-276d, as shown in FIGS. 17A to 17D. The sleeve conforms to the shape of the surface and the features 252 to hold and immobilize the features as the expandable member or other substrate is inflated or otherwise expanded from an initial narrow width or diameter configuration to a radially expanded or inflated configuration.

As shown in FIG. 17A, sleeve 276a may be attached to a cylindrical working length of the balloon or other substrate. In other examples (not shown), a sleeve may be attached to one side only (proximal or distal) of the cylindrical working length of the balloon or other substrate. As shown in FIG. 17B, a sleeve 276b may be attached to a tapered/conical proximal portion and distal portion 278 of the expandable substrate 254. As shown in FIG. 17C, a sleeve 276c is attached at its distal end to a tapered/conical distal tip 280 of the expandable substrate 254. As shown in FIG. 17D, a proximal end 282 of sleeve 276d is attached to a proximal conical/tapered end of the expandable structure 254 or proximal shaft 283.

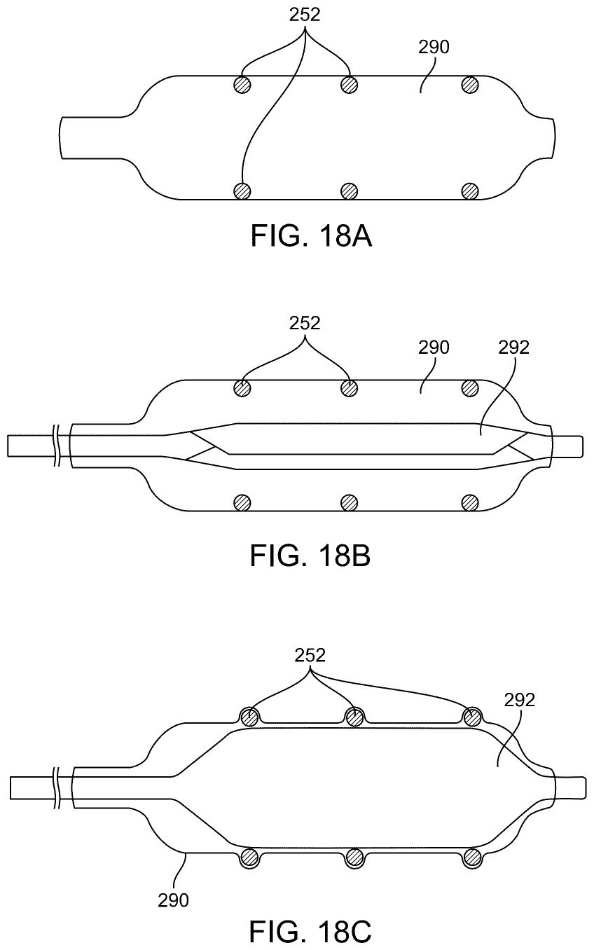

In other embodiments, the stress-applying and stress-applying features 252 of the present invention may be placed on an inner surface of an expandable structure such as an inner surface of an elastic sleeve 290 or other expandable, cage or structure, as shown in FIGS. 18A to 18C. A folded or otherwise unexpanded balloon 292 is advanced into an interior of the sleeve 290, or the sleeve is advanced or retracted over the balloon, prior to inflation or expansion of the balloon, to center the balloon within the interior of the sleeve, as shown in FIG. 18B. The balloon 292 may be inflated causing the features 252 to partially or fully protrude radially outwardly through the sleeve, or balloon membrane, as shown in FIG. 18C.

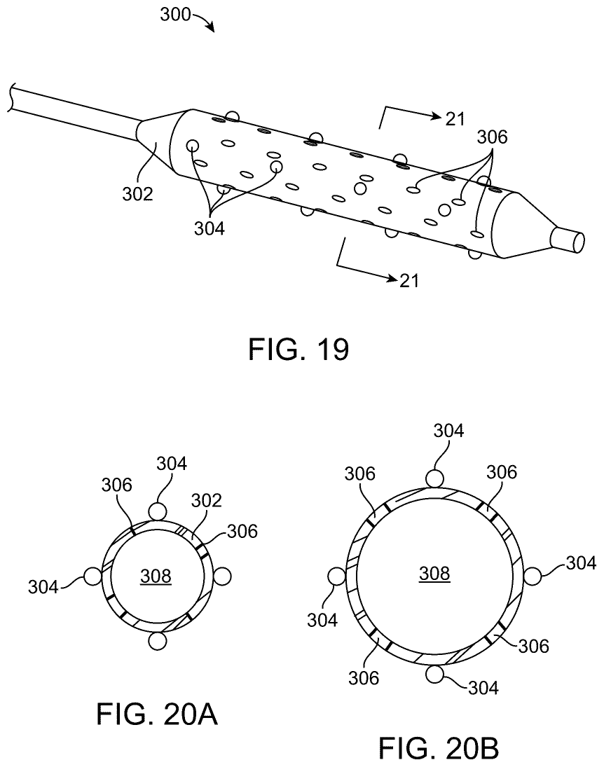

Referring now to FIGS. 19, 20A, and 20B, a drug delivery catheter 300 includes a balloon 302 having both a plurality of stress-applying features 304 and a plurality of expandable ports 306 disposed over an exterior surface thereof. The balloon 302 will typically be elastic or semi-compliant, allowing the ports 306 to expand from a closed or substantially closed configuration, as shown in FIG. 20A, to an open or expanded configuration, as shown in FIG. 20B, in response to balloon inflation. The balloon 302 may be inflated with an inflation medium carrying a drug or other medicament introduced into an interior 308 of the balloon to effect both balloon expansion and release of the medicament medium through the ports 306 as they open in response to pressurization. Ports 306 will typically be configured to open in response to an internal pressure above a minimum threshold value, typically above 3 atm, 5 atm, or 7 atm.

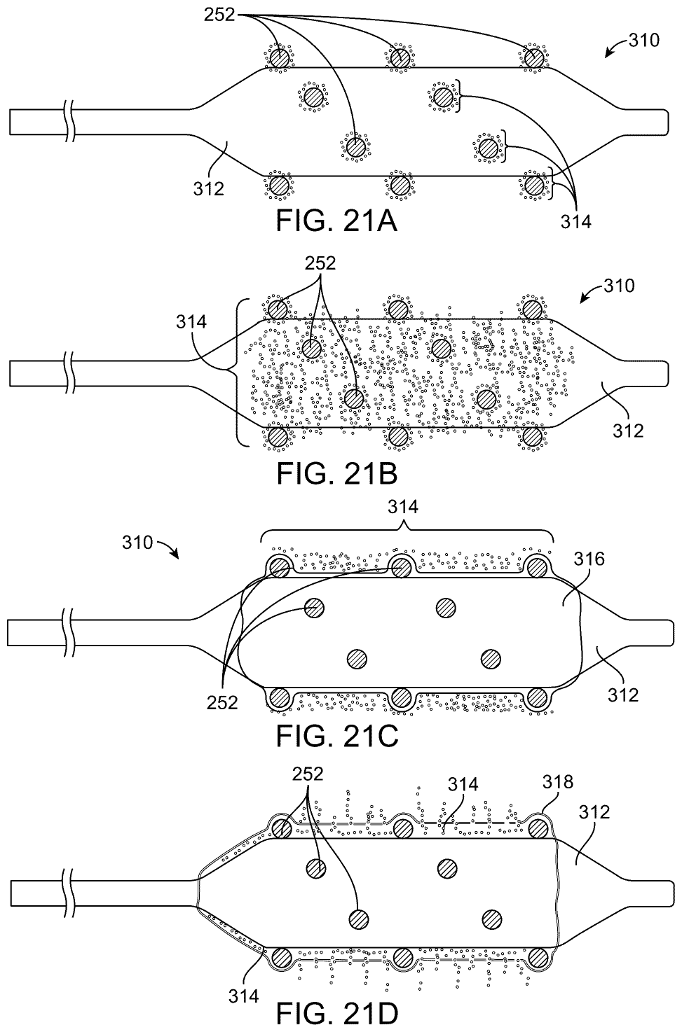

Referring now to FIGS. 21A to 21D, drug delivery catheters 310 carrying the stress-applying and stress-applying features 252 of the present invention and having alternative structures are illustrated. As shown in FIG. 21A, a drug delivery balloon 312 carries a plurality of features 252 which radially outwardly protrude from the balloon's outer surface. Drug particles 314 are coated over and/or incorporated within features 252, e.g., the drug may be absorbed within porous features or may be held within a reservoir or hollow interior of the features. As shown in FIG. 21B, the drug may be coated over the surface and/or be releasably absorbed within the surface of either or both of the features 252 and the delivery balloon 312. As shown in FIG. 21C, the drug 314 may be coated over the surface and/or absorbed within an elastic membrane 316 which in turn is placed over balloon 312 in a manner similar to that described above with reference to FIG. 17A to 17C. In some instances, drug 314 may be held within a porous, elastic membrane 318, as shown in FIG. 21D. In such instances, drug 314 will be released through the porous or perforation in the membrane of elastic member 318 as the balloon 312 is expanded.

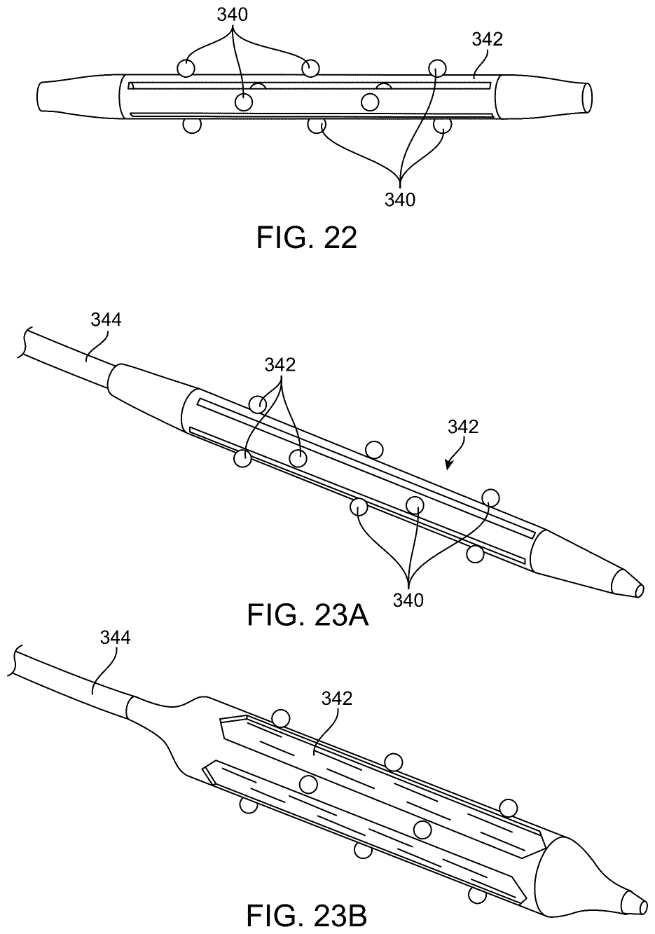

Referring now to FIG. 22, stress-applying features 340 of the present invention may be placed on an exterior surface of a balloon-expandable scaffold or “cage” 342 of a type intended for temporary placement in a target vascular location for stress-applying and subsequent removal. The cage maybe formed from an elastic polymeric material, from an elastic metal such as nickel-titanium alloy, or from another material that expands and contracts when an expandable structure is expanded and deflated (or collapsed) inside the cage.

Referring now to FIG. 23A, the expandable scaffold 342 of FIG. 22 has a central lumen or passage that may be placed over a balloon 342 (FIG. 23B) of a balloon catheter 344 prior to expansion of the balloon.

As shown in FIG. 23B the balloon 342 may be inflated to expand the expandable scaffold 342 to push the stress-applying features 342 on the scaffold radially outwardly to engage plaque. The scaffold will be formed from an elastic material, usually an elastic metal such a nickel-titanium alloy, e.g., Nitinol® or other superelastic metal or polymer, so that it will open with the balloon when the balloon is inflated as well as close radially inwardly over the balloon as the balloon is deflated.

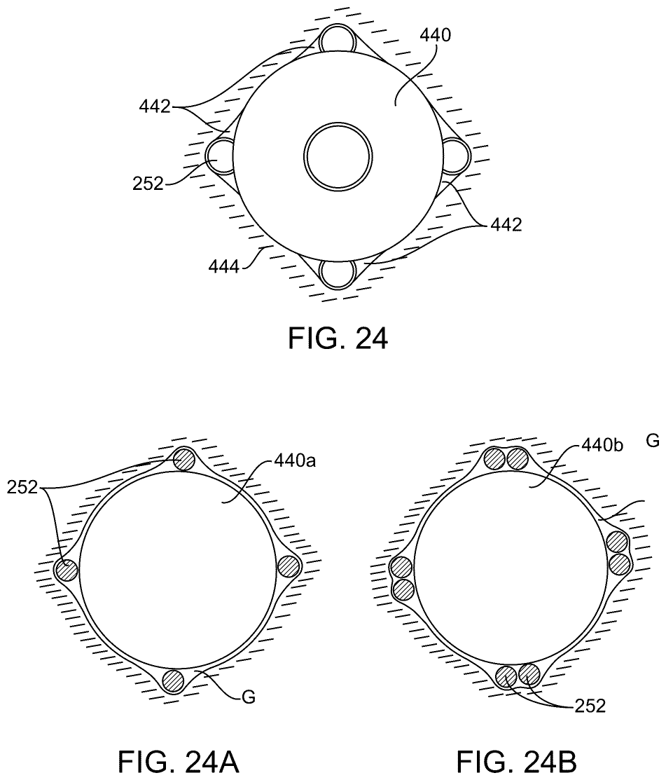

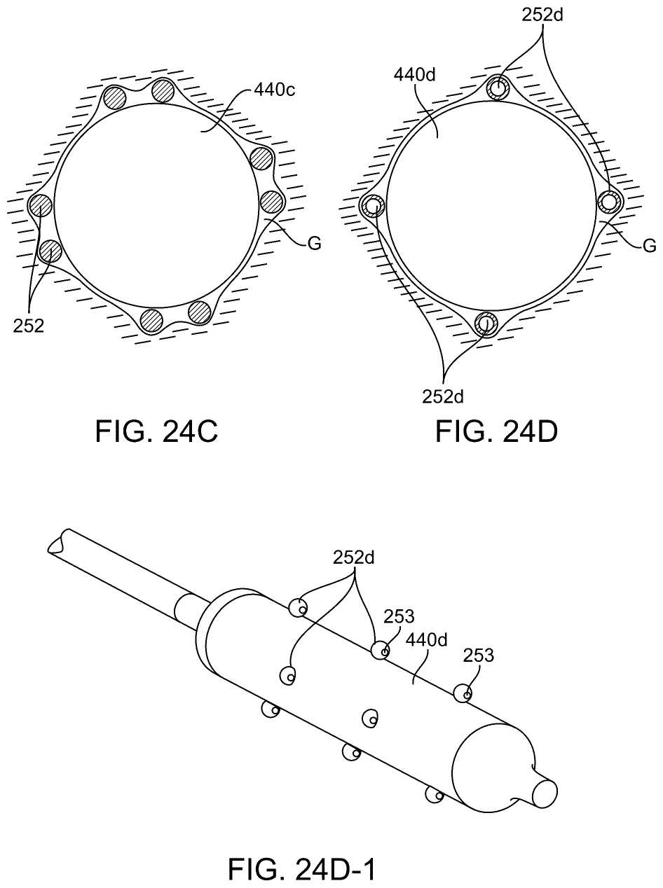

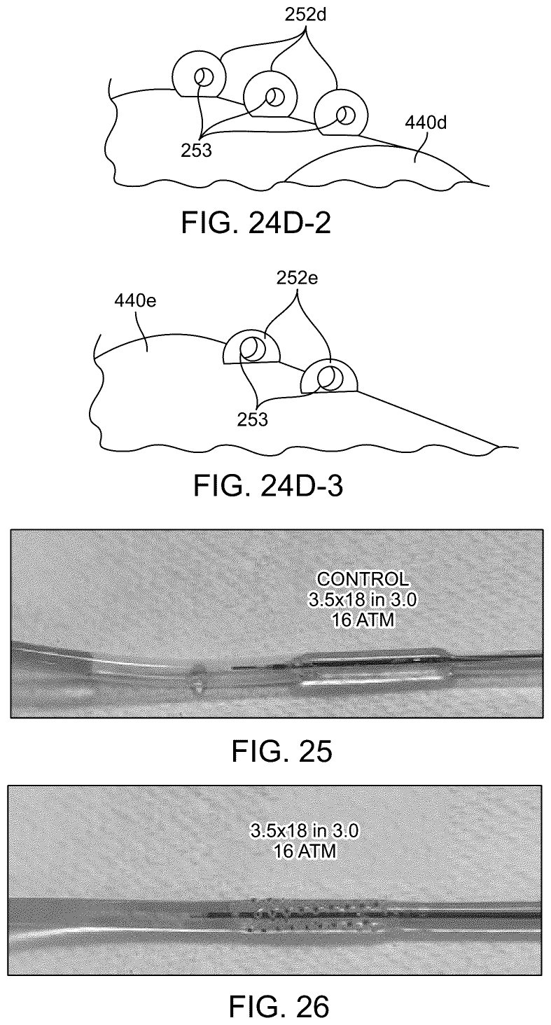

As shown in end view in FIG. 24, a balloon or other expanded member 440 has a plurality of discrete stress-applying or stress-applying features 252 distributed over its circumference and length where the features act as spacers and are arranged in a pattern, density, size, shape, and/or footprint of the features create a plurality of potential pathways 442 along the length of the balloon between an outer surface of the expanded member and inner surface of the lesion 444, providing plurality of channels or gaps which permits the perfusion (flow) of blood, contrast media, drugs, and the like, past the inflated outer surface of the expanded member under expected vascular conditions. In a preferred example, the channels conduct fluid such as contrast fluid delivered at a pressure ranging from 1 psi to 10 psi, preferably at a pressure ranging from 1 psi to 5 psi, more preferably at a pressure ranging from 1 psi to 3 psi. In some instances, the channels may not allow the flow of blood or contrast media at physiologic pressure. In such circumstances, it may be necessary to pressurize the vessel or body lumen by from 1 psi to 3 psi above physiologic pressure. Compare FIG. 25 which shows how an inflated conventional balloon will block the flow of a colored medium in a mock vessel with FIG. 26 where the colored medium will flow past a balloon 440 having the surface features of the present invention.

In some instances, the spacer-features are configured or arranged to allow passage of contrast medium through channels around or adjacent to the features, and/or through holes (see FIGS. 24D, 24D-1, 24D-3, and 24D-3) in the features, at pressures ranging from 10 mmHg to 500 mmHg, preferably from 10 mmHg to 200 mmHg, more preferably from 10 mmHg, to 100 mmHg. Alternatively, the spacer-features may be configured to allow passage of fluid, contrast, and/or medicaments from 0.5 psi to 5 psi, preferably from 0.5 psi to 3 psi, more preferably from 0.5 psi to 2 psi, when the expandable structure is expanded to nominal expanded configuration or nominal diameter.

FIGS. 24A to 24D illustrate balloons 440a to 440d having different feature patterns to create bypass channels or gaps G according to the present invention. Such constructions are particularly advantageous when performing fluoroscopy or medication injection across an expanded member in I blood vessel or other lumen where contrast or a drug solution is injected into the vessel proximal to the expandable member at pressure above physiologic pressures, sufficient to allow the channels to conduct fluid across the axial length of the expanded structure. FIG. 24A shows a plurality of features configured to provide channels sufficient to allow contrast fluid bypass across the axial length of the expandable structure when the structure is in the expanded configuration such as an expanded (fully inflated balloon). FIG. 24B is another example of a dual adjacent feature along the circumference and/or axial length of the expandable structure to provide channels to conduct contrast fluid or medicaments across the axial length of the expandable structure when the structure is in the expanded configuration. FIG. 24C example shows another example of a plurality of features oriented to create channels (gaps) between the features in an axial direction and/or circumferential direction. FIG. 24D shows holes 253 (FIGS. 24D-1 and 24D-2) formed in the features oriented axially (shown) to allow contrast, fluid, and/or medicaments to bypass an expanded structure such as an expanded balloon.

FIGS. 24D-1 to 24D-3 illustrate features of the type illustrated in FIG. 24D having holes 253 through the features 252d which further promote perfusion of contrast media past balloons when inflated in the vasculature. As shown in FIGS. 24D-1 and 24D-2, spherical features 252d have through-holes 253 which further promote and allow the flow of contrast medium past the outer surface of the balloon 440d. Similarly, as shown in FIG. 24D-3, through holes 253 may be provided in hemispherical or other spacer-features 252e on the outer surface of a balloon 440e or other expandable structure. By arranging the spacer features along axial lines, elongate gaps are effectively created along axial paths on the balloon surface. Such axial paths, however, can sometimes be blocked by the spacer features 252d and 252e themselves. Such blockages can be lessened or eliminated by including through or bypass holes in the spacer features, preferably being axially aligned with the balloon.

It has been found that the use of spacers features (even without holes) can be effective in permitting contrast perfusion past balloons inflated at nominal pressures (typically 7 atm to 11 atm or higher) in the vasculature. In particular, the spacer features lower the pressure necessary to flow the contract past the balloon to assist in imaging the vasculature downstream of the balloon and reduce the back flow of contrast into the aorta or elsewhere. The inclusion of through holes has been found to lower the necessary contrast delivery pressure even further, further protecting the patient from excess delivery of toxic contrast media. Similarly, the inclusion of channels through the features as shown, for example, in FIGS. 2H-15 to 2H-21 would similarly promote contrast and blood perfusion in comparison to features lacking such internal flow paths.

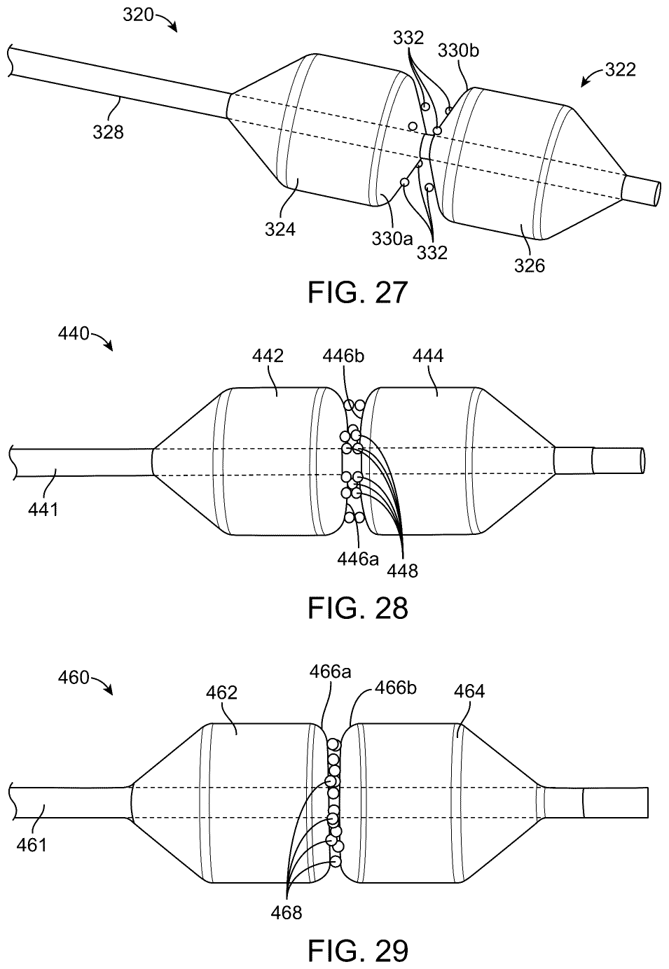

Referring now to FIG. 27, a valvuloplasty catheter 320 includes a segmented balloon structure 322 mounted on a distal end of a catheter shaft 328. The segmented balloon structure 322 includes both a proximal segment 324 and a distal segment 326, and the segments have opposed internal faces 330a and 330b. Each of these opposed surfaces has a plurality of stress-applying features 332 distributed thereover. By inflating each of the segments and capturing calcified cardiac or other valve leaflets therebetween, the calcified plaque may be disrupted in order to improve valve function. In some instances, the segments 324 and 326 will be inflated simultaneously, while in other cases, one of the segments may be inflated initially, the inflated segment drawn against one side of the valve leaflets, and the other segment then inflated to effect disruption of the calcifications.

Valvuloplasty devices having other patterns of stress-applying features are shown in FIGS. 28 to 31B. As shown in FIG. 28, a valvuloplasty catheter 440 includes a catheter shaft 441 having a proximal balloon 442 and a distal balloon 444 having opposed balloon surfaces 446a and 446b with flat faces each having a plurality of stress-applying features 448 distributed thereover. Pairs of stress-applying features 448 are arranged so that features on opposed services directly engage each other to apply force to a cardiac or other valve leaflet trapped therebetween.

Valvuloplasty catheter 460 illustrated in FIG. 29 is similar to that illustrated in FIG. 28 having a proximal balloon 462 and a distal balloon 464 with opposed faces 466a and 466b thereon. The plaque disrupting features 468, however, are arranged so that they do not engage each other and instead directly engage the opposed balloon surface.

The balloons 322, 324, 442, 444, 462, and 464 may be fixedly attached to their respective catheter shafts so that the valve leaflets are entrapped only by inflating the balloons but will more often be slidably mounted relative to each other so that the balloons may be spaced-apart to capture the valve leaflets and then brought together to trap the valve leaflets and apply focused forces through the features to stress and disrupt calcifications and plaque on the leaflets.

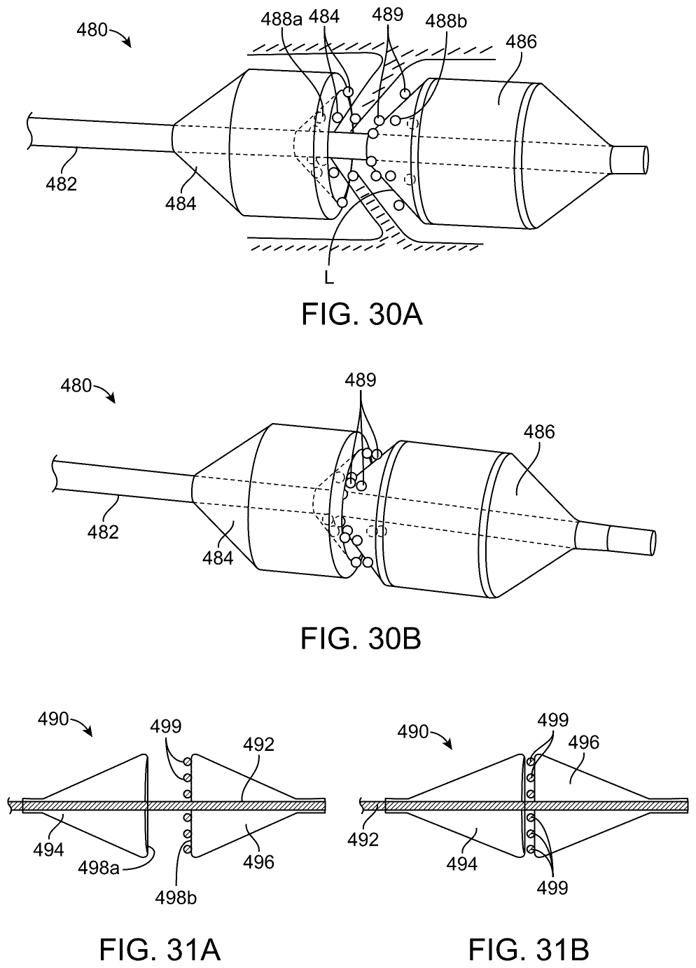

As shown in FIGS. 30A and 30B, a valvuloplasty catheter 480 has a pair of nesting balloons 484 and 486 mounted on a shaft 482. Proximal balloon 484 has a concave conical surface 488a with a plurality of stress-applying features 486 distributed thereover. Distal balloon 486 has a convex conical surface 488b with a plurality of stress-applying features 489 distributed over its surface. A cardiac or other valve leaflet L may be captured between the balloon surfaces 488a and 488b when the surfaces are spaced apart, as shown in FIG. 30A, and plaque or other calcifications on the leaflets may be disrupted by drawing the distal and proximal balloons 486 and 484 together, as shown in FIG. 30B.

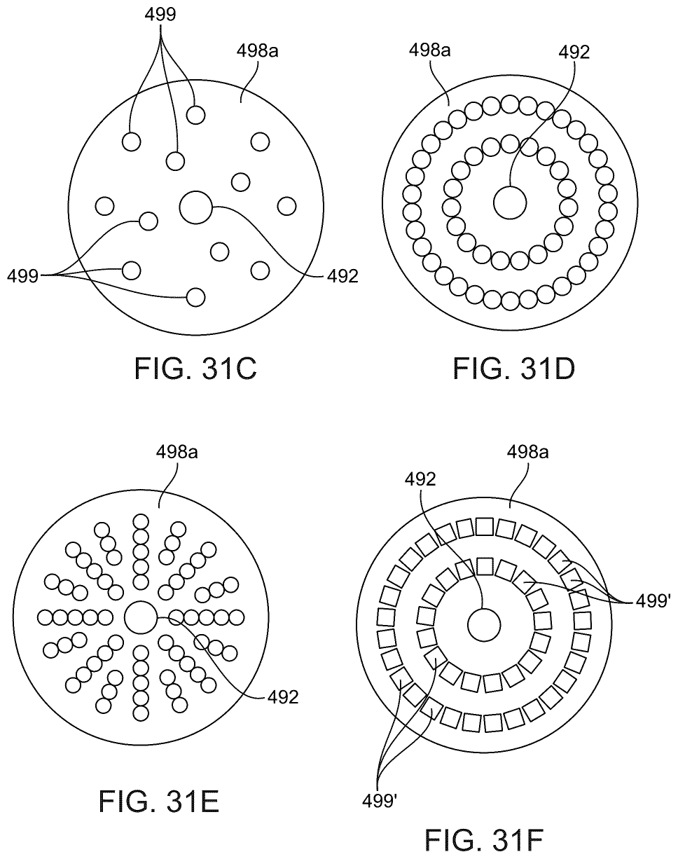

As shown in FIGS. 31A to 31B, valvuloplasty catheter 490 has a proximal balloon 494 and a distal balloon 496 mounted to slide relative to each other on a shaft 492. Opposed surfaces 498a and 498b of the balloons may be separated to capture a valve leaflet and brought together to engage stress-applying features 499 against the leaflets trapped therebetween. As can be seen, only surface 498b carries the stress-applying features and surface 498a is free from such features. The balloon 494 and 496 are shown to be conical which can be advantageous in accessing smaller regions.

FIGS. 31C to 31F illustrate a variety of distribution patterns for the stress-applying features 499 on the opposed surface 498a. For example, in FIG. 31C, the stress-applying features 499 are arranged in a spaced-apart manner in inner and outer rings. In FIG. 31D, the stress-applying features 499 are arranged in a closed-packed manner (with adjacent stress-applying features 499 in contact or spaced very closely together) in inner and outer rings. In FIG. 31E, the stress-applying features 499 are arranged along radial lines, e.g., starburst vectors. While the stress-applying features 499 are typically spherical or hemispheric structure are described elsewhere herein, they can have other geometries, such as rectangular or cubic solids. For example, as shown in FIG. 31F, cubic solid stress-applying features 499′ are arranged in a closed-packed manner (with adjacent stress-applying features 499 in contact or spaced very closely together) in inner and outer rings. While these patterns have been described on surface 498a, they could also be formed on the opposed surface 498b, and the opposed stress-applying features 499 could be in alignment so that they engage each other as the surfaces are brought together and/or may be out of alignment so that the engage the opposed surface and not the opposed stress-applying features 499. Further, while these distribution patterns have been illustrated on the opposed surface 498a, they could also be formed on any other opposed surface illustrated herein, such as surfaces 330a and 330b in FIG. 27, surfaces 446a and 446b in FIG. 28, surfaces 466a and 466b in FIG. 29, and surfaces 488a and 488b in FIGS. 30A and 30B.

Still further, as illustrated herein the opposed valvuloplasty “elements” have all been inflatable balloons and other structures. In other instances, however, the expandable structures may be mechanically expandable cages, scaffolds, lattices, and the like. For example, such structures may be configured to radially expand upon axial foreshortening. Such structures are well known in the medical device arts.

In still other instances, the opposed valvuloplasty “elements” may be non-expandable, e.g., solid structures or rigid hollow structures which are advanced to the target valve annulus in their “packaged” configuration. Such non-expandable designs may have any of the expanded or inflated geometries illustrated above in FIGS. 27 to 31F. Such non-expandable opposed elements may be sized at 10Fr, 12Fr, or larger, and may be delivered intravascularly. Even larger sizes could be introduced in minimally invasive transthoracic procedures and open-chest procedures.

The valvuloplasty balloon structures will typically be configured to compress or “sandwich” the valve leaflet(s) between the opposed surfaces. In some instances, the surfaces will be positioned by inflation only, i.e., the opposed surfaces will deploy and compress the leaflets as they reconfigure in response to inflation alone. In other instances, segments of the balloon structure having the opposed surfaces may be first deployed by inflation or otherwise and then drawn axially together to compress the valve leaflets between the opposed surfaces and engage the stress-applying features. The valvuloplasty balloon structures will typically be configured to compress or “sandwich” the valve leaflet(s) between the opposed surfaces of the balloon or expandable structure axial length.

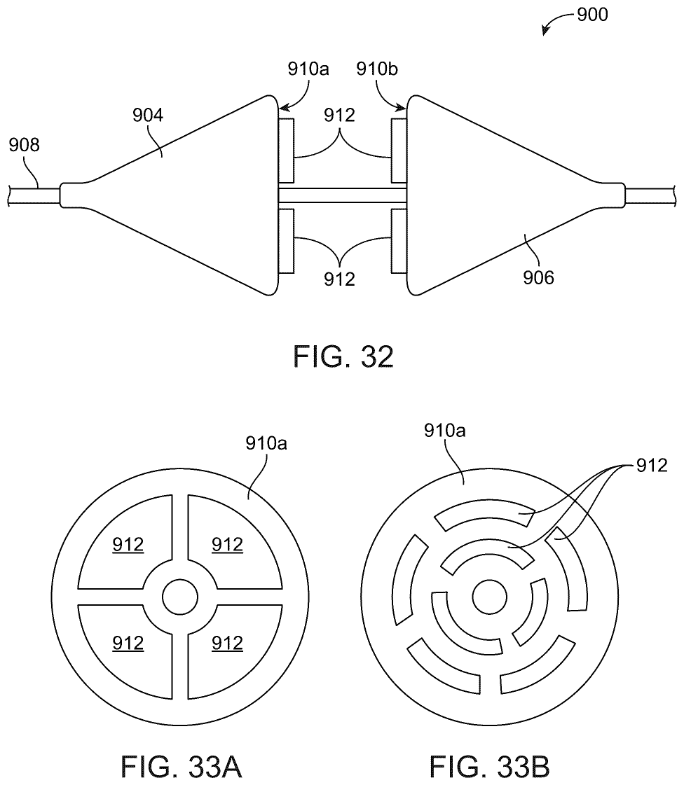

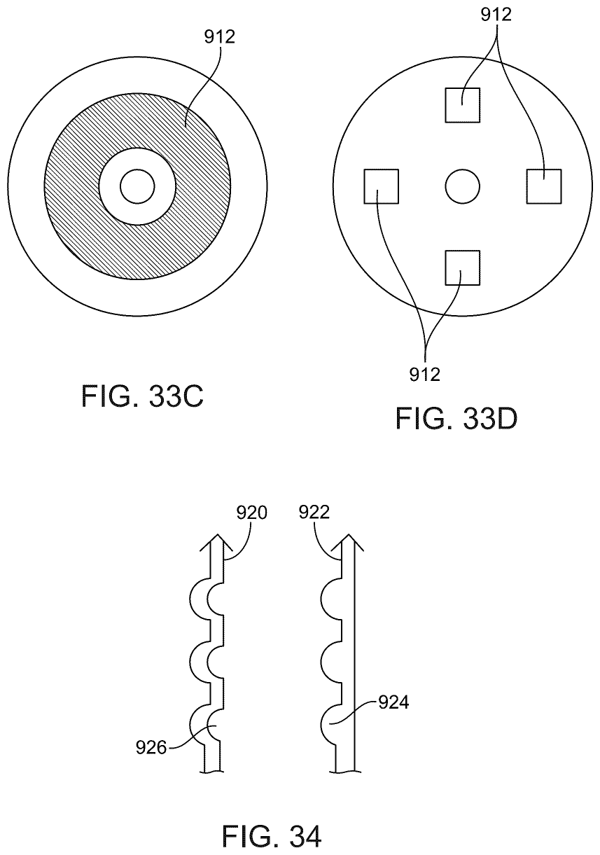

As shown in FIGS. 32 and 33A, valvuloplasty catheter 900 has a proximal balloon 904 and a distal balloon 906 mounted to slide relative to each other on a shaft 908, similar to valvuloplasty catheter 490 in FIGS. 31A to 31B. Opposed surfaces 910a and 910b of the balloons may be separated to capture a valve leaflet and brought together to engage stress-applying features 912 against the leaflets trapped therebetween. The stress-applying features 912 are shown as flat plates but could have concave or convex surfaces as well. Also, the features are shown as orthogonally arranged “quarter disks,” but could have other shapes and distributions, as shown in FIGS. 33B to 33D. The stress-applying features 912 in FIG. 32B comprise a plurality of crescent shaped elements. A single stress-applying feature 912 in FIG. 32C comprise a an annular or “donut-shaped” plate. The stress-applying features 912 in FIG. 32D comprise a plurality of rectangular elements. In all of these instances, the stress-applying features 912 may be fabricated from materials harder than the balloon, such as metals, polymers, ceramics, and the like. As shown in FIG. 32, both surface 910a and 910b carry the stress-applying features 912, and the features are arranged to engage each other as the balloons 904 and 906 are closed together, but in other embodiments one of the surfaces could be free of features and some or all of the features might not have an opposed feature on the opposite balloon.

Alternatively, plaque disrupting features such as those illustrated in FIG. 33A, may be formed as a torus (donut-shaped) on a side wall of an expandable structure, such as balloon, and have an outer surface (exposed to the vessel wall when the balloon is expanded) with a flat, convex, concave, or other geometry.

Additionally, the present invention provides valvuloplasty apparatus for treating a patient valve having calcified leaflets, where the apparatus comprises a catheter body having a proximal end and a distal end and a segmented expandable or non-expandable structure. The structure is disposed at the distal end of the catheter body and has opposed internal walls configured to be deployed or positioned on opposite surfaces of the leaflets to disrupt calcification on the calcified valve when expanded and/or axially drawn together. Features on either or both the opposed internal walls are configured to disrupt calcified plaque on the valve leaflets as the opposed internal walls are deployed and/or axially drawn together. In some instances, the opposed internal walls are configured to close together when the structure is expanded. In other instances, the segments are configured to be drawn together to capture the calcified valve leaflets therebetween. Examples include but not limited to cages, scaffolds, cylindrical tubes formed from polymeric, metallic, or other material.

FIG. 34 shows a further variation where opposed balloon surfaces 920 and 922 have mating protrusions 924 and cavities 926 where a calcified leaflet is captured between the protrusion and the cavity as the balloon are closed together. The protrusions and cavities may be molded or otherwise formed into the opposed balloon surfaces or could be formed by metal or other hardened features or elements that are embedded in or otherwise attached to the balloon surfaces.

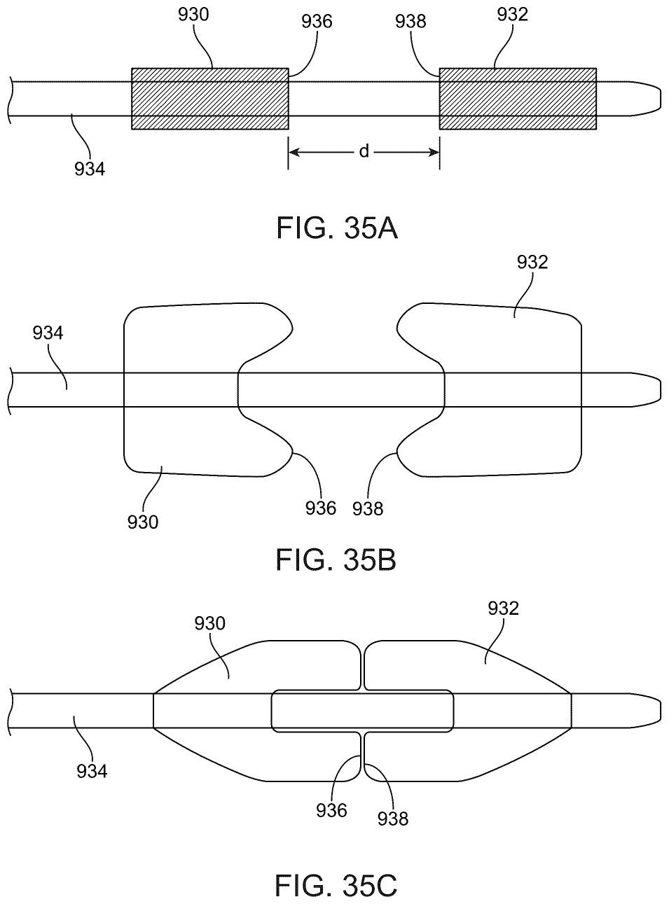

Referring to FIGS. 35A to 35C, opposed balloons 930 and 932 may be mounted on a distal end 934 of a valvuloplasty catheter to close together as the balloons are inflated. There will be no need to have the balloons slide on the catheter. As shown in FIG. 35A, when uninflated, inner edges 936 and 938 of the balloons 930 and 932, respectively, are spaced apart by a distance d. As the balloons 930 and 932 are inflated, as shown in FIG. 35B, the edges evert outwardly to move toward each other to reduce the distance therebetween. Finally, when fully inflated as shown in FIG. 35C, the edges 936 and 938 have closed the gap and “kiss” to apply force to a leaflet captured therebetween. While the opposed surfaces of the balloon have effectively closed together, the attachment points of the balloon on the catheter have not changed. The apparent motion is solely a result of the inflated shapes of the balloons.

A similar effect can be obtained with a single, two-lobed balloon that can be inflated to capture the leaflets between opposed surfaces of the lobes as the lobes are simultaneously or sequentially inflated (not illustrated).

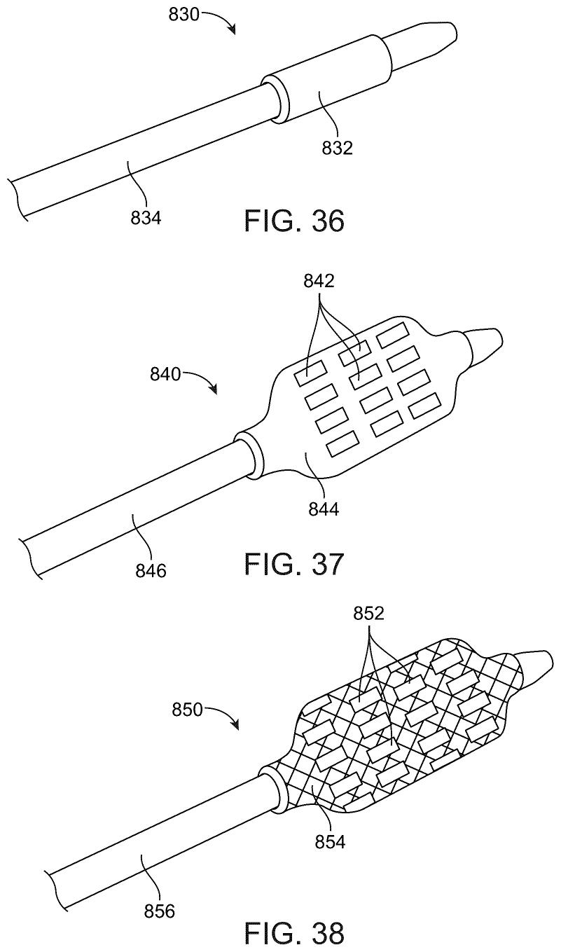

In the event that one or more stress-applying features are lost from a deployment catheter during an intervention, the present invention provides magnetic catheters which are configured to secure and retrieve the lost magnetic and/or magnetizable stress-applying features, e.g., features formed at least in part from a ferromagnetic material which has is magnetic or magnetizable, usually being magnetizable. The magnetic retrieval catheters will carry magnetic retrieval elements which are typically electromagnets or permanent magnets. As shown in FIG. 36, a magnetic retrieval catheter 830 may include a cylindrical magnetic element 832 disposed on a distal portion of a shaft 834. As shown in FIG. 37, a magnetic retrieval catheter 840 may include a plurality of magnetic element 842 distributed over an outer surface of an inflatable balloon 844 mounted on a distal portion of a shaft 846. As shown in FIG. 38, a magnetic retrieval catheter 850 may include a plurality of magnetic element 852 distributed over an outer surface of an expandable cage 854 mounted on a distal portion of a shaft 856. In all cases, the magnetic retrieval catheters may be deployed with a separate sheath (not shown) which may be configured to receive and contain the lost stress-applying features after they are secured by the magnetic retrieval catheter, e.g., retrieval the distal portion of the catheter will be retracted into a lumen of the sheath to constrain the lost stress-applying features as they are removed from the vasculature.

In a specific example, hemispherical stress-applying features are secured to an outer surface of a balloon on an angioplasty, valvuloplasty, or other plaque- or calcification-disrupting catheter as follows. The balloon is inflated to the expanded configuration, typically at least nominal inflated pressure, usually rated burst pressure, cleaned with absolute ethanol from a proximal notch to a distal tip, and force air-dried five times.

A working length of the outer balloon surface, typically from 5 mm to 50 mm, is coated with a “base” layer of light-curable acrylic adhesive, such as Loctite® 3321, manufactured by Henkel A G, Dusseldorf, Germany. For example, the working length of the balloon is coated in axial direction to form a thin, even layer of adhesive, to ensure that entire working length is evenly and entirely coated with two or three passes.

The acrylic adhesive is then cured by rotating exposure to an LED source (1-2 W/cm² at 365 nm or 405 nm) for a time sufficient to achieve the desired curing, typically 60 seconds with the rotational speed at 30 rpm at 20 mm from the source.

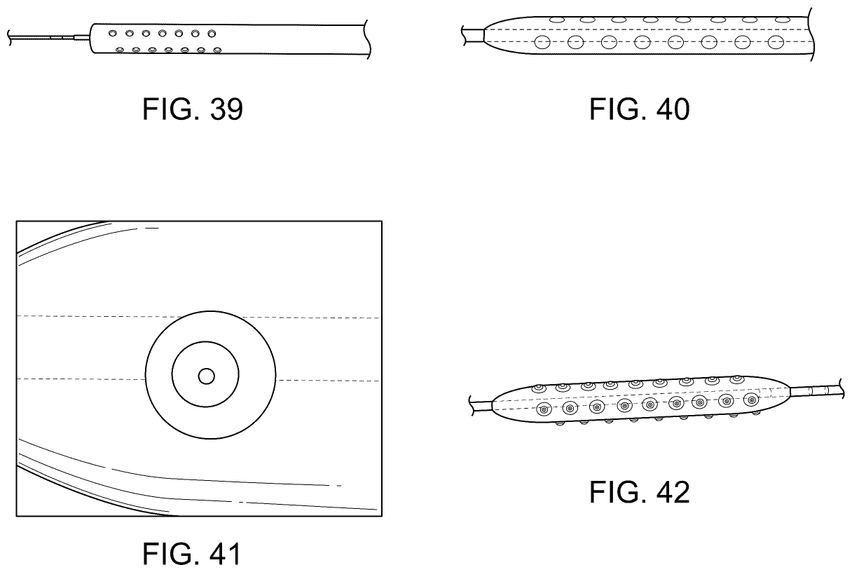

After deflating, the balloon is folded, and the working length is placed inside a tubular indentation template fixture. See, FIG. 39. The balloon is then inflated typically to at least nominal, usually rated burst pressure, within the template fixture. The inflated balloon in the template is then rotated for 15 seconds at a rate of one revolution/5 second within in a “hot box” at 215 F°. The template fixture is then removed and cooled, and the balloon is removed from the template fixture and deflated, forming indentation “marks.”

As shown in FIG. 40, indentation marks are formed over the working length up to the distal taper. The most distal indentation mark(s) should be near or at the junction of the balloon taper and the working length. The individual indentations typically have a depth of about 0.01 mm to 1 mm and a width of about 0.1 mm to 1 mm.

Hemispherical stress-applying features are secured in the indentations on the outer surface of the balloon as follows. The balloon is again re-inflated and drops of a light-curable acrylic adhesive, such as Loctite® 3943, are dispensed into each indentation (over the earlier applied and cured acrylic adhesive layer) stating at the proximal-most indentation. Individual hemispherical stress-applying features, typically having a diameter from about 0.1 mm to 1 mm, are placed flat surface down and pressed into the acrylic-filled indentation, and the acrylic cured with light. The bottoms of the hemispherical stress-applying feature are now secured to the outer balloon surface.

After the bottoms of all the hemispherical stress-applying features are secured to the outer balloon surface, a third “spot” coat or layer of a light-curable acrylic adhesive, such as Loctite® 3943, is dispensed over the feature and the adjacent surrounding area on the balloon, including areas covered by both the first and second cured adhesive layers. The third adhesive layers are the light-cured generally as described above, and the features ae fully encapsulated by the different adhesive layers forming a substantially impervious shell anchored to the first or base adhesive layer which surrounds the entire balloon, greatly reducing the risk of any feature being lost. See, FIGS. 41 and 42.

After all features have been encased in three cured-adhesive layers, as just described, a fourth “topcoat” light-curable acrylic adhesive layer, such as Loctite® 3321, will typically be applied. The topcoat layer may be applied to the application of the base layer as described above identically or differently such as circumferentially, followed by a final light-curing for an extended period, such as 5 minutes to 20 minutes. The balloon may then be deflated, folded, and packaged for subsequent use.

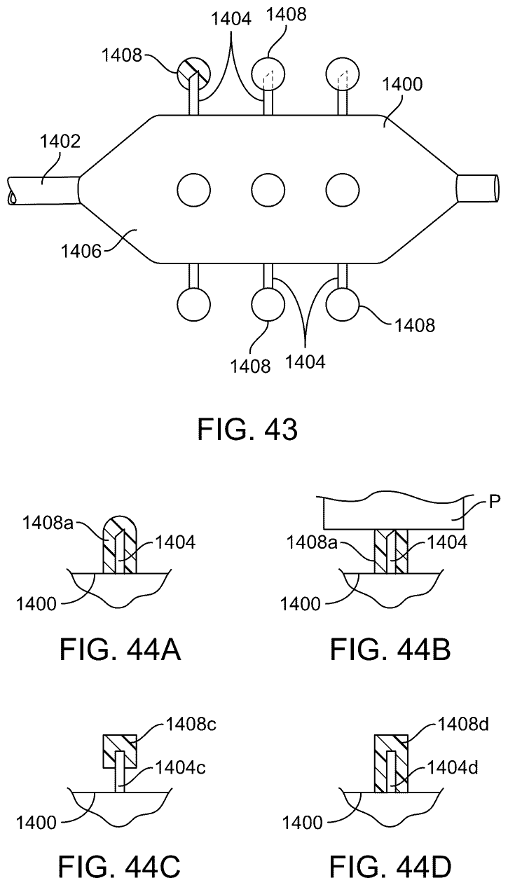

FIG. 43 illustrates a balloon 1400 of a balloon catheter 1402 having needle-like stress-applying features 1404 on an outer surface 1406 of the balloon covered by or coated over with a polymeric or other material to form atraumatic covers 1408. As shown FIG. 43, the atraumatic covers 1408 are spherical and cover only the distal tip of each stress-applying feature 1404. The distal tips are shown to be sharpened but can also be blunt as described below.

FIGS. 44A-44D illustrate different embodiments of the covered stress-applying features of FIG. 43. for example, as shown in FIG. 44A, the needle-like stress-applying feature 1404 is fully covered buy a dome-like cover 1408a. As it shown in FIG. 44B, when the dome-like cover 1408a is pressed against a region of plaque P, the material of the dome-like cover may compress, exposing the sharpened tip of the stress-applying feature so that it may pierce and disrupt the plaque.

The needle-like, stress-applying features need not be sharpened and can instead have blunt tips as shown, for example, as feature 1408c in FIG. 44C. The covers may also have different geometries, with an exemplary cubic cover 1408c shown in FIG. 44C. A still further example is shown in FIG. 44D where a blunt stress applying feature 1404d is covered by a cylindrical cover 1408d.

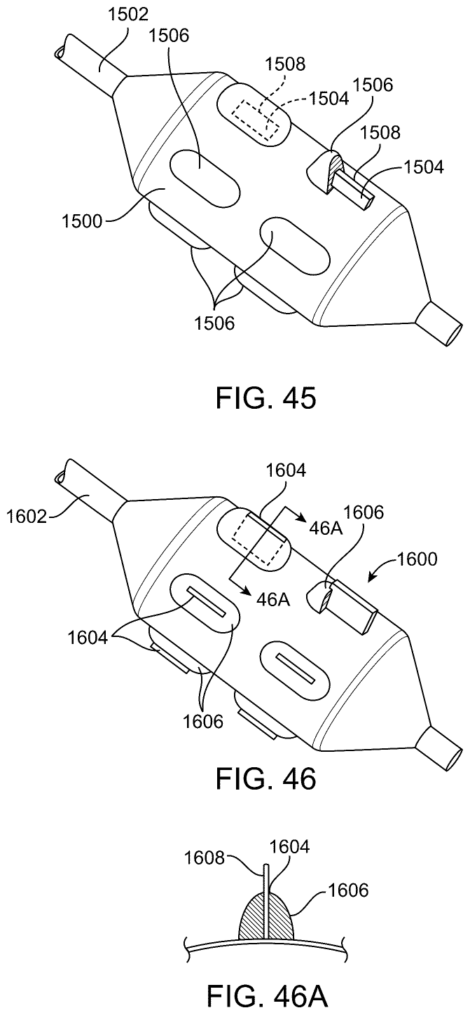

FIG. 45 illustrates a balloon 1500 of a balloon catheter 1502 having a plurality of stress-applying features 1504 in the form of elongated blades covered by or coated over with covers 1506 formed from a polymeric or other compressible material. The cutting blades 1504 will typically have a cutting edge 1508, and the covers 1506 will typically cover the entire cutting blade 1504 including the cutting edge 1508. The cover 1506 will usually be compressible so that the cutting edge can be exposed when the balloon 1500 is inflated within a region of plaque in order to allow the cutting blade to emerge, engage, and fracture the plaque.

FIGS. 46 and 46A illustrate a balloon 1600 of a balloon catheter 1602 having elongated cutting stress-applying features, such as blades 1604, partially covered by domes 1606 formed from polymeric or other compressible materials. Cutting edges 1608 of the blades 1604 are exposed at all times and available for engaging and fracturing plaque when the balloon 1600 is inflated within a region of plaque. After an initial engagement by the cutting edge 1608, the dome 1606 will be compress, allowing the blade to further enter and disrupt the plaque.

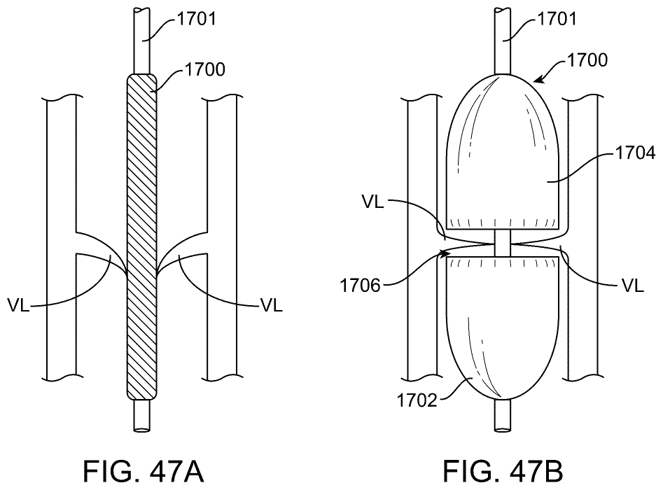

FIGS. 47A and 47B illustrate a single valvuloplasty balloon 1700 comprising distal and proximal segments 1702 and 1704 with a transversely oriented waist or annular channel 1706 formed between the segments and configured to capture valve leaflets VL therebetween. The annular channel 1706 is disposed orthogonally to the axis of the catheter shaft 1701 and is thus able to capture the leaflets while they are disposed across the valve annulus and transverse to the catheter axis. Capturing the valve leaflets in this transverse geometry has been found to be particularly effective in compressing the leaflets VL and applying stress to the calcifications on the leaflets.

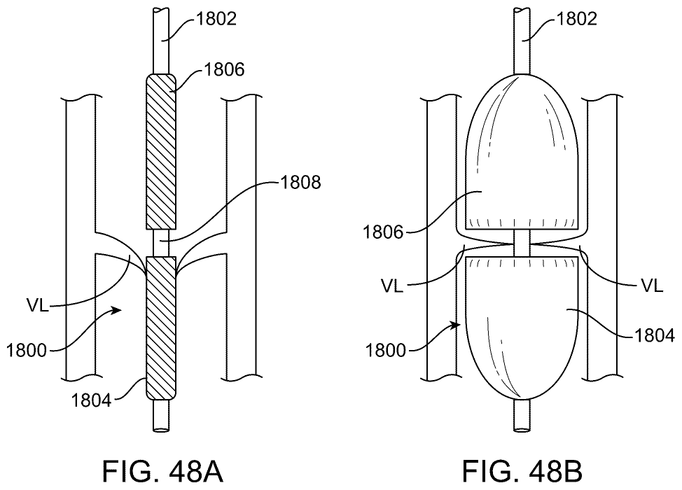

FIGS. 48A and 48B illustrate a double-balloon valvuloplasty structure 1800 mounted at the distal end of a catheter shaft 1802. The balloon structure 1800 includes separately inflatable distal and proximal balloons 1804 and 1806 with a transversely oriented gap 1808 disposed therebetween. and configured to capture valve leaflets between opposed, transversely oriented opposed surfaces thereof. gap 1808 is disposed transversely relative to an axis of the catheter 1802 and this orientation has the same advantages as described previously with respect to the annular channel 1706 the single balloon valvuloplasty catheter 1700.

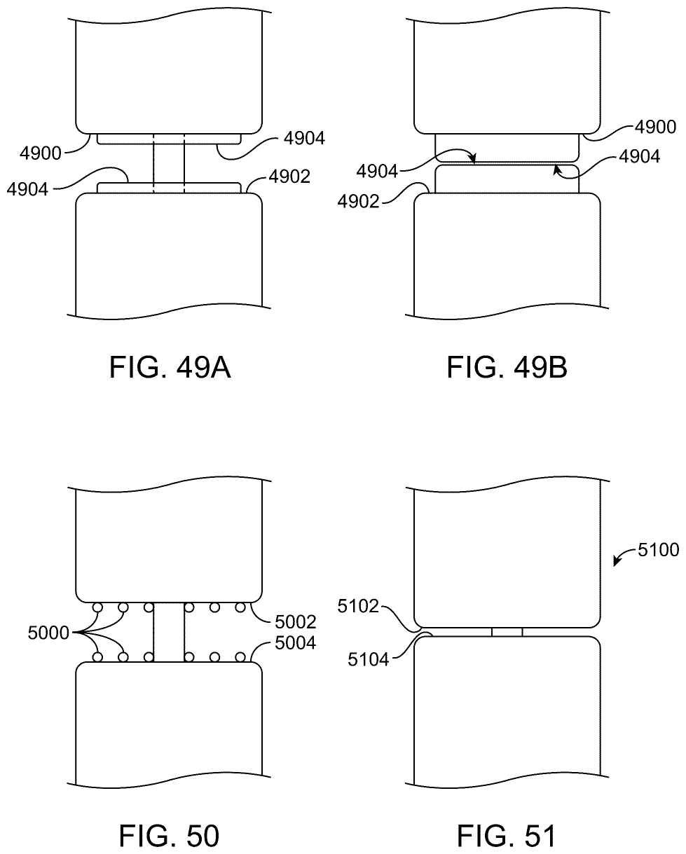

FIGS. 49A, 49B, 50, and 51 illustrate opposed balloon surface configurations suitable for the balloon structures of FIGS. 47A, 47B, 48A, and 48B. For example, as shown in FIGS. 49A and 49B, opposed balloon surfaces 4900 and 4902 may each comprise “incrementally” inflatable balloon elements 4904 which can be expanded (e.g., a higher pressure than initial inflation pressure) to apply additional stress after the valve leaflets have been captured between the opposed balloons or balloon segments with an initial inflation.

FIG. 50 shows stress-applying features 5000 on opposed surfaces 5002 and 5004 to enhance and focus stress when the balloons are inflated/hand or as the balloons are drawn together, as shown for example in FIG. 51 where balloons 5102 and 5104 of a double-balloon assembly 5100 have been drawn together to close any gap there between to apply stress to a captured valve leaflet.

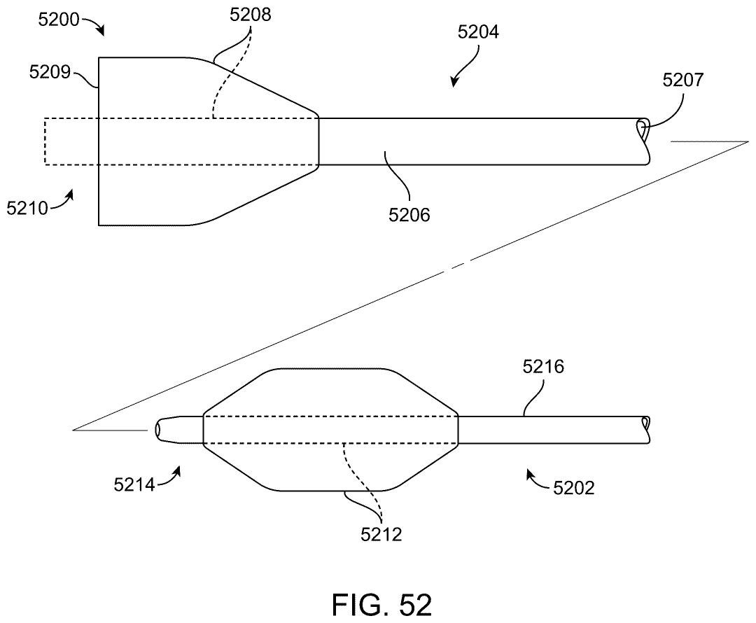

FIG. 52 illustrates a valvuloplasty system 5200 configured to capture and disrupt calcifications on aortic valves. The system comprises a sleeve catheter 5204 having a catheter body 5206 with a central lumen 5207 which receives a valvuloplasty catheter 5202. The valvuloplasty catheter comprises a valvuloplasty element 5212 at a distal end 5214 of catheter body 5216. The valvuloplasty element is typically an inflatable balloon having an inflated (expanded) configuration shown in full line and a deflated (unexpanded) configuration shown in broken line. Alternatively, the valvuloplasty element 5212 could be any other type of conventional valvuloplasty element known in the art, such as a mechanically expandable cage or scaffold, e.g., a cage which radially expands upon axial contraction. Moreover, the outer surface of the valvuloplasty element may comprise anyone or more of the stress-applying elements described elsewhere herein (not shown).

The sleeve catheter 5204 has an expandable sleeve 5208 at its distal end 5210. The expandable sleeve 5208 will typically be self-expanding where it is held in a radially constructed configuration, as shown in broken line, by a sheath or otherwise during delivery, and allowed to expand to the full line configuration in the ascending aorta above the aortic valve. As will be described in greater detail below, the expandable sleeve 5208 will be positioned out the aortic valve so that a leading edge 5209 will contact the individual valve leaflets, preferably on the aortic side of the leaflets to lie between the leaflets and an adjacent wall of the aortic root. After the expandable sleeve has been properly positioned, the valvuloplasty element 5212 can be positioned within the aortic annulus and expanded to capture the valve leaflets between an outer surface of the valvuloplasty element and an inner surface of the expandable sleeve 5208.

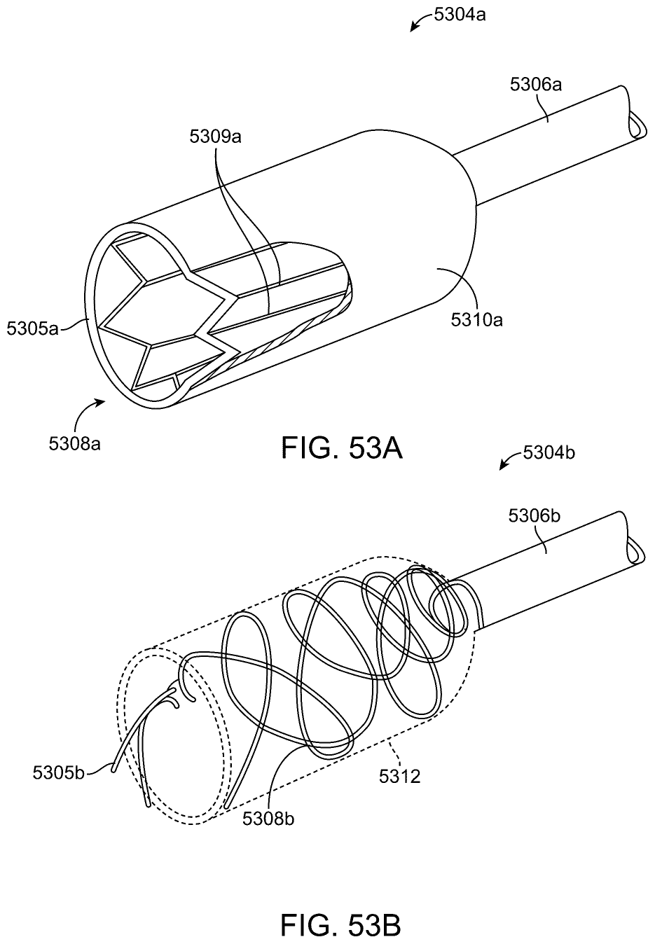

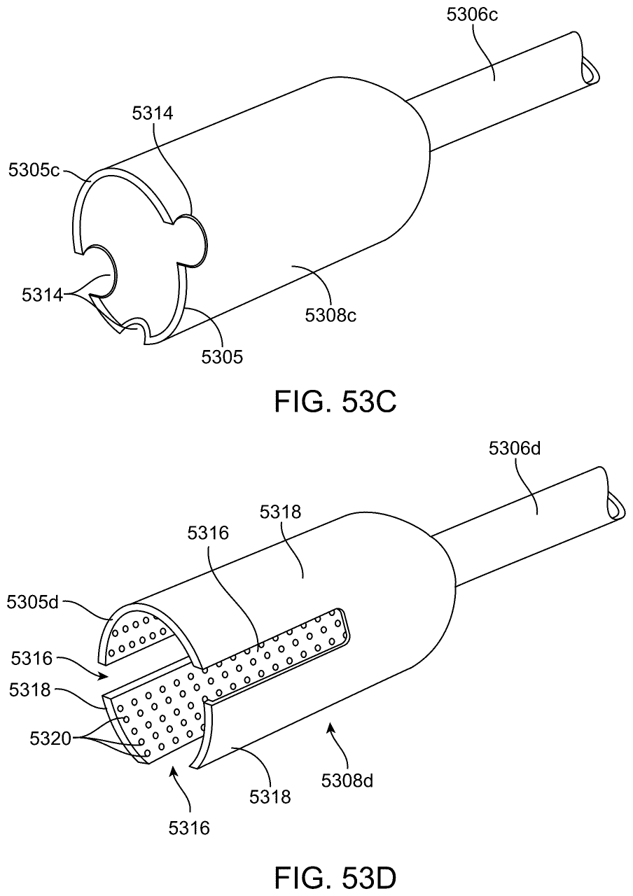

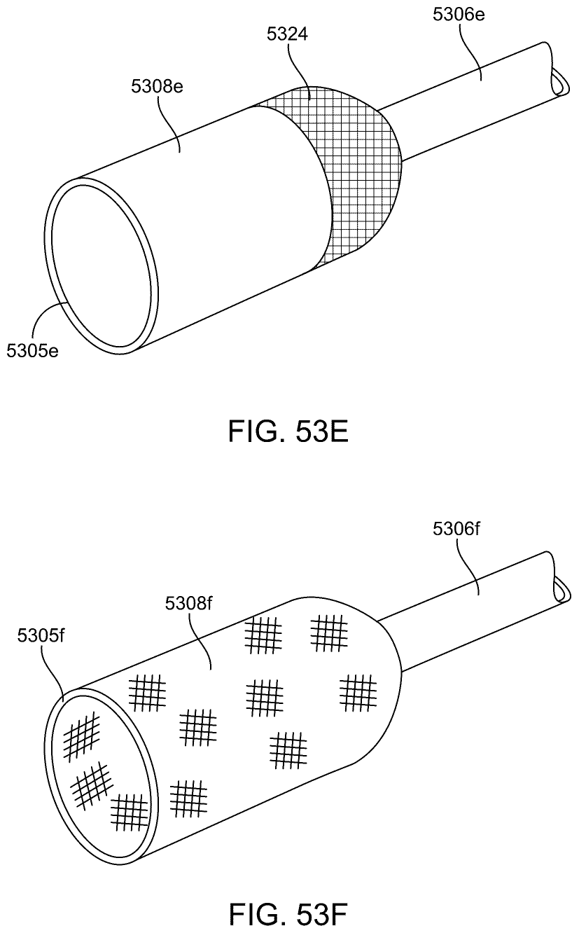

Referring now to FIGS. 53A to 53F, the expandable sleeve 5208 of the sleeve catheter 5204 may have a variety of configurations including some with filter components to capture emboli released during the valvuloplasty procedures. As shown in FIG. 53A, an outer sleeve catheter 5304a comprises an expandable sleeve 5308a which includes and expandable frame 5309a covered by or in embedded in an elastic membrane 5310a. The expandable sleeve 5308a has a generally circular distal edge 5305a with only portions of the membrane being supported by the frame 5309a. These elastic portions of the leading edge will be able to conform to and extend past the commissures of the aortic valve, as will be described in more detail below. The frame 5309a includes a plurality of axial beams or members which provide they backstop surface to enhance fracture of the calcifications on the calcified leaflets when the valvuloplasty element 5212 (FIG. 52) is expanded within the expandable sleeve 5308a.

Referring now to FIG. 53B, an expandable sleeve 5308b may be formed from a plurality of counter wound elastic wires 5312 which self-expand when released from constraint but which do not include a separate membrane or cover. the distal ends of the counter wound elastic wires 5312 terminate in a generally circular arrangement, as shown by broken line 5305b and our sufficiently mobile to engage and move past the commissioners of the aortic valve when the expandable sleeve 5308b is engaged against the aortic side of the aortic valve. The wires 5312 will typically be formed from a metal, such as nitinol or other superelastic metal. The wires 5312 will collectively form a backstop or engagement surface to capture the valve leaflets in cooperation with the valvuloplasty element 5212 when it is expanded within the expandable sleeve 5308b.

Referring now to FIG. 53C, an expandable sleeve 5308c comprises an elastic shell having a distal leading edge 5305c with three cut-outs 5314 formed therein. The cut outs 5314 are spaced so that they will be aligned with the three commissures in a patient's aortic valve when the leading edge 5305c is engaged against the upper surface of the aortic valve. In this way, the remaining portions of the leading distal edge 5305c may penetrate or extend further into the region between the valve leaflets and the inner mall of the ascending aorta or aortic root to provide additional surface to capture and compress the valve leaflets when the valvuloplasty element 5212 is expanded therein.

Referring now to FIG. 53D, an expandable sleeve 5308d comprises an elastic shell having a distal leading edge 5305d with three elongate cutouts 5316 formed therein. The cut outs 5316 are also spaced so that they will be aligned with the three commissures in the patient's aortic valve when the leading edge 5305d is engaged against the upper surface of the aortic valve. The extended length of the cutouts 5316 allows intermediate segments 5318 to extend much further into the region between the valve leaflets and the inner wall of the ascending aorta when performing a valvuloplasty procedure. Additionally, an inner surface of the elastic shell of the expandable sleeve 5308d comprises a plurality of stress-applying elements 5320. Although illustrated as hemispherical elements, such as metal hemispheres, the stress-applying elements 5320 may have any of the configurations described elsewhere herein.

Referring now to FIG. 53E, an expandable sleeve 5308e comprises an elastic shell having a distal leading edge 5305e. The distal leading edge 5305e is shown to be circular but could include cut-outs 5314 or 5316 as described earlier with respect to FIGS. 53C and 53D. The inner surface of the shell could also comprise stress applying elements over all or a portion of its surface. The expandable sleeve 5308e differs from earlier embodiments primarily because it includes an embolic filter 5324 which will allow blood to bypass the expandable sleeve during the valvuloplasty procedure while capturing emboli which will often be released as the calcifications on the leaflets are broken up.

An alternative filter embodiment is shown in FIG. 53F where the entire shell of expandable sleeve 5308f is formed from and elastic filter mesh, typically a two-layer or three-layer shape memory or “super elastic” wire mesh, and the wire mesh body of the expandable sleeve 5308f may have a circular distal edge 5305f or may have cutouts form therein as previously described.

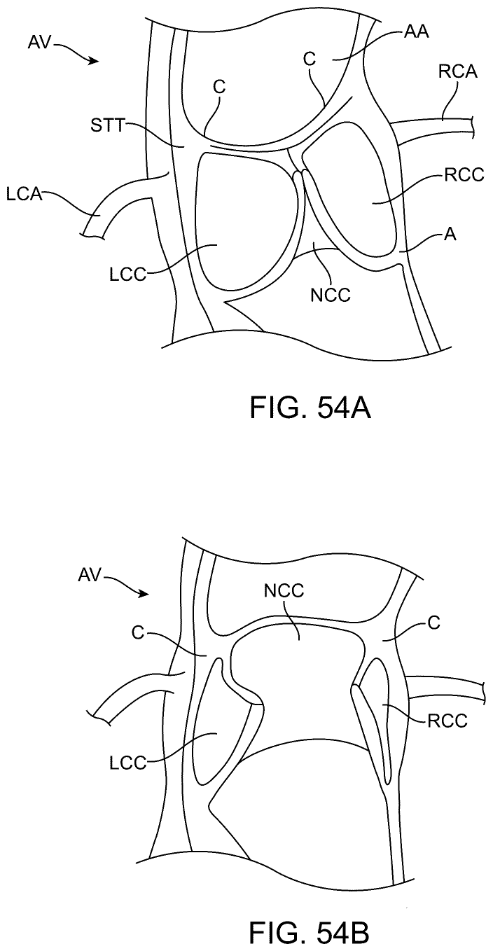

Use of the valvuloplasty system 5200 in treating calcified leaflets in an aortic valve AV will now be described. Referring to FIGS. 54A and 54B, the aortic valve AV is located between a patient's sinotubular junction STJ and aortic annulus A at the bottom of the ascending aorta AA. The aortic valve AV typically comprises three leaflets including the right coronary cuspid RCC, the left coronary cuspid LCC, and the non-coronary cuspid NCC. Adjacent pairs of the leaflets are attached to the wall of the aortic root (which lies between the sinotubular junction and the aortic annulus) at commissures C which are often highly calcified regions. The free ends of the valve leaflets are closed together during diastole, as shown in FIG. 54A when the left ventricle is refilling with blood and open during systole, as shown in FIG. 54B, when the heart is pumping blood to the ascending aorta AA. The valvuloplasty system 5200 of the present invention is intended to insert at least a leading edge of the expandable sleeve and to the region between the outer surfaces of the valve leaflets RCC, LCC, and NCC and the inner wall of the aortic root in order to provide a backing to the valvuloplasty element once expanded within the valve leaflets in their open configuration as shown in FIG. 54B.

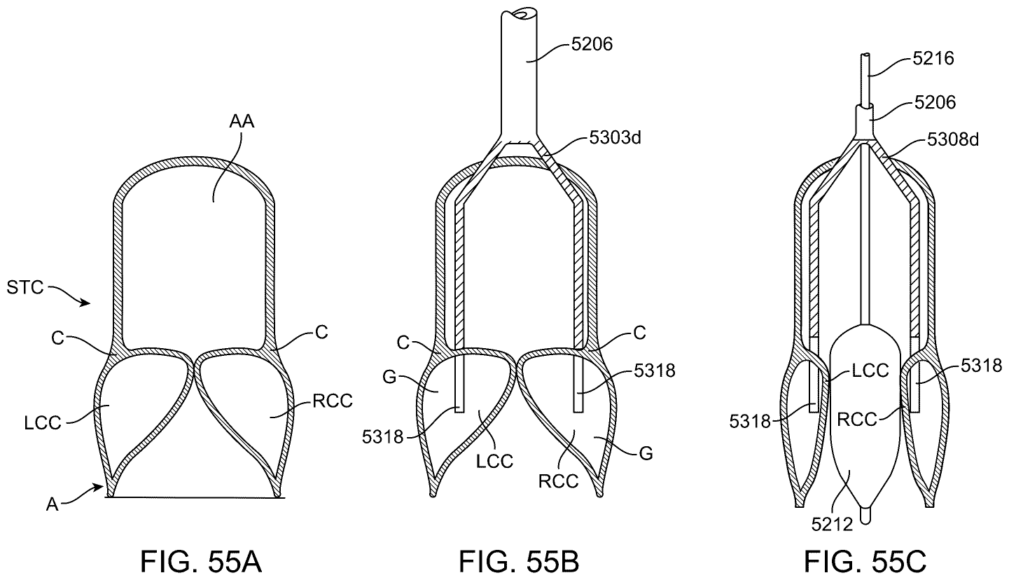

Referring now to FIGS. 55A to 55C, the valvuloplasty system 5200 having an expandable sleeve 5308d, as illustrated in FIG. 53D, can be advanced into the patient's ascending aorta, typically over the patient's aortic arch using a transfemoral approach. The expandable sleeve 5308d maybe rotated about its axis, typically under fluoroscopy or transthoracic ultrasound, or transesophageal echocardiography (TEE), to align the intermediate segments 5318 so that they lie between the commissures C. That is, the elongate gaps 5316 of the expandable sleeve 5308d will be aligned with the commissures C so that the commissures are received within the gap, as shown in FIG. 55B. In this way, the intermediate segments 5318 may be advanced a significant distance into the region between the leaflet and the wall of the aortic root, typically from 1 mm to 25 mm, often from 5 mm to 12 mm. Such depth of penetration provides a large target area for leaflet capture when the valvuloplasty element 5212 is inflated within the valve annulus A, as shown in FIG. 55C.

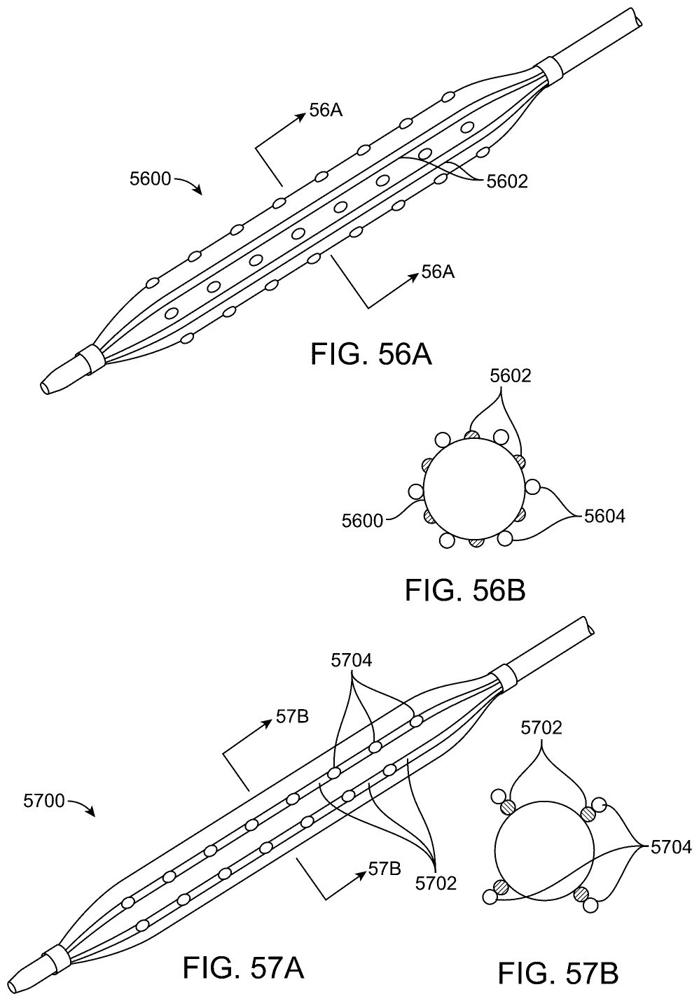

Referring now to FIGS. 56A and 56B, a balloon catheter 5600 has a balloon or other expandable surface that carries both a plurality elongate scoring elements 5602 and a multiplicity of discrete or point-like stress-applying features 5604 disposed between the elongate scoring elements. The combination of an axially distributed stress, as applied by the elongate scoring elements 5602, and the concentrated stress points, applied by the multiplicity of discrete or point-like stress-applying features 5604, will be particularly effective in disrupting hardened, calcified lesions.

Referring to FIGS. 57A and 57B, a balloon catheter 5700 carrying a plurality elongate scoring elements 5702 and a multiplicity of discrete or point-like stress-applying features 5704 disposed along radially outward surfaces of the elongate scoring elements, will also be particularly effective in disrupting hardened, calcified lesions.

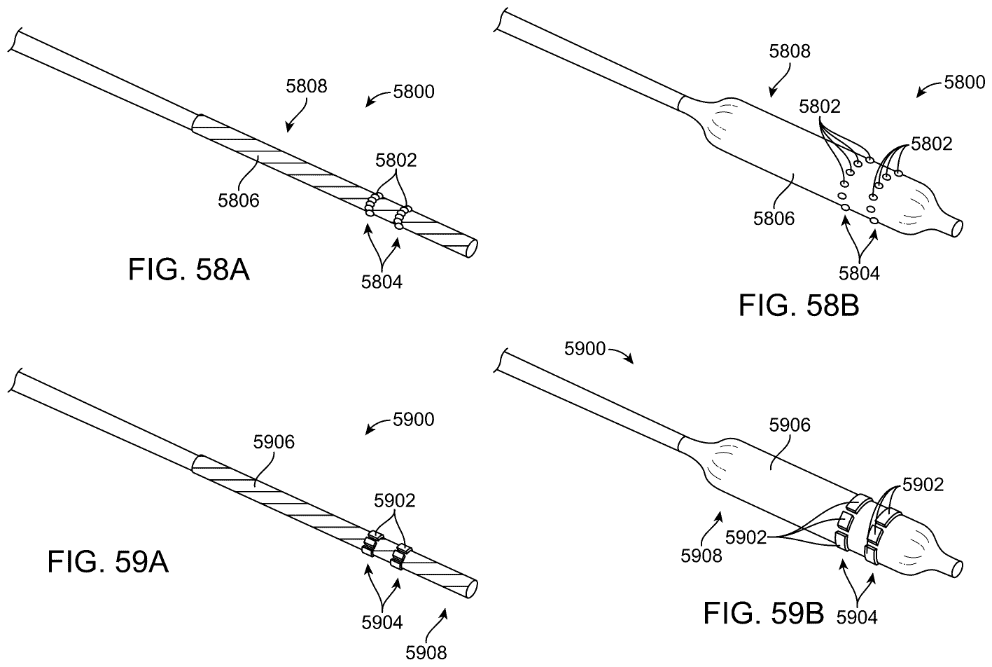

FIGS. 58A and 58B illustrate a balloon catheter 5800 carrying a plurality spherical stress-applying features 5802 disposed in two circumferential rings 5804 over a distal region of an outer surface 5806 of an inflatable valvuloplasty balloon 5808. The spherical stress-applying features 5802 are clustered together when the balloon 5808 is in a deflated state, as shown in FIG. 58A. The spherical stress-applying features 5802 remain closely packed when they deploy into the two circumferential rings 5804, as show in FIG. 58B. This configuration has been found to be particularly effective for performing valvuloplasty procedures where the spherical stress-applying features 5802 are positioned within calcified leaflets and/or the valve annulus as the balloon 5808 is expanded.

FIGS. 59A and 59B illustrate a balloon catheter 5900 carrying a plurality rectangular stress-applying features 5902 disposed in two circumferential rings 5904 over a distal region of an outer surface 5906 of a valvuloplasty balloon 5908. The rectangular stress-applying features 5902 are oriented generally axially and packed closely together when the balloon 5908 is in a deflated state, as shown in FIG. 59A. The rectangular stress-applying features 5902 reorient into the two circumferential rings 5904 as the balloon 5908 is inflated, as show in FIG. 58B. This configuration has also been found to be particularly effective for performing valvuloplasty procedures where the rings 5904 of rectangular stress-applying features 5902 are be positioned within calcified valve leaflets and/or the valve annulus as the balloon 5908 is expanded.

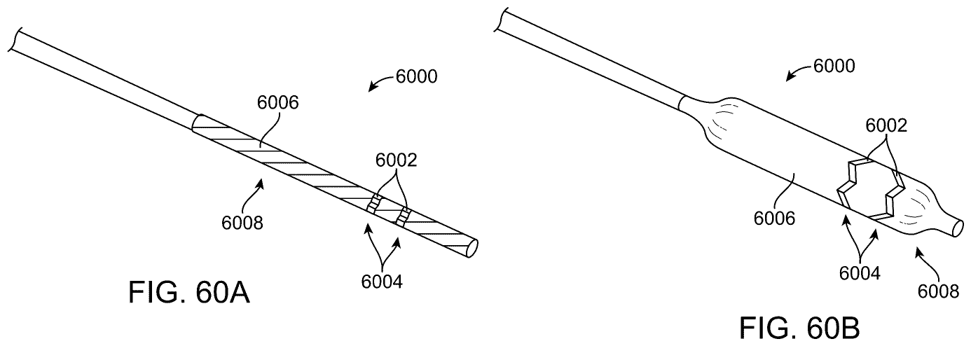

FIGS. 60A and 60B illustrate a balloon catheter 6000 carrying a pair of continuous, zig-zag stress-applying features 6002 disposed in two circumferential rings 6004 over a distal region of an outer surface 6006 of a valvuloplasty balloon 6008. The continuous zig-zag stress-applying features 6002 are in a radially compressed condition when the balloon is in a deflated state, as shown in FIG. 60A and expand into a more open zig-zag configuration as the balloon 6008 is inflated, as shown in FIG. 58B. While the zig-zag ring conformation is exemplary, the continuous, circumferential rings 6404 could also have a serpentine, square wave, omega wave or other known radially expandable configuration pattern. Unlike, spherical, rectangular, and other discrete stress-applying features, the continuous rings can be self-expanding as well as balloon expandable. The zig-zag stress-applying features 6002 which reorient into the two circumferential rings 6004 have also been found to be particularly effective for performing valvuloplasty procedures where the rings 6004 of the zig-zag stress-applying features 6002 are be positioned within calcified valve leaflets and/or the valve annulus as the balloon 6008 is expanded.

While primarily effective in fracturing or otherwise disrupting regions of calcified and hardened plaques, the convex rounded upper surfaces of the rigid features of the present invention have also been found to provide a protective function in the vascular walls adjacent to the calcified plaque. All angioplasty balloons risk damaging healthy vascular tissue adjacent to plaque-hardened regions of the vascular being treated due to an effect referred to as “dog boning” where the proximal and distal ends of the balloon will lie between the distal and region DR and the central region CD typically extend beyond the hardened region of the vessel where they expand to a diameter greater than that within the hardened vessel regions. Such “over expansion” can dissect the wall vascular causing trauma leading to rapid restenosis.

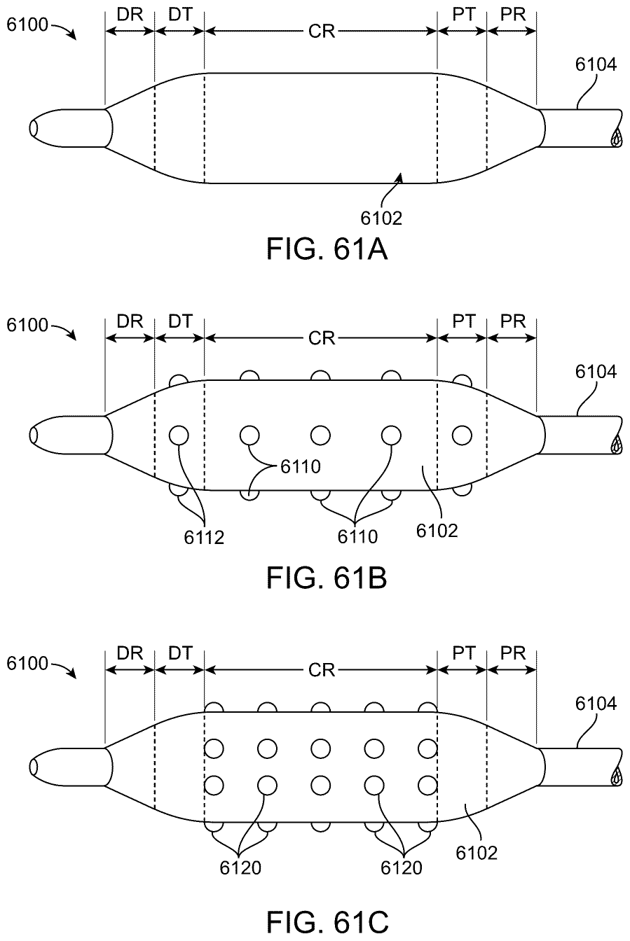

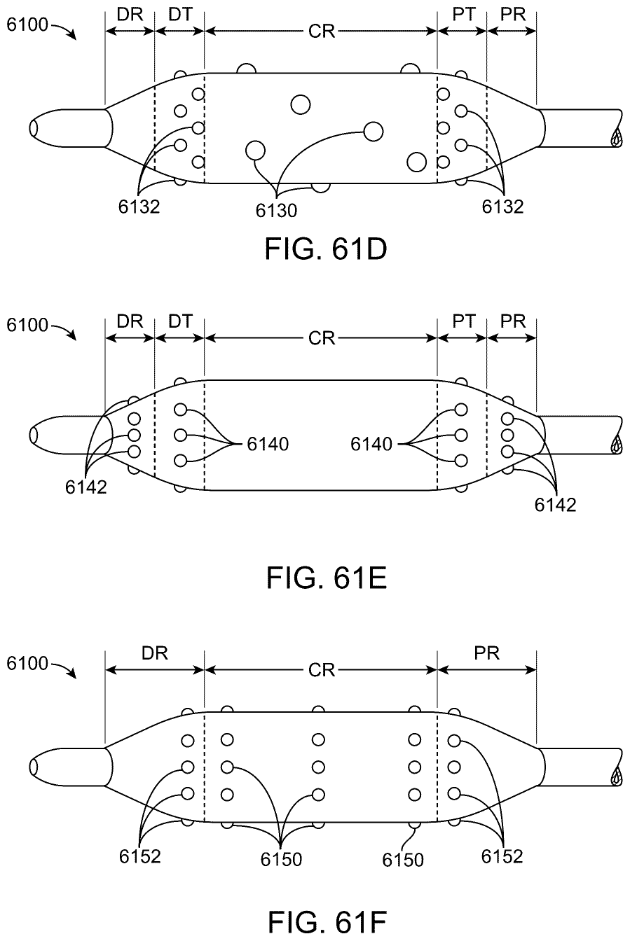

It has been found that the rigid features of the present invention, which act as stress applying features within the calcified regions of plaque, surprisingly provide a “cushioning” or protective function when they engage the healthy regions of the vessel wall which are not encased in plaque. The stress applying feature provide such a protective function when they are located so that they contact the non-calcified vasculature. In order to enhance the likelihood that the features will extend beyond the regions of calcified plaque during treatment, the balloons of the present invention may be provided with additional features over end and transition regions of the balloon, as described in more detail in connection with FIGS. 61A to 61F.

FIGS. 61A to 61F illustrate a balloon catheter having rigid features distributed over portions of one of one or more of (a) a distal transition region, (b) a proximal transition region, (c) a distal tapered region, (d) a proximal tapered region, (e) a distal terminal 2 mm length of the central region, and (f) a proximal terminal 2 mm length of a central region of the outer surface of an inflatable polymeric balloon. As shown in FIG. 61A, a balloon catheter 6100 includes an inflatable polymeric balloon 6102 mounted at the distal end of a catheter shaft 6104. The balloon has a central region CR disposed between a distal end region DR and a proximal end region PR, both of which are typically conical in shape. In at least most instances, a distal transition region DT will lie between the distal and region DR and the central region CD. Similarly, a proximal transition region PT will lie between the distal end region DR and the central region CR. Both the distal and proximal transition regions DT and PR will usually be curved and span a distance from 1 mm to several mm to provide a smooth transition between the central region CR which is typically cylindrical and the and regions DR and DT which are typically conical. In some instances, however, the transition regions may be very short (less than 1 mm) so that they provide a more abrupt transition from the cylindrical to the conical surfaces.

The balloons of the president invention will typically be semi-compliant so that they will expand beyond their nominal expansion diameter when inflated at the high pressures typical of angioplasty of calcified lesions. While the over expansion will be at least somewhat constrained by the calcifications, and regions outside of the calcifications, the balloon will over expand and cause undesirable “dog boning” particularly at the locations where the calcifications end.

As shown in FIG. 61B, stress applying features 6110 may be distributed over the central region CR of the balloon 6102 in any of the patterns described previously. In addition, rigid features 6112 (which may have a structure identical to the structure of the stress applying features) are additionally located in both the distal transition region DT and the proximal transition region PT of balloon 6102. The transition regions DT and PT will typically be located outside a calcified region of a blood vessel being treated when the balloon is expanded. so will contact non-calcified regions of the vasculature. It has been found that features located in the transition region can inhibit damages normally associated with “dog boning.”

Referring now to FIG. 61C, in some instances, a protective effect may be achieved by arranging stress applying features 6120 within the central region CR of the balloon 6102 so that some of the features are positioned immediately adjacent to the distal and/or proximal transition regions DT and PT, typically lying no more than 1 mm from the edge of the transition regions, preferably no more than 0.5 mm, and still preferably less than 0.1 mm. In such instances, placement of additional rigid features within the transition regions DT and PT and end regions DR and PR may be unnecessary.

The stress applying features and protective rigid features need not have the same dimensions, shapes, or other characteristics, and may also be arranged in different patterns over different regions of the balloon, shown in FIG. 61D. For example, stress applying features 6130 may be arranged in a helical pattern over the central region CR of balloon 6102, as shown in FIG. 61D, while protective features 6132 may be arranged in rings or zigzag patterns in the distal and proximal transition regions DT and PT. The stress applying features 6130 are also shown to be larger than the protective features 6132, but in other instances the stress applying features could be smaller than the protective features.

In some instances, as shown in FIG. 61E, protective features 6140 and 6142 may be located in the transition regions DT and PT and the end regions DR and PR While the central region is free stress applying features.

In some instances, the distal and proximal transition regions may be so short that the balloon transitions directly from the distal and proximal end regions DR and PR without any curved or other transition region in between. As shown in FIG. 61F, the central region CR may have stress supplying features 6150 arranged in any desired pattern while protective features 6152 may be located in both the distal and proximal end regions DR and PR.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure. Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

1-33. (canceled)

34. An apparatus for treating calcification on a wall in a patient's body lumen, the apparatus comprising:
a catheter including a catheter body having a proximal end and a distal segment;
an expandable structure disposed at the distal segment of the catheter body, the expandable structure having an outer surface configured to be displaced radially outwardly toward an inner surface of the body lumen wall; and
a plurality of stress-applying features distributed over the outer surface of the expandable structure; and
an energy source configured to deliver energy to at least some of the stress-applying feature to enhance plaque disruption.

35.-275. (canceled)

276. The apparatus of claim 34, wherein the energy source comprises an ultrasonic transducer.

277. The apparatus of claim 276, wherein the ultrasonic transducer is disposed in an interior of the expandable structure.

278. The apparatus of claim 276, wherein the ultrasonic transducer is disposed on the outer surface of the expandable structure.

279. The apparatus of claim 276, wherein the ultrasonic transducer is disposed externally to the expandable structure.

280. The apparatus of claim 34, wherein the energy source is configured to deliver electrical energy and at least some of the stress-applying features comprise piezoelectric structures configured to receive the electrical energy and vibrate in response thereto.

281. The apparatus of claim 34, wherein the energy source is configured to deliver optical energy and is coupled to the stress-applying features by optical fibers or wave guides.

282. The apparatus of claim 34, wherein the energy source is configured to deliver high voltage electrical energy to at least some of the stress-applying features to create arcing at the stress-applying features.
